SEQUENCE LISTING (38 sequences)

SEQ ID NO: 1 - DNA, 834 bp, Unknown
  Description of Unknown Exemplary nucleotide sequence encoding a dominant negative Zap70
atgccagaccccgcggcgcacctgcccttcttctacggcagcatctcgcgtgccgaggccgaggagcacctgaagctggcgggcatggcggacgggctcttcctgctgcgccagtgcctgcgctcgctgggcggctatgtgctgtcgctcgtgcacgatgtgcgcttccaccactttcccatcgagcgccagctcaacggcacctacgccattgccggcggcaaagcgcactgtggaccggcagagctctgcgagttctactcgcgcgaccccgacgggctgccctgcaacctgcgcaagccgtgcaaccggccgtcgggcctcgagccgcagccgggggtcttcgactgcctgcgagacgccatggtgcgtgactacgtgcgccagacgtggaagctggagggcgaggccctggagcaggccatcatcagccaggccccgcaggtggagaagctcattgctacgacggcccacgagcggatgccctggtaccacagcagcctgacgcgtgaggaggccgagcgcaaactttactctggggcgcagaccgacggcaagttcctgctgaggccgcggaaggagcagggcacatacgccctgtccctcatctatgggaagacggtgtaccactacctcatcagccaagacaaggcgggcaagtactgcattcccgagggcaccaagtttgacacgctctggcagctggtggagtatctgaagctgaaggcggacgggctcatctactgcctgaaggaggcctgccccaacagcagtgccagcaacgcctcaggggctgctgctcccacactcccagcccacccatccacgttgacg

SEQ ID NO: 2 - protein, 278 aa, Unknown
  Description of Unknown Exemplary amino acid sequence of a dominant negative ZAP70
MPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSASNASGAAAPTLPAHPSTLT

SEQ ID NO: 3 - DNA, 942 bp, Unknown
  Description of Unknown Exemplary nucleotide sequence encoding ER(T2) polypeptide
tctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacggtc

SEQ ID NO: 4 - protein, 314 aa, Unknown
  Description of Unknown Exemplary amino acid sequence of ER(T2) polypeptide
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPATV

SEQ ID NO: 5 - DNA, 45 bp, Unknown
  Description of Unknown Exemplary nucleotide sequence of a Nuclear Export Signal
aatgaattagccttgaaattagcaggtcttgatatcaacaagaca

SEQ ID NO: 6 - protein, 15 aa, Unknown
  Description of Unknown Exemplary amino acid sequence of a Nuclear Export Signal
NELALKLAGLDINKT

SEQ ID NO: 7 - DNA, 1830 bp, Artificial Sequence
  Description of Artificial Sequence Synthetic polynucleotide
atgaatgaattagccttgaaattagcaggtcttgatatcaacaagacaatgccagaccccgcggcgcacctgcccttcttctacggcagcatctcgcgtgccgaggccgaggagcacctgaagctggcgggcatggcggacgggctcttcctgctgcgccagtgcctgcgctcgctgggcggctatgtgctgtcgctcgtgcacgatgtgcgcttccaccactttcccatcgagcgccagctcaacggcacctacgccattgccggcggcaaagcgcactgtggaccggcagagctctgcgagttctactcgcgcgaccccgacgggctgccctgcaacctgcgcaagccgtgcaaccggccgtcgggcctcgagccgcagccgggggtcttcgactgcctgcgagacgccatggtgcgtgactacgtgcgccagacgtggaagctggagggcgaggccctggagcaggccatcatcagccaggccccgcaggtggagaagctcattgctacgacggcccacgagcggatgccctggtaccacagcagcctgacgcgtgaggaggccgagcgcaaactttactctggggcgcagaccgacggcaagttcctgctgaggccgcggaaggagcagggcacatacgccctgtccctcatctatgggaagacggtgtaccactacctcatcagccaagacaaggcgggcaagtactgcattcccgagggcaccaagtttgacacgctctggcagctggtggagtatctgaagctgaaggcggacgggctcatctactgcctgaaggaggcctgccccaacagcagtgccagcaacgcctcaggggctgctgctcccacactcccagcccacccatccacgttgacgggatcctctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacggtc

SEQ ID NO: 8 - protein, 610 aa, Artificial Sequence
  Description of Artificial Sequence Synthetic polypeptide
MNELALKLAGLDINKTMPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSASNASGAAAPTLPAHPSTLTGSSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPATV

SEQ ID NO: 9 - DNA, 882 bp, Artificial Sequence
  Description of Artificial Sequence Synthetic polynucleotide
atgaatgaattagccttgaaattagcaggtcttgatatcaacaagacaatgccagaccccgcggcgcacctgcccttcttctacggcagcatctcgcgtgccgaggccgaggagcacctgaagctggcgggcatggcggacgggctcttcctgctgcgccagtgcctgcgctcgctgggcggctatgtgctgtcgctcgtgcacgatgtgcgcttccaccactttcccatcgagcgccagctcaacggcacctacgccattgccggcggcaaagcgcactgtggaccggcagagctctgcgagttctactcgcgcgaccccgacgggctgccctgcaacctgcgcaagccgtgcaaccggccgtcgggcctcgagccgcagccgggggtcttcgactgcctgcgagacgccatggtgcgtgactacgtgcgccagacgtggaagctggagggcgaggccctggagcaggccatcatcagccaggccccgcaggtggagaagctcattgctacgacggcccacgagcggatgccctggtaccacagcagcctgacgcgtgaggaggccgagcgcaaactttactctggggcgcagaccgacggcaagttcctgctgaggccgcggaaggagcagggcacatacgccctgtccctcatctatgggaagacggtgtaccactacctcatcagccaagacaaggcgggcaagtactgcattcccgagggcaccaagtttgacacgctctggcagctggtggagtatctgaagctgaaggcggacgggctcatctactgcctgaaggaggcctgccccaacagcagtgccagcaacgcctcaggggctgctgctcccacactcccagcccacccatccacgttgacg

SEQ ID NO: 10 - protein, 294 aa, Artificial Sequence
  Description of Artificial Sequence Synthetic polypeptide
MNELALKLAGLDINKTMPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSASNASGAAAPTLPAHPSTLT

SEQ ID NO: 11 - DNA, 1788 bp, Homo sapiens
atgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggacataacgactatatgtgtccagccaccaaccagtgcaccattgataaaaacaggaggaagagctgccaggcctgccggctccgcaaatgctacgaagtgggaatgatgaaaggtgggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagggaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacggtctga

SEQ ID NO: 12 - protein, 595 aa, Homo sapiens [sequence omitted]

SEQ ID NO: 13 - protein, 314 aa, Artificial Sequence
  Description of Artificial Sequence Synthetic polypeptide
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPLKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPATV

SEQ ID NO: 14 - DNA, 942 bp, Artificial Sequence
  Description of Artificial Sequence Synthetic polynucleotide
tctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccactgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacggtc

SEQ ID NO: 15 - protein, 610 aa, Artificial Sequence
  Description of Artificial Sequence Synthetic polypeptide
MNELALKLAGLDINKTMPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSASNASGAAAPTLPAHPSTLTGSSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPLKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPATV

SEQ ID NO: 16 - DNA, 1833 bp, Artificial Sequence
  Description of Artificial Sequence Synthetic polynucleotide
atgaatgaattagccttgaaattagcaggtcttgatatcaacaagacaatgccagaccccgcggcgcacctgcccttcttctacggcagcatctcgcgtgccgaggccgaggagcacctgaagctggcgggcatggcggacgggctcttcctgctgcgccagtgcctgcgctcgctgggcggctatgtgctgtcgctcgtgcacgatgtgcgcttccaccactttcccatcgagcgccagctcaacggcacctacgccattgccggcggcaaagcgcactgtggaccggcagagctctgcgagttctactcgcgcgaccccgacgggctgccctgcaacctgcgcaagccgtgcaaccggccgtcgggcctcgagccgcagccgggggtcttcgactgcctgcgagacgccatggtgcgtgactacgtgcgccagacgtggaagctggagggcgaggccctggagcaggccatcatcagccaggccccgcaggtggagaagctcattgctacgacggcccacgagcggatgccctggtaccacagcagcctgacgcgtgaggaggccgagcgcaaactttactctggggcgcagaccgacggcaagttcctgctgaggccgcggaaggagcagggcacatacgccctgtccctcatctatgggaagacggtgtaccactacctcatcagccaagacaaggcgggcaagtactgcattcccgagggcaccaagtttgacacgctctggcagctggtggagtatctgaagctgaaggcggacgggctcatctactgcctgaaggaggcctgccccaacagcagtgccagcaacgcctcaggggctgctgctcccacactcccagcccacccatccacgttgacgggatcctctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccactgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacggtctga

SEQ ID NO: 17 - protein, 266 aa, Unknown
  Description of Unknown Exemplary amino acid sequence of LCK(1-266)
MGCGCSSHPEDDWMENIDVCENCHYPIVPLDGKGTLLIRNGSEVRDPLVTYEGSNPPASPLQDNLVIALHSYEPSHDGDLGFEKGEQLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRPCQTQKPQKPWWEDEWEVPRETLKLVERLGAGQFGEVWMGYYNG

SEQ ID NO: 18 - protein, 598 aa, Artificial Sequence
  Description of Artificial Sequence Synthetic polypeptide
MNELALKLAGLDINKTMGCGCSSHPEDDWMENIDVCENCHYPIVPLDGKGTLLIRNGSEVRDPLVTYEGSNPPASPLQDNLVIALHSYEPSHDGDLGFEKGEQLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRPCQTQKPQKPWWEDEWEVPRETLKLVERLGAGQFGEVWMGYYNGGSSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPLKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPATV

SEQ ID NO: 19 - DNA, 798 bp, Unknown
  Description of Unknown Exemplary nucleic acid sequence encoding LCK(1-266)
atgggctgtggctgcagctcacacccggaagatgactggatggaaaacatcgatgtgtgtgagaactgccattatcccatagtcccactggatggcaagggcacgctgctcatccgaaatggctctgaggtgcgggacccactggttacctacgaaggctccaatccgccggcttccccactgcaagacaacctggttatcgctctgcacagctatgagccctctcacgacggagatctgggctttgagaagggggaacagctccgcatcctggagcagagcggcgagtggtggaaggcgcagtccctgaccacgggccaggaaggcttcatccccttcaattttgtggccaaagcgaacagcctggagcccgaaccctggttcttcaagaacctgagccgcaaggacgcggagcggcagctcctggcgcccgggaacactcacggctccttcctcatccgggagagcgagagcaccgcgggatcgttttcactgtcggtccgggacttcgaccagaaccagggagaggtggtgaaacattacaagatccgtaatctggacaacggtggcttctacatctcccctcgaatcacttttcccggcctgcatgaactggtccgccattacaccaatgcttcagatgggctgtgcacacggttgagccgcccctgccagacccagaagccccagaagccgtggtgggaggacgagtgggaggttcccagggagacgctgaagctggtggagcggctgggggctggacagttcggggaggtgtggatggggtactacaacggg

SEQ ID NO: 20 - protein, 282 aa, Artificial Sequence
  Description of Artificial Sequence Synthetic polypeptide
MNELALKLAGLDINKTMGCGCSSHPEDDWMENIDVCENCHYPIVPLDGKGTLLIRNGSEVRDPLVTYEGSNPPASPLQDNLVIALHSYEPSHDGDLGFEKGEQLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRPCQTQKPQKPWWEDEWEVPRETLKLVERLGAGQFGEVWMGYYNG

SEQ ID NO: 21 - DNA, 846 bp, Artificial Sequence
  Description of Artificial Sequence Synthetic polynucleotide
atgaatgaattagccttgaaattagcaggtcttgatatcaacaagacaatgggctgtggctgcagctcacacccggaagatgactggatggaaaacatcgatgtgtgtgagaactgccattatcccatagtcccactggatggcaagggcacgctgctcatccgaaatggctctgaggtgcgggacccactggttacctacgaaggctccaatccgccggcttccccactgcaagacaacctggttatcgctctgcacagctatgagccctctcacgacggagatctgggctttgagaagggggaacagctccgcatcctggagcagagcggcgagtggtggaaggcgcagtccctgaccacgggccaggaaggcttcatccccttcaattttgtggccaaagcgaacagcctggagcccgaaccctggttcttcaagaacctgagccgcaaggacgcggagcggcagctcctggcgcccgggaacactcacggctccttcctcatccgggagagcgagagcaccgcgggatcgttttcactgtcggtccgggacttcgaccagaaccagggagaggtggtgaaacattacaagatccgtaatctggacaacggtggcttctacatctcccctcgaatcacttttcccggcctgcatgaactggtccgccattacaccaatgcttcagatgggctgtgcacacggttgagccgcccctgccagacccagaagccccagaagccgtggtgggaggacgagtgggaggttcccagggagacgctgaagctggtggagcggctgggggctggacagttcggggaggtgtggatggggtactacaacggg

SEQ ID NO: 22 - DNA, 1797 bp, Artificial Sequence
  Description of Artificial Sequence Synthetic polynucleotide
atgaatgaattagccttgaaattagcaggtcttgatatcaacaagacaatgggctgtggctgcagctcacacccggaagatgactggatggaaaacatcgatgtgtgtgagaactgccattatcccatagtcccactggatggcaagggcacgctgctcatccgaaatggctctgaggtgcgggacccactggttacctacgaaggctccaatccgccggcttccccactgcaagacaacctggttatcgctctgcacagctatgagccctctcacgacggagatctgggctttgagaagggggaacagctccgcatcctggagcagagcggcgagtggtggaaggcgcagtccctgaccacgggccaggaaggcttcatccccttcaattttgtggccaaagcgaacagcctggagcccgaaccctggttcttcaagaacctgagccgcaaggacgcggagcggcagctcctggcgcccgggaacactcacggctccttcctcatccgggagagcgagagcaccgcgggatcgttttcactgtcggtccgggacttcgaccagaaccagggagaggtggtgaaacattacaagatccgtaatctggacaacggtggcttctacatctcccctcgaatcacttttcccggcctgcatgaactggtccgccattacaccaatgcttcagatgggctgtgcacacggttgagccgcccctgccagacccagaagccccagaagccgtggtgggaggacgagtgggaggttcccagggagacgctgaagctggtggagcggctgggggctggacagttcggggaggtgtggatggggtactacaacgggggatcctctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccactgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacggtctga

SEQ ID NO: 23 - protein, 386 aa, Unknown
  Description of Unknown Exemplary amino acid sequence of SHP1(210-595)
RQPYYATRVNAADIENRVLELNKKQESEDTAKAGFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQFIETTKKKLEVLQSQKGQESEYGNITYPPAMKNAHAKASRTSSKHKEDVYENLHTKNKREEKVKKQRSADKEKSKGSLKRK

SEQ ID NO: 24 - protein, 718 aa, Artificial Sequence
  Description of Artificial Sequence Synthetic polypeptide
MNELALKLAGLDINKTRQPYYATRVNAADIENRVLELNKKQESEDTAKAGFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQFIETTKKKLEVLQSQKGQESEYGNITYPPAMKNAHAKASRTSSKHKEDVYENLHTKNKREEKVKKQRSADKEKSKGSLKRKGSSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPLKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPATV

SEQ ID NO: 25 - DNA, 1158 bp, Unknown
  Description of Unknown Exemplary nucleic acid sequence encoding SHP1(210-595)
cggcagccgtactatgccacgagggtgaatgcggctgacattgagaaccgagtgttggaactgaacaagaagcaggagtccgaggatacagccaaggctggcttctgggaggagtttgagagtttgcagaagcaggaggtgaagaacttgcaccagcgtctggaagggcagcggccagagaacaagggcaagaaccgctacaagaacattctcccctttgaccacagccgagtgatcctgcagggacgggacagtaacatccccgggtccgactacatcaatgccaactacatcaagaaccagctgctaggccctgatgagaacgctaagacctacatcgccagccagggctgtctggaggccacggtcaatgacttctggcagatggcgtggcaggagaacagccgtgtcatcgtcatgaccacccgagaggtggagaaaggccggaacaaatgcgtcccatactggcccgaggtgggcatgcagcgtgcttatgggccctactctgtgaccaactgcggggagcatgacacaaccgaatacaaactccgtaccttacaggtctccccgctggacaatggagacctgattcgggagatctggcattaccagtacctgagctggcccgaccatggggtccccagtgagcctgggggtgtcctcagcttcctggaccagatcaaccagcggcaggaaagtctgcctcacgcagggcccatcatcgtgcactgcagcgccggcatcggccgcacaggcaccatcattgtcatcgacatgctcatggagaacatctccaccaagggcctggactgtgacattgacatccagaagaccatccagatggtgcgggcgcagcgctcgggcatggtgcagacggaggcgcagtacaagttcatctacgtggccatcgcccagttcattgaaaccactaagaagaagctggaggtcctgcagtcgcagaagggccaggagtcggagtacgggaacatcacctatcccccagccatgaagaatgcccatgccaaggcctcccgcacctcgtccaaacacaaggaggatgtgtatgagaacctgcacactaagaacaagagggaggagaaagtgaagaagcagcggtcagcagacaaggagaagagcaagggttccctcaagaggaag

SEQ ID NO: 26 - DNA, 2157 bp, Artificial Sequence
  Description of Artificial Sequence Synthetic polynucleotide
atgaatgaattagccttgaaattagcaggtcttgatatcaacaagacacggcagccgtactatgccacgagggtgaatgcggctgacattgagaaccgagtgttggaactgaacaagaagcaggagtccgaggatacagccaaggctggcttctgggaggagtttgagagtttgcagaagcaggaggtgaagaacttgcaccagcgtctggaagggcagcggccagagaacaagggcaagaaccgctacaagaacattctcccctttgaccacagccgagtgatcctgcagggacgggacagtaacatccccgggtccgactacatcaatgccaactacatcaagaaccagctgctaggccctgatgagaacgctaagacctacatcgccagccagggctgtctggaggccacggtcaatgacttctggcagatggcgtggcaggagaacagccgtgtcatcgtcatgaccacccgagaggtggagaaaggccggaacaaatgcgtcccatactggcccgaggtgggcatgcagcgtgcttatgggccctactctgtgaccaactgcggggagcatgacacaaccgaatacaaactccgtaccttacaggtctccccgctggacaatggagacctgattcgggagatctggcattaccagtacctgagctggcccgaccatggggtccccagtgagcctgggggtgtcctcagcttcctggaccagatcaaccagcggcaggaaagtctgcctcacgcagggcccatcatcgtgcactgcagcgccggcatcggccgcacaggcaccatcattgtcatcgacatgctcatggagaacatctccaccaagggcctggactgtgacattgacatccagaagaccatccagatggtgcgggcgcagcgctcgggcatggtgcagacggaggcgcagtacaagttcatctacgtggccatcgcccagttcattgaaaccactaagaagaagctggaggtcctgcagtcgcagaagggccaggagtcggagtacgggaacatcacctatcccccagccatgaagaatgcccatgccaaggcctcccgcacctcgtccaaacacaaggaggatgtgtatgagaacctgcacactaagaacaagagggaggagaaagtgaagaagcagcggtcagcagacaaggagaagagcaagggttccctcaagaggaagggatcctctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccactgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacggtctga

SEQ ID NO: 27 - protein, 402 aa, Artificial Sequence
  Description of Artificial Sequence Synthetic polypeptide
MNELALKLAGLDINKTRQPYYATRVNAADIENRVLELNKKQESEDTAKAGFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQFIETTKKKLEVLQSQKGQESEYGNITYPPAMKNAHAKASRTSSKHKEDVYENLHTKNKREEKVKKQRSADKEKSKGSLKRK

SEQ ID NO: 28 - DNA, 1206 bp, Artificial Sequence
  Description of Artificial Sequence Synthetic polynucleotide
atgaatgaattagccttgaaattagcaggtcttgatatcaacaagacacggcagccgtactatgccacgagggtgaatgcggctgacattgagaaccgagtgttggaactgaacaagaagcaggagtccgaggatacagccaaggctggcttctgggaggagtttgagagtttgcagaagcaggaggtgaagaacttgcaccagcgtctggaagggcagcggccagagaacaagggcaagaaccgctacaagaacattctcccctttgaccacagccgagtgatcctgcagggacgggacagtaacatccccgggtccgactacatcaatgccaactacatcaagaaccagctgctaggccctgatgagaacgctaagacctacatcgccagccagggctgtctggaggccacggtcaatgacttctggcagatggcgtggcaggagaacagccgtgtcatcgtcatgaccacccgagaggtggagaaaggccggaacaaatgcgtcccatactggcccgaggtgggcatgcagcgtgcttatgggccctactctgtgaccaactgcggggagcatgacacaaccgaatacaaactccgtaccttacaggtctccccgctggacaatggagacctgattcgggagatctggcattaccagtacctgagctggcccgaccatggggtccccagtgagcctgggggtgtcctcagcttcctggaccagatcaaccagcggcaggaaagtctgcctcacgcagggcccatcatcgtgcactgcagcgccggcatcggccgcacaggcaccatcattgtcatcgacatgctcatggagaacatctccaccaagggcctggactgtgacattgacatccagaagaccatccagatggtgcgggcgcagcgctcgggcatggtgcagacggaggcgcagtacaagttcatctacgtggccatcgcccagttcattgaaaccactaagaagaagctggaggtcctgcagtcgcagaagggccaggagtcggagtacgggaacatcacctatcccccagccatgaagaatgcccatgccaaggcctcccgcacctcgtccaaacacaaggaggatgtgtatgagaacctgcacactaagaacaagagggaggagaaagtgaagaagcagcggtcagcagacaaggagaagagcaagggttccctcaagaggaag

SEQ ID NO: 29 - DNA, 1782 bp, Artificial Sequence
  Description of Artificial Sequence Synthetic polynucleotide
atgccagaccccgcggcgcacctgcccttcttctacggcagcatctcgcgtgccgaggccgaggagcacctgaagctggcgggcatggcggacgggctcttcctgctgcgccagtgcctgcgctcgctgggcggctatgtgctgtcgctcgtgcacgatgtgcgcttccaccactttcccatcgagcgccagctcaacggcacctacgccattgccggcggcaaagcgcactgtggaccggcagagctctgcgagttctactcgcgcgaccccgacgggctgccctgcaacctgcgcaagccgtgcaaccggccgtcgggcctcgagccgcagccgggggtcttcgactgcctgcgagacgccatggtgcgtgactacgtgcgccagacgtggaagctggagggcgaggccctggagcaggccatcatcagccaggccccgcaggtggagaagctcattgctacgacggcccacgagcggatgccctggtaccacagcagcctgacgcgtgaggaggccgagcgcaaactttactctggggcgcagaccgacggcaagttcctgctgaggccgcggaaggagcagggcacatacgccctgtccctcatctatgggaagacggtgtaccactacctcatcagccaagacaaggcgggcaagtactgcattcccgagggcaccaagtttgacacgctctggcagctggtggagtatctgaagctgaaggcggacgggctcatctactgcctgaaggaggcctgccccaacagcagtgccagcaacgcctcaggggctgctgctcccacactcccagcccacccatccacgttgacgggatcctctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacggtc

SEQ ID NO: 30 - protein, 594 aa, Artificial Sequence
  Description of Artificial Sequence Synthetic polypeptide
MPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSASNASGAAAPTLPAHPSTLTGSSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPATV

SEQ ID NO: 31 - protein, 594 aa, Artificial Sequence
  Description of Artificial Sequence Synthetic polypeptide
MPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSASNASGAAAPTLPAHPSTLTGSSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPLKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPATV

SEQ ID NO: 32 - DNA, 1785 bp, Artificial Sequence
  Description of Artificial Sequence Synthetic polynucleotide
atgccagaccccgcggcgcacctgcccttcttctacggcagcatctcgcgtgccgaggccgaggagcacctgaagctggcgggcatggcggacgggctcttcctgctgcgccagtgcctgcgctcgctgggcggctatgtgctgtcgctcgtgcacgatgtgcgcttccaccactttcccatcgagcgccagctcaacggcacctacgccattgccggcggcaaagcgcactgtggaccggcagagctctgcgagttctactcgcgcgaccccgacgggctgccctgcaacctgcgcaagccgtgcaaccggccgtcgggcctcgagccgcagccgggggtcttcgactgcctgcgagacgccatggtgcgtgactacgtgcgccagacgtggaagctggagggcgaggccctggagcaggccatcatcagccaggccccgcaggtggagaagctcattgctacgacggcccacgagcggatgccctggtaccacagcagcctgacgcgtgaggaggccgagcgcaaactttactctggggcgcagaccgacggcaagttcctgctgaggccgcggaaggagcagggcacatacgccctgtccctcatctatgggaagacggtgtaccactacctcatcagccaagacaaggcgggcaagtactgcattcccgagggcaccaagtttgacacgctctggcagctggtggagtatctgaagctgaaggcggacgggctcatctactgcctgaaggaggcctgccccaacagcagtgccagcaacgcctcaggggctgctgctcccacactcccagcccacccatccacgttgacgggatcctctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccactgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacggtctga

SEQ ID NO: 33 - protein, 582 aa, Artificial Sequence
  Description of Artificial Sequence Synthetic polypeptide
MGCGCSSHPEDDWMENIDVCENCHYPIVPLDGKGTLLIRNGSEVRDPLVTYEGSNPPASPLQDNLVIALHSYEPSHDGDLGFEKGEQLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRPCQTQKPQKPWWEDEWEVPRETLKLVERLGAGQFGEVWMGYYNGGSSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPLKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPATV

SEQ ID NO: 34 - DNA, 1749 bp, Artificial Sequence
  Description of Artificial Sequence Synthetic polynucleotide
atgggctgtggctgcagctcacacccggaagatgactggatggaaaacatcgatgtgtgtgagaactgccattatcccatagtcccactggatggcaagggcacgctgctcatccgaaatggctctgaggtgcgggacccactggttacctacgaaggctccaatccgccggcttccccactgcaagacaacctggttatcgctctgcacagctatgagccctctcacgacggagatctgggctttgagaagggggaacagctccgcatcctggagcagagcggcgagtggtggaaggcgcagtccctgaccacgggccaggaaggcttcatccccttcaattttgtggccaaagcgaacagcctggagcccgaaccctggttcttcaagaacctgagccgcaaggacgcggagcggcagctcctggcgcccgggaacactcacggctccttcctcatccgggagagcgagagcaccgcgggatcgttttcactgtcggtccgggacttcgaccagaaccagggagaggtggtgaaacattacaagatccgtaatctggacaacggtggcttctacatctcccctcgaatcacttttcccggcctgcatgaactggtccgccattacaccaatgcttcagatgggctgtgcacacggttgagccgcccctgccagacccagaagccccagaagccgtggtgggaggacgagtgggaggttcccagggagacgctgaagctggtggagcggctgggggctggacagttcggggaggtgtggatggggtactacaacgggggatcctctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccactgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacggtctga

SEQ ID NO: 35 - protein, 702 aa, Artificial Sequence
  Description of Artificial Sequence Synthetic polypeptide
RQPYYATRVNAADIENRVLELNKKQESEDTAKAGFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQFIETTKKKLEVLQSQKGQESEYGNITYPPAMKNAHAKASRTSSKHKEDVYENLHTKNKREEKVKKQRSADKEKSKGSLKRKGSSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPLKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPATV

SEQ ID NO: 36 - DNA, 2109 bp, Artificial Sequence
  Description of Artificial Sequence Synthetic polynucleotide
cggcagccgtactatgccacgagggtgaatgcggctgacattgagaaccgagtgttggaactgaacaagaagcaggagtccgaggatacagccaaggctggcttctgggaggagtttgagagtttgcagaagcaggaggtgaagaacttgcaccagcgtctggaagggcagcggccagagaacaagggcaagaaccgctacaagaacattctcccctttgaccacagccgagtgatcctgcagggacgggacagtaacatccccgggtccgactacatcaatgccaactacatcaagaaccagctgctaggccctgatgagaacgctaagacctacatcgccagccagggctgtctggaggccacggtcaatgacttctggcagatggcgtggcaggagaacagccgtgtcatcgtcatgaccacccgagaggtggagaaaggccggaacaaatgcgtcccatactggcccgaggtgggcatgcagcgtgcttatgggccctactctgtgaccaactgcggggagcatgacacaaccgaatacaaactccgtaccttacaggtctccccgctggacaatggagacctgattcgggagatctggcattaccagtacctgagctggcccgaccatggggtccccagtgagcctgggggtgtcctcagcttcctggaccagatcaaccagcggcaggaaagtctgcctcacgcagggcccatcatcgtgcactgcagcgccggcatcggccgcacaggcaccatcattgtcatcgacatgctcatggagaacatctccaccaagggcctggactgtgacattgacatccagaagaccatccagatggtgcgggcgcagcgctcgggcatggtgcagacggaggcgcagtacaagttcatctacgtggccatcgcccagttcattgaaaccactaagaagaagctggaggtcctgcagtcgcagaagggccaggagtcggagtacgggaacatcacctatcccccagccatgaagaatgcccatgccaaggcctcccgcacctcgtccaaacacaaggaggatgtgtatgagaacctgcacactaagaacaagagggaggagaaagtgaagaagcagcggtcagcagacaaggagaagagcaagggttccctcaagaggaagggatcctctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccactgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacggtctga

SEQ ID NO: 37 - DNA, 1858 bp, Artificial Sequence
  Description of Artificial Sequence Synthetic polynucleotide
ctagctagcatgaatgaattagccttgaaattagcaggtcttgatatcaacaagacaatgccagaccccgcggcgcacctgcccttcttctacggcagcatctcgcgtgccgaggccgaggagcacctgaagctggcgggcatggcggacgggctcttcctgctgcgccagtgcctgcgctcgctgggcggctatgtgctgtcgctcgtgcacgatgtgcgcttccaccactttcccatcgagcgccagctcaacggcacctacgccattgccggcggcaaagcgcactgtggaccggcagagctctgcgagttctactcgcgcgaccccgacgggctgccctgcaacctgcgcaagccgtgcaaccggccgtcgggcctcgagccgcagccgggggtcttcgactgcctgcgagacgccatggtgcgtgactacgtgcgccagacgtggaagctggagggcgaggccctggagcaggccatcatcagccaggccccgcaggtggagaagctcattgctacgacggcccacgagcggatgccctggtaccacagcagcctgacgcgtgaggaggccgagcgcaaactttactctggggcgcagaccgacggcaagttcctgctgaggccgcggaaggagcagggcacatacgccctgtccctcatctatgggaagacggtgtaccactacctcatcagccaagacaaggcgggcaagtactgcattcccgagggcaccaagtttgacacgctctggcagctggtggagtatctgaagctgaaggcggacgggctcatctactgcctgaaggaggcctgccccaacagcagtgccagcaacgcctcaggggctgctgctcccacactcccagcccacccatccacgttgacgggatcctctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacggtctgagcggccgctaaactat

SEQ ID NO: 38 - DNA, 910 bp, Artificial Sequence
  Description of Artificial Sequence Synthetic polynucleotide
ctagctagcatgaatgaattagccttgaaattagcaggtcttgatatcaacaagacaatgccagaccccgcggcgcacctgcccttcttctacggcagcatctcgcgtgccgaggccgaggagcacctgaagctggcgggcatggcggacgggctcttcctgctgcgccagtgcctgcgctcgctgggcggctatgtgctgtcgctcgtgcacgatgtgcgcttccaccactttcccatcgagcgccagctcaacggcacctacgccattgccggcggcaaagcgcactgtggaccggcagagctctgcgagttctactcgcgcgaccccgacgggctgccctgcaacctgcgcaagccgtgcaaccggccgtcgggcctcgagccgcagccgggggtcttcgactgcctgcgagacgccatggtgcgtgactacgtgcgccagacgtggaagctggagggcgaggccctggagcaggccatcatcagccaggccccgcaggtggagaagctcattgctacgacggcccacgagcggatgccctggtaccacagcagcctgacgcgtgaggaggccgagcgcaaactttactctggggcgcagaccgacggcaagttcctgctgaggccgcggaaggagcagggcacatacgccctgtccctcatctatgggaagacggtgtaccactacctcatcagccaagacaaggcgggcaagtactgcattcccgagggcaccaagtttgacacgctctggcagctggtggagtatctgaagctgaaggcggacgggctcatctactgcctgaaggaggcctgccccaacagcagtgccagcaacgcctcaggggctgctgctcccacactcccagcccacccatccacgttgacgtgagcggccgctaaactat

(19) United States
(12) Patent Application Publication
(10) Pub. No.: US 2023/0159619 A1
(43) Pub. Date: May 25, 2023

(54) COMPOSITIONS AND METHODS FOR REGULATING IMMUNE SYSTEM ACTIVITY

(71) Applicant: Olema Pharmaceuticals, Inc., San Francisco, CA (US)

(72) Inventors: Peter J. Kushner, San Francisco, CA (US); Leslie Hodges Gallagher, Alameda, CA (US); Cyrus L. Harmon, Bolinas, CA (US); David C. Myles, Berkeley, CA (US); Richard Sun, San Francisco, CA (US)

(21) Appl. No.: 16/486,099

(22) PCT Filed: Feb. 23, 2018
(86) PCT No.: PCT/US2018/019579

§ 371 Date: Aug. 14, 2019

Related U.S. Application Data
(60) Provisional application No. 62/462,725, filed on Feb. 23, 2017.

(51) Int. Cl. C07K 14/72; C12N 5/0783; C12N 9/12

(52) U.S. Cl. CPC C07K 14/721; C12N 5/0636; C12N 9/12; C07K 2319/90

(57) ABSTRACT

A trigger-responsive immune-inactivating signaling polypeptide disclosed herein can include a modulating domain and an immune-inactivating moiety, such as a dominant negative signaling moiety or constitutively active signaling moiety. A modulating domain can be characterized by an ability to adopt a first state and a second state, and to transition between the first state and the second state when exposed to a trigger. When the modulating domain is in its first state, the immune-inactivating signaling moiety can be inhibited, and when the modulating domain is in its second state, the inhibition can be relieved. Further disclosed herein are compositions for the delivery of a trigger-responsive immune-inactivating signaling polypeptide. Also, methods for using a trigger-responsive immune-inactivating signaling polypeptide, including to regulate an activity of immune system cells, are disclosed.

### CROSS-REFERENCE TO RELATED APPLICATIONS

This application is a National Stage Application based on International Application No. PCT/US2018/019579, filed Feb. 23, 2018, which claims the benefit of U.S. Provisional Application No. 62/462,725, filed on Feb. 23, 2017, the disclosure of each of which is hereby incorporated by reference in its entirety.

### BACKGROUND

In recent years, immunotherapy, or the idea of harnessing the body's immune system to fight a particular condition or diseases (e.g., cancer), has seen great progress. One area of progress has been the development of genetically engineered T cells (e.g., CAR-T cells) that are designed to recognize antigens present on cells associated with the condition or disease, and subsequently, attack and kill the recognized cells.

### SUMMARY

The present disclosure provides technologies for regulating an immune system activity (e.g., activity of monocytes, eosinophils, neutrophils, basophils, macrophages, dendritic cells, natural killer cells, T cells (including helper T cells and cytotoxic T cells), T regulatory cells, and/or B cells). Among other things, the present disclosure encompasses the insight that systems for controlling immune system activation (e.g., T cell activation) and/or activity can greatly enhance therapies that utilize and/or rely on immune system activation, such as T cell activation. The present disclosure recognizes a source of a problem that occurs with various existing therapeutic technologies that utilize and/or rely on immune system cells (e.g., monocytes, eosinophils, neutrophils, basophils, macrophages, dendritic cells, natural killer cells, T cells (including helper T cells and cytotoxic T cells), T regulatory cells, and/or B cells) including, for example, that many such technologies activate these cells or increase the activity of these cells without providing any mechanism to “turn-off” activated cells or reduce cell activity, which, when unregulated, often leads to harmful consequences. Furthermore, the present disclosure appreciates that certain other technologies intended to control an immune system activity, e.g., by controlling T cell activity, do so by destroying the activated immune system cells (e.g., T cells) in order to “turn them off,” thereby terminating the treatment and wasting valuable time and resources.

In some embodiments, the present disclosure provides a particular insight that T cell activation involves a signaling pathway that may provide particularly attractive opportunities to control T cell activity. In particular, the present disclosure provides insights relating to particular strategies for controlling T cell activity by regulating kinase activity, phosphatase activity, GTPase activity, guanine nucleotide exchange factor activity, phospholipase activity, paracaspase activity, and/or protease activity within a T cell activation pathway. In certain embodiments, the present disclosure provides technologies that utilize a dominant negative variant (or relevant moiety thereof) of a phosphatase, GTPase, guanine nucleotide exchange factor, phospholipase, paracaspase, and/or protease in a T cell activation pathway to regulate T cell activity.

The present disclosure provides insights relating to, among other things, use of an immune-inactivating signaling polypeptide to control the activity of immune cells (such as T cells). One common mechanism of immune pathway signaling, among others, is the addition or removal of phosphate groups by, e.g., cellular enzymes. Kinases are a common representative of the group of enzymes that mediate phosphate modifications. Another common representative of this group of enzymes is phosphatases. As mediators of phosphate modification, polypeptide kinases and/or polypeptide phosphatases are significant components of many regulatory pathways, including pathways that regulate immune activity. Because the addition or removal of phosphate groups can have, depending on context and function, various regulatory effects on a particular pathway or activity, a kinase and a phosphatase in a given system may, e.g., regulate a particular downstream function or event in parallel (e.g., both activating or both inactivating) or oppositely (e.g., one activating and another inactivating). Accordingly, the present application relates to regulatory mechanisms of phosphorylation generally, as well as to kinase polypeptides and phosphatase polypeptides that are exemplary thereof.

In some instances, an immune-inactivating signaling polypeptide can be a dominant negative variant of a kinase within an immune activity pathway, or can be a variant of a phosphatase that constitutively inhibits an immune activity pathway. In the context of an immune activity pathway, dominant negative kinase activity and constitutive phosphatase activity can both reduce immune activity by physical or other regulatory interaction with the immune activity pathway (i.e., are “immune-inactivating”).

In certain particular embodiments, the present disclosure provides a trigger-responsive dominant negative polypeptide—i.e., a construct in which a dominant negative signaling moiety is fused to a modulating domain that blocks the function of the dominant negative signaling moiety, except when the modulating domain is itself inactivated by provision of the trigger.

More specifically, the present disclosure provides insights relating to particular strategies for controlling T cell activity by regulating kinase activity within a T cell activation pathway. Still further, the present disclosure appreciates that dominant negative variants of kinases within a T cell activation pathway are available and/or can be readily generated. In certain embodiments, the present disclosure provides technologies that utilize a dominant negative variant (or relevant moiety thereof) of a kinase in a T cell activation pathway to regulate T cell activity. In certain particular embodiments, the present disclosure provides a trigger-responsive dominant negative polypeptide—i.e., a construct in which a dominant negative signaling moiety is fused to a modulating domain that blocks the function of the dominant negative signaling moiety, except when the modulating domain is itself inactivated by provision of the trigger.

The present disclosure also specifically provides insights relating to particular strategies for controlling T cell activity by regulating phosphatase activity within a T cell activation pathway. Still further, the present disclosure appreciates that variants of phosphatases that constitutively inhibit (constitutively active phosphatase polypeptides) a T cell activation pathway are available and/or can be readily generated. In certain embodiments, the present disclosure provides technologies that utilize a constitutively active variant (or relevant moiety thereof) of a phosphatase in a T cell activation pathway to regulate T cell activity. In certain particular embodiments, the present disclosure provides a constitutively active phosphatase polypeptide—i.e., a construct in which a constitutively active phosphatase moiety is fused to a modulating domain that blocks the function of the constitutively active phosphatase moiety, except when the modulating domain is itself inactivated by provision of the trigger.

In some embodiments, the present disclosure provides technologies in which a trigger-responsive immune-inactivating signaling polypeptide (such as a trigger-responsive dominant negative signaling polypeptide or trigger-responsive constitutively active signaling polypeptide), is exposed to a trigger for a limited period of time (e.g., due to removal, expiration, inactivation, and/or destruction of the trigger) in order to put a “brake” on an activity of immune system cells, e.g., engineered T cell activity. The present disclosure provides an insight that reversibility of signaling activity that inhibits an activity of immune system cells, e.g., dominant negative activity, according to such technologies offers unique advantages for regulation of an immune system activity (e.g., T cell activity). For example, advantages include among other things avoiding difficulties associated with alternative approaches for regulating T cells, where T cell activity, once turned off, cannot be turned back on. Indeed, in some embodiments, the present disclosure provides systems that permit not simply “turn-off” control of an immune system activity (e.g., T cell activity), but potentially adjustable “dial-up/dial-down” control.

Embodiments of the invention provide a trigger-responsive immune-inactivating signaling polypeptide. Typically, the immune-inactivating moiety of an immune-inactivating signaling polypeptide operates on an immune activity pathway to inhibit immune activity. In some embodiments, a trigger-responsive immune-inactivating signaling polypeptide includes a modulating domain and an immune-inactivating moiety, e.g., where the modulating domain regulates the operation of the immune-inactivating moiety on an immune activity pathway. In some embodiments, a modulating domain is characterized by an ability to adopt a first state and a second state, and to transition between the first state and the second state when exposed to a trigger. In some embodiments, when a modulating domain of a trigger-responsive immune-inactivating signaling polypeptide is in its first state, an immune-inactivating moiety of the trigger-responsive immune-inactivating polypeptide is inhibited, and when the modulating domain is in its second state, the inhibition is relieved.

Embodiments of the invention provide a trigger-responsive dominant negative signaling polypeptide. In some embodiments, a trigger-responsive dominant negative signaling polypeptide includes a modulating domain and a dominant negative signaling moiety. In some embodiments, a modulating domain is characterized by an ability to adopt a first state and a second state, and to transition between the first state and the second state when exposed to a trigger. In some embodiments, when a modulating domain of a trigger-responsive dominant negative signaling polypeptide is in its first state, a dominant negative signaling moiety of the trigger-responsive dominant negative signaling polypeptide is inhibited, and when the modulating domain is in its second state, the inhibition is relieved.

In certain particular instances, a dominant negative signaling moiety is a variant of a kinase, which moiety typically operates on an immune cell activity pathway to inhibit immune cell activity. In certain such instances in which the dominant negative signaling moiety is operatively linked to a modulating domain, the modulating domain regulates the operation of the dominant negative signaling polypeptide on the immune cell activity pathway. For instance, in some embodiments, in the absence of a trigger, the modulating domain inhibits operation of the dominant negative signaling moiety on the immune cell activity pathway, such that in the absence of a trigger immune cell activity is not inhibited. Further, in certain such embodiments, the presence of a trigger mediates a transition of the modulating domain into an alternative state, in which alternative state the modulating domain does not inhibit operation of the dominant negative signaling polypeptide on an immune cell activity, such that in the presence of a trigger an immune cell activity is inhibited. Embodiments of the invention provide a trigger-responsive constitutively active signaling polypeptide. In some embodiments, a trigger-responsive constitutively active signaling polypeptide includes a modulating domain and a constitutively active signaling moiety. In some embodiments, a modulating domain is characterized by an ability to adopt a first state and a second state, and to transition between the first state and the second state when exposed to a trigger. In some embodiments, when a modulating domain of a trigger-responsive constitutively active signaling polypeptide is in its first state, a constitutively active signaling moiety of the trigger-responsive constitutively active signaling polypeptide is inhibited, and when the modulating domain is in its second state, the inhibition is relieved.

In certain particular instances, a constitutively active signaling moiety is a variant of a phosphatase, which moiety typically operates on an immune cell activity pathway to inhibit immune cell activity. In certain such instances in which the constitutively active signaling moiety is operatively linked to a modulating domain, the modulating domain regulates the operation of the constitutively active signaling polypeptide on the immune cell activity pathway. For instance, in some embodiments, in the absence of a trigger, the modulating domain inhibits operation of the constitutively active signaling moiety on the immune cell activity pathway, such that in the absence of a trigger immune cell activity is not inhibited. Further, in certain such embodiments, the presence of a trigger mediates a transition of the modulating domain into an alternative state, in which alternative state the modulating domain does not inhibit operation of the constitutively active signaling polypeptide on an immune cell activity, such that in the presence of a trigger an immune cell activity is inhibited.

In certain embodiments, a modulating domain includes a nuclear receptor or a portion of a nuclear receptor. In some embodiments, a portion of a nuclear receptor includes a ligand binding domain of a nuclear receptor. Exemplary nuclear receptors include a steroid hormone receptor, a thyroid hormone receptor, a retinoic acid receptor, a vitamin D receptor, peroxisome proliferator-activated receptor, farnesoid X receptor, and liver X receptor. In some embodiments, a modulating domain includes a hormone receptor or a portion of a hormone receptor. In some embodiments, a portion of a hormone receptor includes a ligand binding domain of a hormone receptor, e.g., a steroid hormone receptor. An exemplary hormone receptors is an estrogen receptor, e.g., an estrogen receptor-α. In some embodiments, a nuclear receptor and/or a hormone receptor is a mammalian receptor, e.g., a human receptor.

In some embodiments, a modulating domain includes an amino acid sequence that has at least 80%, at least 85%, at least 90%, at least 95%, at least 98%, or at least 99% sequence identity with an amino acid sequence that starts at residue 251, 282, or 305 of SEQ ID NO: 12 and ends at residue 545 or 595 of SEQ ID NO: 12. In some embodiments, a modulating domain includes an amino acid sequence that has at least 80%, at least 85%, at least 90%, at least 95%, at least 98%, or at least 99% sequence identity with SEQ ID NO: 4. In some embodiments, a modulating domain includes an amino acid sequence that has at least 80%, at least 85%, at least 90%, at least 95%, at least 98%, or at least 99% sequence identity with SEQ ID NO: 13.

In certain embodiments, a modulating domain can be a wild-type or mutant variant of a nuclear receptor (e.g., a hormone receptor). In some embodiments, a modulating domain can be a mutant variant of a hormone receptor, e.g., an estrogen receptor. In some embodiments, a modulating domain includes mutations that confer on the modulating domain a reduced affinity to at least one naturally occurring estrogen (e.g., estradiol (e.g., 17-beta estradiol)), a preferential binding to at least one synthetic estrogen receptor ligand (e.g., tamoxifen, endoxifen, 4-hydroxytamoxifen, fulvestrant, OP-1250, OP-1074, or OP-1124), and/or an increased affinity for at least one chaperone protein (e.g., HSP90).

In some embodiments, a modulating domain includes an estrogen receptor or fragment thereof comprising at least one mutation selected from the group consisting of G400V, G400M, G400A, G400L, G400I, G521R, G521T, L539A, L540A, M543A and L544A, wherein the residue numbering is based on SEQ ID NO: 12.

In certain embodiments, a modulating domain includes an estrogen receptor or fragment thereof comprising a first mutation selected from G400V, G400M, G400A, G400L, G400I, G521R, G521T, L539A, L540A, M543A and L544A, and at least a second mutation selected from G400V, G400M, G400A, G400L, G400I, G521R, G521T, L539A, L540A, M543A and L544A (residue numbering is based on SEQ ID NO: 12).

In certain embodiments, a modulating domain includes an estrogen receptor or fragment thereof comprising a first mutation selected from G400V, G400M, G400A, G400L, G400I, G521R, G521T, L539A, L540A, M543A and L544A, a second mutation selected from G400V, G400M, G400A, G400L, G400I, G521R, G521T, L539A, L540A, M543A and L544A, and at least a third mutation selected from G400V, G400M, G400A, G400L, G400I, G521R, G521T, L539A, L540A, M543A and L544A (residue numbering is based on SEQ ID NO: 12).

In certain embodiments, a modulating domain includes an estrogen receptor or fragment thereof comprising a first mutation selected from G400V, G400M, G400A, G400L, G400I, G521R, G521T, L539A, L540A, M543A and L544A, a second mutation selected from G400V, G400M, G400A, G400L, G400I, G521R, G521T, L539A, L540A, M543A and L544A, a third mutation selected from G400V, G400M, G400A, G400L, G400I, G521R, G521T, L539A, L540A, M543A and L544A, and at least a fourth mutation selected from G400V, G400M, G400A, G400L, G400I, G521R, G521T, L539A, L540A, M543A and L544A (residue numbering is based on SEQ ID NO: 12).

In certain embodiments, a modulating domain includes an estrogen receptor or fragment thereof comprising a first mutation selected from G400V, G400M, G400A, G400L, G400I, G521R, G521T, L539A, L540A, M543A and L544A, a second mutation selected from G400V, G400M, G400A, G400L, G400I, G521R, G521T, L539A, L540A, M543A and L544A, a third mutation selected from G400V, G400M, G400A, G400L, G400I, G521R, G521T, L539A, L540A, M543A and L544A, a fourth mutation selected from G400V, G400M, G400A, G400L, G400I, G521R, G521T, L539A, L540A, M543A and L544A, and at least a fifth mutation selected from G400V, G400M, G400A, G400L, G400I, G521R, G521T, L539A, L540A, M543A and L544A (residue numbering is based on SEQ ID NO: 12).

In certain embodiments, a modulating domain includes an estrogen receptor or fragment thereof comprising a first mutation selected from G400V, G400M, G400A, G400L, G400I, G521R, G521T, L539A, L540A, M543A and L544A, a second mutation selected from G400V, G400M, G400A, G400L, G400I, G521R, G521T, L539A, L540A, M543A and L544A, a third mutation selected from G400V, G400M, G400A, G400L, G400I, G521R, G521T, L539A, L540A, M543A and L544A, a fourth mutation selected from G400V, G400M, G400A, G400L, G400I, G521R, G521T, L539A, L540A, M543A and L544A, a fifth mutation selected from G400V, G400M, G400A, G400L, G400I, G521R, G521T, L539A, L540A, M543A and L544A, and at least a sixth mutation selected from G400V, G400M, G400A, G400L, G400I, G521R, G521T, L539A, L540A, M543A and L544A (residue numbering is based on SEQ ID NO: 12).

In certain embodiments, a modulating domain includes an estrogen receptor or fragment thereof comprising at least one mutation that is either G400V or G400L, wherein the residue numbering is based on SEQ ID NO: 12. In some embodiments, a modulating domain comprises an estrogen receptor or fragment thereof that (i) includes an amino acid sequence that has at least 80%, at least 85%, at least 90%, at least 95%, at least 98%, or at least 99% sequence identity with an amino acid sequence that starts at residue 251, 282, or 305 of SEQ ID NO: 12 and ends at residue 545 or 595 of SEQ ID NO: 12, and (ii) includes at least one mutation selected from the group consisting of G400V, G400M, G400A, G400L, G400I, G521R, G521T, L539A, L540A, M543A and L544A, wherein the residue numbering is based on SEQ ID NO: 12.

In some embodiments, a modulating domain includes an estrogen receptor or fragment thereof comprising at least one mutation selected from the group consisting of G400V, G400M, G400A, G400L, G400I, G521R, and G521T, wherein the residue numbering is based on SEQ ID NO: 12. In some embodiments, a modulating domain includes an estrogen receptor or fragment thereof comprising at least one mutation that is either G400V or G400L, wherein the residue numbering is based on SEQ ID NO: 12. In some embodiments, a modulating domain includes an estrogen receptor or fragment thereof comprising one or more additional mutations selected from L539A and L540A, wherein the residue numbering is based on SEQ ID NO: 12. In some embodiments, the estrogen receptor or fragment thereof of the modulating domain comprises one or more additional mutations selected from M543A and L544A, wherein the residue numbering is based on SEQ ID NO: 12. In some embodiments, a modulating domain includes an estrogen receptor or fragment thereof comprising at least one mutation selected from the group consisting of G400V, G400M, G400A, G400L, G400I, G521R, and G521T, and one or more additional mutations selected from (i) L539A and L540A, or (ii) M543A and L544A, wherein the.

In various embodiments, a modulating domain includes an estrogen receptor or fragment thereof including a combination of mutations identified in Table 1 below:

TABLE 1
Combinations of non-limiting mutations that can be present in
a modulating domain that includes an estrogen receptor or fragment
thereof (residue numbering based on SEQ ID NO: 12)

G400V, G521R, L539A, L540A, M543A, L544A
G400V, G521R, L539A, L540A, M543A
G400V, G521R, L539A, L540A, L544A
G400V, G521R, L539A, L540A
G400V, G521R, L539A, M543A, L544A
G400V, G521R, L539A, M543A
G400V, G521R, L539A, L544A
G400V, G521R, L539A
G400V, G521R, L540A, M543A, L544A
G400V, G521R, L540A, M543A
G400V, G521R, L540A, L544A
G400V, G521R, L540A
G400V, G521R, M543A, L544A
G400V, G521R, M543A
G400V, G521R, L544A
G400V, G521R
G400V, G521T, L539A, L540A, M543A, L544A
G400V, G521T, L539A, L540A, M543A
G400V, G521T, L539A, L540A, L544A
G400V, G521T, L539A, L540A
G400V, G521T, L539A, M543A, L544A
G400V, G521T, L539A, M543A
G400V, G521T, L539A, L544A
G400V, G521T, L539A,
G400V, G521T, L540A, M543A, L544A
G400V, G521T, L540A, M543A
G400V, G521T, L540A, L544A
G400V, G521T, L540A
G400V, G521T, M543A, L544A
G400V, G521T, M543A
G400V, G521T, L544A
G400V, G521T
G400M, G521R, L539A, L540A, M543A, L544A
G400M, G521R, L539A, L540A, M543A
G400M, G521R, L539A, L540A, L544A
G400M, G521R, L539A, L540A
G400M, G521R, L539A, M543A, L544A
G400M, G521R, L539A, M543A
G400M, G521R, L539A, L544A
G400M, G521R, L539A
G400M, G521R, L540A, M543A, L544A
G400M, G521R, L540A, M543A
G400M, G521R, L540A, L544A
G400M, G521R, L540A
G400M, G521R, M543A, L544A
G400M, G521R, M543A
G400M, G521R, L544A
G400M, G521R
G400M, G521T, L539A, L540A, M543A, L544A
G400M, G521T, L539A, L540A, M543A
G400M, G521T, L539A, L540A, L544A
G400M, G521T, L539A, L540A
G400M, G521T, L539A, M543A, L544A
G400M, G521T, L539A, M543A
G400M, G521T, L539A, L544A
G400M, G521T, L539A
G400M, G521T, L540A, M543A, L544A
G400M, G521T, L540A, M543A
G400M, G521T, L540A, L544A
G400M, G521T, L540A
G400M, G521T, M543A, L544A
G400M, G521T, M543A
G400M, G521T, L544A
G400M, G521T
G400A, G521R, L539A, L540A, M543A, L544A
G400A, G521R, L539A, L540A, M543A
G400A, G521R, L539A, L540A, L544A
G400A, G521R, L539A, L540A
G400A, G521R, L539A, M543A, L544A
G400A, G521R, L539A, M543A
G400A, G521R, L539A, L544A
G400A, G521R, L539A
G400A, G521R, L540A, M543A, L544A
G400A, G521R, L540A, M543A
G400A, G521R, L540A, L544A
G400A, G521R, L540A
G400A, G521R, M543A, L544A
G400A, G521R, M543A
G400A, G521R, L544A
G400A, G521R
G400A, G521T, L539A, L540A, M543A, L544A
G400A, G521T, L539A, L540A, M543A
G400A, G521T, L539A, L540A, L544A
G400A, G521T, L539A, L540A
G400A, G521T, L539A, M543A, L544A
G400A, G521T, L539A, M543A
G400A, G521T, L539A, L544A
G400A, G521T, L539A
G400A, G521T, L540A, M543A, L544A
G400A, G521T, L540A, M543A
G400A, G521T, L540A, L544A
G400A, G521T, L540A
G400A, G521T, M543A, L544A
G400A, G521T, M543A
G400A, G521T, L544A
G400A, G521T
G400L, G521R, L539A, L540A, M543A, L544A
G400L, G521R, L539A, L540A, M543A
G400L, G521R, L539A, L540A, L544A
G400L, G521R, L539A, L540A
G400L, G521R, L539A, M543A, L544A
G400L, G521R, L539A, M543A
G400L, G521R, L539A, L544A
G400L, G521R, L539A
G400L, G521R, L540A, M543A, L544A
G400L, G521R, L540A, M543A
G400L, G521R, L540A, L544A
G400L, G521R, L540A
G400L, G521R, M543A, L544A
G400L, G521R, M543A
G400L, G521R, L544A
G400L, G521R
G400L, G521T, L539A, L540A, M543A, L544A
G400L, G521T, L539A, L540A, M543A
G400L, G521T, L539A, L540A, L544A
G400L, G521T, L539A, L540A
G400L, G521T, L539A, M543A, L544A
G400L, G521T, L539A, M543A
G400L, G521T, L539A, L544A
G400L, G521T, L539A
G400L, G521T, L540A, M543A, L544A
G400L, G521T, L540A, M543A
G400L, G521T, L540A, L544A
G400L, G521T, L540A
G400L, G521T, M543A, L544A
G400L, G521T, M543A
G400L, G521T, L544A
G400L, G521T
G400I, G521R, L539A, L540A, M543A, L544A
G400I, G521R, L539A, L540A, M543A
G400I, G521R, L539A, L540A, L544A
G400I, G521R, L539A, L540A
G400I, G521R, L539A, M543A, L544A
G400I, G521R, L539A, M543A
G400I, G521R, L539A, L544A
G400I, G521R, L539A
G400I, G521R, L540A, M543A, L544A
G400I, G521R, L540A, M543A
G400I, G521R, L540A, L544A
G400I, G521R, L540A
G400I, G521R, M543A, L544A
G400I, G521R, M543A
G400I, G521R, L544A
G400I, G521R
G400I, G521T, L539A, L540A, M543A, L544A
G400I, G521T, L539A, L540A, M543A
G400I, G521T, L539A, L540A, L544A
G400I, G521T, L539A, L540A
G400I, G521T, L539A, M543A, L544A
G400I, G521T, L539A, M543A
G400I, G521T, L539A, L544A
G400I, G521T, L539A
G400I, G521T, L540A, M543A, L544A
G400I, G521T, L540A, M543A
G400I, G521T, L540A, L544A
G400I, G521T, L540A
G400I, G521T, M543A, L544A
G400I, G521T, M543A
G400I, G521T, L544A
G400I, G521T
G400V, L539A, L540A, M543A, L544A
G400V, L539A, L540A, M543A
G400V, L539A, L540A, L544A
G400V, L539A, L540A
G400V, L539A, M543A, L544A
G400V, L539A, M543A
G400V, L539A, L544A
G400V, L539A
G400V, L540A, M543A, L544A
G400V, L540A, M543A
G400V, L540A, L544A
G400V, L540A
G400V, M543A, L544A
G400V, M543A
G400V, L544A
G400V
G400M, L539A, L540A, M543A, L544A
G400M, L539A, L540A, M543A
G400M, L539A, L540A, L544A
G400M, L539A, L540A
G400M, L539A, M543A, L544A
G400M, L539A, M543A
G400M, L539A, L544A
G400M, L539A
G400M, L540A, M543A, L544A
G400M, L540A, M543A
G400M, L540A, L544A
G400M, L540A
G400M, M543A, L544A
G400M, M543A
G400M, L544A
G400M
G400A, L539A, L540A, M543A, L544A
G400A, L539A, L540A, M543A
G400A, L539A, L540A, L544A
G400A, L539A, L540A
G400A, L539A, M543A, L544A
G400A, L539A, M543A
G400A, L539A, L544A
G400A, L539A
G400A, L540A, M543A, L544A
G400A, L540A, M543A
G400A, L540A, L544A
G400A, L540A
G400A, M543A, L544A
G400A, M543A
G400A, L544A
G400A
G400L, L539A, L540A, M543A, L544A
G400L, L539A, L540A, M543A
G400L, L539A, L540A, L544A
G400L, L539A, L540A
G400L, L539A, M543A, L544A
G400L, L539A, M543A
G400L, L539A, L544A
G400L, L539A
G400L, L540A, M543A, L544A
G400L, L540A, M543A
G400L, L540A, L544A
G400L, L540A
G400L, M543A, L544A
G400L, M543A
G400L, L544A
G400L
G400I, L539A, L540A, M543A, L544A
G400I, L539A, L540A, M543A
G400I, L539A, L540A, L544A
G400I, L539A, L540A
G400I, L539A, M543A, L544A
G400I, L539A, M543A
G400I, L539A, L544A
G400I, L539A
G400I, L540A, M543A, L544A
G400I, L540A, M543A
G400I, L540A, L544A
G400I, L540A
G400I, M543A, L544A
G400I, M543A
G400I, L544A
G400I
G521R, L539A, L540A, M543A, L544A
G521R, L539A, L540A, M543A
G521R, L539A, L540A, L544A
G521R, L539A, L540A
G521R, L539A, M543A, L544A
G521R, L539A, M543A
G521R, L539A, L544A
G521R, L539A
G521R, L540A, M543A, L544A
G521R, L540A, M543A
G521R, L540A, L544A
G521R, L540A
G521R, M543A, L544A
G521R, M543A
G521R, L544A
G521R
G521T, L539A, L540A, M543A, L544A
G521T, L539A, L540A, M543A
G521T, L539A, L540A, L544A
G521T, L539A, L540A
G521T, L539A, M543A, L544A
G521T, L539A, M543A
G521T, L539A, L544A
G521T, L539A,
G521T, L540A, M543A, L544A
G521T, L540A, M543A
G521T, L540A, L544A
G521T, L540A
G521T, M543A, L544A
G521T, M543A
G521T, L544A
G521T
L539A, L540A, M543A, L544A
L539A, L540A, M543A
L539A, L540A, L544A
L539A, L540A
L539A, M543A, L544A
L539A, M543A
L539A, L544A
L539A
L540A, M543A, L544A
L540A, M543A
L540A, L544A
L540A
M543A, L544A
M543A
L544A
G521R
L539A, L540A, M543A, L544A
L539A, L540A, M543A
L539A, L540A, L544A
L539A, L540A
L539A, M543A, L544A
L539A, M543A
L539A, L544A
L539A,
L540A, M543A, L544A
L540A, M543A
L540A, L544A
L540A
M543A, L544A
M543A
L544A
G521T

In certain embodiments, a dominant negative signaling moiety includes a dominant negative kinase moiety, a dominant negative phosphatase moiety, a dominant negative GTPase moiety, a dominant negative guanine nucleotide exchange factor moiety, a dominant negative phospholipase moiety, a dominant negative paracaspase moiety, and/or a dominant negative protease moiety. In certain embodiments, a dominant negative kinase moiety includes a dominant negative relative to a kinase that regulates or mediates cell proliferation or function. In certain embodiments, a constitutively active signaling moiety includes a constitutively active phosphatase moiety, a constitutively active phosphatase moiety, a d constitutively active GTPase moiety, a constitutively active guanine nucleotide exchange factor moiety, a constitutively active phospholipase moiety, a constitutively active paracaspase moiety, and/or a constitutively active protease moiety. In certain embodiments, a constitutively active phosphatase moiety can be constitutively active relative to a phosphatase that regulates or mediates cell proliferation or function. In some embodiments, a cell proliferation or function includes an immune cell proliferation or function, e.g., T cell proliferation or function. In some embodiments, a cell proliferation or function includes a helper, effector, regulatory, or antigen-presenting immune cell proliferation or function. In some embodiments, a cell proliferation or function includes phagocyte proliferation or function.

In certain embodiments, a dominant negative kinase moiety is dominant negative relative to a Zap70 kinase. In certain embodiments, a dominant negative kinase moiety is a dominant negative variant of a Zap70 kinase. In some embodiments, a dominant negative Zap70 kinase moiety has a sequence that has at least 80%, at least 85%, at least 90%, at least 95%, at least 98%, or at least 99% sequence identity to SEQ ID NO: 2.

In certain embodiments, a dominant negative kinase moiety is dominant negative relative to a LCK kinase. In certain embodiments, a dominant negative kinase moiety is a dominant negative variant of a LCK kinase. In some embodiments, a dominant negative LCK kinase moiety has a sequence that has at least 80%, at least 85%, at least 90%, at least 95%, at least 98%, or at least 99% sequence identity to SEQ ID NO: 17.

In certain embodiments, a constitutively active phosphatase moiety is constitutively active relative to tyrosine phosphatase SH2-domain containing phosphatase 1 (SHP1). In certain embodiments, a constitutively active phosphatase moiety is a constitutively active variant of a SHP1 phosphatase. In some embodiments, a constitutively active SHP1 phosphatase moiety has a sequence that has at least 80%, at least 85%, at least 90%, at least 95%, at least 98%, or at least 99% sequence identity to SEQ ID NO: 23. In some embodiments, a modulating domain includes a ligand binding domain of a receptor and a trigger includes binding of a ligand to the ligand binding domain. In some embodiments, a modulating domain comprises a ligand binding domain of an estrogen receptor and a ligand is an agent that binds the estrogen receptor ligand binding domain, e.g., an estrogen agent. An estrogen agent can include an estrogen agonist, antagonist or mixed agonist-antagonist of a ligand binding domain of an estrogen receptor.

Some embodiments provide a nucleic acid that encodes a trigger-responsive immune-inactivating signaling polypeptide as described herein. In some embodiments, a vector includes a nucleic acid encoding a trigger-responsive immune-inactivating signaling polypeptide as described herein. In some embodiments, a cell includes one or more of a trigger-responsive immune-inactivating signaling polypeptide as described herein, a nucleic acid as described herein, and a vector as described herein. Some embodiments provide a nucleic acid that encodes a trigger-responsive dominant negative signaling polypeptide as described herein. In some embodiments, a vector includes a nucleic acid encoding a trigger-responsive dominant negative signaling polypeptide as described herein. In some embodiments, a cell includes one or more of a trigger-responsive dominant negative signaling polypeptide as described herein, a nucleic acid as described herein, and a vector as described herein. Some embodiments provide a nucleic acid that encodes a trigger-responsive constitutively active signaling polypeptide as described herein. In some embodiments, a vector includes a nucleic acid encoding a trigger-responsive constitutively active signaling polypeptide as described herein. In some embodiments, a cell includes one or more of a trigger-responsive constitutively active signaling polypeptide as described herein, a nucleic acid as described herein, and a vector as described herein. In some embodiments, a cell is an immune system cell, e.g., a monocyte, eosinophil, neutrophil, basophil, macrophage, dendritic cell, natural killer cell, T cell (e.g., helper T cell and cytotoxic T cell), T regulatory cell, or B cell. In some embodiments, a cell is an autologous cell. In some embodiments, a cell is an allogenic cell. In some embodiments, a cell is a T cell (e.g., a helper T cell and cytotoxic T cell).

In some embodiments, a cell is a genetically modified cell. In some instances, a genetically modified cells is a genetically modified T cell. In some embodiments, a genetically modified T cell can include a T cell receptor variant (e.g., a modified T cell receptor, a chimeric T cell receptor, or a T cell receptor including one or more mutations). In some embodiments, a genetically modified T cell is a chimeric antigen receptor T cell (CAR-T cell).

Some embodiments provide a composition (e.g., a pharmaceutical composition) that delivers a trigger-responsive immune-inactivating signaling polypeptide. In some embodiments, a composition that delivers a trigger-responsive immune-inactivating signaling polypeptide includes a trigger-responsive immune-inactivating signaling polypeptide as described herein, a nucleic acid as described herein, a vector as described herein, and/or a cell as described herein.

In some embodiments, the present disclosure provides a method of regulating an activity of an immune system. In some embodiments, a method can include regulating an activity of immune system cells in vivo. Immune system cells can include one or more of a monocyte, eosinophil, neutrophil, basophil, macrophage, dendritic cell, natural killer cell, T cell (e.g., helper T cell and cytotoxic T cell), T regulatory cell, and B cell. In some embodiments, a method of regulating an activity of immune system cells in vivo includes a step of administering a composition that delivers a trigger-responsive immune-inactivating signaling polypeptide to a subject. In some embodiments, a composition that delivers a trigger-responsive immune-inactivating signaling polypeptide includes a trigger-responsive immune-inactivating signaling polypeptide as described herein, a nucleic acid as described herein, the vector as described herein, and/or a cell as described herein. In some embodiments, a method of regulating an activity of immune system cells in vivo includes administering a trigger to a subject.

Particular embodiments provide a method of regulating activity of T cells in vivo. In some embodiments, a method of regulating activity of T cells in vivo includes a step of administering a composition that delivers a trigger-responsive immune-inactivating signaling polypeptide to a subject. In some embodiments, a composition that delivers a trigger-responsive immune-inactivating signaling polypeptide includes a trigger-responsive immune-inactivating signaling polypeptide as described herein, a nucleic acid as described herein, the vector as described herein, and/or a cell as described herein. In some embodiments, a method of regulating activity of T cells in vivo includes administering a trigger to a subject. In some embodiments, a method of regulating activity of T cells in vivo includes administering a genetically modified T cell (e.g., a chimeric antigen receptor T cell (CAR-T cell)) to a subject.

Certain embodiments provide a method of preventing or treating cytokine dysregulation. In some embodiments, a method of preventing or treating cytokine dysregulation includes a step of administering a composition that delivers a trigger-responsive immune-inactivating signaling polypeptide. In some embodiments, a composition that delivers a trigger-responsive immune-inactivating signaling polypeptide includes a trigger-responsive immune-inactivating signaling polypeptide as described herein, a nucleic acid as described herein, a vector as described herein, and/or a cell as described herein to a subject. In some embodiments, a method of preventing or treating cytokine dysregulation includes administering a trigger to a subject. In some embodiments, a method of preventing or treating cytokine dysregulation includes administering a genetically modified immune system cell (e.g., a monocyte, eosinophil, neutrophil, basophil, macrophage, dendritic cell, natural killer cell, T cell (e.g., a helper T cell and cytotoxic T cell), T regulatory cell, or B cell) to a subject. In some embodiments, a method of preventing or treating cytokine dysregulation includes administering a genetically modified T cell (e.g., a chimeric antigen receptor T cell (CAR-T cell)) to a subject. In some embodiments, a cytokine dysregulation includes hypercytokinemia, e.g., hypercytokinemia associated with graft-versus-host disease.

Some embodiments provide a method of treating cancer. In some instances, a method of treating cancer includes a step of administering a composition that delivers a trigger-responsive immune-inactivating signaling polypeptide. In some embodiments, a composition that delivers a trigger-responsive immune-inactivating signaling polypeptide includes a trigger-responsive immune-inactivating signaling polypeptide as described herein, a nucleic acid as described herein, the vector as described herein, and/or a cell as described herein to a subject. In some embodiments, a method of treating cancer includes administering a trigger to a subject. In some embodiments, a method of treating cancer includes administering a genetically modified immune system cell (e.g., a monocyte, eosinophil, neutrophil, basophil, macrophage, dendritic cell, natural killer cell, T cell (e.g., a helper T cell and cytotoxic T cell), T regulatory cell, or B cell) to a subject. In some embodiments, a method of preventing or treating cytokine dysregulation includes administering a genetically modified T cell (e.g., a chimeric antigen receptor T cell (CAR-T cell)) to a subject. In some embodiments, a cancer is a leukemia or a lymphoma.

In some embodiments, a method of manufacturing a trigger-responsive immune-inactivating signaling polypeptide as described herein includes a step of expressing the trigger-responsive immune-inactivating signaling polypeptide from a nucleic acid or a vector in a host cell. In some embodiments, a method of manufacturing a trigger-responsive immune-inactivating signaling polypeptide as described herein includes a step of recovering a trigger-responsive immune-inactivating signaling polypeptide, e.g., from a host cell.

Certain embodiments provide a method of manufacturing a genetically modified T cell that includes a trigger-responsive immune-inactivating signaling polypeptide as described herein. In some embodiments, such a method includes a step of comprising introducing a nucleic acid or a vector encoding a trigger-responsive immune-inactivating signaling polypeptide as described herein into an immune system cell (e.g., a monocyte, eosinophil, neutrophil, basophil, macrophage, dendritic cell, natural killer cell, T cell (e.g., a helper T cell and cytotoxic T cell), T regulatory cell, or B cell). A cell of an immune system can be autologous or allogenic.

These, and other aspects encompassed by the present disclosure, are described in more detail below and in the claims.

### Definitions

About: The term “about,” when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referenced value.

Administration: As used herein, the term “administration” typically refers to administration of a composition to a subject or system to achieve delivery of an agent that is, or is included in, the composition. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Adoptive cell therapy: As used herein, “adoptive cell therapy” or “ACT” involves transfer of immune cells with antitumour activity into a subject, e.g., cancer patients. In some embodiments, ACT is a treatment approach that involves the use of lymphocytes with antitumour activity, the in vitro expansion of these cells to large numbers and their infusion into a cancer-bearing host.

Agent: As used herein, the term “agent,” may refer to a compound, molecule, or entity of any chemical class including, for example, a small molecule, polypeptide, nucleic acid, saccharide, lipid, metal, or a combination or complex thereof. In some embodiments, the term “agent” may refer to a compound, molecule, or entity that comprises a polymer. In some embodiments, the term may refer to a compound or entity that comprises one or more polymeric moieties. In some embodiments, the term “agent” may refer to a compound, molecule, or entity that is substantially free of a particular polymer or polymeric moiety. In some embodiments, the term may refer to a compound, molecule, or entity that lacks or is substantially free of any polymer or polymeric moiety.

Amino acid: In its broadest sense, as used herein, “amino acid” refers to any compound and/or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Analog: As used herein, the term “analog” refers to a substance that shares one or more particular structural features, elements, components, or moieties with a reference substance. Typically, an “analog” shows significant structural similarity with the reference substance, for example sharing a core or consensus structure, but also differs in certain discrete ways. In some embodiments, an analog is a substance that can be generated from the reference substance, e.g., by chemical manipulation of the reference substance. In some embodiments, an analog is a substance that can be generated through performance of a synthetic process substantially similar to (e.g., sharing a plurality of steps with) one that generates the reference substance. In some embodiments, an analog is or can be generated through performance of a synthetic process different from that used to generate the reference substance.

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch.” Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present invention include glycosylated Fc domains, including Fc domains with modified or engineered such glycosylation. For purposes of the present invention, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc, as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-Bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]

Antibody agent: As used herein, the term “antibody agent” refers to an agent that specifically binds to a particular antigen. In some embodiments, the term encompasses any polypeptide or polypeptide complex that includes immunoglobulin structural elements sufficient to confer specific binding. Exemplary antibody agents include, but are not limited to monoclonal antibodies or polyclonal antibodies. In some embodiments, an antibody agent may include one or more constant region sequences that are characteristic of mouse, rabbit, primate, or human antibodies. In some embodiments, an antibody agent may include one or more sequence elements that are humanized, primatized, chimeric, etc, as is known in the art. In many embodiments, the term “antibody agent” is used to refer to one or more of the art-known or developed constructs or formats for utilizing antibody structural and functional features in alternative presentation. For example, in some embodiments, an antibody agent utilized in accordance with the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-Bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]. In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments an included CDR is substantially identical to a reference CDR in that it shows at least 96%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments an included CDR is substantially identical to a reference CDR in that at least one amino acid within the included CDR is substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is similar (e.g., homologous) or largely similar to an immunoglobulin-binding domain.

Antibody fragment: As used herein, an “antibody fragment” refers to a portion of an antibody or antibody agent as described herein, and typically refers to a portion that includes an antigen-binding portion or variable region thereof. An antibody fragment may be produced by any means. For example, in some embodiments, an antibody fragment may be enzymatically or chemically produced by fragmentation of an intact antibody or antibody agent. Alternatively, in some embodiments, an antibody fragment may be recombinantly produced (i.e., by expression of an engineered nucleic acid sequence. In some embodiments, an antibody fragment may be wholly or partially synthetically produced. In some embodiments, an antibody fragment (particularly an antigen-binding antibody fragment) may have a length of at least about 50, 60, 70, 80, 90, 100, 110, 120, 130, 140, 150, 160, 170, 180, 190 amino acids or more, in some embodiments at least about 200 amino acids.

Associated with: Two events or entities are “associated” with one another, as that term is used herein, if the presence, level and/or form of one is correlated with that of the other. For example, a particular entity (e.g., polypeptide, genetic signature, metabolite, microbe, etc) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Binding: It will be understood that the term “binding,” as used herein, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently or otherwise associated with a carrier entity and/or in a biological system or cell).

Cancer: The terms “cancer,” “malignancy,” “neoplasm,” “tumor,” and “carcinoma,” are used interchangeably herein to refer to cells that exhibit relatively abnormal, uncontrolled, and/or autonomous growth, so that they exhibit an aberrant growth phenotype characterized by a significant loss of control of cell proliferation. In general, cells of interest for detection or treatment in the present application include precancerous (e.g., benign), malignant, pre-metastatic, metastatic, and non-metastatic cells. The teachings of the present disclosure may be relevant to any and all cancers. To give but a few, non-limiting examples, in some embodiments, teachings of the present disclosure are applied to one or more cancers such as, for example, hematopoietic cancers including leukemias, lymphomas (Hodgkins and non-Hodgkins), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, and the like.

Characteristic portion: As used herein, the term “characteristic portion,” in the broadest sense, refers to a portion of a substance whose presence (or absence) correlates with presence (or absence) of a particular feature, attribute, or activity of the substance. In some embodiments, a characteristic portion of a substance is a portion that is found in the substance and in related substances that share the particular feature, attribute or activity, but not in those that do not share the particular feature, attribute or activity. In certain embodiments, a characteristic portion shares at least one functional characteristic with the intact substance. For example, in some embodiments, a “characteristic portion” of a protein or polypeptide is one that contains a continuous stretch of amino acids, or a collection of continuous stretches of amino acids, that together are characteristic of a protein or polypeptide. In some embodiments, each such continuous stretch generally contains at least 2, 5, 10, 15, 20, 50, or more amino acids. In general, a characteristic portion of a substance (e.g., of a protein, antibody, etc.) is one that, in addition to the sequence and/or structural identity specified above, shares at least one functional characteristic with the relevant intact substance. In some embodiments, a characteristic portion may be biologically active.

Characteristic sequence element: As used herein, the phrase “characteristic sequence element” refers to a sequence element found in a polymer (e.g., in a polypeptide or nucleic acid) that represents a characteristic portion of that polymer. In some embodiments, presence of a characteristic sequence element correlates with presence or level of a particular activity or property of the polymer. In some embodiments, presence (or absence) of a characteristic sequence element defines a particular polymer as a member (or not a member) of a particular family or group of such polymers. A characteristic sequence element typically comprises at least two monomers (e.g., amino acids or nucleotides). In some embodiments, a characteristic sequence element includes at least 2, 3, 4, 5, 6, 7, 8, 9, 10, 11, 12, 13, 14, 15, 20, 25, 30, 35, 40, 45, 50, or more monomers (e.g., contiguously linked monomers). In some embodiments, a characteristic sequence element includes at least first and second stretches of contiguous monomers spaced apart by one or more spacer regions whose length may or may not vary across polymers that share the sequence element.

Chimeric antigen receptor: “Chimeric antigen receptor” or “CAR” or “CARs” as used herein refers to engineered receptors, which graft an antigen specificity (e.g., an antigen specific moiety) onto cells (for example T cells such as naive T cells, central memory T cells, effector memory T cells or combination thereof). CARs are also known as artificial T cell receptors, chimeric T cell receptors or chimeric immunoreceptors. In some embodiments, CARs comprise an antigen-specific targeting regions, an extracellular domain, a transmembrane domain, one or more co-stimulatory domains, and an intracellular signaling domain. A T cell that has been genetically engineered to express a chimeric antigen receptors may be referred to as a CAR T cell.

Combination therapy: As used herein, the term “combination therapy” refers to those situations in which a subject is simultaneously exposed to two or more therapeutic regimens (e.g., two or more therapeutic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

Comparable: As used herein, the term “comparable” refers to two or more agents, entities, situations, sets of conditions, etc., that may not be identical to one another but that are sufficiently similar to permit comparison therebetween so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Corresponding to: As used herein, the term “corresponding to” may be used to designate the position/identity of a structural element in a compound or composition through comparison with an appropriate reference compound or composition. For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190^{th} amino acid in a particular amino acid chain but rather corresponds to the residue found at 190 in the reference polypeptide; those of ordinary skill in the art readily appreciate how to identify “corresponding” amino acids. For example, those skilled in the art will be aware of various sequence alignment strategies, including software programs such as, for example, BLAST, CS-BLAST, CUDASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that can be utilized, for example, to identify “corresponding” residues in polypeptides and/or nucleic acids in accordance with the present disclosure.

Designed: As used herein, the term “designed” refers to an agent (i) whose structure is or was selected by the hand of man; (ii) that is produced by a process requiring the hand of man; and/or (iii) that is distinct from natural substances and other known agents.

Domain: The term “domain” as used herein refers to a section or portion of an entity. In some embodiments, a “domain” is associated with a particular structural and/or functional feature of the entity so that, when the domain is physically separated from the rest of its parent entity, it substantially or entirely retains the particular structural and/or functional feature. Alternatively or additionally, a domain may be or include a portion of an entity that, when separated from that (parent) entity and linked with a different (recipient) entity, substantially retains and/or imparts on the recipient entity one or more structural and/or functional features that characterized it in the parent entity. In some embodiments, a domain is a section or portion of a molecule (e.g., a small molecule, carbohydrate, lipid, nucleic acid, or polypeptide). In some embodiments, a domain is a section of a polypeptide; in some such embodiments, a domain is characterized by a particular structural element (e.g., a particular amino acid sequence or sequence motif, □-helix character, □-sheet character, coiled-coil character, random coil character, etc.), and/or by a particular functional feature (e.g., binding activity, enzymatic activity, folding activity, signaling activity, etc.). In some embodiments, a domain is or includes a characteristic portion or characteristic sequence element.

Dosage form or unit dosage form: Those skilled in the art will appreciate that the term “dosage form” may be used to refer to a physically discrete unit of an active agent (e.g., a therapeutic or diagnostic agent) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular subject is determined by one or more attending physicians and may involve administration of multiple dosage forms.

Dosing regimen: Those skilled in the art will appreciate that the term “dosing regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount In some embodiments, a dosing regimen is correlated with a desired or beneficial outcome when administered across a relevant population (i.e., is a therapeutic dosing regimen).

Engineered: In general, the term “engineered” refers to the aspect of having been manipulated by the hand of man. For example, a polynucleotide is considered to be “engineered” when two or more sequences, that are not linked together in that order in nature, are manipulated by the hand of man to be directly linked to one another in the engineered polynucleotide. For example, in some embodiments of the present invention, an engineered polynucleotide comprises a regulatory sequence that is found in nature in operative association with a first coding sequence but not in operative association with a second coding sequence, is linked by the hand of man so that it is operatively associated with the second coding sequence. Comparably, a cell or organism is considered to be “engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). As is common practice and is understood by those in the art, progeny of an engineered polynucleotide or cell are typically still referred to as “engineered” even though the actual manipulation was performed on a prior entity. In some embodiments, “engineered” refers to an entity that has been designed and produced.

Excipient: As used herein, “excipient” refers to a non-therapeutic agent that may be included in a pharmaceutical composition, for example to provide or contribute to a desired consistency or stabilizing effect. In some embodiments, suitable pharmaceutical excipients may include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Fragment: A “fragment” of a material or entity as described herein has a structure that includes a discrete portion of the whole. In some embodiments, a fragment lacks one or more moieties found in the whole. In some embodiments, a fragment consists of such a discrete portion. In some embodiments, a fragment consists of or comprises a characteristic structural element, domain or moiety found in the whole. In some embodiments, a polymer fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) as found in the whole polymer. In some embodiments, a polymer fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the whole polymer. The whole material or entity may in some embodiments be referred to as the “parent” of the whole.

Fusion polypeptide: As used herein, the term “fusion polypeptide” generally refers to a polypeptide including at least two segments. Typically, a polypeptide containing at least two such segments is considered to be a fusion polypeptide if the two segments are moieties that (1) are not included in nature in the same peptide, and/or (2) have not previously been linked to one another in a single polypeptide, and/or (3) have been linked to one another through action of the hand of man.

Gene product or expression product: As used herein, the term “gene product” or “expression product” generally refers to an RNA transcribed from the gene (pre- and/or post-processing) or a polypeptide (pre- and/or post-modification) encoded by an RNA transcribed from the gene.

Host cell: As used herein, “host cell” refers to a cell into which exogenous DNA (recombinant or otherwise) has been introduced. Persons of skill upon reading this disclosure will understand that such terms refer not only to the particular subject cell, but also to the progeny of such a cell. Because certain modifications may occur in succeeding generations due to either mutation or environmental influences, such progeny (e.g., a clone cell) may not, in fact, be identical to the parent cell, but are still included within the scope of the term “host cell” as used herein. In some embodiments, host cells include prokaryotic and eukaryotic cells selected from any of the Kingdoms of life that are suitable for expressing an exogenous DNA (e.g., a recombinant nucleic acid sequence). In some embodiments, a host cell comprises one or more viral genes. In some embodiments, the introduction of exogenous into a host cell occurs via a transfection, transformation or a transduction. A transfection, transformation or a transduction can either be a transient transfection or a stable transfection, and one skilled in the art would be aware of various techniques for achieving transient or stable transfections, transformations or transductions. In some embodiments, a stable transfection, transformation or a transduction includes integration of the exogenous DNA into endogenous DNA of a host cell.

“Improve,” “increase,” “inhibit,” or “reduce”: As used herein, the terms “improve,” “increase,” “inhibit,” “reduce,” or grammatical equivalents thereof, indicate values that are relative to a baseline or other reference measurement. In some embodiments, an appropriate reference measurement may be or comprise a measurement in a particular system (e.g., in a single individual) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment.

Inhibitory agent: As used herein, the term “inhibitory agent” refers to an entity, condition, or event whose presence, level, or degree correlates with decreased level or activity of a target). In some embodiments, an inhibitory agent may be act directly (in which case it exerts its influence directly upon its target, for example by binding to the target); in some embodiments, an inhibitory agent may act indirectly (in which case it exerts its influence by interacting with and/or otherwise altering a regulator of the target, so that level and/or activity of the target is reduced). In some embodiments, an inhibitory agent is one whose presence or level correlates with a target level or activity that is reduced relative to a particular reference level or activity (e.g., that observed under appropriate reference conditions, such as presence of a known inhibitory agent, or absence of the inhibitory agent in question, etc).

Isolated: As used herein, “isolated” refers to a substance and/or entity that has been (1) separated from at least some of the components with which it was associated when initially produced (whether in nature and/or in an experimental setting), and/or (2) designed, produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polynucleotide that occurs in nature is considered to be “isolated” when, a) by virtue of its origin or source of derivation is not associated with some or all of the components that accompany it in its native state in nature; b) it is substantially free of other polypeptides or nucleic acids of the same species from the species that produces it in nature; c) is expressed by or is otherwise in association with components from a cell or other expression system that is not of the species that produces it in nature. Thus, for instance, in some embodiments, a polypeptide that is chemically synthesized or is synthesized in a cellular system different from that which produces it in nature is considered to be an “isolated” polypeptide. Alternatively or additionally, in some embodiments, a polypeptide that has been subjected to one or more purification techniques may be considered to be an “isolated” polypeptide to the extent that it has been separated from other components a) with which it is associated in nature; and/or b) with which it was associated when initially produced.

Linker: As used herein, “linker” is used to refer to that portion of a multi-element agent that connects different elements to one another. For example, those of ordinary skill in the art appreciate that a polypeptide whose structure includes two or more functional or organizational domains often includes a stretch of amino acids between such domains that links them to one another. In some embodiments, a polypeptide comprising a linker element has an overall structure of the general form S1-L-S2, wherein S1 and S2 may be the same or different and represent two domains associated with one another by the linker. In some embodiments, a polypeptide linker is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more amino acids in length. In some embodiments, a linker is characterized in that it tends not to adopt a rigid three-dimensional structure, but rather provides flexibility to the polypeptide. A variety of different linker elements that can appropriately be used when engineering polypeptides (e.g., fusion polypeptides) known in the art (see e.g., Holliger, P., et al. (1993) Proc. Natl. Acad. Sci. USA 90:6444-6448; Poljak, R. J., et al. (1994) Structure 2: 1121-1123).

Modulator: The term “modulator” is used to refer to an entity whose presence or level in a system in which an activity of interest is observed correlates with a change in level and/or nature of that activity as compared with that observed under otherwise comparable conditions when the modulator is absent. In some embodiments, a modulator is an activator, in that activity is increased in its presence as compared with that observed under otherwise comparable conditions when the modulator is absent. In some embodiments, a modulator is an antagonist or inhibitor, in that activity is reduced in its presence as compared with otherwise comparable conditions when the modulator is absent. In some embodiments, a modulator interacts directly with a target entity whose activity is of interest. In some embodiments, a modulator interacts indirectly (i.e., directly with an intermediate agent that interacts with the target entity) with a target entity whose activity is of interest. In some embodiments, a modulator affects level of a target entity of interest; alternatively or additionally, in some embodiments, a modulator affects activity of a target entity of interest without affecting level of the target entity. In some embodiments, a modulator affects both level and activity of a target entity of interest, so that an observed difference in activity is not entirely explained by or commensurate with an observed difference in level.

Nucleic acid: As used herein, in its broadest sense, “nucleic acid” refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has enzymatic activity.

Operably linked: As used herein, “operably linked” refers to a juxtaposition where the components described are in a relationship permitting them to function in their intended manner. For example, a control element “operably linked” to a functional element is associated in such a way that expression and/or activity of the functional element is achieved under conditions compatible with the control element. In some embodiments, “operably linked” control elements are contiguous (e.g., covalently linked) with the coding elements of interest; in some embodiments, control elements act in trans to or otherwise at a from the functional element of interest.

Pharmaceutical composition: As used herein, the term “pharmaceutical composition” refers to a composition in which an active agent is formulated together with one or more pharmaceutically acceptable carriers. In some embodiments, the active agent is present in unit dose amount appropriate for administration in a therapeutic regimen that shows a statistically significant probability of achieving a predetermined therapeutic effect when administered to a relevant population. In some embodiments, a pharmaceutical composition may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; jocularly; transdermally; or nasally, pulmonary, and to other mucosal surfaces.

Polypeptide: As used herein, “polypeptide” refers to any polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence similarity (e.g., homology) or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence similarity (e.g., homology) or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a useful polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Prevent or prevention: As used herein, “prevent” or “prevention,” when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Recombinant: As used herein, “recombinant” is intended to refer to polypeptides that are designed, engineered, prepared, expressed, created, manufactured, and/or or isolated by recombinant means, such as polypeptides expressed using a recombinant expression vector transfected into a host cell; polypeptides isolated from a recombinant, combinatorial human polypeptide library; polypeptides isolated from an animal (e.g., a mouse, rabbit, sheep, fish, etc) that is transgenic for or otherwise has been manipulated to express a gene or genes, or gene components that encode and/or direct expression of the polypeptide or one or more component(s), portion(s), element(s), or domain(s) thereof; and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of the polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc).

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Risk: As will be understood from context, “risk” of a disease, disorder, and/or condition refers to a likelihood that a particular individual will develop the disease, disorder, and/or condition. In some embodiments, risk is expressed as a percentage. In some embodiments, risk is from 0, 1, 2, 3, 4, 5, 6, 7, 8, 9, 10, 20, 30, 40, 50, 60, 70, 80, 90 up to 100%. In some embodiments risk is expressed as a risk relative to a risk associated with a reference sample or group of reference samples. In some embodiments, a reference sample or group of reference samples have a known risk of a disease, disorder, condition and/or event. In some embodiments a reference sample or group of reference samples are from individuals comparable to a particular individual. In some embodiments, relative risk is 0, 1, 2, 3, 4, 5, 6, 7, 8, 9, 10, or more.

Sample: As used herein, the term “sample” typically refers to an aliquot of material obtained or derived from a source of interest, as described herein. In some embodiments, a source of interest is a biological or environmental source. In some embodiments, a source of interest may be or comprise a cell or an organism, such as a microbe, a plant, or an animal (e.g., a human). In some embodiments, a source of interest is or comprises biological tissue or fluid. In some embodiments, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secretions, vitreous humour, vomit, and/or combinations or component(s) thereof. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological fluid may be or comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., broncheoalveolar, ductal, nasal, ocular, oral, uterine, vaginal, or other washing or lavage). In some embodiments, a biological sample is or comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc.

Small molecule: As used herein, the term “small molecule” means a low molecular weight organic and/or inorganic compound. In general, a “small molecule” is a molecule that is less than about 5 kilodaltons (kD) in size. In some embodiments, a small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, a small molecule is not a polymer. In some embodiments, a small molecule does not include a polymeric moiety. In some embodiments, a small molecule is not and/or does not comprise a protein or polypeptide (e.g., is not an oligopeptide or peptide). In some embodiments, a small molecule is not and/or does not comprise a polynucleotide (e.g., is not an oligonucleotide). In some embodiments, a small molecule is not and/or does not comprise a polysaccharide; for example, in some embodiments, a small molecule is not a glycoprotein, proteoglycan, glycolipid, etc.). In some embodiments, a small molecule is not a lipid. In some embodiments, a small molecule is a modulating agent (e.g., is an inhibiting agent or an activating agent). In some embodiments, a small molecule is biologically active. In some embodiments, a small molecule is detectable (e.g., comprises at least one detectable moiety). In some embodiments, a small molecule is a therapeutic agent. Those of ordinary skill in the art, reading the present disclosure, will appreciate that certain small molecule compounds described herein may be provided and/or utilized in any of a variety of forms such as, for example, crystal forms, salt forms, protected forms, pro-drug forms, ester forms, isomeric forms (e.g., optical and/or structural isomers), isotopic forms, etc. Those of skill in the art will appreciate that certain small molecule compounds have structures that can exist in one or more stereoisomeric forms. In some embodiments, such a small molecule may be utilized in accordance with the present disclosure in the form of an individual enantiomer, diastereomer or geometric isomer, or may be in the form of a mixture of stereoisomers; in some embodiments, such a small molecule may be utilized in accordance with the present disclosure in a racemic mixture form. Those of skill in the art will appreciate that certain small molecule compounds have structures that can exist in one or more tautomeric forms. In some embodiments, such a small molecule may be utilized in accordance with the present disclosure in the form of an individual tautomer, or in a form that interconverts between tautomeric forms. Those of skill in the art will appreciate that certain small molecule compounds have structures that permit isotopic substitution (e.g., ²H or ³H for H; ¹¹C, ¹³C or ¹⁴C for 12C; ¹³N or ¹⁵N for 14N; ¹⁷O or ¹⁸O for 16O; ³⁶Cl for XXC; ¹⁸F for XXF; 131I for XXXI; etc). In some embodiments, such a small molecule may be utilized in accordance with the present disclosure in one or more isotopically modified forms, or mixtures thereof. In some embodiments, reference to a particular small molecule compound may relate to a specific form of that compound. In some embodiments, a particular small molecule compound may be provided and/or utilized in a salt form (e.g., in an acid-addition or base-addition salt form, depending on the compound); in some such embodiments, the salt form may be a pharmaceutically acceptable salt form. In some embodiments, where a small molecule compound is one that exists or is found in nature, that compound may be provided and/or utilized in accordance in the present disclosure in a form different from that in which it exists or is found in nature. Those of ordinary skill in the art will appreciate that, in some embodiments, a preparation of a particular small molecule compound that contains an absolute or relative amount of the compound, or of a particular form thereof, that is different from the absolute or relative (with respect to another component of the preparation including, for example, another form of the compound) amount of the compound or form that is present in a reference preparation of interest (e.g., in a primary sample from a source of interest such as a biological or environmental source) is distinct from the compound as it exists in the reference preparation or source. Thus, in some embodiments, for example, a preparation of a single stereoisomer of a small molecule compound may be considered to be a different form of the compound than a racemic mixture of the compound; a particular salt of a small molecule compound may be considered to be a different form from another salt form of the compound; a preparation that contains only a form of the compound that contains one conformational isomer ((Z) or (E)) of a double bond may be considered to be a different form of the compound from one that contains the other conformational isomer ((E) or (Z)) of the double bond; a preparation in which one or more atoms is a different isotope than is present in a reference preparation may be considered to be a different form; etc.

Solid Tumor: As used herein, the term “solid tumor” refers to an abnormal mass of tissue that usually does not contain cysts or liquid areas. In some embodiments, a solid tumor may be benign; in some embodiments, a solid tumor may be malignant. Those skilled in the art will appreciate that different types of solid tumors are typically named for the type of cells that form them. Examples of solid tumors are carcinomas, lymphomas, and sarcomas. In some embodiments, solid tumors may be or comprise adrenal, bile duct, bladder, bone, brain, breast, cervix, colon, endometrium, esophagus, eye, gall bladder, gastrointestinal tract, kidney, larynx, liver, lung, nasal cavity, nasopharynx, oral cavity, ovary, penis, pituitary, prostate, retina, salivary gland, skin, small intestine, stomach, testis, thymus, thyroid, uterine, vaginal, and/or vulval tumors.

Specific binding: As used herein, the term “specific binding” refers to an ability to discriminate between possible binding partners in the environment in which binding is to occur. A binding agent that interacts with one particular target when other potential targets are present is said to “bind specifically” to the target with which it interacts. In some embodiments, specific binding is assessed by detecting or determining degree of association between the binding agent and its partner; in some embodiments, specific binding is assessed by detecting or determining degree of dissociation of a binding agent-partner complex; in some embodiments, specific binding is assessed by detecting or determining ability of the binding agent to compete an alternative interaction between its partner and another entity. In some embodiments, specific binding is assessed by performing such detections or determinations across a range of concentrations.

Stage of cancer: As used herein, the term “stage of cancer” refers to a qualitative or quantitative assessment of the level of advancement of a cancer. In some embodiments, criteria used to determine the stage of a cancer may include, but are not limited to, one or more of where the cancer is located in a body, tumor size, whether the cancer has spread to lymph nodes, whether the cancer has spread to one or more different parts of the body, etc. In some embodiments, cancer may be staged using the so-called TNM System, according to which T refers to the size and extent of the main tumor, usually called the primary tumor; N refers to the number of nearby lymph nodes that have cancer; and M refers to whether the cancer has metastasized. In some embodiments, a cancer may be referred to as Stage 0 (abnormal cells are present but have not spread to nearby tissue, also called carcinoma in situ, or CIS; CIS is not cancer, but it may become cancer), Stage I-III (cancer is present; the higher the number, the larger the tumor and the more it has spread into nearby tissues), or Stage IV (the cancer has spread to distant parts of the body). In some embodiments, a cancer may be assigned to a stage selected from the group consisting of: in situ (abnormal cells are present but have not spread to nearby tissue); localized (cancer is limited to the place where it started, with no sign that it has spread); regional (cancer has spread to nearby lymph nodes, tissues, or organs): distant (cancer has spread to distant parts of the body); and unknown (there is not enough information to figure out the stage).

Subject: As used herein, the term “subject” refers an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Substantial sequence similarity: The phrase “substantial sequence similarity” is used herein to refer to a comparison between amino acid or nucleic acid sequences. As will be appreciated by those of ordinary skill in the art, two sequences are generally considered to be “substantially similar” if they contain a conservative amino acid substitution in corresponding positions. A conservative substitution is one in which an amino acid has been replaced by a non-identical residue having appropriately similar structural and/or functional characteristics. For example, as is well known by those of ordinary skill in the art, certain amino acids are typically classified as “hydrophobic” or “hydrophilic” amino acids, and/or as having “polar” or “non-polar” side chains. Substitution of one amino acid for another of the same type may often be considered a conservative substitution. Typical amino acid categorizations are summarized in Tables 2 and 3 below:

TABLE 2

| | | | | | |
|---|---|---|---|---|---|
| Alanine | Ala | A | nonpolar | neutral | 1.8 |
| Arginine | Arg | R | polar | positive | −4.5 |
| Asparagine | Asn | N | polar | neutral | −3.5 |
| Aspartic acid | Asp | D | polar | negative | −3.5 |
| Cysteine | Cys | C | nonpolar | neutral | 2.5 |
| Glutamic acid | Glu | E | polar | negative | −3.5 |
| Glutamine | Gln | Q | polar | neutral | −3.5 |
| Glycine | Gly | G | nonpolar | neutral | −0.4 |
| Histidine | His | H | polar | positive | −3.2 |
| Isoleucine | Ile | I | nonpolar | neutral | 4.5 |
| Leucine | Leu | L | nonpolar | neutral | 3.8 |
| Lysine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

TABLE 3

| Ambiguous Amino Acids | 3-Letter | 1-Letter |
|---|---|---|
| Asparagine or aspartic acid | Asx | B |
| Glutamine or glutamic acid | Glx | Z |
| Leucine or Isoleucine | Xle | J |
| Unspecified or unknown amino acid | Xaa | X |

As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215(3): 403-410, 1990; Altschul, et al., Methods in Enzymology; Altschul, et al., “Gapped BLAST and PSI-BLAST: a new generation of protein database search programs,” Nucleic Acids Res. 25:3389-3402, 1997; Baxevanis, et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying similar sequences, the programs mentioned above typically provide an indication of the degree of similarity. In some embodiments, two sequences are considered to be substantially similarity if at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or more of their corresponding residues are similar and/or identical over a relevant stretch of residues. In some embodiments, the relevant stretch is a complete sequence. In some embodiments, the relevant stretch is at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 225, at least 250, at least 275, at least 300, at least 325, at least 350, at least 375, at least 400, at least 425, at least 450, at least 475, at least 500 or more residues. As would be appreciated by one of ordinary skill in the art sequences with substantial sequence similarity may be homologs of one another.

Substantial sequence identity: As used herein, the phrase “substantial sequence identity” refers to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul et al., Basic local alignment search tool, J. Mol. Biol., 215(3): 403-410, 1990; Altschul et al., Methods in Enzymology; Altschul et al., Nucleic Acids Res. 25:3389-3402, 1997; Baxevanis et al., Bioinformatics: A Practical Guide to the Analysis of Genes and Proteins, Wiley, 1998; and Misener, et al, (eds.), Bioinformatics Methods and Protocols (Methods in Molecular Biology, Vol. 132), Humana Press, 1999. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

Substantial structural similarity: As used herein, the term “substantial structural similarity” refers to presence of shared structural features such as presence and/or identity of particular amino acids at particular positions (see definitions of “shared sequence similarity” and “shared sequence identity”). In some embodiments the term “substantial structural similarity” refers to presence and/or identity of structural elements (for example: loops, sheets, helices, H-bond donors, H-bond acceptors, glycosylation patterns, salt bridges, and disulfide bonds). In some embodiments, the term “substantial structural similarity” refers to three dimensional arrangement and/or orientation of atoms or moieties relative to one another (for example: distance and/or angles between or among them between an agent of interest and a reference agent).

Susceptible to: An individual who is “susceptible to” a disease, disorder, or condition is at risk for developing the disease, disorder, or condition. In some embodiments, an individual who is susceptible to a disease, disorder, or condition does not display any symptoms of the disease, disorder, or condition. In some embodiments, an individual who is susceptible to a disease, disorder, or condition has not been diagnosed with the disease, disorder, and/or condition. In some embodiments, an individual who is susceptible to a disease, disorder, or condition is an individual who has been exposed to conditions associated with development of the disease, disorder, or condition. In some embodiments, a risk of developing a disease, disorder, and/or condition is a population-based risk (e.g., family members of individuals suffering from the disease, disorder, or condition).

Symptoms are reduced: According to the present invention, “symptoms are reduced” when one or more symptoms of a particular disease, disorder or condition is reduced in magnitude (e.g., intensity, severity, etc.) and/or frequency. For purposes of clarity, a delay in the onset of a particular symptom is considered one form of reducing the frequency of that symptom.

T cell receptor: As used herein, a “T cell receptor” or “TCR” refers to the antigen-recognition molecules present on the surface of T cells. During normal T cell development, each of the four TCR genes, α, β, γ, and δ, can rearrange leading to highly diverse TCR proteins.

Therapeutic agent: As used herein, the phrase “therapeutic agent” in general refers to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is a substance that can be used to alleviate, ameliorate, relieve, inhibit, prevent, delay onset of, reduce severity of, and/or reduce incidence of one or more symptoms or features of a disease, disorder, and/or condition. In some embodiments, a “therapeutic agent” is an agent that has been or is required to be approved by a government agency before it can be marketed for administration to humans. In some embodiments, a “therapeutic agent” is an agent for which a medical prescription is required for administration to humans.

Therapeutic regimen: A “therapeutic regimen,” as that term is used herein, refers to a dosing regimen whose administration across a relevant population may be correlated with a desired or beneficial therapeutic outcome.

Therapeutically effective amount: As used herein, is meant an amount that produces the desired effect for which it is administered. In some embodiments, the term refers to an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount of a particular agent or therapy may be formulated and/or administered in a single dose. In some embodiments, a therapeutically effective agent may be formulated and/or administered in a plurality of doses, for example, as part of a dosing regimen.

Treatment: As used herein, the term “treatment” (also “treat” or “treating”) refers to any administration of a therapy that partially or completely alleviates, ameliorates, relives, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

Variant: As used herein in the context of molecules, e.g., nucleic acids, proteins, or small molecules, the term “variant” refers to a molecule that shows significant structural identity with a reference molecule but differs structurally from the reference molecule, e.g., in the presence or absence or in the level of one or more chemical moieties as compared to the reference entity. In some embodiments, a variant also differs functionally from its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule. To give but a few examples, a polypeptide may have a characteristic sequence element comprised of a plurality of amino acids having designated positions relative to one another in linear or three-dimensional space and/or contributing to a particular structural motif and/or biological function; a nucleic acid may have a characteristic sequence element comprised of a plurality of nucleotide residues having designated positions relative to on another in linear or three-dimensional space. In some embodiments, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. In some embodiments, a variant polypeptide or nucleic acid does not share at least one characteristic sequence element with a reference polypeptide or nucleic acid. In some embodiments, a reference polypeptide or nucleic acid has one or more biological activities. In some embodiments, a variant polypeptide or nucleic acid shares one or more of the biological activities of the reference polypeptide or nucleic acid. In some embodiments, a variant polypeptide or nucleic acid lacks one or more of the biological activities of the reference polypeptide or nucleic acid. In some embodiments, a variant polypeptide or nucleic acid shows a reduced level of one or more biological activities as compared to the reference polypeptide or nucleic acid. In some embodiments, a polypeptide or nucleic acid of interest is considered to be a “variant” of a reference polypeptide or nucleic acid if it has an amino acid or nucleotide sequence that is identical to that of the reference but for a small number of sequence alterations at particular positions. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide or nucleic acid comprises about 10, about 9, about 8, about 7, about 6, about 5, about 4, about 3, about 2, or about 1 substituted residues as compared to a reference. Often, a variant polypeptide or nucleic acid comprises a very small number (e.g., fewer than about 5, about 4, about 3, about 2, or about 1) number of substituted, inserted, or deleted, functional residues (i.e., residues that participate in a particular biological activity) relative to the reference. In some embodiments, a variant polypeptide or nucleic acid comprises not more than about 5, about 4, about 3, about 2, or about 1 addition or deletion, and, in some embodiments, comprises no additions or deletions, as compared to the reference. In some embodiments, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, a reference polypeptide or nucleic acid is one found in nature. In some embodiments, a reference polypeptide or nucleic acid is a human polypeptide or nucleic acid.

All literature and similar material cited in this application, including, but not limited to, patents, patent applications, articles, books, treatises, and web pages, regardless of the format of such literature and similar materials, are expressly incorporated by reference in their entirety. In the event that one or more of the incorporated literature and similar materials differs from or contradicts this application, including but not limited to defined terms, term usage, described techniques, or the like, this application controls. The section headings used herein are for organizational purposes only and are not to be construed as limiting the subject matter described in any way.

### BRIEF DESCRIPTION OF THE DRAWING

FIGS. 1A-1D are schematics of pathways in a T cell receptor signaling cascade as illustrated by Huse, M., “The T-cell-receptor signaling network,” Journal of Cell Science, 122, p. 1269-1273 (2009).

FIG. 2 includes a schematic of a vector encoding a trigger-responsive dominant negative signaling polypeptide, as well as nucleotide and amino acid sequences for a trigger-responsive dominant negative signaling polypeptide encoded by the vector and portions thereof.

FIG. 3 includes a schematic of a vector encoding a dominant negative signaling moiety, as well as nucleotide and amino acid sequences for a dominant negative signaling moiety encoded by the vector and portions thereof.

FIGS. 4A-4D includes schematics of different arrangements contemplated for a trigger-responsive dominant negative signaling polypeptide as described herein. “L” in FIGS. 4A-4D refers to a linker.

FIGS. 5A-5H includes schematics of different arrangements contemplated for a trigger-responsive dominant negative signaling polypeptide as described herein. “L” in FIGS. 5A-5H refers to a linker.

FIGS. 6A and 6B include schematics of a nuclear receptor, e.g., an estrogen receptor. In FIGS. 6A and 6B, “AF-1” refers to an activation function 1 domain, “DBD” refers to a DNA binding domain, “LBD” refers to a ligand binding domain, and “AF-2” refers to an activation function 2 domain. Further, in FIG. 6A, “H” refers to a hinge region.

FIG. 7 includes a bar graph demonstrating inhibition of NFAT-luciferase expression by an endoxifen-responsive dominant negative Zap-70 polypeptide in Jurkat E6.1 cells that have been transiently transfected with DNA encoding an endoxifen-responsive dominant negative Zap-70 polypeptide. Each bar represents the mean activity from quadruplicate wells and error bars represent the standard error of the mean.

FIG. 8 includes a dose response curve showing that the activity of a dominant negative Zap-70 moiety included in an endoxifen-responsive dominant negative Zap-70 polypeptide was regulated by endoxifen is a dose dependent manner. Each point represents the mean activity from quadruplicate wells and error bars represent the standard error of the mean.

FIG. 9 includes a bar graph demonstrating inhibition of NFAT-luciferase expression by an endoxifen-responsive dominant negative Zap-70 polypeptide in Jurkat E6.1 cells that have been transiently transfected with DNA encoding an endoxifen-responsive dominant negative Zap-70 polypeptide. Each bar represents the mean activity from triplicate wells and error bars represent the standard error of the mean.

FIG. 10 includes a bar graph demonstrating inhibition of NFAT-luciferase expression by an endoxifen-responsive dominant negative Zap-70 polypeptide in Jurkat E6.1 cells that have been stably transfected with DNA encoding an endoxifen-responsive dominant negative Zap-70 polypeptide. Each bar represents the mean activity from triplicate wells and error bars represent the standard error of the mean.

FIG. 11 includes a line graph showing that the activity of a dominant negative Zap-70 moiety included in an endoxifen-responsive dominant negative Zap-70 polypeptide was dependent on the amount of DNA encoding the endoxifen-responsive dominant negative Zap-70 polypeptide that is present in the cells. Each point represents the mean activity from quadruplicate wells and error bars represent the standard error of the mean.

FIG. 12 includes two plots showing that endoxifen-responsive dominant negative Zap-70 polypeptides, each including either a G400V or a G400L mutation, were able to inhibit the T cell activation cascade and expression of luciferase from an NFAT-luciferase construct in an endoxifen dose dependent manner. Each point represents the activity of individual replicates with line denoting the mean from quadruplicate wells and error bars representing SEM.

FIG. 13 includes a line graph showing endoxifen dose response curves for endoxifen-responsive dominant negative Zap-70 polypeptides that included either a G400V or a G400L mutation. FIG. 13 also includes pIC50 calculated based on those dose response curves. Each point represents the mean activity from sextuplicate wells and error bars represent the standard error of the mean.

FIG. 14 includes a schematic of a nucleic acid sequence encoding a ZAP70dn(1-278)-ER(T12) trigger-responsive dominant negative signaling polypeptide. The sequence includes and/or encodes a start codon, a nuclear export signal, a ZAP70dn(1-278) dominant negative signaling polypeptide, a BamHI restriction site, an ER(T12) modulating domain, and a stop codon.

FIG. 15 includes a schematic of the amino acid sequence of a ZAP70dn(1-278)-ER(T12) trigger-responsive dominant negative signaling polypeptide. The polypeptide includes a methionine amino acid having been encoded by a start codon, a nuclear export signal, a ZAP70dn(1-278) dominant negative signaling polypeptide, amino acids having been encoded by a BamHI restriction site, and an ER(T12) modulating domain.

FIG. 16 includes a schematic of two constructs encoding respective polypeptides. Expression of each construct is driven by a CMV promoter. The polypeptides encoded by the two constructs differ in their modulating domains, as one includes an ER(T2) modulating domain and the other includes an ER(T12) modulating domain.

FIG. 17 includes a schematic of an expression construct capable of expressing a Zap70dn(1-278)-ER(T12) trigger-responsive dominant negative signaling polypeptide.

FIG. 18 includes a graph showing induction of NFAT-Luciferase in cells co-transfected with an expression construct encoding an NFAT-Luciferase reporter and an expression construct encoding either ZAP70dn(1-278)-ER(T2) or ZAP70dn(1-278)-ER(T12) in the absence or presence of varying amounts of endoxifen.

FIG. 19 includes a chart showing relative light units (RLU) resulting from expression of NFAT-Luciferase reporter in cells co-transfected with an expression construct encoding an NFAT-Luciferase reporter and (a) empty vector control; (b) an expression construct encoding ZAP70dn(1-278); or (c) an expression construct encoding ZAP70dn(1-278)-ER(T12), in the absence or presence of varying amounts of endoxifen.

FIG. 20 includes a schematic of a nucleic acid sequence encoding a LCKdn(1-266)-ER(T12) trigger-responsive dominant negative signaling polypeptide. The sequence includes and/or encodes a start codon, a nuclear export signal, a LCKdn(1-266) dominant negative signaling polypeptide, a BamHI restriction site, an ER(T12) modulating domain, and a stop codon.

FIG. 21 includes a schematic of the amino acid sequence of a LCKdn(1-266)-ER(T12) trigger-responsive dominant negative signaling polypeptide. The polypeptide includes a methionine amino acid having been encoded by a start codon, a nuclear export signal, a LCKdn(1-266) dominant negative signaling polypeptide, amino acids having been encoded by a BamHI restriction site, and an ER(T12) modulating domain.

FIG. 22 includes a schematic of a construct encoding a LCK(1-266)-(ER(T12) trigger-responsive dominant negative signaling polypeptide. Expression of the polypeptide is driven by a CMV promoter.

FIG. 23 includes a schematic of an expression construct capable of expressing a LCK(1-266)-ER(T12) trigger-responsive dominant negative signaling polypeptide.

FIG. 24 includes a graph showing induction of NFAT-Luciferase in cells co-transfected with an expression construct encoding an NFAT-Luciferase reporter and (a) empty vector control; (b) an expression construct encoding LCK(1-266); or (c) an expression construct encoding LCK(1-266)-ER(T12), in the absence or presence of endoxifen.

FIG. 25 includes a schematic of a nucleic acid sequence encoding a SHP1(210-595)-ER(T12) trigger-responsive dominant negative signaling polypeptide. The sequence includes and/or encodes a start codon, a nuclear export signal, a SHP1(210-595) constitutively active signaling polypeptide, a BamHI restriction site, an ER(T12) modulating domain, and a stop codon.

FIG. 26 includes a schematic of the amino acid sequence of a SHP1(210-595)-ER(T12) trigger-responsive dominant negative signaling polypeptide. The polypeptide includes a methionine amino acid having been encoded by a start codon, a nuclear export signal, a SHP1(210-595) constitutively active signaling polypeptide, amino acids having been encoded by a BamHI restriction site, and an ER(T12) modulating domain.

FIG. 27 includes a schematic of a construct encoding SHP1(210-595)-ER(T12) trigger-responsive constitutively active signaling polypeptide. Expression of the polypeptide is driven by a CMV promoter.

FIG. 28 includes a schematic of an expression construct capable of expressing a SHP1(210-595)-ER(T12) trigger-responsive constitutively active signaling polypeptide.

FIG. 29 includes a graph showing induction of IL2-Luciferase in cells co-transfected with an expression construct encoding an IL2-Luciferase reporter and (a) empty vector control; (b) an expression construct encoding SHP1(210-595); or (c) an expression construct encoding SHP1(210-595)-ER(T12), in the absence or presence of endoxifen.

FIG. 30 includes a schematic of a signaling cascade. SHP1 inhibits T-cell activation after it is released from an inactive form. SHP1 is constitutively associated with inhibitory receptor LAIR-1, which, in turn, is constitutively phosphorylated by LCK, although SHP1 may also be activated by other ITIM-containing inhibitory receptors. Activation of SHP1 allows SHP1 to inhibit antigen-induced TCR signaling through dephosphorylation of the TCR chain and/or dephosphorylation of adaptor proteins such as LCK and ZAP70. Activating phosphate groups are shown as stars.

FIG. 31 is a pair of graphs showing dose response of the SHP1(210-595)-ER(T12) to the presence of endoxifen, as detected using either an NFAT-Luciferase reporter or an IL2-Luciferase reporter.

### DETAILED DESCRIPTION OF CERTAIN EMBODIMENTS

The present disclosure provides technologies for regulating the activity of immune system cells (e.g., monocytes, eosinophils, neutrophils, basophils, macrophages, dendritic cells, natural killer cells, T cells (including helper T cells and cytotoxic T cells), T regulatory cells, and/or B cells). Particularly, the present disclosure provides technologies for regulating T cell activity and encompasses the insight that systems for controlling T cell activation and/or activity can greatly enhance therapies that utilize and/or rely on T cells.

### Adoptive T Cell Therapy

Adoptive T Cell Therapy (ATCT) is one current approach that shows promise in treating various conditions and/or diseases (e.g., cancer). ATCT entails collection and isolation of T cells from a subject (e.g., a patient). Isolated T cells are then clonally enriched, modified, and/or engineered to achieve a T cell population having desired properties and/or characteristics. The T cell population can then be expanded through ex-vivo growth and reintroduced into the subject to allow the enriched, modified, and/or engineered T cells to specifically attack cells of interest.

One type of ATCT that has been particularly effective in treating cancers (such as leukemias and lymphomas) utilizes T cells that have been engineered to express a chimeric antigen receptor (a “CAR”); such T cells are often referred to as CAR-T cells.

While initial CAR T cell approaches did not produce the desired clinical results, second-generation CAR T cells that were engineered to express, e.g., a chimeric fusion protein that contains an extracellular domain that recognizes antigens present on tumor cells, a hinge/transmembrane domain, a costimulatory domain, and a CD3 zeta chain, showed promise. For instance, a group at the University of Pennsylvania and the pharmaceutical company Novartis reported positive clinical results in patients with Chronic Leukocytic Lymphoma (CLL) (see Porter, et al., Chimeric Antigen Receptor—Modified T Cells in Chronic Lymphoid Leukemia, NEJM (2011)), Acute Lymphocytic Leukemia (ALL) (see Maude, et al., Chimeric Antigen Receptor T Cells for Sustained Remissions in Leukemia, NEJM (2014)), and Non-Hodgkins Lymphoma (NHL)(see Schuster, S. J., “183 Sustained Remissions Following Chimeric Antigen Receptor Modified T Cells Directed Against CD19 (CTL019) in Patients with Relapsed or Refractory CD19+ Lymphomas,” ASH 57^{th} Annual Meeting & Exposition, Session 624, Poster, (2015)). Subsequently, many groups have rushed into the field to develop optimized CAR T cells, and other related T cell based therapies.

Another type of ATCT that has been effective involves use of specific high-affinity T Cell Receptors (TCRs) to bind target antigens. Such high-affinity TCRs can be used, e.g., in place of a CAR, and can bind to cell surface proteins, such as CD19.

Despite the extremely promising effectiveness of ATCTs, the usefulness of the method has been hampered by potential adverse effects of the treatment. A prominent adverse effect has been the occurrence of cytokine storm in many patients, which can involve damage to multiple organs, fever, neurotoxicity, and/or death. Another adverse event has been tumor lysis syndrome. Still another adverse effect has been allogenic therapy graft versus host disease.

Efforts to minimize the chances of such side effects have included introduction of control elements into engineered T cells. One example of such an approach is described by Wendell Lim, et al. (Wu, et al., 2015, “Remote control of therapeutic T cells through a small molecule-gated chimeric receptor,” Science, (350) 6258), in which a CAR is divided into two pieces that are inactive unless brought together by a small molecule compound that can mediate association of the two pieces. Unfortunately, the small molecule compound used to demonstrate the method is not practical to use on human patients for a number of reasons, including a short half-life.

As another example, Bellicum, Inc. has employed a so-called “suicide switch” that, when activated by a small molecule drug, leads to programmed cell death of engineered T cells (Di Stasi, A., et al., “Inducible apoptosis as a safety switch for adoptive cell therapy,” N Engl J Med., 365(18):1673-83; doi: 10.1056/NEJMoa1106152 (2011)). This suicide switch may be activated, for example, if the engineered T cells are mediating graft versus host disease. This approach can be effective to “turn off” the engineered T cells. However, the present disclosure appreciates that there is a problem with the strategy, as it inactivates engineered T cells by destroying them, which wastes time and resources and also may result in the subject (e.g., patient) having to undergo additional procedures to replace the destroyed T cells, which can be painful, expensive and time consuming.

### Trigger-Responsive Modulation of T Cell Activity

The present disclosure provides a system that can allow for fine-tuned regulation of T cell activity, including specifically of CAR-T and TCR T cell activity using a trigger, for example, an innocuous, practical, and approved small molecule. In some embodiments, such regulation is reversible (e.g., by alternating initiation and termination of exposure to the trigger). Alternatively or additionally, in some embodiments, such regulation may be sensitive to degree of exposure to the trigger (e.g., to trigger concentration and/or frequency, etc). In some embodiments, exposure to a trigger can “dial down” cytokine release and/or one or more other activities of T cells, including of reintroduced and/or engineered T cells.

In some embodiments, exposure to a trigger involves administration of a trigger agent (e.g., a small molecule agent). In some embodiments, exposure to a trigger may be for a finite (and/or predetermined) period of time, for example due to clearance (e.g., by degradation, removal, sequestering, or other means etc) of the trigger agent. In some embodiments, cessation of exposure to the agent relieves the modification of T cell activity that occurred during exposure to the agent.

In some particular embodiments, administration of a trigger agent results in a decrease in one or more hallmarks of T cell activity (e.g., cytokine release). In some embodiments, such decrease may be commensurate with concentration (e.g., local concentration and/or plasma concentration) of administered agent, and/or with frequency and/or magnitude of dose administration). Alternatively or additionally, in some embodiments, clearance of the agent (e.g., via natural mechanisms or by induced removal or degradation, for example as may be achieved by administration of a follow-on agent that stimulates clearance of the trigger agent) relieves the decrease. In some embodiments, subsequent administration of the trigger agent re-establishes the decrease. In some embodiments, the system remains sensitive to multiple cycles of administration and clearance of the trigger agent.

In some embodiments, the present disclosure achieves regulation of T cell activity through use of a immune-inactivating moiety of a T cell activation pathway component. Moreover, in some embodiments, the present disclosure provides an insight that association of such an immune-inactivating moiety with a modulating domain whose inhibitory or masking action can be relieved by a trigger creates an agent that can regulate T cell activity in a trigger-responsive, and, in many embodiments, reversible (even serially reversible) fashion.

In some embodiments, the present disclosure achieves regulation of T cell activity through use of a dominant negative signaling moiety of a T cell activation pathway component. Moreover, in some embodiments, the present disclosure provides an insight that association of such a dominant negative signaling moiety with a modulating domain whose inhibitory or masking action can be relieved by a trigger creates an agent that can regulate T cell activity in a trigger-responsive, and, in many embodiments, reversible (even serially reversible) fashion.

In some embodiments, the present disclosure achieves regulation of T cell activity through use of a constitutively active signaling moiety of a T cell activation pathway component. Moreover, in some embodiments, the present disclosure provides an insight that association of such a constitutively active signaling moiety with a modulating domain whose inhibitory or masking action can be relieved by a trigger creates an agent that can regulate T cell activity in a trigger-responsive, and, in many embodiments, reversible (even serially reversible) fashion.

In particular embodiments, the present disclosure provides insights that connect technologies from disparate fields to provide new strategies for regulating T cell activity that achieve surprising advantages relative to existing approaches. For example, among other things, the present disclosure appreciates that developments providing immune-inactivating moieties of T cell activation pathway components (such as a dominant negative kinase moiety or a constitutively active phosphatase moiety) can be combined with features of ligand-responsive nuclear receptors to provide a system for trigger-responsive regulation of T cell activity. Furthermore, the present disclosure appreciates that application of such a system to ATCT technologies, including CAR-T and/or TCR T cells, provides new and remarkably useful therapeutic T cell modalities.

Approaches for regulation of T cell activity described herein provide a variety of advantages relative to available systems including, for example, that T cell activity can be inhibited without destroying T cells. Furthermore, in many embodiments, provided systems provide for reversible inhibition of T cell activity. Thus, the present disclosure provides systems in which activity of a T cell population (which may be a maintained T cell population) can be reversibly decreased and increased through application and removal of a trigger. Combining trigger-responsiveness with maintenance of T cell levels (and, in at least some embodiments, reversibility and/or tunability through adjustment of trigger “intensity”—e.g., concentration, level and/or frequency of application, etc) provides a remarkably sophisticated and effective system that, moreover, is applicable to any of a variety of T cell populations including, for example, existing ATCT (e.g., CAR-T and/or TCR) T cell populations. Yet another advantage of provided systems is that they utilize and/or impact existing T cell biological cascades, rather than requiring that a new signaling cascade be introduced as is required, for example, for the recently-reported Notch-signaling-based system developed by Lim, et al. (see, for example, Roybal, et al., Cell 167:419, “Engineering T Cells with Customized Therapeutic Response Programs Using Synthetic Notch Receptors,” Oct. 6, 2016).

Those of skill in the art will appreciate from the present disclosure that a further advantage of regulating T cell activity is that methods and compositions of the present disclosure can be used to treat T cell exhaustion of T cells (e.g., T cells introduced into a subject, e.g., CAR-T cells) that include, express, or encode a trigger-responsive immune-inactivating polypeptide. For instance, in certain embodiments, exposure of subject including one or more exhausted T cells that include, express, or encode a trigger-responsive immune-inactivating polypeptide to a trigger can treat exhaustion of T cells in the subject. In such instances, treatment of T cell exhaustion can include a change in the state of one or more exhausted T cells such that the T cells are capable of functioning as non-exhausted T cells, e.g., during or after cessation of exposure of the T cell to trigger.

Thus, among other things, the present disclosure provides trigger-responsive T cell activity modulating agents that comprise a immune-inactivating moiety (i.e., a moiety that, when present in a T cell that includes a functional T cell activation signaling cascade, interferes with the cascade such that T cell activation signaling is disrupted) and a modulating domain that, in many embodiments, is characterized by an ability to adopt a first state and a second state, and to transition between the first state and the second state when exposed to a trigger. When such a modulating domain is in its first state, the immune-inactivating moiety with which it is associated is inhibited, and when the modulating domain is in its second state, the inhibition is relieved. In accordance with the present disclosure, introduction of such a trigger-responsive T cell activity modulator into a T cell (e.g., by introduction and/or expression of a nucleic acid that encodes it) renders activity of the T cell responsive to presence of the trigger: when the trigger is absent, the modulating domain adopts its first state and the immune-inactivating moiety is inhibited so that the T cell activation cascade is functional; when the trigger is present, the modulating domain adopts its second state and the immune-inactivating moiety is active so that the T cell activation cascade is inhibited. Those of skill in the art will appreciate that, in some embodiments, degree of inhibition (or functionality) of the T cell activation cascade may be tuned through adjustment of level and/or frequency of trigger exposure (e.g., by concentration of the trigger) and/moreover, that such inhibition (or functionality) may, in many embodiments, be reversible, optionally through several cycles.

Thus, among other things, the present disclosure provides trigger-responsive T cell activity modulating agents that comprise a dominant negative signaling moiety (i.e., a moiety that, when present in a T cell that includes a functional T cell activation signaling cascade, interferes with the cascade such that T cell activation signaling is disrupted) and a modulating domain that, in many embodiments, is characterized by an ability to adopt a first state and a second state, and to transition between the first state and the second state when exposed to a trigger. When such a modulating domain is in its first state, the dominant negative signaling moiety with which it is associated is inhibited, and when the modulating domain is in its second state, the inhibition is relieved. In accordance with the present disclosure, introduction of such a trigger-responsive T cell activity modulator into a T cell (e.g., by introduction and/or expression of a nucleic acid that encodes it) renders activity of the T cell responsive to presence of the trigger: when the trigger is absent, the modulating domain adopts its first state and the dominant negative signaling moiety is inhibited so that the T cell activation cascade is functional; when the trigger is present, the modulating domain adopts its second state and the dominant negative signaling moiety is active so that the T cell activation cascade is inhibited. Those of skill in the art will appreciate that, in some embodiments, degree of inhibition (or functionality) of the T cell activation cascade may be tuned through adjustment of level and/or frequency of trigger exposure (e.g., by concentration of the trigger) and/moreover, that such inhibition (or functionality) may, in many embodiments, be reversible, optionally through several cycles.

The present disclosure provides trigger-responsive T cell activity modulating agents that comprise a constitutively active signaling moiety (i.e., a moiety that, when present in a T cell that includes a functional T cell activation signaling cascade, interferes with the cascade such that T cell activation signaling is disrupted) and a modulating domain that, in many embodiments, is characterized by an ability to adopt a first state and a second state, and to transition between the first state and the second state when exposed to a trigger. When such a modulating domain is in its first state, the constitutively active signaling moiety with which it is associated is inhibited, and when the modulating domain is in its second state, the inhibition is relieved. In accordance with the present disclosure, introduction of such a trigger-responsive T cell activity modulator into a T cell (e.g., by introduction and/or expression of a nucleic acid that encodes it) renders activity of the T cell responsive to presence of the trigger: when the trigger is absent, the modulating domain adopts its first state and the constitutively active signaling moiety is inhibited so that the T cell activation cascade is functional; when the trigger is present, the modulating domain adopts its second state and the constitutively active signaling moiety is active so that the T cell activation cascade is inhibited. Those of skill in the art will appreciate that, in some embodiments, degree of inhibition (or functionality) of the T cell activation cascade may be tuned through adjustment of level and/or frequency of trigger exposure (e.g., by concentration of the trigger) and/moreover, that such inhibition (or functionality) may, in many embodiments, be reversible, optionally through several cycles.

In some embodiments, a dominant negative signaling moiety may be or comprise a dominant negative signaling moiety of a T cell activation pathway component. In some embodiments, a dominant negative signaling moiety may be or comprise a dominant negative kinase moiety (e.g., of a kinase that operates in a T cell activation pathway).

In some embodiments, a constitutively active signaling moiety may be or comprise a constitutively active signaling moiety of a T cell activation pathway component. In some embodiments, a constitutively active signaling moiety may be or comprise a constitutively active phosphatase moiety (e.g., of a phosphatase that operates in a T cell activation pathway).

In some embodiments, a modulating domain can be or comprise a nuclear receptor (e.g., a hormone receptor) or portion thereof (e.g., a ligand binding domain thereof). For example, in some embodiments, a modulating domain can be or comprise a ligand binding domain of an estrogen receptor, e.g., an estrogen receptor in which mutations have been introduced. In some embodiments, mutations are introduced in an estrogen receptor to increase its ability to form inactivating complexes with heat shock proteins, to lose affinity to estrogen, and/or to retain affinity for synthetic ligands such as raloxifene, tamoxifen, 4-hydroxy tamoxifen and endoxifen (e.g., in ER(T2) or ER(T12)).

### Signaling Moieties

### T Cell Activation Pathway and Associated Signaling Entities

A T cell-receptor (TCR) is a polypeptide complex found on the surface of T cells. A TCR comprises a heterodimer of α and β polypeptide chains that is non-covalently associated with a CD3 dimer of γε, δε, or ζζ polypeptide chains. Each of the γ, δ, ε, and ζ polypeptides includes at least one polypeptides include three) so-called immunoreceptor tyrosine-based activation motifs (ITAMs) characterized by two tyrosine residues flanking a series of amino acids that include key leucine/isoleucine residues with stereotypic spacing.

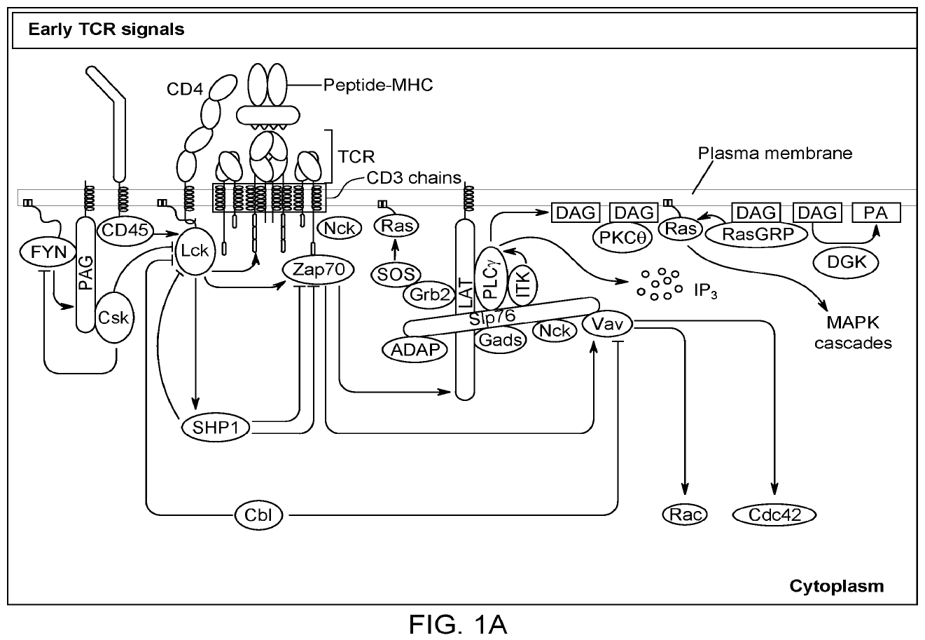

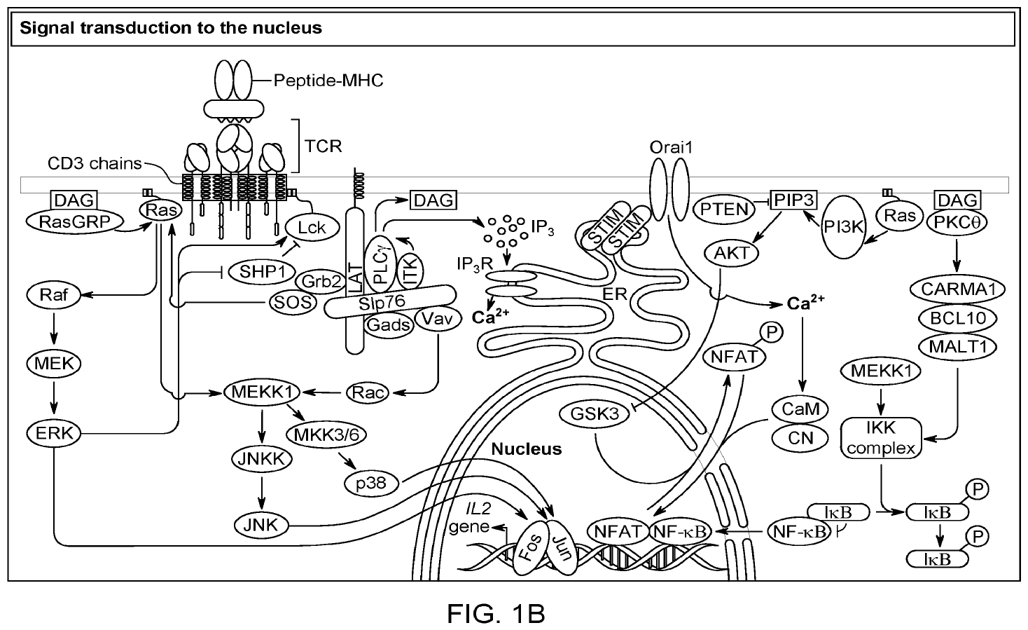

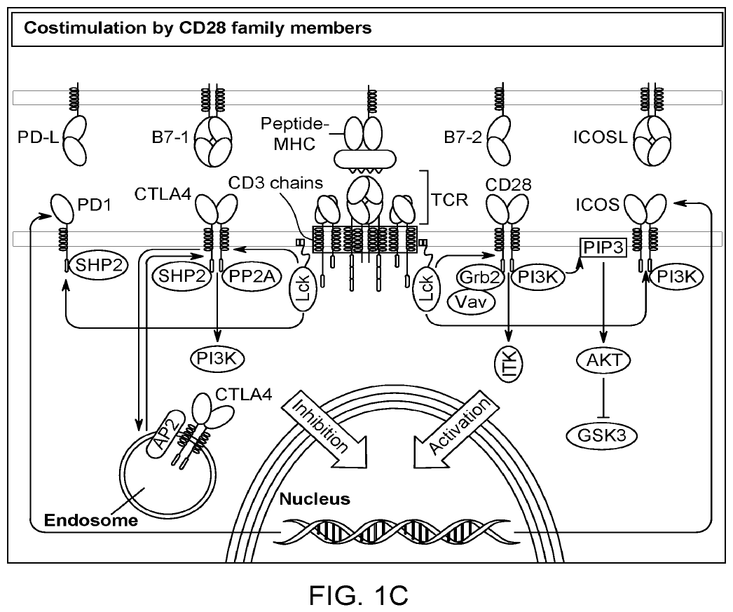

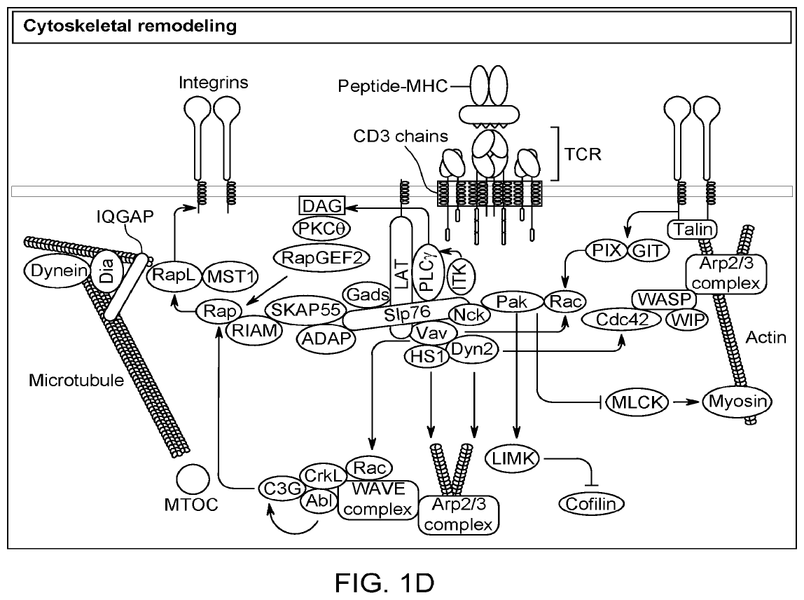

TCR signaling in response to antigen recognition initiates T cell activation, which plays a central role in the adaptive immune response. As explained by, e.g., Huse, M., “The T-cell-receptor signaling network,” Journal of Cell Science, 122, p. 1269-1273 (2009) and shown in FIGS. 1A-1D, T cell activation sets off a network of signaling cascades. In particular, recognition of cognate antigenic peptide in the context of major histocompatibility complex (peptide-MHC) by a TCR can induce conformational changes within the associated CD3 chains that facilitate their phosphorylation and association with downstream proteins. ITAMs of the CD3 δ-, γ-, ε- and ζ-chains are phosphorylated by a Src family kinase leukocyte-specific tyrosine kinase (Lck) upon ligand recognition by a TCR. A significant proportion of Lck in a cell constitutively associates with a co-receptor CD4. Because CD4 also interacts with MHC molecules, it recruits Lck to regions that contain TCR complexes. Phosphorylated CD3 ITAMs recruit a Syk family kinase zeta-activated protein 70 kDa (Zap70) via Src Homology-2 (SH2)-domain interactions. An adaptor protein Nck also associates directly with polyproline sequences within CD3 ε.

Upon localization to a TCR complex, Zap70 phosphorylates multiple tyrosine residues within Linker for the Activation of T cells (LAT), a membrane-associated scaffolding protein. Phosphorylated LAT recruits a second molecular scaffold, SH2-domain-containing leukocyte protein of 76 kDa (Slp76), which binds to LAT via an intervening protein Gads (Grb2-related adapter protein 2 or GRAP2). Slp76 is then phosphorylated by Zap70, and the resulting LAT-Slp76 complex acts as a platform for recruitment of signaling effectors, many of which bind directly to phosphotyrosine-based motifs. One of the signaling effectors is phospholipase C-γ (PLCγ), which can interact directly with both LAT and Slp76. PLCγ transduces TCR signals by hydrolyzing phosphatidylinositol bisphosphate (PIP2) to yield diacylglycerol (DAG), a membrane-associated lipid, and inositol trisphosphate (IP3), a diffusible second messenger. DAG recruits a number of downstream proteins to the membrane, among them protein kinase C-θ (PKCθ) and RasGRP (RAS guanyl nucleotide-releasing protein), which is a guanine nucleotide exchange factor (GEF). RasGRP activates the small GTPase, Ras, an activator of mitogen-activated protein kinase (MAPK) signaling pathways in many cell types. Ras can also be activated by the exchange factor son of sevenless (SOS), which is recruited to LAT via the adaptor molecule Grb2 (growth-factor-receptor-bound protein 2).

Phosphorylated Slp76 binds directly to the Tec family kinase interleukin inducible T cell kinase (ITK). Together with Zap70 and Lck, ITK has an essential role in the phosphorylation and activation of PLCγ. In addition, Slp76 recruits the GEF, Vav, which activates the small GTPases, Rac and Cdc42. The adaptor proteins Nck and adhesion- and degranulation-promoting adaptor protein (ADAP) are also recruited into the complex. The LAT-Slp76 complex may be a highly cooperative signalosome. Many of its constituent proteins interact with several partners, and the loss of any one protein disrupts signaling through other effectors. This cooperative behavior may be important for coordinating and coupling different branches of the TCR signaling network.

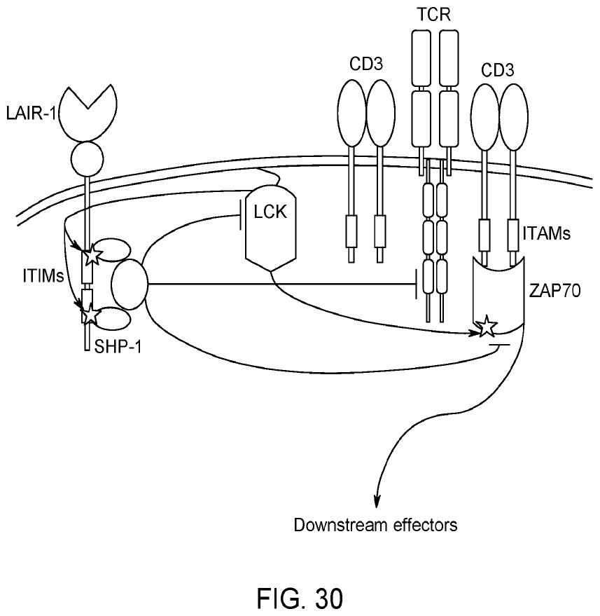

These early membrane-proximal signaling steps are subject to inhibition on a number of levels. The tyrosine phosphatase SH2-domain containing phosphatase 1 (SHP1) dephosphorylates and deactivates both Zap70 and Lck (see, e.g., FIG. 30). In addition, the E3 ubiquitin ligase Cbl targets several proteins for proteasomal degradation, including Lck, Zap70 and Vav. PLCγ-mediated signaling is attenuated by diacylglycerol kinases (DGKs), which phosphorylate DAG to yield phosphatidic acid (PA). The tyrosine kinase C-terminal Src kinase (Csk) inhibits proximal TCR signaling by phosphorylating a tyrosine motif in the C-terminal tail of Lck. Csk is recruited to the plasma membrane in a phosphotyrosine-dependent manner by the scaffolding molecule phosphoprotein associated with glycosphingolipid-enriched microdomains (PAG), which is maintained in a phosphorylated state by the Src kinase Fyn. In addition to targeting Lck, Csk also phosphorylates the inhibitory C-terminal tail of Fyn, which provides negative feedback by reducing PAG phosphorylation.

Lck tail phosphorylation is removed by CD45, a tyrosine phosphatase, which restores TCR signaling. Under certain conditions, however, CD45 can inhibit Lck and other effectors by dephosphorylating phosphotyrosine residues that are required for their optimal activity.

In addition to early TCR signals, TCR stimulation results in signal transduction to the nucleus, which leads to profound changes in gene expression. Many of these changes are mediated by the transcription factors activator protein 1 (AP1, a heterodimer of Fos and Jun), nuclear factor of activated T cells (NFAT) and nuclear factor-κB (NF-κB). These three factors act together to activate transcription of the interleukin-2 gene.

Activation of Fos and Jun occurs as a downstream event of three MAPK signaling pathways. Each pathway consists of an effector MAPK [extracellular signal-regulated kinase (Erk), Jun kinase (JNK) and protein of 38 kDa (p38)], an upstream MAPK kinase [MAPK or ERK kinase (MEK), JNK kinase (JNKK) and MAPK kinase 3/6 (MKK3/6)], and a MAPK kinase kinase [MEK kinase 1 (MEKK1) and Raf]. The Erk pathway is stimulated by the association of active Ras with Raf, whereas the JNK and p38 pathways respond to activated Rac in addition to Ras. MAPK signaling cascades stimulate AP1 activity via the upregulation of Fos and Jun transcription, and also by direct phosphorylation of the Fos and Jun proteins. In addition, Erk engages in positive feedback by phosphorylating Lck. This phosphorylation event blocks inhibitory interactions between Lck and SHP1.

NFAT activity is regulated by intracellular Ca²⁺ concentration. When Ca²⁺ levels are low, phosphorylation by a kinase known as glycogen synthase kinase 3 (GSK3) induces nuclear export of NFAT. Increases in intracellular Ca²⁺ lead to dephosphorylation and nuclear import of NFAT. NFAT dephosphorylation is mediated by the phosphatase calcineurin (CN), which is activated by its association with the Ca²⁺-binding protein calmodulin (CaM). Cytoplasmic Ca²⁺ levels are coupled to TCR activation through PLCγ. Production of IP3 by PLCγ stimulates the opening of Ca²⁺-permeable ion channels known as IP3 receptors (IP3Rs) in the endoplasmic reticulum (ER). This leads to the depletion of Ca²⁺ from the ER, which induces the aggregation of the Ca²⁺ sensors stromal interaction molecule 1 (STIM1) and STIM2 in regions of close ER-plasma-membrane apposition. These STIM clusters are thought to trigger the opening of Orail channels in the cell membrane, leading to a large and sustained influx of Ca²⁺ into the cytoplasm. This second, Orail-dependent, rise in Ca²⁺ drives NFAT into the nucleus.

NFAT translocation is also regulated by phosphatidylinositol 3-kinase (PI3K), which is activated downstream of several TCR signaling effectors, including Ras. PI3K phosphorylates PIP2 to yield PIP3, a phospholipid that recruits a variety of cytoplasmic proteins to the cell membrane. One of the most important of these is the kinase AKT, which promotes cell survival via several distinct pathways. AKT phosphorylates GSK3, thereby inhibiting the phosphorylation of NFAT and promoting its nuclear translocation. PI3K signaling is regulated by the opposing activity of the phosphatase and tensin homolog (PTEN).

Under resting conditions, NF-κB is sequestered in the cytoplasm by inhibitor of κB (IκB). Phosphorylation of IκB by the IκB kinase (IKK) complex leads to the ubiquitylation and degradation of IκB, allowing NF-κB to translocate to the nucleus. IKK is activated by MEKK1 and also by a protein complex comprising the adaptors caspase recruitment domain containing membrane-associated guanylate kinase protein 1 (CARMA1), B-cell lymphoma 10 (Bcl10) and mucosa-associated lymphoid tissue lymphoma translocation gene 1 (MALT1). This complex functions downstream of PKCθ, which is recruited to the cell membrane by DAG. Thus, both NFAT and NF-κB rely on different branches of the PLCγ signaling pathway for their activation.

Optimal T cell stimulation that leads to proliferation and other effector functions requires that a second, ‘costimulatory’ signal be delivered through a distinct cell-surface receptor. Although several transmembrane proteins, including LFA1 and CD2, can provide costimulation in certain contexts, the archetypal costimulatory receptor is CD28. CD28 binds to B7-1 (also known as CD80) and B7-2 (also known as CD86), which are highly expressed by antigen presenting cells (APCs), such as dendritic cells. Ligand binding of CD28 induces the phosphorylation of tyrosine-containing sequences in its cytoplasmic tail by Src-family kinases. This event leads to the recruitment of several downstream proteins, including PI3K, Grb2, Vav and ITK.

The inhibitory receptor cytotoxic T-lymphocyte antigen 4 (CTLA4) is closely related to CD28 and also binds to B7-1 and B7-2, but with significantly higher affinity than CD28. In resting T cells, almost all CTLA4 is sequestered in intracellular compartments such as endosomes via a mechanism that depends on the sorting adaptor AP2 (adaptor protein 2). TCR stimulation induces the trafficking of CTLA4 to the cell surface, where it can bind to its ligand and trigger signals that attenuate TCR signaling. Similarly to CD28, CTLA4 is phosphorylated by Src kinases at tyrosine residues in its cytoplasmic tail. The phosphatases protein phosphatase 2A (PP2A) and Src-homology 2 domain-containing phosphatase 2 (SHP2) both bind to phosphorylated CTLA4, as does PI3K. PP2A and SHP2 might inhibit TCR signaling by dephosphorylating membrane-proximal effectors, although it is also possible that CTLA4 mediates its inhibitory effects by competing with CD28 for binding to B7 ligands that are common to both receptors, which would crowd CD28 out of the immunological synapse.

TCR signaling stimulates the expression of two other CD28 family members known as inducible costimulatory molecule (ICOS) and programmed cell death 1 (PD1). After trafficking to the surface, both of these proteins can regulate the sustained phase of T cell signaling when activated by their respective ligands. ICOS enhances T cell effector functions but, unlike CD28, does not stimulate proliferation. By contrast, PD1 is a potent inhibitor of TCR signaling, similarly to CTLA4. It appears to act in different contexts than CTLA4, however, because PD1 ligand (PD-L) is expressed by different cell types than those that express B7-1 and B7-2.

TCR signaling also induces dramatic changes in cytoskeletal architecture. Antigen recognition by the T cell stimulates a burst of actin polymerization at the immunological synapse, generating a lamellapodial sheet structure that spreads over the surface of the APC. The actin-related protein 2/3 (Arp2/3) complex, which stimulates the growth of branched actin arrays, has a central role in this process. Arp2/3 is coupled to the LAT-Slp76 signalosome through Vav, which activates Cdc42 and Rac. Cdc42 triggers Arp2/3 activation by recruiting and activating the Wiskott-Aldrich syndrome protein (WASP), whereas Rac activates Arp2/3 through the WAVE (WASP family verprolin-homologous protein) complex. Actin polymerization is also stimulated by the cortactin homolog HS1 (hematopoietic lineage cell-specific protein 1), as well as the GTPase dynamin 2 (Dyn2), both of which interact with Vav.

TCR-stimulated actin polymerization is temporally correlated with an increase in integrin-mediated adhesion, which occurs via an ‘inside-out’ signaling mechanism. The upregulation of the function of integrins, primarily of the αLβ2 integrin LFA1 (lymphocyte function-associated antigen 1) is directly affected by Vav, PLCγ and other components of the LAT-Slp76 complex. Vav-dependent actin polymerization can induce integrin activation via recruitment of the cytoskeletal linker talin, which binds directly to integrin tails. PLCγ, for its part, activates integrins via the small GTPase, Rap. This occurs through the generation of DAG by PLCγ, which stimulates Rap by recruiting a protein complex containing PKCθ and the Rap exchange factor RapGEF2. Rap can also be activated by the exchange factor C3G (RapGEF1), which is recruited together with the tyrosine kinase Abl to the WAVE complex. Once Rap is loaded with GTP, it associates with LAT-Slp76 through a protein complex that contains ADAP, Src kinase-associated phosphoprotein of 55 kDa (SKAP55) and Rap-GTPinteracting adapter molecule (RIAM) and mediates integrin activation.

Integrin activation promotes enhanced adhesion of the T cell to the APC, facilitating the establishment of a long-lived T cell-APC contact. Activated integrins also induce intracellular signals that promote further cytoskeletal remodeling. For example, the exchange factor p21-activated kinase (PAK)-interacting exchange factor (PIX), which is associated with the adaptor G-protein-coupled receptor kinase interactor (GIT), is activated downstream of integrin adhesion. PIX-mediated activation of Rac in this context stimulates the kinase activity of PAK, which phosphorylates LIM kinase (LIMK) and myosin light chain kinase (MLCK). PAK phosphorylation activates LIMK, which promotes actin polymerization by phosphorylating and inhibiting the actin-severing protein cofilin. Phosphorylation of MLCK inhibits its kinase activity, and thereby its ability to promote myosin-based contraction. Taken together, these effects promote the growth and maintenance of actin-based structures in the cell.

TCR signaling also induces the polarization of the microtubule-organizing center (MTOC) to the immunological synapse. MTOC reorientation appears to depend on the negatively directed microtubule motor dynein. Microtubules radiate from the MTOC with positive ends facing outwards and negative ends facing inwards. Therefore, dynein that is localized at the immunological synapse can bind to microtubule tips and ‘reel’ the MTOC in towards itself.

### Moieties Based on T Cell Activation Cascade Signaling Entities

The present disclosure provides a particular insight that T cell activation involves signaling pathways that may provide particularly attractive opportunities to control T cell activation and/or activity, which can greatly enhance therapies that utilize and/or rely on T cells. For example, a number of kinases and phosphatases play a role in T cell activation. Still further, the present disclosure appreciates that dominant negative moieties based on signaling entities, e.g., kinases and/or phosphatases, within a T cell activation pathway are available and/or can be readily generated.

In certain embodiments, the present disclosure provides technologies that utilize a dominant negative signaling moiety based on a kinase in a T cell activation pathway to regulate T cell activity. In certain particular embodiments, the present disclosure provides trigger-responsive dominant negative signaling polypeptides—i.e., constructs that can adopt at least first and second conformations, and can switch from one to the other in response to a particular trigger—in which a dominant negative signaling moiety (e.g., kinase moiety) is inhibited in one state relative to the other state. In particular embodiments, such a trigger-responsive dominant negative signaling polypeptide comprises a dominant negative variant based on a signaling entity (e.g., a kinase) that participates in a T cell activation pathway operably linked with a modulating domain as described herein.

In certain embodiments, the present disclosure provides technologies that utilize a constitutively active signaling moiety based on a phosphatase in a T cell activation pathway to regulate T cell activity. In certain particular embodiments, the present disclosure provides trigger-responsive constitutively active signaling polypeptides—i.e., constructs that can adopt at least first and second conformations, and can switch from one to the other in response to a particular trigger—in which a constitutively active signaling moiety (e.g., phosphatase moiety) is inhibited in one state relative to the other state. In particular embodiments, such a trigger-responsive constitutively active signaling polypeptide comprises a constitutively active variant based on a signaling entity (e.g., a phosphatase) that participates in a T cell activation pathway operably linked with a modulating domain as described herein.

A list of exemplary signaling entities that play a role in a T cell activation pathway, which are included in Table 4 (below).

TABLE 4
Exemplary Kinases in a T cell Activation Pathway
zeta-chain-associated protein kinase 70 (“Zap-70”)
lymphocyte-specific protein tyrosine kinase (“Lck”)
phosphatidylinositol-4,5-bisphosphate 3-kinase (“PI3K”)
pyruvate dehydrogenase lipoamide kinase isozyme 1 (“PDK1”)
protein kinase C theta (“PKCθ”)
serine/threonine-protein kinase (“Raf”)
mitogen-activated protein kinase kinase 1 (“MEK1” or “MAP2K1”)
mitogen-activated protein kinase kinase 2 (“MEK2” or “MAP2K2”)
mitogen-activated protein kinase 3 (“ERK1” or “MAPK3”)
mitogen-activated protein kinase 1 (“ERK2” or “MAPK1”)
mitogen-activated protein kinase kinase kinase 1 (“MEKK1” or
“MAP3K1”)
mitogen-activated protein kinase kinase 4 (“MKK4” or “MAP2K4” or
“JNKK”)
mitogen-activated protein kinase kinase 7 (“MKK7” or “MAP2K7”)
mitogen-activated protein kinase 3/6 (“MAPK 3/6”)
c-Jun N-terminal kinase 1 (“JNK1”)
p38 mitogen-activated protein kinase (“p38 MAPK”)
c-Jun N-terminal kinase 2 (“JNK2”)
inhibitor of nuclear factor kappa-B kinase subunit gamma (“IKKγ”)
inhibitor of nuclear factor kappa-B kinase subunit beta (“IKKβ”)
inhibitor of nuclear factor kappa-B kinase subunit alpha (“IKKα”)
protein kinase B (“Akt” or “PKB”)
mechanistic target of rapamycin (“mTOR”)
calcium/calmodulin-dependent protein kinase type IV (“CaMKIV”)
mitogen-activated protein kinase kinase kinase kinase 1 (“HPK1”
or “MAP4K1”)
TGF-beta-activated kinase 1 (“TAK1” or “MAP3K7”)
inducible T cell kinase (“ITK”)
C-terminal Src kinase (“Csk”)
glycogen synthase kinase 3 (“GSK3”)
Other Exemplary Enzymes in a T cell Activation Pathway
calcineurin (“CaN”)
Calpain
phospholipase Cγ1 (“PLCγ1”)
cell division control protein 42 homolog (“Cdc42”)
ras-related C3 botulinum toxin substrate (“Rac”)
Ras
Mucosa-associated lymphoid tissue lymphoma translocation protein 1
(“MALT1”)
CD45 receptor tyrosine phosphatase
Tyrosine phosphatase SH2-domain containing phosphatase 1 (SHP1)
phosphatases protein phosphatase 2A (PP2A)

In some embodiments, a dominant negative signaling moiety based on a signaling entity in a T cell activation pathway (e.g., as listed in Table 4) can be used in a trigger-responsive dominant negative signaling polypeptide described here. Dominant negative moieties based on kinases within a T cell activation pathway are available and/or can be readily generated. As one specific example, a dominant negative form of Zeta-associated Protein (Zap)-70 has been described by Qian, et al. (Qian, D., et al., “Dominant-negative Zeta-associated Protein 70 Inhibits T Cell Antigen Receptor Signaling,” J. Exp. Med., Vol. 183, p. 611-620 (1996)). As discussed above, Zap-70 is a cytoplasmic protein tyrosine kinase that is essential for T cell activity. In wild type cells, a T cell Receptor (TCR) binds antigens and recruits a CD3-zeta chain protein, leading to phosphorylation of ITAMs on CD3 and recruitment and activation of Zap-70. Activation of Zap-70 triggers an intracellular signaling cascade that drives T Cell activity. Qian, et al. made dominant negative mutants of Zap-70 that inactivated the kinase activity of Zap-70, and therefore, were able to disrupt Zap-70 signaling. Qian, et al. achieved the inactivation of Zap-70 kinase activity using two general approaches: point mutations or a truncation of the kinase domain.

In some embodiments, a dominant negative Zap70 moiety can be encoded by DNA having a nucleotide sequence according to SEQ ID NO: 1. As disclosed herein, SEQ ID NO: 1 represents an exemplary nucleotide sequence encoding a dominant negative Zap70 moiety. In some embodiments, a dominant negative Zap70 moiety can be encoded by DNA having a nucleotide sequence substantially similar to SEQ ID NO: 1. In some embodiments, a dominant negative Zap70 moiety can be encoded by DNA having a nucleotide sequence with at least 75%, at least 80%, at least 85%, at least 90%, at least 95%, at least 96%, at least 97%, at least 98%, or at least 99% sequence identity to the nucleotide sequence of SEQ ID NO: 1.

SEQ ID NO: 1
ATGCCAGACCCCGCGGCGCACCTGCCCTTCTTCTACGGCAGCATCTCGCG
TGCCGAGGCCGAGGAGCACCTGAAGCTGGCGGGCATGGCGGACGGGCTCT
TCCTGCTGCGCCAGTGCCTGCGCTCGCTGGGCGGCTATGTGCTGTCGCTC
GTGCACGATGTGCGCTTCCACCACTTTCCCATCGAGCGCCAGCTCAACGG
CACCTACGCCATTGCCGGCGGCAAAGCGCACTGTGGACCGGCAGAGCTCT
GCGAGTTCTACTCGCGCGACCCCGACGGGCTGCCCTGCAACCTGCGCAAG
CCGTGCAACCGGCCGTCGGGCCTCGAGCCGCAGCCGGGGGTCTTCGACTG
CCTGCGAGACGCCATGGTGCGTGACTACGTGCGCCAGACGTGGAAGCTGG
AGGGCGAGGCCCTGGAGCAGGCCATCATCAGCCAGGCCCCGCAGGTGGAG
AAGCTCATTGCTACGACGGCCCACGAGCGGATGCCCTGGTACCACAGCAG
CCTGACGCGTGAGGAGGCCGAGCGCAAACTTTACTCTGGGGCGCAGACCG
ACGGCAAGTTCCTGCTGAGGCCGCGGAAGGAGCAGGGCACATACGCCCTG
TCCCTCATCTATGGGAAGACGGTGTACCACTACCTCATCAGCCAAGACAA
GGCGGGCAAGTACTGCATTCCCGAGGGCACCAAGTTTGACACGCTCTGGC
AGCTGGTGGAGTATCTGAAGCTGAAGGCGGACGGGCTCATCTACTGCCTG
AAGGAGGCCTGCCCCAACAGCAGTGCCAGCAACGCCTCAGGGGCTGCTGC
TCCCACACTCCCAGCCCACCCATCCACGTTGACG

In some embodiments, a dominant negative Zap70 moiety can have an amino acid sequence according to SEQ ID NO: 2. As disclosed herein, SEQ ID NO: 2 represents an exemplary amino acid sequence of a dominant negative Zap70 moiety. In some embodiments, a dominant negative Zap70 moiety can have an amino acid sequence substantially similar to SEQ ID NO: 2. In some embodiments, a dominant negative Zap70 moiety can have at least 75%, at least 80%, at least 85%, at least 90%, at least 95%, at least 96%, at least 97%, at least 98%, or at least 99% sequence identity to the amino acid sequence of SEQ ID NO: 2.

SEQ ID NO: 2
M P D P A A H L P F F Y G S I S R A E A E E H L K
L A G M A D G L F L L R Q C L R S L G G Y V L S L
V H D V R F H H F P I E R Q L N G T Y A I A G G K
A H C G P A E L C E F Y S R D P D G L P C N L R K
P C N R P S G L E P Q P G V F D C L R D A M V R D
Y V R Q T W K L E G E A L E Q A I I S Q A P Q V E
K L I A T T A H E R M P W Y H S S L T R E E A E R
K L Y S G A Q T D G K F L L R P R K E Q G T Y A L
S L I Y G K T V Y H Y L I S Q D K A G K Y C I P E
G T K F D T L W Q L V E Y L K L K A D G L I Y C L
K E A C P N S S A S N A S G A A A P T L P A H P S
T L T

In some embodiments, a dominant negative LCK moiety can be encoded by DNA having a nucleotide sequence according to SEQ ID NO: 19. As disclosed herein, SEQ ID NO: 19 represents an exemplary nucleotide sequence encoding a dominant negative LCK moiety. In some embodiments, a dominant negative LCK moiety can be encoded by DNA having a nucleotide sequence substantially similar to SEQ ID NO: 19. In some embodiments, a dominant negative LCK moiety can be encoded by DNA having a nucleotide sequence with at least 75%, at least 80%, at least 85%, at least 90%, at least 95%, at least 96%, at least 97%, at least 98%, or at least 99% sequence identity to the nucleotide sequence of SEQ ID NO: 19.

SEQ ID NO: 19
ATGGGCTGTGGCTGCAGCTCACACCCGGAAGATGACTGGATGGAAAACAT
CGATGTGTGTGAGAACTGCCATTATCCCATAGTCCCACTGGATGGCAAGG
GCACGCTGCTCATCCGAAATGGCTCTGAGGTGCGGGACCCACTGGTTACC
TACGAAGGCTCCAATCCGCCGGCTTCCCCACTGCAAGACAACCTGGTTAT
CGCTCTGCACAGCTATGAGCCCTCTCACGACGGAGATCTGGGCTTTGAGA
AGGGGGAACAGCTCCGCATCCTGGAGCAGAGCGGCGAGTGGTGGAAGGCG
CAGTCCCTGACCACGGGCCAGGAAGGCTTCATCCCCTTCAATTTTGTGGC
CAAAGCGAACAGCCTGGAGCCCGAACCCTGGTTCTTCAAGAACCTGAGCC
GCAAGGACGCGGAGCGGCAGCTCCTGGCGCCCGGGAACACTCACGGCTCC
TTCCTCATCCGGGAGAGCGAGAGCACCGCGGGATCGTTTTCACTGTCGGT
CCGGGACTTCGACCAGAACCAGGGAGAGGTGGTGAAACATTACAAGATCC
GTAATCTGGACAACGGTGGCTTCTACATCTCCCCTCGAATCACTTTTCCC
GGCCTGCATGAACTGGTCCGCCATTACACCAATGCTTCAGATGGGCTGTG
CACACGGTTGAGCCGCCCCTGCCAGACCCAGAAGCCCCAGAAGCCGTGGT
GGGAGGACGAGTGGGAGGTTCCCAGGGAGACGCTGAAGCTGGTGGAGCGG
CTGGGGGCTGGACAGTTCGGGGAGGTGTGGATGGGGTACTACAACGGG

In some embodiments, a dominant negative LCK moiety can have an amino acid sequence according to SEQ ID NO: 17. As disclosed herein, SEQ ID NO: 17 represents an exemplary amino acid sequence of a dominant negative LCK moiety. In some embodiments, a dominant negative LCK moiety can have an amino acid sequence substantially similar to SEQ ID NO: 17. In some embodiments, a dominant negative LCK moiety can have at least 75%, at least 80%, at least 85%, at least 90%, at least 95%, at least 96%, at least 97%, at least 98%, or at least 99% sequence identity to the amino acid sequence of SEQ ID NO: 17.

SEQ ID NO: 17
MGCGCSSHPEDDWMENIDVCENCHYPIVPLDGKGTLLIRNGSEVRDPLVT
YEGSNPPASPLQDNLVIALHSYEPSHDGDLGFEKGEQLRILEQSGEWWKA
QSLTTGQEGFIPFNFVAKANSLEPEPWFFKNLSRKDAERQLLAPGNTHGS
FLIRESESTAGSFSLSVRDFDQNQGEVVKHYKIRNLDNGGFYISPRITFP
GLHELVRHYTNASDGLCTRLSRPCQTQKPQKPWWEDEWEVPRETLKLVER
LGAGQFGEVWMGYYNG

Additional examples of dominant negative forms of signaling entities in a T cell activation pathway have been described and could be utilized in dominant negative signal moieties according to the present disclosure. See, e.g., Herskowitz, Functional inactivation of genes by dominant negative mutations,” Nature, Vo. 329 (1987). Examples of such dominant negative forms of signaling entities in a T cell activation pathway include: Lck (see, e.g., Levin, et al., A dominant-negative transgene defines a roles for p56lck in thymopoiesis, EMBO, 12(4), 1671-1680 (1993)), Ras (see, e.g., Stoll, et al., Dominant negative inhibitors of signaling through the phophoinositol 3-kinase pathway for gene therapy of pancreatic cancer, Gut, 54, 109-116 (2005)), PI3K (see, e.g., Pugazhenthi, S., et al., “Akt/Protein Kinase B Up-regulates Bcl-2 Expression through cAMP-response Element-binding Protein,” J Biol Chem, 275(15), 10761-10766 (2000)), PDK1 (see, e.g., Nirula, A., et al., “Phosphoinositide-dependent kinase 1 targets protein kinase A in a pathway that regulates interleukin 4,” JEM, 203(7), 1733-1744 (2006)), p38 MAPK (see, e.g., Somwar, R., et al., “A Dominant-negative p38 MAPK Mutant and Novel Selective Inhibitors of p38 MAPK Reduce Insulin-stimulated Glucose Uptake in 3T3-L1 Adipocytes without Affecting GLUT4 Translocation,” J Biol Chem, 277(52), 50386-50395 (2002)), MEK1 (Bastow, E., et al., “Selective Activation of the MEK-ERK Pathway Is Regulated by Mechanical Stimuli in Forming Joints and Promotes Pericellular Matrix Formation,” 280(12), 11749-11758 (2005)), and JNK1 and c-Raf (Chen, Y., et al., “The Role of c-Jun N-terminal Kinase (JNK) in Apoptosis Induced by Ultraviolet C and γ Radiation,” J Biol Chem, 271(50), 31929-31936 (1996)). In some embodiments, a dominant negative signaling moiety of a signaling entity may or may not correspond to an entire signaling entity. In other words, a dominant negative signaling moiety of a signaling entity may correspond to an entire signaling entity or a portion of a signaling entity (e.g., a fragment, a domain, a moiety, etc.). For example, a dominant negative signaling moiety of an enzymatic signaling entity may correspond to an entire enzymatic signaling entity, a fragment of an enzymatic signaling entity or a portion of an enzymatic signaling entity (e.g., a moiety of an enzymatic signaling entity (e.g., including an enzymatic domain) or an enzymatic domain).

In some embodiments, a dominant negative signaling moiety of a signaling entity may be produced by mutating a sequence (e.g., amino acid or nucleic acid sequence) of a signaling entity. Exemplary mutations include point mutations, additions and/or truncations. Mutations can be made in portions of a signaling entity associated with an activity (e.g., an enzymatic domain, such as a kinase domain); however, mutations are not limited to those portions of a signaling entity and may be made in a portion of a signaling entity that impacts, e.g., the conformation or cellular localization of a signaling entity.

In some embodiments, a dominant negative signaling moiety of a signaling entity may be produced by making post-translational modifications. Post-translational modifications can include, but are not limited to, ubiquitination, phosphorylation, acetylation, glycosylation (N- and O-linked), glycation, myristolyation, palmitoylation, prenylation, amidation, akylation, hydroxylation, biotinylation, pegylation, methylation, sulfation, SUMOylation, dephosphorylation, deacetylation, deglycosylation, deamidation, dihydroxylation, demethylation, deubiquitination, and/or desulfation. Post-translational modifications can be made in portions of a signaling entity associated with an activity (e.g., an enzymatic domain, such as a kinase domain); however, post-translational modifications may also be made in a portion of a signaling entity that impacts, e.g., the conformation or cellular localization of a signaling entity.

In some embodiments, a constitutively active signaling moiety based on a signaling entity in a T cell activation pathway (e.g., as listed in Table 4) can be used in a trigger-responsive constitutively active signaling polypeptide described here. Constitutively active moieties based on phosphatases within a T cell activation pathway are available and/or can be readily generated. SHP1 is a tyrosine phosphatase that dephosphorylates and deactivates both Zap70 and LCK.

In some embodiments, a constitutively active SHP1 moiety can be encoded by DNA having a nucleotide sequence according to SEQ ID NO: 25. As disclosed herein, SEQ ID NO: 25 represents an exemplary nucleotide sequence encoding a constitutively active SHP1 moiety. In some embodiments, a constitutively active SHP1 moiety can be encoded by DNA having a nucleotide sequence substantially similar to SEQ ID NO: 25. In some embodiments, a constitutively active SHP1 moiety can be encoded by DNA having a nucleotide sequence with at least 75%, at least 80%, at least 85%, at least 90%, at least 95%, at least 96%, at least 97%, at least 98%, or at least 99% sequence identity to the nucleotide sequence of SEQ ID NO: 25.

SEQ ID NO: 25
CGGCAGCCGTACTATGCCACGAGGGTGAATGCGGCTGACATTGAGAACCG
AGTGTTGGAACTGAACAAGAAGCAGGAGTCCGAGGATACAGCCAAGGCTG
GCTTCTGGGAGGAGTTTGAGAGTTTGCAGAAGCAGGAGGTGAAGAACTTG
CACCAGCGTCTGGAAGGGCAGCGGCCAGAGAACAAGGGCAAGAACCGCTA
CAAGAACATTCTCCCCTTTGACCACAGCCGAGTGATCCTGCAGGGACGGG
ACAGTAACATCCCCGGGTCCGACTACATCAATGCCAACTACATCAAGAAC
CAGCTGCTAGGCCCTGATGAGAACGCTAAGACCTACATCGCCAGCCAGGG
CTGTCTGGAGGCCACGGTCAATGACTTCTGGCAGATGGCGTGGCAGGAGA
ACAGCCGTGTCATCGTCATGACCACCCGAGAGGTGGAGAAAGGCCGGAAC
AAATGCGTCCCATACTGGCCCGAGGTGGGCATGCAGCGTGCTTATGGGCC
CTACTCTGTGACCAACTGCGGGGAGCATGACACAACCGAATACAAACTCC
GTACCTTACAGGTCTCCCCGCTGGACAATGGAGACCTGATTCGGGAGATC
TGGCATTACCAGTACCTGAGCTGGCCCGACCATGGGGTCCCCAGTGAGCC
TGGGGGTGTCCTCAGCTTCCTGGACCAGATCAACCAGCGGCAGGAAAGTC
TGCCTCACGCAGGGCCCATCATCGTGCACTGCAGCGCCGGCATCGGCCGC
ACAGGCACCATCATTGTCATCGACATGCTCATGGAGAACATCTCCACCAA
GGGCCTGGACTGTGACATTGACATCCAGAAGACCATCCAGATGGTGCGGG
CGCAGCGCTCGGGCATGGTGCAGACGGAGGCGCAGTACAAGTTCATCTAC
GTGGCCATCGCCCAGTTCATTGAAACCACTAAGAAGAAGCTGGAGGTCCT
GCAGTCGCAGAAGGGCCAGGAGTCGGAGTACGGGAACATCACCTATCCCC
CAGCCATGAAGAATGCCCATGCCAAGGCCTCCCGCACCTCGTCCAAACAC
AAGGAGGATGTGTATGAGAACCTGCACACTAAGAACAAGAGGGAGGAGAA
AGTGAAGAAGCAGCGGTCAGCAGACAAGGAGAAGAGCAAGGGTTCCCTCA
AGAGGAAG

In some embodiments, a constitutively active SHP1 can have an amino acid sequence according to SEQ ID NO: 23. As disclosed herein, SEQ ID NO: 23 represents an exemplary amino acid sequence of a constitutively active SHP1 moiety. In some embodiments, a constitutively active SHP1 moiety can have an amino acid sequence substantially similar to SEQ ID NO: 23. In some embodiments, a constitutively active SHP1 moiety can have at least 75%, at least 80%, at least 85%, at least 90%, at least 95%, at least 96%, at least 97%, at least 98%, or at least 99% sequence identity to the amino acid sequence of SEQ ID NO: 23.

SEQ ID NO: 23
RQPYYATRVNAADIENRVLELNKKQESEDTAKAGFWEEFESLQKQEVKNL
HQRLEGQRPENKGKNRYKNILPFDHSRVILQGRDSNIPGSDYINANYIKN
QLLGPDENAKTYIASQGCLEATVNDFWQMAWQENSRVIVMTTREVEKGRN
KCVPYWPEVGMQRAYGPYSVTNCGEHDTTEYKLRTLQVSPLDNGDLIREI
WHYQYLSWPDHGVPSEPGGVLSFLDQINQRQESLPHAGPIIVHCSAGIGR
TGTIIVIDMLMENISTKGLDCDIDIQKTIQMVRAQRSGMVQTEAQYKFIY
VAIAQFIETTKKKLEVLQSQKGQESEYGNITYPPAMKNAHAKASRTSSKH
KEDVYENLHTKNKREEKVKKQRSADKEKSKGSLKRK

In some embodiments, a constitutively active signaling moiety of a signaling entity may or may not correspond to an entire signaling entity. In other words, a constitutively active signaling moiety of a signaling entity may correspond to an entire signaling entity or a portion of a signaling entity (e.g., a fragment, a domain, a moiety, etc.). For example, a constitutively active signaling moiety of an enzymatic signaling entity may correspond to an entire enzymatic signaling entity, a fragment of an enzymatic signaling entity or a portion of an enzymatic signaling entity (e.g., a moiety of an enzymatic signaling entity (e.g., including an enzymatic domain) or an enzymatic domain).

In some embodiments, a constitutively active signaling moiety of a signaling entity may be produced by mutating a sequence (e.g., amino acid or nucleic acid sequence) of a signaling entity. Exemplary mutations include point mutations, additions and/or truncations. Mutations can be made in portions of a signaling entity associated with an activity (e.g., an enzymatic domain, such as a phosphatase domain); however, mutations are not limited to those portions of a signaling entity and may be made in a portion of a signaling entity that impacts, e.g., the conformation or cellular localization of a signaling entity.

In some embodiments, a constitutively active signaling moiety of a signaling entity may be produced by making post-translational modifications. Post-translational modifications can include, but are not limited to, ubiquitination, phosphorylation, acetylation, glycosylation (N- and O-linked), glycation, myristolyation, palmitoylation, prenylation, amidation, akylation, hydroxylation, biotinylation, pegylation, methylation, sulfation, SUMOylation, dephosphorylation, deacetylation, deglycosylation, deamidation, dihydroxylation, demethylation, deubiquitination, and/or desulfation. Post-translational modifications can be made in portions of a signaling entity associated with an activity (e.g., an enzymatic domain, such as a phosphatase domain); however, post-translational modifications may also be made in a portion of a signaling entity that impacts, e.g., the conformation or cellular localization of a signaling entity.

### Trigger-Responsive Immune-Inactivating Signaling Polypeptides

Among other things, the present disclosure provides a trigger-responsive immune-inactivating signaling polypeptide, which can adopt at least first and second state (e.g., conformations), and can switch from one to the other in response to a particular trigger. In some embodiments, a trigger-responsive immune-inactivating signaling polypeptide is inhibited in one state relative to the other state. In some embodiments, when a trigger-responsive immune-inactivating signaling polypeptide is in its second state, the inhibition is relieved. A trigger-responsive immune-inactivating signaling polypeptide can transition between the first state and the second state when exposed to a trigger.

In some embodiments, a trigger-responsive immune-inactivating signaling polypeptide (which can be, for example, a fusion polypeptide) can include a modulating domain. A modulating domain can be characterized by an ability to adopt a first state and a second state. In some embodiments, a modulating domain can transition between the first state and the second state when exposed to a trigger. A modulating domain can be a portion of the trigger-responsive immune-inactivating signaling polypeptide that can change conformations, e.g., between a first and second conformation, preferably in response to a particular trigger. The present disclosure recognizes that a modulating domain can be utilized to inhibit, mask and/or inactivate, in a trigger responsive manner, an immune-inactivating moiety.

Among other things, the present disclosure provides a trigger-responsive dominant negative signaling polypeptide, which can adopt at least first and second state (e.g., conformations), and can switch from one to the other in response to a particular trigger. In some embodiments, a trigger-responsive dominant negative signaling polypeptide is inhibited in one state relative to the other state. In some embodiments, when a trigger-responsive dominant negative signaling polypeptide is in its second state, the inhibition is relieved. A trigger-responsive dominant negative signaling polypeptide can transition between the first state and the second state when exposed to a trigger.

In some embodiments, a trigger-responsive dominant negative signaling polypeptide (which can be, for example, a fusion polypeptide) can include a modulating domain. A modulating domain can be characterized by an ability to adopt a first state and a second state. In some embodiments, a modulating domain can transition between the first state and the second state when exposed to a trigger. A modulating domain can be a portion of the trigger-responsive dominant negative signaling polypeptide that can change conformations, e.g., between a first and second conformation, preferably in response to a particular trigger. The present disclosure recognizes that a modulating domain can be utilized to inhibit, mask and/or inactivate, in a trigger responsive manner, a dominant negative signaling moiety.

Among other things, the present disclosure also provides a trigger-responsive constitutively active signaling polypeptide, which can adopt at least first and second state (e.g., conformations), and can switch from one to the other in response to a particular trigger. In some embodiments, a trigger-responsive constitutively active signaling polypeptide is inhibited in one state relative to the other state. In some embodiments, when a trigger-responsive constitutively active signaling polypeptide is in its second state, the inhibition is relieved. A trigger-responsive constitutively active signaling polypeptide can transition between the first state and the second state when exposed to a trigger.

In some embodiments, a trigger-responsive constitutively active signaling polypeptide (which can be, for example, a fusion polypeptide) can include a modulating domain. A modulating domain can be characterized by an ability to adopt a first state and a second state. In some embodiments, a modulating domain can transition between the first state and the second state when exposed to a trigger. A modulating domain can be a portion of the trigger-responsive constitutively active signaling polypeptide that can change conformations, e.g., between a first and second conformation, preferably in response to a particular trigger. The present disclosure recognizes that a modulating domain can be utilized to inhibit, mask and/or inactivate, in a trigger responsive manner, a constitutively active signaling moiety.

### Modulating Domain

The present disclosure utilizes the insight that ligand binding domains of certain nuclear receptors have been demonstrated to effectively mask or inactivate, in a ligand-binding-dependent-manner, activity of polypeptide agents with which they are operatively associated. For example, Feil, et al. demonstrated the use of an ER(T2) mutated ligand binding domain fragment of human estrogen receptor-α to control the activity of a fusion protein that also included CRE recombinase. (Fiel, et al., Regulation of Cre Recombinase Activity by Mutated Estrogen Receptor Ligand-Binding Domains, Biochem and Biophys Research Comms, 752-757 (1997)). The fusion protein of Feil, et al. has been used to perform tamoxifen mediated excision of target genes in mice and other organisms. There has also been a report of the use of ER(T2) to allow for tamoxifen control the activity of a protein that is located in the cytoplasm, the BRAF kinase (see, for example, Ortiz et al. Genesis 51:448, June 2013; epub Mar. 28, 2013).

The present disclosure encompasses the recognition that association of such a modulating domain with a dominant negative signaling moiety (e.g., a dominant negative kinase moiety) as described herein can create a trigger-responsive dominant negative signaling polypeptide (e.g., a trigger-responsive dominant negative kinase polypeptide) useful, e.g., to allow for trigger (e.g., ligand) mediated control of activity of a dominant negative signaling moiety (e.g., such as in modulating T cell activity) either in the nucleus or the cytoplasm. Such an application requires a large dynamic range of regulation. For example, it may be necessary for a dominant negative signaling moiety to be mostly or completely inactive in the absence of a trigger and be highly active in the presence of a trigger, e.g., to overcome the activity of a corresponding signaling entity (e.g., a wild-type or endogenous signaling entity). In certain embodiments, an activity of a dominant negative signaling moiety in a trigger-responsive dominant negative signaling polypeptide is regulated in a trigger dose-dependent manner. Among other things, present disclosure utilizes the discovery that trigger-responsive dominant negative signaling polypeptide described herein (e.g., including a dominant negative Zap70 moiety operatively linked to an ER(T2) or ER(T12) domain) has a dynamic range needed to effectively regulate T cells activated by antigen in a finely-tuned manner.

The present disclosure also encompasses the recognition that association of such a modulating domain with a constitutively active signaling moiety (e.g., a constitutively active phosphatase moiety) as described herein can create a trigger-responsive constitutively active signaling polypeptide (e.g., a trigger-responsive constitutively active phosphatase polypeptide) useful, e.g., to allow for trigger (e.g., ligand) mediated control of activity of a constitutively active signaling moiety (e.g., such as in modulating T cell activity) either in the nucleus or the cytoplasm. Such an application requires a large dynamic range of regulation. For example, it may be necessary for a constitutively active signaling moiety to be mostly or completely inactive in the absence of a trigger and be highly active in the presence of a trigger. In certain embodiments, an activity of a constitutively active signaling moiety in a trigger-responsive constitutively active signaling polypeptide is regulated in a trigger dose-dependent manner. Among other things, present disclosure utilizes the discovery that trigger-responsive constitutively active signaling polypeptide described herein (e.g., including a constitutively active SHP1 moiety operatively linked to an ER(T2) or ER(T12) domain) has a dynamic range needed to effectively regulate T cells activated by antigen in a finely-tuned manner.

In some embodiments, a modulating domain for use in accordance with the present disclosure comprises a nuclear receptor or a portion thereof. In some embodiments, a nuclear receptor can include a thyroid hormone receptor (e.g. a thyroid hormone receptor-α or a thyroid hormone receptor-ß), a retinoic acid receptor (e.g., a retinoic acid receptor-α, a retinoic acid receptor-ß, or a retinoic acid receptor-γ), a peroxisome proliferator-activated receptor (e.g., a peroxisome proliferator-activated receptor-α, a peroxisome proliferator-activated receptor-ß, or a peroxisome proliferator-activated receptor-γ), a Rev-ErbA receptor, a RAR-related orphan receptor (e.g., a RAR-related orphan receptor-α, a RAR-related orphan receptor-ß, or a RAR-related orphan receptor-γ), a liver X receptor (e.g., a liver X receptor-α or a liver X receptor-ß), a farnesoid X receptor (e.g., a farnesoid X receptor-α or a farnesoid X receptor-ß), a vitamin D receptor, a pregnane X receptor, an androstane receptor, a hepatocyte nuclear factor-4 receptor (e.g., hepatocyte nuclear factor-4-α receptor or hepatocyte nuclear factor-4-γ receptor), a retinoid X receptor (e.g., a retinoid X receptor-α, a retinoid X receptor-ß, or a retinoid X receptor-γ), a testicular receptor (e.g., a testicular receptor 2 or a testicular receptor 4), an estrogen receptor (e.g., an estrogen receptor-α or an estrogen receptor-ß), an estrogen-related receptor (e.g., an estrogen-related receptor-α, an estrogen-related receptor-ß, or an estrogen-related receptor-γ), a glucocorticoid receptor, a mineralocorticoid receptor, a progesterone receptor, or an androgen receptor. In some embodiments, a modulating domain includes a steroid hormone receptor or a portion thereof. In certain embodiments, a modulating domain includes an estrogen receptor or portion thereof; in some such embodiments, a modulating domain includes an estrogen receptor-α or portion thereof.

In some embodiments, a nuclear receptor is a mammalian nuclear receptor, preferably, a human nuclear receptor. In some embodiments, a nuclear receptor can be a mammalian wild-type nuclear receptor, for example, a human wild-type nuclear receptor. In some embodiments, a nuclear receptor is a homolog of a human nuclear receptor. In some embodiments, a nuclear receptor can be a nuclear receptor variant.

Canonical nucleotide sequences that encode for nuclear receptors (e.g., wild-type nuclear receptors) are known to those of skill in the art. In some embodiments, a nuclear receptor can be encoded by DNA having a nucleotide sequence substantially similar to a canonical nucleotide sequence encoding for the nuclear receptor. In some embodiments, a nuclear receptor can be encoded by DNA having a nucleotide sequence with at least 75%, at least 80%, at least 85%, at least 90%, at least 95%, at least 96%, at least 97%, at least 98%, or at least 99% sequence identity to a canonical nucleotide sequence for the nuclear receptor.

In some embodiments, a nuclear receptor can be a hormone receptor. In some embodiments, a hormone receptor can be an estrogen receptor-α, e.g., a human estrogen receptor-α. In some embodiments, an estrogen receptor-α can be encoded by DNA having a nucleotide sequence according to SEQ ID NO: 11. As disclosed herein, SEQ ID NO: 11 represents an exemplary nucleotide sequence encoding an estrogen receptor-α. In some embodiments, an estrogen receptor-α can be encoded by DNA having a nucleotide sequence substantially similar to SEQ ID NO: 11. In some embodiments, an estrogen receptor-α can be encoded by DNA having a nucleotide sequence with at least 75%, at least 80%, at least 85%, at least 90%, at least 95%, at least 96%, at least 97%, at least 98%, or at least 99% sequence identity to the nucleotide sequence of SEQ ID NO: 11.

SEQ ID NO: 11
ATGACCATGACCCTCCACACCAAAGCATCTGGGATGGCCCTACTGCATCA
GATCCAAGGGAACGAGCTGGAGCCCCTGAACCGTCCGCAGCTCAAGATCC
CCCTGGAGCGGCCCCTGGGCGAGGTGTACCTGGACAGCAGCAAGCCCGCC
GTGTACAACTACCCCGAGGGCGCCGCCTACGAGTTCAACGCCGCGGCCGC
CGCCAACGCGCAGGTCTACGGTCAGACCGGCCTCCCCTACGGCCCCGGGT
CTGAGGCTGCGGCGTTCGGCTCCAACGGCCTGGGGGGTTTCCCCCCACTC
AACAGCGTGTCTCCGAGCCCGCTGATGCTACTGCACCCGCCGCCGCAGCT
GTCGCCTTTCCTGCAGCCCCACGGCCAGCAGGTGCCCTACTACCTGGAGA
ACGAGCCCAGCGGCTACACGGTGCGCGAGGCCGGCCCGCCGGCATTCTAC
AGGCCAAATTCAGATAATCGACGCCAGGGTGGCAGAGAAAGATTGGCCAG
TACCAATGACAAGGGAAGTATGGCTATGGAATCTGCCAAGGAGACTCGCT
ACTGTGCAGTGTGCAATGACTATGCTTCAGGCTACCATTATGGAGTCTGG
TCCTGTGAGGGCTGCAAGGCCTTCTTCAAGAGAAGTATTCAAGGACATAA
CGACTATATGTGTCCAGCCACCAACCAGTGCACCATTGATAAAAACAGGA
GGAAGAGCTGCCAGGCCTGCCGGCTCCGCAAATGCTACGAAGTGGGAATG
ATGAAAGGTGGGATACGAAAAGACCGAAGAGGAGGGAGAATGTTGAAACA
CAAGCGCCAGAGAGATGATGGGGAGGGCAGGGGTGAAGTGGGGTCTGCTG
GAGACATGAGAGCTGCCAACCTTTGGCCAAGCCCGCTCATGATCAAACGC
TCTAAGAAGAACAGCCTGGCCTTGTCCCTGACGGCCGACCAGATGGTCAG
TGCCTTGTTGGATGCTGAGCCCCCCATACTCTATTCCGAGTATGATCCTA
CCAGACCCTTCAGTGAAGCTTCGATGATGGGCTTACTGACCAACCTGGCA
GACAGGGAGCTGGTTCACATGATCAACTGGGCGAAGAGGGTGCCAGGCTT
TGTGGATTTGACCCTCCATGATCAGGTCCACCTTCTAGAATGTGCCTGGC
TAGAGATCCTGATGATTGGTCTCGTCTGGCGCTCCATGGAGCACCCAGGG
AAGCTACTGTTTGCTCCTAACTTGCTCTTGGACAGGAACCAGGGAAAATG
TGTAGAGGGCATGGTGGAGATCTTCGACATGCTGCTGGCTACATCATCTC
GGTTCCGCATGATGAATCTGCAGGGAGAGGAGTTTGTGTGCCTCAAATCT
ATTATTTTGCTTAATTCTGGAGTGTACACATTTCTGTCCAGCACCCTGAA
GTCTCTGGAAGAGAAGGACCATATCCACCGAGTCCTGGACAAGATCACAG
ACACTTTGATCCACCTGATGGCCAAGGCAGGCCTGACCCTGCAGCAGCAG
CACCAGCGGCTGGCCCAGCTCCTCCTCATCCTCTCCCACATCAGGCACAT
GAGTAACAAAGGCATGGAGCATCTGTACAGCATGAAGTGCAAGAACGTGG
TGCCCCTCTATGACCTGCTGCTGGAGATGCTGGACGCCCACCGCCTACAT
GCGCCCACTAGCCGTGGAGGGGCATCCGTGGAGGAGACGGACCAAAGCCA
CTTGGCCACTGCGGGCTCTACTTCATCGCATTCCTTGCAAAAGTATTACA
TCACGGGGGAGGCAGAGGGTTTCCCTGCCACGGTCTGA

Canonical amino acid sequences for nuclear receptors (e.g., wild-type nuclear receptors) are known to those of skill in the art. In some embodiments, a nuclear receptor can have an amino acid sequence substantially similar to a canonical amino acid sequence for the nuclear receptor. In some embodiments, a nuclear receptor can have an amino acid sequence with at least 75%, at least 80%, at least 85%, at least 90%, at least 95%, at least 96%, at least 97%, at least 98%, or at least 99% sequence identity to a canonical amino acid sequence for the nuclear receptor.

In some embodiments, a nuclear receptor can be a hormone receptor. In some embodiments, a hormone receptor can be an estrogen receptor-α, e.g., a human estrogen receptor-α. In some embodiments, an estrogen receptor-α can have an amino acid sequence according to SEQ ID NO: 12. As disclosed herein, SEQ ID NO: 12 represents an exemplary amino acid sequence of an estrogen receptor-α. In some embodiments, an estrogen receptor-α can have an amino acid sequence substantially similar to SEQ ID NO: 12. In some embodiments, an estrogen receptor-α can have at least 75%, at least 80%, at least 85%, at least 90%, at least 95%, at least 96%, at least 97%, at least 98%, or at least 99% sequence identity to the amino acid sequence of SEQ ID NO: 12.

SEQ ID NO: 12
MTMTLHTKASGMALLHQIQGNELEPLNRPQLKIPLERPLGEVYLDSSKPA
VYNYPEGAAYEFNAAAAANAQVYGQTGLPYGPGSEAAAFGSNGLGGFPPL
NSVSPSPLMLLHPPPQLSPFLQPHGQQVPYYLENEPSGYTVREAGPPAFY
RPNSDNRRQGGRERLASTNDKGSMAMESAKETRYCAVCNDYASGYHYGVW
SCEGCKAFFKRSIQGHNDYMCPATNQCTIDKNRRKSCQACRLRKCYEVGM
MKGGIRKDRRGGRMLKHKRQRDDGEGRGEVGSAGDMRAANLWPSPLMIKR
SKKNSLALSLTADQMVSALLDAEPPILYSEYDPTRPFSEASMMGLLTNLA
DRELVHMINWAKRVPGFVDLTLHDQVHLLECAWLEILMIGLVWRSMEHPG
KLLFAPNLLLDRNQGKCVEGMVEIFDMLLATSSRFRMMNLQGEEFVCLKS
IILLNSGVYTFLSSTLKSLEEKDHIHRVLDKITDTLIHLMAKAGLTLQQQ
HQRLAQLLLILSHIRHMSNKGMEHLYSMKCKNVVPLYDLLLEMLDAHRLH
APTSRGGASVEETDQSHLATAGSTSSHSLQKYYITGEAEGFPATV

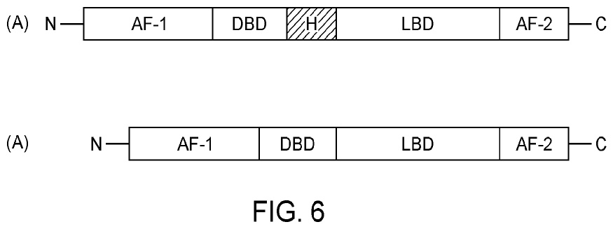

In some embodiments, a modulating domain is a portion of a nuclear receptor. In some embodiments, a modulating domain can comprise one or more domains of a nuclear receptor. Generally, nuclear receptors are characterized as including five domains: an activation function 1 domain, a DNA binding domain, a hinge domain, a ligand binding domain, and an activation function 2 domain, as shown in FIG. 6A.

In some embodiments, a modulating domain can include a ligand binding domain of a nuclear receptor. In certain embodiments, a modulating domain includes an estrogen receptor ligand binding domain, preferably, an estrogen receptor-α ligand binding domain.

Canonical nucleotide sequences that encode for ligand binding domains of nuclear receptors (e.g., wild-type nuclear receptors) are known to those of skill in the art. In some embodiments, a ligand binding domain of a nuclear receptor can be encoded by DNA having a nucleotide sequence substantially similar to a canonical nucleotide sequence encoding for a ligand binding domain of the nuclear receptor. For example, a ligand binding domain of an estrogen receptor-α of the present disclosure can be encoded by DNA having a nucleotide sequence substantially similar to a canonical nucleotide sequence encoding for a ligand binding domain of an estrogen receptor-α. In some embodiments, a nuclear receptor can be encoded by DNA having a nucleotide sequence with at least 75%, at least 80%, at least 85%, at least 90%, at least 95%, at least 96%, at least 97%, at least 98%, or at least 99% sequence identity to a canonical nucleotide sequence for a ligand binding domain of the nuclear receptor. In some embodiments, a ligand binding domain of an estrogen receptor-α of the present disclosure can be encoded by DNA having a nucleotide sequence substantially similar to a nucleotide sequence comprising or consisting essentially of nucleotides 984 to 1784 of SEQ ID NO: 11. In some embodiments, a nuclear receptor can be encoded by DNA having a nucleotide sequence with at least 75%, at least 80%, at least 85%, at least 90%, at least 95%, at least 96%, at least 97%, at least 98%, or at least 99% sequence identity to a nucleotide sequence comprising or consisting essentially of nucleotides 984 to 1784 of SEQ ID NO: 11.

Canonical amino acid sequences for ligand binding domains of nuclear receptors (e.g., wild-type nuclear receptors) are known to those of skill in the art. In some embodiments, a ligand binding domain of a nuclear receptor can have an amino acid sequence substantially similar to a canonical amino acid sequence for a ligand binding domain of the nuclear receptor. For example, a ligand binding domain of an estrogen receptor-α of the present disclosure can have an amino acid sequence substantially similar to a canonical amino acid sequence of a ligand binding domain of an estrogen receptor-α. In some embodiments, a nuclear receptor can have an amino acid sequence with at least 75%, at least 80%, at least 85%, at least 90%, at least 95%, at least 96%, at least 97%, at least 98%, or at least 99% sequence identity to a canonical amino acid sequence for a ligand binding domain of the nuclear receptor. In some embodiments, a ligand binding domain of an estrogen receptor-α of the present disclosure can have an amino acid sequence substantially similar to an amino acid sequence comprising or consisting essentially of amino acids 302 to 595 of SEQ ID NO: 12. In some embodiments, a nuclear receptor can have an amino acid sequence with at least 75%, at least 80%, at least 85%, at least 90%, at least 95%, at least 96%, at least 97%, at least 98%, or at least 99% sequence identity to an amino acid sequence comprising or consisting essentially of amino acids 302 to 595 of SEQ ID NO: 12.

In some embodiments, a modulating domain includes an estrogen receptor ligand binding domain variant. In some embodiments, a modulating domain includes an estrogen receptor-α ligand binding domain variant, such as ER(T2) or ER(T12).

The present disclosure provides insight that estrogen receptor variants or fragments thereof are effective modulating domains. Furthermore, the present disclosure provides the insight that modulating domains that include estrogen receptor or fragment thereof with a mutation at residue 400 in SEQ ID NO: 12 (which corresponds, e.g., to residue 119 in SEQ ID NO: 4 and residue 415 in SEQ ID NO: 8) may be particularly useful. In some embodiments, a modulating domain includes an estrogen receptor or fragment thereof comprising an amino acid substitution at position G400 of SEQ ID NO: 12. In some embodiments, a modulating domain includes an estrogen receptor or fragment thereof comprising an amino acid substitution at position G400 of SEQ ID NO: 12 with V, M, A, L, or I.

In some embodiments, a modulating domain includes an estrogen receptor or fragment thereof comprising at least one mutation at a residue corresponding to residue 400, residue 521, residue 539, residue 540, residue 543, and/or residue 544 of SEQ ID NO: 12. Residue 521 of SEQ ID NO: 12 corresponds to residue 240 in SEQ ID NO: 4 and residue 536 in SEQ ID NO: 8. Residue 539 of SEQ ID NO: 12 corresponds to residue 258 in SEQ ID NO: 4 and residue 554 in SEQ ID NO: 8. Residue 540 of SEQ ID NO: 12 corresponds to residue 259 in SEQ ID NO: 4 and residue 555 in SEQ ID NO: 8. Residue 543 of SEQ ID NO: 12 corresponds to residue 262 in SEQ ID NO: 4 and residue 558 in SEQ ID NO: 8, and residue 544 of SEQ ID NO: 12 corresponds to residue 263 in SEQ ID NO: 4 and residue 559 in SEQ ID NO: 8. In some embodiments, a modulating domain includes an estrogen receptor or fragment thereof comprising at least one mutation selected from the group consisting of G400V, G400M, G400A, G400L, G400I, G521R, G521T, L539A, L540A, M543A and L544A, wherein the residue numbering is based on SEQ ID NO: 12. In some embodiments, a modulating domain includes an estrogen receptor or fragment thereof comprising at least one mutation that is either G400V or G400L, wherein the residue numbering is based on SEQ ID NO: 12.

The present disclosure provides the insight that certain combinations of mutations in an estrogen receptor or fragment thereof are particularly advantageous. For example, in some embodiments, a modulating domain includes an estrogen receptor or fragment thereof comprising at least one mutation selected from the group consisting of G400V, G400M, G400A, G400L, G400I, G521R, and G521T, wherein the residue numbering is based on SEQ ID NO: 12. In certain embodiments, a modulating domain includes an estrogen receptor or fragment thereof comprising at least one mutation that is either G400V or G400L, wherein the residue numbering is based on SEQ ID NO: 12. Without wishing to be bound to any particular theory, mutations at residues corresponding to residues 400 and/or 521 of SEQ ID NO: 12 can facilitate an interaction with heat shock proteins, such as, Hsp90. In some embodiments, a modulating domain includes an estrogen receptor or fragment thereof comprising a second mutation selected from L539A and L540A, wherein the residue numbering is based on SEQ ID NO: 12. In some embodiments, the estrogen receptor or fragment thereof of the modulating domain comprises a second or additional mutation selected from M543A and L544A, wherein the residue numbering is based on SEQ ID NO: 12. Without wishing to be bound to any particular theory, mutations at residues corresponding to residues 539, 540, 543, and/or 544 of SEQ ID NO: 12 can abolish or diminish binding of the binding between estradiol (e.g., 17-beta estradiol) and a ligand binding domain of an estrogen receptor, without affecting or minimally affecting binding between endoxifen or other tamoxifen metabolites and a ligand binding domain of an estrogen receptor.

In some embodiments, mutation(s) in an estrogen receptor or fragment thereof confer increased affinity for at least one chaperone protein, e.g., Hsp27, Hsp70, and Hsp90. In some embodiments, an estrogen receptor ligand binding domain variant includes mutations that confer on the estrogen receptor ligand binding domain a reduced affinity to at least one naturally occurring estrogen, e.g., estradiol (e.g., 17-beta estradiol), estrone, or estriol. In some embodiments, an estrogen receptor ligand binding domain variant includes mutations that confer on the estrogen receptor ligand binding domain preferential binding to at least one synthetic estrogen receptor ligand, e.g., tamoxifen, endoxifen, or 4-hydroxytamoxifen.

In some embodiments, an ER(T2) domain can be encoded by DNA having a nucleotide sequence according to SEQ ID NO: 3. As disclosed herein, SEQ ID NO: 3 represents an exemplary nucleotide sequence encoding an ER(T2) domain. In some embodiments, an ER(T2) domain can be encoded by DNA having a nucleotide sequence substantially similar to SEQ ID NO: 3. In some embodiments, an ER(T2) domain can be encoded by DNA having a nucleotide sequence with at least 75%, at least 80%, at least 85%, at least 90%, at least 95%, at least 96%, at least 97%, at least 98%, or at least 99% sequence identity to the nucleotide sequence of SEQ ID NO: 3.

SEQ ID NO: 3
TCTGCTGGAGACATGAGAGCTGCCAACCTTTGGCC
AAGCCCGCTCATGATCAAACGCTCTAAGAAGAACA
GCCTGGCCTTGTCCCTGACGGCCGACCAGATGGTC
AGTGCCTTGTTGGATGCTGAGCCCCCCATACTCTA
TTCCGAGTATGATCCTACCAGACCCTTCAGTGAAG
CTTCGATGATGGGCTTACTGACCAACCTGGCAGAC
AGGGAGCTGGTTCACATGATCAACTGGGCGAAGAG
GGTGCCAGGCTTTGTGGATTTGACCCTCCATGATC
AGGTCCACCTTCTAGAATGTGCCTGGCTAGAGATC
CTGATGATTGGTCTCGTCTGGCGCTCCATGGAGCA
CCCAGTGAAGCTACTGTTTGCTCCTAACTTGCTCT
TGGACAGGAACCAGGGAAAATGTGTAGAGGGCATG
GTGGAGATCTTCGACATGCTGCTGGCTACATCATC
TCGGTTCCGCATGATGAATCTGCAGGGAGAGGAGT
TTGTGTGCCTCAAATCTATTATTTTGCTTAATTCT
GGAGTGTACACATTTCTGTCCAGCACCCTGAAGTC
TCTGGAAGAGAAGGACCATATCCACCGAGTCCTGG
ACAAGATCACAGACACTTTGATCCACCTGATGGCC
AAGGCAGGCCTGACCCTGCAGCAGCAGCACCAGCG
GCTGGCCCAGCTCCTCCTCATCCTCTCCCACATCA
GGCACATGAGTAACAAAGGCATGGAGCATCTGTAC
AGCATGAAGTGCAAGAACGTGGTGCCCCTCTATGA
CCTGCTGCTGGAGGCGGCGGACGCCCACCGCCTAC
ATGCGCCCACTAGCCGTGGAGGGGCATCCGTGGAG
GAGACGGACCAAAGCCACTTGGCCACTGCGGGCTC
TACTTCATCGCATTCCTTGCAAAAGTATTACATCA
CGGGGGAGGCAGAGGGTTTCCCTGCCACGGTC

In some embodiments, an ER(T2) domain can have an amino acid sequence according to SEQ ID NO: 4. As disclosed herein, SEQ ID NO: 4 represents an exemplary amino acid sequence of an ER(T2) domain. In some embodiments, an ER(T2) domain can have an amino acid sequence substantially similar to SEQ ID NO: 4. In some embodiments, an ER(T2) domain can have at least 75%, at least 80%, at least 85%, at least 90%, at least 95%, at least 96%, at least 97%, at least 98%, or at least 99% sequence identity to the amino acid sequence of SEQ ID NO: 4.

SEQ ID NO: 4
S A G D M R A A N L W P S P L M I K
R S K K N S L A L S L T A D Q M V S
A L L D A E P P I L Y S E Y D P T R
P F S E A S M M G L L T N L A D R E
L V H M I N W A K R V P G F V D L T
L H D Q V H L L E C A W L E I L M I
G L V W R S M E H P V K L L F A P N
L L L D R N Q G K C V E G M V E I F
D M L L A T S S R F R M M N L Q G E
E F V C L K S I I L L N S G V Y T F
L S S T L K S L E E K D H I H R V L
D K I T D T L I H L M A K A G L T L
Q Q Q H Q R L A Q L L L I L S H I R
H M S N K G M E H L Y S M K C K N V
V P L Y D L L L E A A D A H R L H A
P T S R G G A S V E E T D Q S H L A
T A G S T S S H S L Q K Y Y I T G E
A E G F P A T V

In some embodiments, an ER(T12) domain can be encoded by DNA having a nucleotide sequence according to SEQ ID NO: 14. As disclosed herein, SEQ ID NO: 14 represents an exemplary nucleotide sequence encoding an ER(T12) domain. In some embodiments, an ER(T12) domain can be encoded by DNA having a nucleotide sequence substantially similar to SEQ ID NO: 14. In some embodiments, an ER(T12) domain can be encoded by DNA having a nucleotide sequence with at least 75%, at least 80%, at least 85%, at least 90%, at least 95%, at least 96%, at least 97%, at least 98%, or at least 99% sequence identity to the nucleotide sequence of SEQ ID NO: 14.

SEQ ID NO: 14
TCTGCTGGAGACATGAGAGCTGCCAACCTTTGGCC
AAGCCCGCTCATGATCAAACGCTCTAAGAAGAACA
GCCTGGCCTTGTCCCTGACGGCCGACCAGATGGTC
AGTGCCTTGTTGGATGCTGAGCCCCCCATACTCTA
TTCCGAGTATGATCCTACCAGACCCTTCAGTGAAG
CTTCGATGATGGGCTTACTGACCAACCTGGCAGAC
AGGGAGCTGGTTCACATGATCAACTGGGCGAAGAG
GGTGCCAGGCTTTGTGGATTTGACCCTCCATGATC
AGGTCCACCTTCTAGAATGTGCCTGGCTAGAGATC
CTGATGATTGGTCTCGTCTGGCGCTCCATGGAGCA
CCCActgAAGCTACTGTTTGCTCCTAACTTGCTCT
TGGACAGGAACCAGGGAAAATGTGTAGAGGGCATG
GTGGAGATCTTCGACATGCTGCTGGCTACATCATC
TCGGTTCCGCATGATGAATCTGCAGGGAGAGGAGT
TTGTGTGCCTCAAATCTATTATTTTGCTTAATTCT
GGAGTGTACACATTTCTGTCCAGCACCCTGAAGTC
TCTGGAAGAGAAGGACCATATCCACCGAGTCCTGG
ACAAGATCACAGACACTTTGATCCACCTGATGGCC
AAGGCAGGCCTGACCCTGCAGCAGCAGCACCAGCG
GCTGGCCCAGCTCCTCCTCATCCTCTCCCACATCA
GGCACATGAGTAACAAAGGCATGGAGCATCTGTAC
AGCATGAAGTGCAAGAACGTGGTGCCCCTCTATGA
CCTGCTGCTGGAGgcggcgGACGCCCACCGCCTAC
ATGCGCCCACTAGCCGTGGAGGGGCATCCGTGGAG
GAGACGGACCAAAGCCACTTGGCCACTGCGGGCTC
TACTTCATCGCATTCCTTGCAAAAGTATTACATCA
CGGGGGAGGCAGAGGGTTTCCCTGCCACGGTC

In some embodiments, an ER(T12) domain can have an amino acid sequence according to SEQ ID NO: 13. As disclosed herein, SEQ ID NO: 13 represents an exemplary amino acid sequence of an ER(T12) domain. In some embodiments, an ER(T12) domain can have an amino acid sequence substantially similar to SEQ ID NO: 13. In some embodiments, an ER(T12) domain can have at least 75%, at least 80%, at least 85%, at least 90%, at least 95%, at least 96%, at least 97%, at least 98%, or at least 99% sequence identity to the amino acid sequence of SEQ ID NO: 13.

SEQ ID NO: 13
SAGDMRAANLWPSPLMIKRSKKNSLALSLTADQMV
SALLDAEPPILYSEYDPTRPFSEASMMGLLTNLAD
RELVHMINWAKRVPGFVDLTLHDQVHLLECAWLEI
LMIGLVWRSMEHPLKLLFAPNLLLDRNQGKCVEGM
VEIFDMLLATSSRFRMMNLQGEEFVCLKSIILLNS
GVYTFLSSTLKSLEEKDHIHRVLDKITDTLIHLMA
KAGLTLQQQHQRLAQLLLILSHIRHMSNKGMEHLY
SMKCKNVVPLYDLLLEAADAHRLHAPTSRGGASVE
ETDQSHLATAGSTSSHSLQKYYITGEAEGFPATV

In some embodiments, a modulating domain can include an amino acid sequence that starts at residue 251, 282, or 305 of SEQ ID NO: 12 and ends at residue 545 or 595 of SEQ ID NO: 12. In some embodiments, a modulating domain can have an amino acid sequence substantially similar to an amino acid sequence that starts at residue 251, 282, or 305 of SEQ ID NO: 12 and ends at residue 545 or 595 of SEQ ID NO: 12. In some embodiments, a modulating domain can have an amino acid sequence that is least 75%, at least 80%, at least 85%, at least 90%, at least 95%, at least 96%, at least 97%, at least 98%, or at least 99% sequence identity to an amino acid sequence that starts at residue 251, 282, or 305 of SEQ ID NO: 12 and ends at residue 545 or 595 of SEQ ID NO: 12.

In some embodiments, a modulating domain does not include a hinge domain of a nuclear receptor (see, e.g., FIG. 6B). Without wishing to be bound to any theory, deletion of a hinge domain from a nuclear receptor or portion thereof acting as a modulating domain can minimize basal activity of the nuclear receptor or portion thereof. As a result, a modulating domain may be able to more effectively mask or inhibit the activity of an associated dominant negative signaling moiety or constitutively active signaling moiety in the absence of a trigger. In some embodiments, a hinge region (also called a D domain) starts at residue 250 of SEQ ID NO: 12 and ends at residue 301 of SEQ ID NO: 12.

### Arrangement

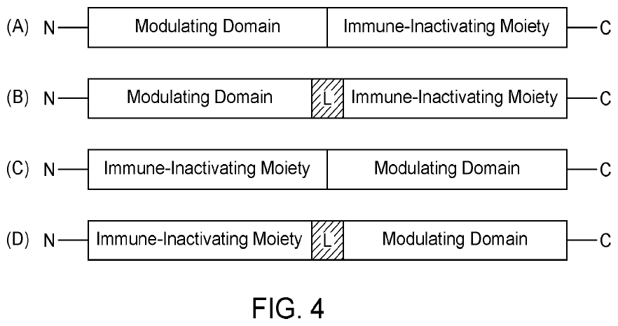

The present disclosure recognizes that there are multiple configurations in which an immune-inactivating moiety (such as a dominant negative signaling moiety or constitutively active signaling moiety) can be operatively associated with a modulating domain to form a trigger-responsive immune-inactivating signaling polypeptide. As one example, a trigger-responsive immune-inactivating signaling polypeptide can have an N-terminus and a C-terminus. If a first entity (e.g., a variant, portion, domain or moiety) is “upstream” of a second entity, the first entity is closer to the N-terminus than the second entity. Conversely, if a first entity is “downstream” of a second entity, the first entity is closer to the C-terminus than the second entity. In some embodiments, an immune-inactivating signaling moiety can be upstream of a modulating domain in a trigger-responsive immune-inactivating signaling polypeptide of the present disclosure (see, e.g., FIGS. 4C and 4D). In some embodiments, a dominant immune-inactivating moiety can be downstream of a modulating domain in a trigger-responsive immune-inactivating signaling polypeptide of the present disclosure (see, e.g., FIGS. 4A and 4B).

In some embodiments, a trigger-responsive immune-inactivating signaling polypeptide can include one or more immune-inactivating signaling moieties. For example, a trigger-responsive immune-inactivating signaling polypeptide can include one, two, or three immune-inactivating signaling moieties. In some embodiments, the one or more immune-inactivating signaling moieties of a trigger-responsive immune-inactivating signaling polypeptide are the same immune-inactivating signaling moiety or are different immune-inactivating signaling moieties.

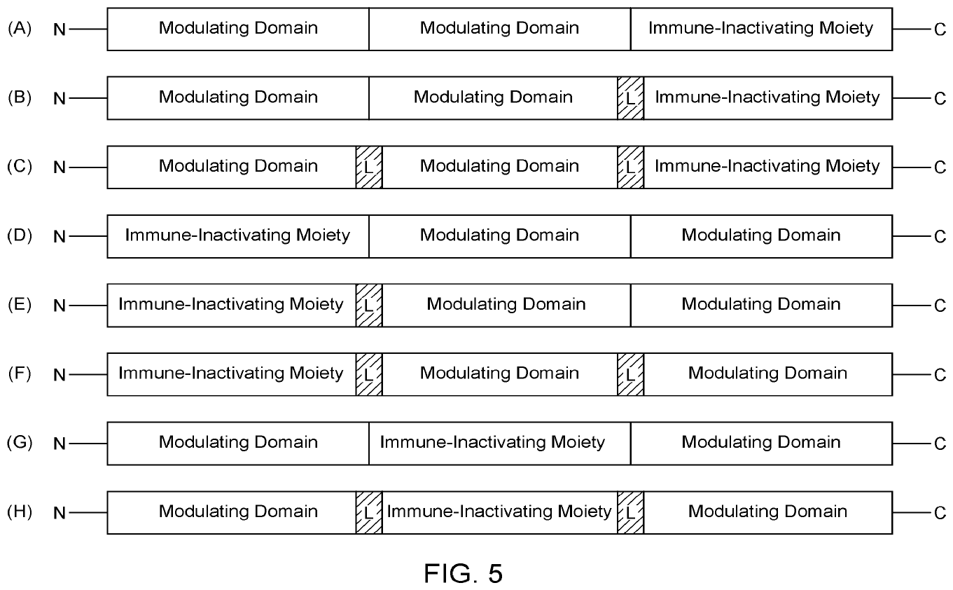

In some embodiments, a trigger-responsive immune-inactivating signaling polypeptide can include one or more modulating domains (see, e.g., FIGS. 5A-5H). For example, a trigger-responsive immune-inactivating signaling polypeptide can include one, two, three, four or more modulating domains. In some embodiments, the one or more modulating domains of a trigger-responsive immune-inactivating signaling polypeptide are the same modulating domain. In some embodiments, the one or more modulating domains of a trigger-responsive immune-inactivating signaling polypeptide are different modulating domains.

In some embodiments, a trigger-responsive immune-inactivating signaling polypeptide can include an immune-inactivating signaling moiety between modulating domains (see, e.g., FIGS. 5G and 5H). Without wishing to be bound by any theory, including at least one modulating domain upstream and at least one modulating domain downstream of an immune-inactivating signaling moiety can enhance the ability of the modulating domains to mask or inhibit the activity of the immune-inactivating signaling moiety and prevent “leakiness” or unintended activity from the immune-inactivating signaling moiety, particularly in the absence of a trigger.

In some embodiments, an immune-inactivating signaling moiety can be operatively linked to a modulating domain directly (see, e.g., FIGS. 4A and 4C; FIGS. 5A, 5B, 5D, 5E, and 5G). In other embodiments, an immune-inactivating signaling moiety can be operatively linked to a modulating domain indirectly, e.g., via a linker (see, e.g., FIGS. 4B and 4D; FIGS. 5B, 5C, 5E, 5F, and 5H).

In some embodiments, one or more immune-inactivating signaling moieties can be operatively linked to one another directly. In other embodiments, one or more immune-inactivating signaling moieties can be operatively linked to one another indirectly, e.g., via a linker. In some embodiments, a linker can comprise a polyalanine (including, e.g., 1-10 alanines).

In some embodiments, one or more modulating domains can be operatively linked to one another directly (see, e.g., FIGS. 5A, 5B, 5D, and 5E). In other embodiments, one or more immune-inactivating signaling moieties can be operatively linked to one another indirectly, e.g., via a linker (see, e.g., FIGS. 5C and 5F).

### Additional Moieties

In addition to an immune-inactivating signaling moiety and/or a modulating domain, a trigger-responsive immune-inactivating signaling polypeptide can include additional moieties, such as regulatory elements, signal sequences, and tags. In some embodiments, a trigger-responsive immune-inactivating signaling polypeptide includes a nuclear export signal (NES). A nuclear export signal can be a short amino acid sequence that targets an associated polypeptide for export from the cell nucleus to the cytoplasm through the nuclear pore complex using nuclear transport. In some embodiments, a nuclear export signal includes at least four hydrophobic residues. SEQ ID NO: 5 includes a nucleotide sequence encoding an exemplary nuclear export signal. In some embodiments, a nuclear export signal can have an amino acid sequence according to SEQ ID NO: 6.

SEQ ID NO: 5
AATGAATTAGCCTTGAAATTAGCAGGTCTTGATA
TCAACAAGACA
SEQ ID NO: 6
N E L A L K L A G L D I N K T

### Triggers

The present disclosure encompasses trigger-responsive immune-inactivating signaling polypeptides that can adopt at least first and second state, and can switch from one to the other in response to a particular trigger. The present disclosure utilizes a trigger as mechanism to tightly control the activity of an immune-inactivating signaling moiety in a trigger-responsive immune-inactivating signaling polypeptide via a modulating domain. In some embodiments, the present disclosure provides technologies in which a trigger-responsive immune-inactivating signaling polypeptide, is exposed to a trigger for a limited period of time (e.g., due to removal, expiration, inactivation, and/or destruction of the trigger). The present disclosure provides an insight that reversibility of immune-inactivating activity according to such technologies offers unique advantages for regulation of T cell activity, among other things avoiding difficulties associated with alternative approaches for regulating T cells where T cell activity, once triggered, cannot readily be shut back off Indeed, in some embodiments, the present disclosure provides systems that permit not simply “on-off” control of T cell activity, but potentially adjustable “dial-up/dial-down” control (e.g., based on concentration, intensity, or frequency of trigger).

In some embodiments, a trigger can be a condition, e.g., a local condition. For example, a trigger can be a particular pH range, temperature range, range of oxygen levels, etc. In some embodiments, a trigger can be an entity, such as a molecule, e.g., a small molecule or a macromolecule (e.g., a polypeptide, nucleic acid or carbohydrate).

In some embodiments, when a trigger is not present or is present at a level below a threshold value, a modulating domain can be in a first state. In some embodiments, when a trigger is present or is present at a level above a threshold value, a modulating domain can be in a second state. In some embodiments, a trigger can be introduced, for example, by adding a trigger to a sample (e.g., cells) or administering a trigger to a subject (e.g., a human). In certain embodiments, a trigger-responsive immune-inactivating signaling polypeptide is only exposed to or in the presence of a trigger when its switch between a first state and a second state is desired.

In some embodiments, a trigger has a temporal nature. In some embodiments, a trigger can have a relatively short-half life in a system (e.g., cells, tissue, subject (e.g., human)) to which the trigger has been introduced. For example, a trigger can have a half-life of no more than 1 hour, no more than 2 hours, no more than 5 hours, no more than 12 hours, no more than 24 hours, or no more than two days.

In some embodiments, a trigger can have a relatively rapid clearance from a system (e.g., cells, tissue, subject (e.g., human)) to which the trigger has been introduced. For example, a trigger can have 95% clearance from a system in less than 30 min, in less than an hour, in less than 2 hours, in less than 5 hours, in less than 12 hours, in less than 24 hours, or in less than two days.

As discussed above, a trigger-responsive immune-inactivating signaling polypeptide can include a modulating domain, which can be the portion of the trigger-responsive immune-inactivating signaling polypeptide that adopts at least a first and a second state, and can switch from one to the other in response to a particular trigger. In some embodiments, a modulating domain can include a nuclear receptor or a portion thereof. In embodiments in which a trigger-responsive immune-inactivating signaling polypeptide includes a modulating domain comprising a ligand binding domain of a nuclear receptor, a trigger can be a ligand or other agent that binds to the ligand binding domain. In some embodiments, a ligand can be a natural ligand of a ligand binding domain. In some embodiments, a ligand can be a synthetic ligand designed to bind a ligand binding domain. Exemplary ligands that bind to ligand binding domains of select nuclear receptors are shown in Table 5 below.

TABLE 5

| Nuclear Receptor | Exemplary Ligands |
|---|---|
| thyroid hormone receptor (e.g. a thyroid | Thyroid hormone |
| hormone receptor-α or a thyroid hormone | Thyroid hormone analogs |
| receptor-β) | Thyroid hormone derivatives |
| retinoic acid receptor (e.g., a retinoic acid | Vitamin A |
| receptor-α, a retinoic acid receptor-β, or a | Vitamin A analogs |
| retinoic acid receptor-γ) | Vitamin A derivatives, including retinoic acid |
| peroxisome proliferator-activated receptor | Prostaglandin |
| (e.g., a peroxisome proliferator-activated | Fatty Acids |
| receptor-α, a peroxisome proliferator-activated | Eicosanoids |
| receptor-β, or a peroxisome proliferator- | 5-oxo-15(S)-HETE |
| activated receptor-γ) | 5-oxo-ETE |
| | 15(S)-HETE |
| | 15(R)-HETE |
| | 15-HpETE |
| | Leukotriene B4 |
| Rev-ErbA receptor | Heme |
| RAR-related orphan receptor (e.g., a RAR- | Cholesterol |
| related orphan receptor-α, a RAR-related | Cholesterol derivatives |
| orphan receptor-β, or a RAR-related orphan | Tretinoin |
| receptor-γ) | Melatonin |
| liver X receptor | Oxysterols, including 22(R)- |
| (e.g., a liver X receptor-α or a liver | hydroxycholesterol, 24(S)-hydroxycholesterol, |
| X receptor-β) | 27-hydroxycholesterol, and cholestenoic acid |
| farnesoid X receptor | Oxysterols |
| (e.g., a farnesoid X receptor-α or a | Chenodeoxycholic acid |
| farnesoid X receptor-β) | |
| vitamin D receptor | Vitamin D |
| androstane receptor | Androstane |
| hepatocyte nuclear factor-4 receptor (e.g., | Fatty Acids |
| hepatocyte nuclear factor-4-α receptor or | Linoleic acid |
| hepatocyte nuclear factor-4-γ receptor) | |
| retinoid X receptor (e.g., a retinoid X | Retinoids, including 9-cis retinoic acid |
| receptor-α, a retinoid X receptor-β, or a | and 9-cis-13,14-dihydro-retinoic acid |
| retinoid X receptor-γ) | |
| estrogen receptor (e.g., an estrogen | Estradiol (e.g., 17-beta estradiol) |
| receptor-α or an estrogen receptor-β) | Estrone |
| | Estriol |
| | Raloxifene |
| | Genistein |
| | Endoxifen |
| | Tamoxifen |
| | 4-hydroxytamoxifen |
| | Fulvestrant |
| | OP-1250 |
| | OP-1124 |
| | OP-1074 |
| | AZD-9496 |
| | ARN-810 |
| | SRN-927 |
| | SERMs and SERDs |
| | Estrogen analogs |
| glucocorticoid receptor | Glucocorticoids, including cortisol |
| mineralocorticoid receptor | Mineralocorticoids, including aldosterone |
| | and deoxycorticosterone |
| | Glucocorticoids, including cortisol |
| | Sprionolactone |
| | Eplerenone |
| progesterone receptor | Progesterone |
| | Progesterone analogs |
| | Progesterone derivatives |
| androgen receptor | Testosterone |
| | Testosterone analogs |
| | Testosterone derivatives |
| | 2-Quinolones |
| | Phthalamides |
| | Bicalutamides |
| | Coumarins |
| | Nonsteroidal SARMS |

### Pharmaceutical Compositions

In some embodiments, a trigger can be included in a pharmaceutical composition. In some embodiments, a pharmaceutical composition can include physiologically acceptable carrier or excipient. Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, sugars such as mannitol, sucrose, or others, dextrose, magnesium stearate, talc, silicic acid, viscous paraffin, perfume oil, fatty acid esters, hydroxymethylcellulose, polyvinyl pyrrolidone, etc., as well as combinations thereof. A pharmaceutical composition can, if desired, be mixed with auxiliary agents (e.g., lubricants, preservatives, stabilizers, wetting agents, emulsifiers, salts for influencing osmotic pressure, buffers, coloring, flavoring and/or aromatic substances and the like), which do not deleteriously react with the active compounds or interfere with their activity. In certain embodiments, a water-soluble carrier suitable for intravenous administration is used. In some embodiments, a pharmaceutical composition can be sterile.

A suitable pharmaceutical composition, if desired, can also contain minor amounts of wetting or emulsifying agents, or pH buffering agents. A pharmaceutical composition can be a liquid solution, suspension, emulsion, tablet, pill, capsule, sustained release formulation, or powder. A pharmaceutical composition can also be formulated as a suppository, with traditional binders and carriers such as triglycerides. Oral pharmaceutical compositions can include standard carriers, such as pharmaceutical grades of mannitol, lactose, starch, magnesium stearate, polyvinyl pyrrolidone, sodium saccharine, cellulose, magnesium carbonate, etc.

A pharmaceutical composition can be formulated in accordance with the routine procedures as a pharmaceutical composition adapted for administration to human beings. The formulation of a pharmaceutical composition should suit the mode of administration. For example, in some embodiments, a composition for intravenous administration typically is a solution in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where a pharmaceutical composition is to be administered by infusion, it can be dispensed with an infusion bottle containing sterile pharmaceutical grade water, saline or dextrose/water. Where a pharmaceutical composition is administered by injection, an ampule of sterile water for injection or saline can be provided so that the ingredients may be mixed prior to administration.

A trigger described herein can be formulated as neutral or salt forms in a pharmaceutical composition. A trigger can include pharmaceutical composition that has received regulatory approval.

### Nucleic Acids

Among other things, the present disclosure provides nucleic acids encoding a trigger-responsive immune-inactivating signaling polypeptide described herein. Such nucleic acids can be DNA or RNA.

In a certain embodiment, a trigger-responsive dominant negative signaling polypeptide is an endoxifen-responsive dominant negative Zap-70 polypeptide. In some embodiments, an endoxifen-responsive dominant negative Zap-70 polypeptide is encoded by a nucleotide sequence according to SEQ ID NO: 7 or SEQ ID NO: 29. As disclosed herein, SEQ ID NO: 7 and SEQ ID NO: 29 represent exemplary nucleotide sequences encoding endoxifen-responsive dominant negative Zap-70 polypeptides. In some embodiments, an endoxifen-responsive dominant negative Zap-70 polypeptide can be encoded by DNA having a nucleotide sequence substantially similar to SEQ ID NO: 7 or SEQ ID NO: 29. In some embodiments, an endoxifen-responsive dominant negative Zap-70 polypeptide can be encoded by DNA having a nucleotide sequence with at least 75%, at least 80%, at least 85%, at least 90%, at least 95%, at least 96%, at least 97%, at least 98%, or at least 99% sequence identity to the nucleotide sequence of SEQ ID NO: 7 or the nucleotide sequence of SEQ ID NO: 29.

SEQ ID NO: 7
ATGAATGAATTAGCCTTGAAATTAGCAGGTCTTGA
TATCAACAAGACAATGCCAGACCCCGCGGCGCACC
TGCCCTTCTTCTACGGCAGCATCTCGCGTGCCGAG
GCCGAGGAGCACCTGAAGCTGGCGGGCATGGCGGA
CGGGCTCTTCCTGCTGCGCCAGTGCCTGCGCTCGC
TGGGCGGCTATGTGCTGTCGCTCGTGCACGATGTG
CGCTTCCACCACTTTCCCATCGAGCGCCAGCTCAA
CGGCACCTACGCCATTGCCGGCGGCAAAGCGCACT
GTGGACCGGCAGAGCTCTGCGAGTTCTACTCGCGC
GACCCCGACGGGCTGCCCTGCAACCTGCGCAAGCC
GTGCAACCGGCCGTCGGGCCTCGAGCCGCAGCCGG
GGGTCTTCGACTGCCTGCGAGACGCCATGGTGCGT
GACTACGTGCGCCAGACGTGGAAGCTGGAGGGCGA
GGCCCTGGAGCAGGCCATCATCAGCCAGGCCCCGC
AGGTGGAGAAGCTCATTGCTACGACGGCCCACGAG
CGGATGCCCTGGTACCACAGCAGCCTGACGCGTGA
GGAGGCCGAGCGCAAACTTTACTCTGGGGCGCAGA
CCGACGGCAAGTTCCTGCTGAGGCCGCGGAAGGAG
CAGGGCACATACGCCCTGTCCCTCATCTATGGGAA
GACGGTGTACCACTACCTCATCAGCCAAGACAAGG
CGGGCAAGTACTGCATTCCCGAGGGCACCAAGTTT
GACACGCTCTGGCAGCTGGTGGAGTATCTGAAGCT
GAAGGCGGACGGGCTCATCTACTGCCTGAAGGAGG
CCTGCCCCAACAGCAGTGCCAGCAACGCCTCAGGG
GCTGCTGCTCCCACACTCCCAGCCCACCCATCCAC
GTTGACGGGATCCTCTGCTGGAGACATGAGAGCTG
CCAACCTTTGGCCAAGCCCGCTCATGATCAAACGC
TCTAAGAAGAACAGCCTGGCCTTGTCCCTGACGGC
CGACCAGATGGTCAGTGCCTTGTTGGATGCTGAGC
CCCCCATACTCTATTCCGAGTATGATCCTACCAGA
CCCTTCAGTGAAGCTTCGATGATGGGCTTACTGAC
CAACCTGGCAGACAGGGAGCTGGTTCACATGATCA
ACTGGGCGAAGAGGGTGCCAGGCTTTGTGGATTTG
ACCCTCCATGATCAGGTCCACCTTCTAGAATGTGC
CTGGCTAGAGATCCTGATGATTGGTCTCGTCTGGC
GCTCCATGGAGCACCCAGTGAAGCTACTGTTTGCT
CCTAACTTGCTCTTGGACAGGAACCAGGGAAAATG
TGTAGAGGGCATGGTGGAGATCTTCGACATGCTGC
TGGCTACATCATCTCGGTTCCGCATGATGAATCTG
CAGGGAGAGGAGTTTGTGTGCCTCAAATCTATTAT
TTTGCTTAATTCTGGAGTGTACACATTTCTGTCCA
GCACCCTGAAGTCTCTGGAAGAGAAGGACCATATC
CACCGAGTCCTGGACAAGATCACAGACACTTTGAT
CCACCTGATGGCCAAGGCAGGCCTGACCCTGCAGC
AGCAGCACCAGCGGCTGGCCCAGCTCCTCCTCATC
CTCTCCCACATCAGGCACATGAGTAACAAAGGCAT
GGAGCATCTGTACAGCATGAAGTGCAAGAACGTGG
TGCCCCTCTATGACCTGCTGCTGGAGGCGGCGGAC
GCCCACCGCCTACATGCGCCCACTAGCCGTGGAGG
GGCATCCGTGGAGGAGACGGACCAAAGCCACTTGG
CCACTGCGGGCTCTACTTCATCGCATTCCTTGCAA
AAGTATTACATCACGGGGGAGGCAGAGGGTTTCCC
TGCCACGGTC

In some embodiments, an endoxifen-responsive dominant negative Zap-70 polypeptide is encoded by a nucleotide sequence according to SEQ ID NO: 16 or SEQ ID NO: 32. As disclosed herein, SEQ ID NO: 16 and SEQ ID NO: 32 represent exemplary nucleotide sequences encoding endoxifen-responsive dominant negative Zap-70 polypeptides. In some embodiments, an endoxifen-responsive dominant negative Zap-70 polypeptide can be encoded by DNA having a nucleotide sequence substantially similar to SEQ ID NO: 16 or SEQ ID NO: 32. In some embodiments, an endoxifen-responsive dominant negative Zap-70 polypeptide can be encoded by DNA having a nucleotide sequence with at least 75%, at least 80%, at least 85%, at least 90%, at least 95%, at least 96%, at least 97%, at least 98%, or at least 99% sequence identity to the nucleotide sequence of SEQ ID NO: 16 or the nucleotide sequence of SEQ ID NO: 32.

SEQ ID NO: 16
ATGAATGAATTAGCCTTGAAATTAGCAGGTCTTGA
TATCAACAAGACAATGCCAGACCCCGCGGCGCACC
TGCCCTTCTTCTACGGCAGCATCTCGCGTGCCGAG
GCCGAGGAGCACCTGAAGCTGGCGGGCATGGCGGA
CGGGCTCTTCCTGCTGCGCCAGTGCCTGCGCTCGC
TGGGCGGCTATGTGCTGTCGCTCGTGCACGATGTG
CGCTTCCACCACTTTCCCATCGAGCGCCAGCTCAA
CGGCACCTACGCCATTGCCGGCGGCAAAGCGCACT
GTGGACCGGCAGAGCTCTGCGAGTTCTACTCGCGC
GACCCCGACGGGCTGCCCTGCAACCTGCGCAAGCC
GTGCAACCGGCCGTCGGGCCTCGAGCCGCAGCCGG
GGGTCTTCGACTGCCTGCGAGACGCCATGGTGCGT
GACTACGTGCGCCAGACGTGGAAGCTGGAGGGCGA
GGCCCTGGAGCAGGCCATCATCAGCCAGGCCCCGC
AGGTGGAGAAGCTCATTGCTACGACGGCCCACGAG
CGGATGCCCTGGTACCACAGCAGCCTGACGCGTGA
GGAGGCCGAGCGCAAACTTTACTCTGGGGCGCAGA
CCGACGGCAAGTTCCTGCTGAGGCCGCGGAAGGAG
CAGGGCACATACGCCCTGTCCCTCATCTATGGGAA
GACGGTGTACCACTACCTCATCAGCCAAGACAAGG
CGGGCAAGTACTGCATTCCCGAGGGCACCAAGTTT
GACACGCTCTGGCAGCTGGTGGAGTATCTGAAGCT
GAAGGCGGACGGGCTCATCTACTGCCTGAAGGAGG
CCTGCCCCAACAGCAGTGCCAGCAACGCCTCAGGG
GCTGCTGCTCCCACACTCCCAGCCCACCCATCCAC
GTTGACGGGATCCTCTGCTGGAGACATGAGAGCTG
CCAACCTTTGGCCAAGCCCGCTCATGATCAAACGC
TCTAAGAAGAACAGCCTGGCCTTGTCCCTGACGGC
CGACCAGATGGTCAGTGCCTTGTTGGATGCTGAGC
CCCCCATACTCTATTCCGAGTATGATCCTACCAGA
CCCTTCAGTGAAGCTTCGATGATGGGCTTACTGAC
CAACCTGGCAGACAGGGAGCTGGTTCACATGATCA
ACTGGGCGAAGAGGGTGCCAGGCTTTGTGGATTTG
ACCCTCCATGATCAGGTCCACCTTCTAGAATGTGC
CTGGCTAGAGATCCTGATGATTGGTCTCGTCTGGC
GCTCCATGGAGCACCCACTGAAGCTACTGTTTGCT
CCTAACTTGCTCTTGGACAGGAACCAGGGAAAATG
TGTAGAGGGCATGGTGGAGATCTTCGACATGCTGC
TGGCTACATCATCTCGGTTCCGCATGATGAATCTG
CAGGGAGAGGAGTTTGTGTGCCTCAAATCTATTAT
TTTGCTTAATTCTGGAGTGTACACATTTCTGTCCA
GCACCCTGAAGTCTCTGGAAGAGAAGGACCATATC
CACCGAGTCCTGGACAAGATCACAGACACTTTGAT
CCACCTGATGGCCAAGGCAGGCCTGACCCTGCAGC
AGCAGCACCAGCGGCTGGCCCAGCTCCTCCTCATC
CTCTCCCACATCAGGCACATGAGTAACAAAGGCAT
GGAGCATCTGTACAGCATGAAGTGCAAGAACGTGG
TGCCCCTCTATGACCTGCTGCTGGAGGCGGCGGAC
GCCCACCGCCTACATGCGCCCACTAGCCGTGGAGG
GGCATCCGTGGAGGAGACGGACCAAAGCCACTTGG
CCACTGCGGGCTCTACTTCATCGCATTCCTTGCAA
AAGTATTACATCACGGGGGAGGCAGAGGGTTTCCC
TGCCACGGTCTGA

In a certain embodiment, a trigger-responsive dominant negative signaling polypeptide is an endoxifen-responsive dominant negative LCK polypeptide. In some embodiments, an endoxifen-responsive dominant negative LCK polypeptide is encoded by a nucleotide sequence according to SEQ ID NO: 22 or SEQ ID NO: 34. As disclosed herein, SEQ ID NO: 22 and SEQ ID NO: 34 represent exemplary nucleotide sequences encoding endoxifen-responsive dominant negative LCK polypeptides. In some embodiments, an endoxifen-responsive dominant negative LCK polypeptide can be encoded by DNA having a nucleotide sequence substantially similar to SEQ ID NO: 22 or SEQ ID NO: 34. In some embodiments, an endoxifen-responsive dominant negative LCK polypeptide can be encoded by DNA having a nucleotide sequence with at least 75%, at least 80%, at least 85%, at least 90%, at least 95%, at least 96%, at least 97%, at least 98%, or at least 99% sequence identity to the nucleotide sequence of SEQ ID NO: 22 or the nucleotide sequence of SEQ ID NO: 34.

SEQ ID NO: 22
ATGAATGAATTAGCCTTGAAATTAGCAGGTCTTGA
TATCAACAAGACAATGGGCTGTGGCTGCAGCTCAC
ACCCGGAAGATGACTGGATGGAAAACATCGATGTG
TGTGAGAACTGCCATTATCCCATAGTCCCACTGGA
TGGCAAGGGCACGCTGCTCATCCGAAATGGCTCTG
AGGTGCGGGACCCACTGGTTACCTACGAAGGCTCC
AATCCGCCGGCTTCCCCACTGCAAGACAACCTGGT
TATCGCTCTGCACAGCTATGAGCCCTCTCACGACG
GAGATCTGGGCTTTGAGAAGGGGGAACAGCTCCGC
ATCCTGGAGCAGAGCGGCGAGTGGTGGAAGGCGCA
GTCCCTGACCACGGGCCAGGAAGGCTTCATCCCCT
TCAATTTTGTGGCCAAAGCGAACAGCCTGGAGCCC
GAACCCTGGTTCTTCAAGAACCTGAGCCGCAAGGA
CGCGGAGCGGCAGCTCCTGGCGCCCGGGAACACTC
ACGGCTCCTTCCTCATCCGGGAGAGCGAGAGCACC
GCGGGATCGTTTTCACTGTCGGTCCGGGACTTCGA
CCAGAACCAGGGAGAGGTGGTGAAACATTACAAGA
TCCGTAATCTGGACAACGGTGGCTTCTACATCTCC
CCTCGAATCACTTTTCCCGGCCTGCATGAACTGGT
CCGCCATTACACCAATGCTTCAGATGGGCTGTGCA
CACGGTTGAGCCGCCCCTGCCAGACCCAGAAGCCC
CAGAAGCCGTGGTGGGAGGACGAGTGGGAGGTTCC
CAGGGAGACGCTGAAGCTGGTGGAGCGGCTGGGGG
CTGGACAGTTCGGGGAGGTGTGGATGGGGTACTAC
AACGGGGGATCCTCTGCTGGAGACATGAGAGCTGC
CAACCTTTGGCCAAGCCCGCTCATGATCAAACGCT
CTAAGAAGAACAGCCTGGCCTTGTCCCTGACGGCC
GACCAGATGGTCAGTGCCTTGTTGGATGCTGAGCC
CCCCATACTCTATTCCGAGTATGATCCTACCAGAC
CCTTCAGTGAAGCTTCGATGATGGGCTTACTGACC
AACCTGGCAGACAGGGAGCTGGTTCACATGATCAA
CTGGGCGAAGAGGGTGCCAGGCTTTGTGGATTTGA
CCCTCCATGATCAGGTCCACCTTCTAGAATGTGCC
TGGCTAGAGATCCTGATGATTGGTCTCGTCTGGCG
CTCCATGGAGCACCCACTGAAGCTACTGTTTGCTC
CTAACTTGCTCTTGGACAGGAACCAGGGAAAATGT
GTAGAGGGCATGGTGGAGATCTTCGACATGCTGCT
GGCTACATCATCTCGGTTCCGCATGATGAATCTGC
AGGGAGAGGAGTTTGTGTGCCTCAAATCTATTATT
TTGCTTAATTCTGGAGTGTACACATTTCTGTCCAG
CACCCTGAAGTCTCTGGAAGAGAAGGACCATATCC
ACCGAGTCCTGGACAAGATCACAGACACTTTGATC
CACCTGATGGCCAAGGCAGGCCTGACCCTGCAGCA
GCAGCACCAGCGGCTGGCCCAGCTCCTCCTCATCC
TCTCCCACATCAGGCACATGAGTAACAAAGGCATG
GAGCATCTGTACAGCATGAAGTGCAAGAACGTGGT
GCCCCTCTATGACCTGCTGCTGGAGGCGGCGGACG
CCCACCGCCTACATGCGCCCACTAGCCGTGGAGGG
GCATCCGTGGAGGAGACGGACCAAAGCCACTTGGC
CACTGCGGGCTCTACTTCATCGCATTCCTTGCAAA
AGTATTACATCACGGGGGAGGCAGAGGGTTTCCCT
GCCACGGTCTGA

In a certain embodiment, a trigger-responsive constitutively active signaling polypeptide is an endoxifen-responsive constitutively active SHP1 polypeptide. In some embodiments, an endoxifen-responsive constitutively active SHP1 polypeptide is encoded by a nucleotide sequence according to SEQ ID NO: 26 or SEQ ID NO: 36. As disclosed herein, SEQ ID NO: 26 and or SEQ ID NO: 36 represent exemplary nucleotide sequences encoding endoxifen-responsive constitutively active SHP1 polypeptides. In some embodiments, an endoxifen-responsive constitutively active SHP1 polypeptide can be encoded by DNA having a nucleotide sequence substantially similar to SEQ ID NO: 26 or SEQ ID NO: 36. In some embodiments, an endoxifen-responsive constitutively active SHP1 polypeptide can be encoded by DNA having a nucleotide sequence with at least 75%, at least 80%, at least 85%, at least 90%, at least 95%, at least 96%, at least 97%, at least 98%, or at least 99% sequence identity to the nucleotide sequence of SEQ ID NO: 26 or the nucleotide sequence of SEQ ID NO: 36.

SEQ ID NO: 26
ATGAATGAATTAGCCTTGAAATTAGCAGGTCTTGA
TATCAACAAGACACGGCAGCCGTACTATGCCACGA
GGGTGAATGCGGCTGACATTGAGAACCGAGTGTTG
GAACTGAACAAGAAGCAGGAGTCCGAGGATACAGC
CAAGGCTGGCTTCTGGGAGGAGTTTGAGAGTTTGC
AGAAGCAGGAGGTGAAGAACTTGCACCAGCGTCTG
GAAGGGCAGCGGCCAGAGAACAAGGGCAAGAACCG
CTACAAGAACATTCTCCCCTTTGACCACAGCCGAG
TGATCCTGCAGGGACGGGACAGTAACATCCCCGGG
TCCGACTACATCAATGCCAACTACATCAAGAACCA
GCTGCTAGGCCCTGATGAGAACGCTAAGACCTACA
TCGCCAGCCAGGGCTGTCTGGAGGCCACGGTCAAT
GACTTCTGGCAGATGGCGTGGCAGGAGAACAGCCG
TGTCATCGTCATGACCACCCGAGAGGTGGAGAAAG
GCCGGAACAAATGCGTCCCATACTGGCCCGAGGTG
GGCATGCAGCGTGCTTATGGGCCCTACTCTGTGAC
CAACTGCGGGGAGCATGACACAACCGAATACAAAC
TCCGTACCTTACAGGTCTCCCCGCTGGACAATGGA
GACCTGATTCGGGAGATCTGGCATTACCAGTACCT
GAGCTGGCCCGACCATGGGGTCCCCAGTGAGCCTG
GGGGTGTCCTCAGCTTCCTGGACCAGATCAACCAG
CGGCAGGAAAGTCTGCCTCACGCAGGGCCCATCAT
CGTGCACTGCAGCGCCGGCATCGGCCGCACAGGCA
CCATCATTGTCATCGACATGCTCATGGAGAACATC
TCCACCAAGGGCCTGGACTGTGACATTGACATCCA
GAAGACCATCCAGATGGTGCGGGCGCAGCGCTCGG
GCATGGTGCAGACGGAGGCGCAGTACAAGTTCATC
TACGTGGCCATCGCCCAGTTCATTGAAACCACTAA
GAAGAAGCTGGAGGTCCTGCAGTCGCAGAAGGGCC
AGGAGTCGGAGTACGGGAACATCACCTATCCCCCA
GCCATGAAGAATGCCCATGCCAAGGCCTCCCGCAC
CTCGTCCAAACACAAGGAGGATGTGTATGAGAACC
TGCACACTAAGAACAAGAGGGAGGAGAAAGTGAAG
AAGCAGCGGTCAGCAGACAAGGAGAAGAGCAAGGG
TTCCCTCAAGAGGAAGGGATCCTCTGCTGGAGACA
TGAGAGCTGCCAACCTTTGGCCAAGCCCGCTCATG
ATCAAACGCTCTAAGAAGAACAGCCTGGCCTTGTC
CCTGACGGCCGACCAGATGGTCAGTGCCTTGTTGG
ATGCTGAGCCCCCCATACTCTATTCCGAGTATGAT
CCTACCAGACCCTTCAGTGAAGCTTCGATGATGGG
CTTACTGACCAACCTGGCAGACAGGGAGCTGGTTC
ACATGATCAACTGGGCGAAGAGGGTGCCAGGCTTT
GTGGATTTGACCCTCCATGATCAGGTCCACCTTCT
AGAATGTGCCTGGCTAGAGATCCTGATGATTGGTC
TCGTCTGGCGCTCCATGGAGCACCCACTGAAGCTA
CTGTTTGCTCCTAACTTGCTCTTGGACAGGAACCA
GGGAAAATGTGTAGAGGGCATGGTGGAGATCTTCG
ACATGCTGCTGGCTACATCATCTCGGTTCCGCATG
ATGAATCTGCAGGGAGAGGAGTTTGTGTGCCTCAA
ATCTATTATTTTGCTTAATTCTGGAGTGTACACAT
TTCTGTCCAGCACCCTGAAGTCTCTGGAAGAGAAG
GACCATATCCACCGAGTCCTGGACAAGATCACAGA
CACTTTGATCCACCTGATGGCCAAGGCAGGCCTGA
CCCTGCAGCAGCAGCACCAGCGGCTGGCCCAGCTC
CTCCTCATCCTCTCCCACATCAGGCACATGAGTAA
CAAAGGCATGGAGCATCTGTACAGCATGAAGTGCA
AGAACGTGGTGCCCCTCTATGACCTGCTGCTGGAG
GCGGCGGACGCCCACCGCCTACATGCGCCCACTAG
CCGTGGAGGGGCATCCGTGGAGGAGACGGACCAAA
GCCACTTGGCCACTGCGGGCTCTACTTCATCGCAT
TCCTTGCAAAAGTATTACATCACGGGGGAGGCAGA
GGGTTTCCCTGCCACGGTCTGA

In some embodiments, RNA encoding a trigger-responsive immune-inactivating signaling polypeptide described herein can be transcribed from one of the nucleic acid sequences described herein.

In certain instances, recombinant DNA techniques can be used to produce a trigger-responsive immune-inactivating signaling polypeptide. The process of cloning DNA (e.g., cDNA) segments and sequences that encode the respective polypeptides, polypeptide fragments, domains, and/or moieties (e.g., a modulating domain and an immune-inactivating signaling moiety), the production of DNA sequences encoding any of various peptide linkers, the ligation of different DNA (e.g., cDNA sequences), the construction of the expression vectors (e.g., plasmid, bacteriophage, phagemid, or viral vector), and the protein expression and purification of a resulting recombinant polypeptide (e.g., a fusion polypeptide) can be performed by conventional recombinant molecular biology and protein biochemistry techniques such as those described in Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Current Protocols in Molecular Biology (CPMB), Frederick M. Ausubel (ed.), John Wiley and Sons (2014) (ISBN 047150338X, 9780471503385), Current Protocols in Protein Science (CPPS), John E. Coligan (ed.), John Wiley and Sons, Inc. (2005).

Expression of a trigger-responsive immune-inactivating signaling polypeptide can include construction of an expression vector containing a polynucleotide that encodes a trigger-responsive immune-inactivating signaling polypeptide described herein. An expression vector polynucleotide can further include sequences that encode additional amino acids for the purpose of protein purification, or identifying or locating a trigger-responsive immune-inactivating signaling polypeptide in the expression system or during the protein purification process. Once a polynucleotide encoding a trigger-responsive immune-inactivating signaling polypeptide has been obtained, the vector for the production of a trigger-responsive immune-inactivatingsignaling polypeptide can be produced by recombinant DNA technology using techniques well known in the art. In addition to including a polynucleotide that encodes a trigger-responsive immune-inactivatingsignaling polypeptide, an expression vector can also include, e.g., appropriate replication, transcriptional and translational control signals.

In one aspect, provided herein is a vector comprising a nucleic acid sequence encoding a trigger-responsive immune-inactivatingsignaling polypeptide described herein. In some embodiments, a vector can include one or more regulatory elements (e.g., viral arms, origins of replication, integration elements, etc.) that permit transfer from one context to another and/or delivery to a particular context of interest. In some embodiments, the vector can be a plasmid, a bacteriophage, a phagemid, a cosmid, a viral vector, or a viral particle. These vectors are known in the art. In one embodiment, provided herein is a plasmid comprising a nucleic acid sequence encoding a trigger-responsive immune-inactivatingsignaling polypeptide described herein. For example, the plasmid is a bacterial plasmid. In one embodiment of a vector described, the vector is an expression vector. For example, the plasmid (vector) is an expression plasmid for the recombinant protein expression in a bacteria, e.g., Escherichia coli. In one embodiment of an expression vector described, the expression vector is a bacterial expression vector. In one embodiment of an expression vector described, the expression vector is a prokaryotic expression vector. In one embodiment of an expression vector described, the expression vector is an eukaryotic expression vector. In one embodiment of an expression vector described, the expression vector is a mammalian expression vector. In one embodiment, the expression vector is a yeast expression vector.

The expression vector can be transferred to a host cell by conventional techniques and the transfected cells can then be cultured by conventional techniques to produce a trigger-responsive immune-inactivatingsignaling polypeptide of the present disclosure. Thus, the present disclosure encompasses host cells containing a polynucleotide encoding a trigger-responsive immune-inactivatingsignaling polypeptide, operably linked to a promoter. Various regulatory sequences or elements may be incorporated in a vector suitable for the present invention. Exemplary regulatory sequences or elements include, but are not limited to, promoters, enhancers, repressors or suppressors, 5′ untranslated (or noncoding) sequences, introns, 3′ untranslated (or non-coding) sequences, terminators, and splice elements.

As used herein, a “promoter” or “promoter sequence” is a DNA regulatory region capable of binding an RNA polymerase in a cell (e.g., directly or through other promoter bound proteins or substances) and initiating transcription of a coding sequence. A promoter sequence is, in general, bound at its 3′ terminus by the transcription initiation site and extends upstream (5′ direction) to include the minimum number of bases or elements necessary to initiate transcription at any level. The promoter may be operably associated with or operably linked to the expression control sequences, including enhancer and repressor sequences or with a nucleic acid to be expressed. In some embodiments, the promoter may be inducible. In some embodiments, the inducible promoter may be unidirectional or bio-directional. In some embodiments, the promoter may be a constitutive promoter. In some embodiments, the promoter can be a hybrid promoter, in which the sequence containing the transcriptional regulatory region is obtained from one source and the sequence containing the transcription initiation region is obtained from a second source. Systems for linking control elements to coding sequence within a transgene are well known in the art (general molecular biological and recombinant DNA techniques are described in Sambrook, Fritsch, and Maniatis, Molecular Cloning: A Laboratory Manual, Second Edition, Cold Spring Harbor Laboratory Press, Cold Spring Harbor, N.Y., 1989). Commercial vectors suitable for inserting a transgene for expression in various host cells under a variety of growth and induction conditions are also well known in the art.

In some embodiments, a specific promoter may be used to control expression of a nucleic acid encoding a trigger-responsive immune-inactivatingsignaling polypeptide in a mammalian host cell such as, but are not limited to, SRα-promoter (Takebe, et al., Molec. and Cell. Bio. 8:466-472 (1988)), the human CMV immediate early promoter (Boshart, et al., Cell 41:521-530 (1985); Foecking, et al., Gene 45:101-105 (1986)), human CMV promoter, the human CMV5 promoter, the murine CMV immediate early promoter, the EF1-α-promoter, a hybrid CMV promoter for liver specific expression (e.g., made by conjugating CMV immediate early promoter with the transcriptional promoter elements of either human α-1-antitrypsin (HAT) or albumin (HAL) promoter), or promoters for hepatoma specific expression (e.g., wherein the transcriptional promoter elements of either human albumin (HAL; about 1000 bp) or human α-1-antitrypsin (HAT, about 2000 bp) are combined with a 145 long enhancer element of human α-1-microglobulin and bikunin precursor gene (AMBP); HAL-AMBP and HAT-AMBP); the SV40 early promoter region (Benoist, et al., Nature 290:304-310 (1981)), the Orgyia pseudotsugata immediate early promoter, the herpes thymidine kinase promoter (Wagner, et al., Proc. Natl. Acad. Sci. USA 78:1441-1445 (1981)); or the regulatory sequences of the metallothionein gene (Brinster, et al., Nature 296:39-42 (1982)). In some embodiments, the mammalian promoter is a is a constitutive promoter such as, but not limited to, the hypoxanthine phosphoribosyl transferase (HPTR) promoter, the adenosine deaminase promoter, the pyruvate kinase promoter, the beta-actin promoter as well as other constitutive promoters known to those of ordinary skill in the art.

In some embodiments, a specific promoter may be used to control expression of a nucleic acid encoding a trigger-responsive immune-inactivatingsignaling polypeptide in a prokaryotic host cell such as, but are not limited to, the β-lactamase promoter (Villa-Komaroff, et al., Proc. Natl. Acad. Sci. USA 75:3727-3731 (1978)); the tac promoter (DeBoer, et al., Proc. Natl. Acad. Sci. USA 80:21-25 (1983)); the T7 promoter, the T3 promoter, the M13 promoter or the M16 promoter; in a yeast host cell such as, but are not limited to, the GAL1, GAL4 or GAL10 promoter, the ADH (alcohol dehydrogenase) promoter, PGK (phosphoglycerol kinase) promoter, alkaline phosphatase promoter, glyceraldehyde-3-phosphate dehydrogenase III (TDH3) promoter, glyceraldehyde-3-phosphate dehydrogenase II (TDH2) promoter, glyceraldehyde-3-phosphate dehydrogenase I (TDH1) promoter, pyruvate kinase (PYK), enolase (ENO), or triose phosphate isomerase (TPI).

In some embodiments, the promoter may be a viral promoter, many of which are able to regulate expression of a nucleic acid encoding a trigger-responsive immune-inactivatingsignaling polypeptide in several host cell types, including mammalian cells. Viral promoters that have been shown to drive constitutive expression of coding sequences in eukaryotic cells include, for example, simian virus promoters, herpes simplex virus promoters, papilloma virus promoters, adenovirus promoters, human immunodeficiency virus (HIV) promoters, Rous sarcoma virus promoters, cytomegalovirus (CMV) promoters, the long terminal repeats (LTRs) of Moloney murine leukemia virus and other retroviruses, the thymidine kinase promoter of herpes simplex virus as well as other viral promoters known to those of ordinary skill in the art.

In some embodiments, the gene control elements of an expression vector may also include 5′ non-transcribing and 5′ non-translating sequences involved with the initiation of transcription and translation, respectively, such as a TATA box, capping sequence, CAAT sequence, Kozak sequence and the like. Enhancer elements can optionally be used to increase expression levels of a polypeptide or protein to be expressed. Examples of enhancer elements that have been shown to function in mammalian cells include the SV40 early gene enhancer, as described in Dijkema, et al., EMBO J. (1985) 4: 761 and the enhancer/promoter derived from the long terminal repeat (LTR) of the Rous Sarcoma Virus (RSV), as described in Gorman, et al., Proc. Natl. Acad. Sci. USA (1982b) 79:6777 and human cytomegalovirus, as described in Boshart, et al., Cell (1985) 41:521. Genetic control elements of an expression vector will also include 3′ non-transcribing and 3′ non-translating sequences involved with the termination of transcription and translation. Respectively, such as a poly polyadenylation (polyA) signal for stabilization and processing of the 3′ end of an mRNA transcribed from the promoter. Poly A signals included, for example, the rabbit beta globin polyA signal, bovine growth hormone polyA signal, chicken beta globin terminator/polyA signal, or SV40 late polyA region.

Expression vectors will preferably, but optionally, be include at least one selectable marker. In some embodiments, the selectable maker is a nucleic acid sequence encoding a resistance gene operably linked to one or more genetic regulatory elements, to bestow upon a host cell the ability to maintain viability when grown in the presence of a cytotoxic chemical and/or drug. In some embodiments, a selectable agent may be used to maintain retention of the expression vector within the host cell. In some embodiments, the selectable agent is may be used to prevent modification (i.e. methylation) and/or silencing of the transgene sequence within the expression vector. In some embodiments, a selectable agent is used to maintain episomal expression of the vector within the host cell. In some embodiments, the selectable agent is used to promote stable integration of the transgene sequence into the host cell genome. In some embodiments, an agent and/or resistance gene may include, but is not limited to, methotrexate (MTX), dihydrofolate reductase (DHFR, U.S. Pat. Nos. 4,399,216; 4,634,665; 4,656,134; 4,956,288; 5,149,636; 5,179,017, ampicillin, neomycin (G418), zeomycin, mycophenolic acid, or glutamine synthetase (GS, U.S. Pat. Nos. 5,122,464; 5,770,359; 5,827,739) for eukaryotic host cell; tetracycline, ampicillin, kanamycin or chloramphenicol for a prokaryotic host cell; and URA3, LEU2, HIS3, LYS2, HIS4, ADE8, CUP1 or TRP1 for a yeast host cell.

Expression vectors may be transfected, transformed or transduced into a host cell. As used herein, the terms “transfection,” “transformation” and “transduction” all refer to the introduction of an exogenous nucleic acid sequence into a host cell. In some embodiments, expression vectors containing nucleic acid sequences encoding for I2S and/or FGE are transfected, transformed or transduced into a host cell at the same time. In some embodiments, expression vectors containing nucleic acid sequences encoding for I2S and/or FGE are transfected, transformed or transduced into a host cell sequentially. For example, a vector encoding an I2S protein may be transfected, transformed or transduced into a host cell first, followed by the transfection, transformation or transduction of a vector encoding an FGE protein, and vice versa. Examples of transformation, transfection and transduction methods, which are well known in the art, include liposome delivery, i.e., Lipofectamine™ (Gibco BRL) Method of Hawley-Nelson, Focus 15:73 (1193), electroporation, CaPO4 delivery method of Graham and van der Erb, Virology, 52:456-457 (1978), DEAE-Dextran medicated delivery, microinjection, biolistic particle delivery, polybrene mediated delivery, cationic mediated lipid delivery, transduction, and viral infection, such as, e.g., retrovirus, lentivirus, adenovirus adenoassociated virus and Baculovirus (Insect cells). General aspects of cell host transformations have been described in the art, such as by Axel in U.S. Pat. No. 4,399,216; Sambrook, supra, Chapters 1-4 and 16-18; Ausubel, supra, chapters 1, 9, 13, 15, and 16. For various techniques for transforming mammalian cells, see Keown et al., Methods in Enzymology (1989), Keown et al., Methods in Enzymology, 185:527-537 (1990), and Mansour et al., Nature, 336:348-352 (1988).

For long-term, high-yield production of recombinant proteins, stable expression is often preferred. For example, cell lines which stably express a trigger-responsive immune-inactivatingsignaling polypeptide can be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells can be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express a trigger-responsive immune-inactivatingsignaling polypeptide. Such engineered cell lines can be particularly useful in screening and evaluation of compounds that interact directly or indirectly with a trigger-responsive immune-inactivatingsignaling polypeptide.

A number of selection systems can be used, including but not limited to the herpes simplex virus thymidine kinase (Wigler, et al., Cell, 11:223 (1977)), hypoxanthine-guanine phosphoribosyltransferase (Szybalska & Szybalski, Proc. Natl. Acad. Sci. USA, 48:202 (1992)), and adenine phosphoribosyltransferase (Lowy, et al., Cell, 22:817 (1980)) genes can be employed in tk-, hgprt- or aprt-cells, respectively. Also, anti-metabolite resistance can be used as the basis of selection for the following genes: dhfr, which confers resistance to methotrexate (Wigler, et al., Proc. Natl. Acad. Sci. USA, 77:357 (1980); O'Hare, et al., Proc. Natl. Acad. Sci. USA, 78:1527 (1981)); gpt, which confers resistance to mycophenolic acid (Mulligan & Berg, Proc. Natl. Acad. Sci. USA, 78:2072 (1981)); neo, which confers resistance to the aminoglycoside G-418; Wu and Wu, Biotherapy, 3:87-95 (1991); Tolstoshev, Ann. Rev. Pharmacol. Toxicol., 32:573-596 (1993); Mulligan, Science, 260:926-932 (1993); and Morgan and Anderson, Ann. Rev. Biochem., 62:191-217 (1993); Can, 1993, TIB TECH 11(5):155-215); and hygro, which confers resistance to hygromycin (Santerre et al., Gene, 30:147 (1984)). Methods commonly known in the art of recombinant DNA technology can be routinely applied to select the desired recombinant clone, and such methods are described, for example, in Current Protocols in Molecular Biology, Ausubel, et al., eds. (John Wiley & Sons, N Y 1993); Kriegler, Gene Transfer and Expression, A Laboratory Manual (Stockton Press, N Y 1990); and Current Protocols in Human Genetics, Dracopoli, et al., eds. (John Wiley & Sons, N Y 1994), Chapters 12 and 13; Colberre-Garapin, et al., J. Mol. Biol., 150:1 (1981).

The expression levels of a trigger-responsive immune-inactivatingsignaling polypeptide described herein can be increased by vector amplification (for a review, see Bebbington and Hentschel, “The use of vectors based on gene amplification for the expression of cloned genes in mammalian cells in DNA cloning,” Vol. 3. (Academic Press, New York (1987)). When a marker in the vector system expressing a trigger-responsive immune-inactivatingsignaling polypeptide is amplifiable, increase in the level of inhibitor present in culture of host cell will increase the number of copies of the marker gene. Since the amplified region is associated with the nucleic acid sequence encoding a trigger-responsive immune-inactivatingsignaling polypeptide described herein, production of a trigger-responsive immune-inactivatingsignaling polypeptide can also increase (Crouse, et al., Mol. Cell. Biol., 3:257 (1983)).

### Polypeptides

Among other things, the present disclosure provides a trigger-responsive dominant negative signaling polypeptide described herein.

In certain embodiments, a trigger-responsive dominant negative signaling polypeptide is an endoxifen-responsive dominant negative Zap-70 polypeptide. In some embodiments, an endoxifen-responsive dominant negative Zap-70 polypeptide has an amino acid sequence according to SEQ ID NO: 8 or SEQ ID NO: 30. As disclosed herein, SEQ ID NO: 8 and SEQ ID NO: 30 represent exemplary amino acid sequences of endoxifen-responsive dominant negative Zap-70 polypeptides. In some embodiments, an endoxifen-responsive dominant negative Zap-70 polypeptide can have an amino acid sequence substantially similar to SEQ ID NO: 8 or SEQ ID NO: 30. In some embodiments, an endoxifen-responsive dominant negative Zap-70 polypeptide can have an amino acid sequence with at least 75%, at least 80%, at least 85%, at least 90%, at least 95%, at least 96%, at least 97%, at least 98%, or at least 99% sequence identity to the nucleotide sequence of SEQ ID NO: 8 or the nucleotide sequence of SEQ ID NO: 30.

SEQ ID NO: 8
M N E L A L K L A G L D I N K T M
P D P A A H L P F F Y G S I S R A
E A E E H L K L A G M A D G L F L
L R Q C L R S L G G Y V L S L V H
D V R F H H F P I E R Q L N G T Y
A I A G G K A H C G P A E L C E F
Y S R D P D G L P C N L R K P C N
R P S G L E P Q P G V F D C L R D
A M V R D Y V R Q T W K L E G E A
L E Q A I I S Q A P Q V E K L I A
T T A H E R M P W Y H S S L T R E
E A E R K L Y S G A Q T D G K F L
L R P R K E Q G T Y A L S L I Y G
K T V Y H Y L I S Q D K A G K Y C
I P E G T K F D T L W Q L V E Y L
K L K A D G L I Y C L K E A C P N
S S A S N A S G A A A P T L P A H
P S T L T G S S A G D M R A A N L
W P S P L M I K R S K K N S L A L
S L T A D Q M V S A L L D A E P P
I L Y S E Y D P T R P F S E A S M
M G L L T N L A D R E L V H M I N
W A K R V P G F V D L T L H D Q V
H L L E C A W L E I L M I G L V W
R S M E H P V K L L F A P N L L L
D R N Q G K C V E G M V E I F D M
L L A T S S R F R M M N L Q G E E
F V C L K S I I L L N S G V Y T F
L S S T L K S L E E K D H I H R V
L D K I T D T L I H L M A K A G L
T L Q Q Q H Q R L A Q L L L I L S
H I R H M S N K G M E H L Y S M K
C K N V V P L Y D L L L E A A D A
H R L H A P T S R G G A S V E E T
D Q S H L A T A G S T S S H S L Q
K Y Y I T G E A E G F P A T V

In some embodiments, an endoxifen-responsive dominant negative Zap-70 polypeptide has an amino acid sequence according to SEQ ID NO: 15 or SEQ ID NO: 31. As disclosed herein, SEQ ID NO: 15 and SEQ ID NO: 31 represent exemplary amino acid sequences of endoxifen-responsive dominant negative Zap-70 polypeptides. In some embodiments, an endoxifen-responsive dominant negative Zap-70 polypeptide can have an amino acid sequence substantially similar to SEQ ID NO: 15 or SEQ ID NO: 31. In some embodiments, an endoxifen-responsive dominant negative Zap-70 polypeptide can have an amino acid sequence with at least 75%, at least 80%, at least 85%, at least 90%, at least 95%, at least 96%, at least 97%, at least 98%, or at least 99% sequence identity to the nucleotide sequence of SEQ ID NO: 15 or the nucleotide sequence of SEQ ID NO: 31.

SEQ ID NO: 15
MNELALKLAGLDINKTMPDPAAHLPFFYGSISRAE
AEEHLKLAGMADGLFLLRQCLRSLGGYVLSLVHDV
RFHHFPIERQLNGTYAIAGGKAHCGPAELCEFYSR
DPDGLPCNLRKPCNRPSGLEPQPGVFDCLRDAMVR
DYVRQTWKLEGEALEQAIISQAPQVEKLIATTAHE
RMPWYHSSLTREEAERKLYSGAQTDGKFLLRPRKE
QGTYALSLIYGKTVYHYLISQDKAGKYCIPEGTKF
DTLWQLVEYLKLKADGLIYCLKEACPNSSASNASG
AAAPTLPAHPSTLTGSSAGDMRAANLWPSPLMIKR
SKKNSLALSLTADQMVSALLDAEPPILYSEYDPTR
PFSEASMMGLLTNLADRELVHMINWAKRVPGFVDL
TLHDQVHLLECAWLEILMIGLVWRSMEHPLKLLFA
PNLLLDRNQGKCVEGMVEIFDMLLATSSRFRMMNL
QGEEFVCLKSIILLNSGVYTFLSSTLKSLEEKDHI
HRVLDKITDTLIHLMAKAGLTLQQQHQRLAQLLLI
LSHIRHMSNKGMEHLYSMKCKNVVPLYDLLLEAAD
AHRLHAPTSRGGASVEETDQSHLATAGSTSSHSLQ
KYYITGEAEGFPATV

In certain embodiments, a trigger-responsive dominant negative signaling polypeptide is an endoxifen-responsive dominant negative LCK polypeptide. In some embodiments, an endoxifen-responsive dominant negative LCK polypeptide has an amino acid sequence according to SEQ ID NO: 18 or SEQ ID NO: 33. As disclosed herein, SEQ ID NO: 18 and SEQ ID NO: 33 represent exemplary amino acid sequences of endoxifen-responsive dominant negative LCK polypeptides. In some embodiments, an endoxifen-responsive dominant negative LCK polypeptide can have an amino acid sequence substantially similar to SEQ ID NO: 18 or SEQ ID NO: 33. In some embodiments, an endoxifen-responsive dominant negative LCK polypeptide can have an amino acid sequence with at least 75%, at least 80%, at least 85%, at least 90%, at least 95%, at least 96%, at least 97%, at least 98%, or at least 99% sequence identity to the nucleotide sequence of SEQ ID NO: 18 or the nucleotide sequence of SEQ ID NO: 33.

SEQ ID NO: 18
MNELALKLAGLDINKTMGCGCSSHPEDDWMENIDV
CENCHYPIVPLDGKGTLLIRNGSEVRDPLVTYEGS
NPPASPLQDNLVIALHSYEPSHDGDLGFEKGEQLR
ILEQSGEWWKAQSLTTGQEGFIPFNFVAKANSLEP
EPWFFKNLSRKDAERQLLAPGNTHGSFLIRESEST
AGSFSLSVRDFDQNQGEVVKHYKIRNLDNGGFYIS
PRITFPGLHELVRHYTNASDGLCTRLSRPCQTQKP
QKPWWEDEWEVPRETLKLVERLGAGQFGEVWMGYY
NGGSSAGDMRAANLWPSPLMIKRSKKNSLALSLTA
DQMVSALLDAEPPILYSEYDPTRPFSEASMMGLLT
NLADRELVHMINWAKRVPGFVDLTLHDQVHLLECA
WLEILMIGLVWRSMEHPLKLLFAPNLLLDRNQGKC
VEGMVEIFDMLLATSSRFRMMNLQGEEFVCLKSII
LLNSGVYTFLSSTLKSLEEKDHIHRVLDKITDTLI
HLMAKAGLTLQQQHQRLAQLLLILSHIRHMSNKGM
EHLYSMKCKNVVPLYDLLLEAADAHRLHAPTSRGG
ASVEETDQSHLATAGSTSSHSLQKYYITGEAEGFP
ATV

In certain embodiments, a trigger-responsive constitutively active signaling polypeptide is an endoxifen-responsive constitutively active SHP1 polypeptide. In some embodiments, an endoxifen-responsive constitutively active SHP1 polypeptide has an amino acid sequence according to SEQ ID NO: 24 or SEQ ID NO: 35. As disclosed herein, SEQ ID NO: 24 and SEQ ID NO: 35 represent exemplary amino acid sequences of endoxifen-responsive constitutively active SHP1 polypeptides. In some embodiments, an endoxifen-responsive constitutively active SHP1 polypeptide can have an amino acid sequence substantially similar to SEQ ID NO: 24 or SEQ ID NO: 35. In some embodiments, an endoxifen-responsive constitutively active SHP1 polypeptide can have an amino acid sequence with at least 75%, at least 80%, at least 85%, at least 90%, at least 95%, at least 96%, at least 97%, at least 98%, or at least 99% sequence identity to the nucleotide sequence of SEQ ID NO: 24 or the nucleotide sequence of SEQ ID NO: 35.

SEQ ID NO: 24
MNELALKLAGLDINKTRQPYYATRVNAADIENRVL
ELNKKQESEDTAKAGFWEEFESLQKQEVKNLHQRL
EGQRPENKGKNRYKNILPFDHSRVILQGRDSNIPG
SDYINANYIKNQLLGPDENAKTYIASQGCLEATVN
DFWQMAWQENSRVIVMTTREVEKGRNKCVPYWPEV
GMQRAYGPYSVTNCGEHDTTEYKLRTLQVSPLDNG
DLIREIWHYQYLSWPDHGVPSEPGGVLSFLDQINQ
RQESLPHAGPIIVHCSAGIGRTGTIIVIDMLMENI
STKGLDCDIDIQKTIQMVRAQRSGMVQTEAQYKFI
YVAIAQFIETTKKKLEVLQSQKGQESEYGNITYPP
AMKNAHAKASRTSSKHKEDVYENLHTKNKREEKVK
KQRSADKEKSKGSLKRKGSSAGDMRAANLWPSPLM
IKRSKKNSLALSLTADQMVSALLDAEPPILYSEYD
PTRPFSEASMMGLLTNLADRELVHMINWAKRVPGF
VDLTLHDQVHLLECAWLEILMIGLVWRSMEHPLKL
LFAPNLLLDRNQGKCVEGMVEIFDMLLATSSRFRM
MNLQGEEFVCLKSIILLNSGVYTFLSSTLKSLEEK
DHIHRVLDKITDTLIHLMAKAGLTLQQQHQRLAQL
LLILSHIRHMSNKGMEHLYSMKCKNVVPLYDLLLE
AADAHRLHAPTSRGGASVEETDQSHLATAGSTSSH
SLQKYYITGEAEGFPATV

Once a trigger-responsive immune-inactivating signaling polypeptide of the invention has been expressed, it can be purified by any method known in the art for protein purification for example, by chromatography (e.g., ion exchange, affinity, particularly by affinity for the specific antigen after Protein A, and sizing column chromatography), centrifugation, differential solubility, or by any other standard technique for the purification of proteins. In addition, a trigger-responsive immune-inactivating signaling polypeptide described herein can be fused to heterologous polypeptide sequences described herein or otherwise known in the art, to facilitate purification.

For the purpose of affinity purification, relevant matrices for affinity chromatography, such as glutathione-, amylase-, and nickel- or cobalt-conjugated resins can be used. Many of such matrices are available in “kit” form, such as the Pharmacia GST purification system and the QIAexpress™ system (Qiagen®) useful with histidine-tagged proteins. Tags can also facilitate the detection of a trigger-responsive immune-inactivating signaling polypeptide. Examples of such tags can include the various fluorescent proteins (e.g., GFP), as well as “epitope tags,” which are usually short peptide sequences for which a specific antibody is available. Well-known epitope tags for which specific monoclonal antibodies are readily available include FLAG, influenza virus haemagluttinin (HA), and c-myc tags.

In view of the above, one aspect provided herein is a method of producing or manufacturing a trigger-responsive immune-inactivating signaling polypeptide described herein. In some embodiments, a method of producing or manufacturing a trigger-responsive immune-inactivating signaling polypeptide described herein includes expressing the trigger-responsive immune-inactivating signaling polypeptide from a nucleic acid or a vector that encodes the trigger-responsive immune-inactivating signaling polypeptide in a cell (e.g., a host cell). In some embodiments, a method further comprises recovering the trigger-responsive immune-inactivating signaling polypeptide.

In some embodiments, a method can include (a) culturing a cell comprising a nucleic acid sequence encoding a trigger-responsive immune-inactivating signaling polypeptide described herein, or a vector (e.g., a plasmid) comprising a nucleic acid sequence encoding a trigger-responsive immune-inactivating signaling polypeptide described herein, or a viral particle comprising such a nucleic acid or a vector, where the culturing is performed under conditions such that the trigger-responsive immune-inactivating signaling polypeptide is expressed; and (b) recovering the trigger-responsive immune-inactivating signaling polypeptide.

In some embodiments, a method of manufacturing a trigger-responsive immune-inactivating signaling polypeptide can include expressing the trigger-responsive immune-inactivating signaling polypeptide from the nucleic acid or the vector described herein in a host cell. In some embodiments, a method of manufacturing a trigger-responsive immune-inactivating signaling polypeptide can include recovering an expressed trigger-responsive immune-inactivating signaling polypeptide from a host cell.

In some embodiments, a method of manufacture can include introducing a nucleic acid or a vector described herein into a T cell.

### Viral Particle

Among other things, the present disclosure provides a viral particle comprising a nucleic acid sequence encoding a trigger-responsive immune-inactivating signaling polypeptide described herein or a trigger-responsive immune-inactivating signaling polypeptide described herein. In some embodiments, a viral particle can include an adenoviral particle, retroviral particle, lentiviral particle, and/or combinations thereof.

### Cells

Among other things, the present disclosure provides a cell comprising a nucleic acid sequence (such as a vector) encoding a trigger-responsive immune-inactivating signaling polypeptide described herein or a viral particle described herein. A variety of technologies are known to those skilled in the art for engineering any of a variety of cells to contain and/or express a nucleic acid (e.g., a nucleic acid vector) encoding a trigger-responsive immune-inactivating signaling polypeptide as described herein (see, for example, Green & Sambrook Molecular Cloning, Cold Spring Harbor Laboratory Press). To give but a few examples, available technologies for introducing nucleic acids into mammalian cells include transfection (e.g., mediated by cationic lipid reagents, by calcium phosphate, by DEAE-Dextran, by DOTMA/DOGS, by electroporation, and/or by combinations thereof) and use of viral vectors (e.g., adenoviral vectors, retroviral vectors, lentiviral vectors, and/or combinations thereof).

In some embodiments, a provided cell may (e.g., may be engineered to) transiently contain and/or express a nucleic acid that encodes trigger-responsive immune-inactivating signaling polypeptide; in some embodiments, a provided cell may (e.g., may be engineered to) stably contain and/or express a nucleic acid that encodes trigger-responsive immune-inactivating signaling polypeptide. In some embodiments, a provided cell may (e.g., may be engineered to) contain and/or express multiple (e.g., 2, 3, 4, 5, 6, 7, 8, 9, 10, 11, 12, 13, 14, 15, 16, 17, 18, 19, 20, 21, 22, 23, 24, 25, or more copies or instances of a nucleic acid that encodes trigger-responsive immune-inactivating signaling polypeptide; in some embodiments, a provided cell may (e.g., may be engineered to) contain and/or express only a single copy of a nucleic acid that encodes trigger-responsive immune-inactivating signaling polypeptide.

In some embodiments, a cell as provided herein may be designed, engineered and/or utilized for production and/or secretion of a trigger-responsive immune-inactivating signaling polypeptide as described herein. A variety of host-expression vector systems can be utilized to express a trigger-responsive immune-inactivating signaling polypeptide described herein. Such host-expression systems represent vehicles by which the coding sequences of interest can be produced and subsequently purified, but also represent cells which can, when transformed or transfected with the appropriate nucleotide coding sequences, express a trigger-responsive immune-inactivating signaling polypeptide described herein in situ. These include but are not limited to microorganisms such as prokaryotic bacteria (e.g., attenuated Bacillus anthracis strains, E. coli, B. subtilis) transformed with recombinant bacteriophage DNA, plasmid DNA or cosmid DNA expression vectors containing a trigger-responsive immune-inactivating signaling polypeptide coding sequence; yeast (e.g., Saccharomyces, Pichia) transformed with recombinant yeast expression vectors containing a trigger-responsive immune-inactivating signaling polypeptide coding sequence; insect cell systems infected with recombinant virus expression vectors (e.g., baculovirus) containing a trigger-responsive immune-inactivating signaling polypeptide coding sequence; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing a trigger-responsive immune-inactivating signaling polypeptide coding sequences; or mammalian cell systems (e.g., COS, CHO, BHK, NS0, 293, or 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). In some embodiments, a cell may be or comprise a human cell (e.g., a T cell such as a CAR-T or TCR-T cell as described herein).

A host cell can be chosen that modulates the expression of an inserted sequence, or modifies and processes a gene product in the specific fashion desired. In some embodiments, modifications (e.g., glycosylation) and processing (e.g., cleavage) of polypeptide products (e.g., a trigger-responsive immune-inactivating signaling polypeptide) can be important for the function of the protein. Different host cells have characteristic and specific mechanisms for the post-translational processing and modification of proteins and gene products. Appropriate cell lines or host systems can be chosen to ensure the correct modification and processing of the foreign protein expressed.

In some embodiments, mammalian host cells can include, but are not limited to, CHO, VERY, BHK, HeLa, COS, MDCK, 293, 3T3, W138, BT483, Hs578T, HTB2, BT20 and T47D, NS0 (a murine myeloma cell line that does not endogenously produce any immunoglobulin chains), CRL7O3O and HsS78Bst cells. In mammalian host cells, a number of viral-based expression systems can be utilized. In cases where an adenovirus is used as an expression vector, the coding sequence of trigger-responsive immune-inactivating signaling polypeptide can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) can result in a recombinant virus that is viable and capable of expressing a trigger-responsive immune-inactivating signaling polypeptide in infected hosts. (See, e.g., Logan & Shenk, Proc. Natl. Acad. Sci. USA, 81:355-359 (1984)). Specific initiation signals can also be required for efficient translation of inserted trigger-responsive immune-inactivating signaling polypeptide coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see Bittner, et al., Methods in Enzymol., 153:51-544 (1987)).

In some embodiments, a host cell can be a cell of the immune system (e.g., a monocyte, eosinophil, neutrophil, basophil, macrophage, dendritic cell, natural killer cell, T cell (e.g., helper T cell and cytotoxic T cell), T regulatory cell, or B cell). In some embodiments, a host cell can be a T cell, e.g., primary T cell or an immortal T cell line. An immortal T cell line can be a Jurkat cell line, for example, Neo Jurkat cells, BCL2 Jurkat cells, Jurkat E6.1 cells, J.RT3-T3.5 cells, Daudi cells, HuT78 cells, I9.2 cells, or Loucy cells. In some embodiments, a T cell can be a wild-type T cell. In some embodiments, a T cell can be an engineered T cell, e.g., a CAR-T cell.

CAR-T cells are T cells that have been engineered to express a chimeric antigen receptor (a “CAR”). Typically, CARs are composed of an extracellular antigen-recognition moiety that is linked, via spacer/hinge and transmembrane domains, to an intracellular signaling domain that can include costimulatory domains and T cell activation moieties. In some embodiments, CARs can recognize unprocessed antigens independently of their expression of major histocompatibility antigens, which is one example of how CARs can differ from wild-type TCRs. In some embodiments, a CAR can be characterized by its ability to bind to a protein, a polypeptide, a carbohydrate, a ganglioside, a proteoglycan, and or a glycosylated protein.

In bacterial systems, a number of expression vectors can be advantageously selected depending upon the use intended for a trigger-responsive immune-inactivating signaling polypeptide being expressed. For example, when a large quantity of such a protein is to be produced, for the generation of pharmaceutical compositions of a trigger-responsive immune-inactivating signaling polypeptide, vectors which direct the expression of high levels of a trigger-responsive immune-inactivating signaling polypeptide product that can be readily purified can be desirable. Such vectors include, but are not limited, to the E. coli expression vector pUR278 (Ruther, et al., EMBO J., 2:1791 (1983)), in which a trigger-responsive immune-inactivating signaling polypeptide coding sequence can be ligated individually into the vector in frame with the lacZ coding region so that a fusion protein is produced; pIN vectors (Inouye & Inouye, Nucleic Acids Res., 13:3101-3109 (1985); Van Heeke & Schuster, J. Biol. Chem., 24:5503-5509 (1989)); and the like pGEX vectors can also be used to express foreign polypeptides as fusion proteins with glutathione S-transferase (GST). In general, such trigger-responsive immune-inactivating signaling polypeptides are soluble and can easily be purified from lysed cells by adsorption and binding to matrix glutathione-agarose beads followed by elution in the presence of free glutathione. The pGEX vectors are designed to include thrombin or factor Xa protease cleavage sites so that the cloned target gene product can be released from the GST moiety. Alternately, the pET expression vectors can be used for producing histidine-tagged recombinant proteins, where the histidine-tagged recombinant proteins can be affinity purified by a nickel column. Expression of recombinant proteins in Pichia pastoris is described by Holliger, P., Meth. Mol. Biol., 178:349-57 (2002). In some embodiments, expression of a trigger-responsive immune-inactivating signaling polypeptide can be under the control of an inducible expression system, e.g., IPTG-inducible expression in E. coli, baculovirus expression, or methanol-inducible AOX1-directed expression in P. pastoris.

In an insect system, Autographa californica nuclear polyhedrosis virus (AcNPV) can be used as a vector to express foreign genes. A virus can grow in Spodoptera frugiperda cells. A trigger-responsive immune-inactivating signaling polypeptide coding sequence can be cloned individually into non-essential regions (for example the polyhedrin gene) of the virus and placed under control of an AcNPV promoter (for example the polyhedrin promoter).

Large scale expression of heterologous proteins in the algae Chlamydomonas reinhardtii are described by Griesbeck, C., et al., Mol. Biotechnol. 34:213-33 (2006); Manuell, A L, et al. Plant Biotechnol J. Eprint (2007); Franklin S E and Mayfield S P, Expert Opin Biol Ther. 5(2):225-35 (2007); Mayfield S P and Franklin S E, Vaccine, 23(15):1828-32 (2005); and Fuhrmann M., Methods Mol Med. 94:191-5 (2004). Foreign heterologous coding sequences can be inserted into the genome of the nucleus, chloroplast and mitochondria by homologous recombination. The chloroplast expression vector p64 carrying the most versatile chloroplast selectable marker aminoglycoside adenyl transferase (aadA), which confers resistance to spectinomycin or streptomycin, can be used to express foreign protein in the chloroplast. A biolistic gene gun method can be used to introduce the vector in the algae. Upon its entry into chloroplasts, the foreign DNA can be released from the gene gun particles and integrates into the chloroplast genome through homologous recombination.

Compositions that Deliver a Trigger-Responsive Immune-Inactivating Signaling Polypeptide

In accordance with the present disclosure, any of a variety of modalities may be utilized to deliver a trigger-responsive immune-inactivating signaling polypeptide described herein. To give but a few examples, in some embodiments, an immune-inactivating signaling polypeptide as described herein is administered (i.e., to a subject or system). In some embodiments, a nucleic acid that encodes an immune-inactivating signaling polypeptide may be administered; in some such embodiments, the encoding nucleic acid may be associated with one or more elements that directs its expression. In some embodiments, a cell containing and/or expressing an immune-inactivating signaling polypeptide and/or a nucleic acid that encodes it is administered; in some such embodiments, the cell is an immune system cell, e.g., a monocyte, eosinophil, neutrophil, basophil, macrophage, dendritic cell, natural killer cell, T cell (e.g., helper T cell and cytotoxic T cell), T regulatory cell, or B cell. In some embodiments, the cell is a T cell (e.g., a CAR-T or TCR T cell). In some embodiments, a T cell (e.g., a CAR-T or TCR T cell) that has been engineered to contain (e.g., to express) a trigger-responsive immune-inactivating signaling polypeptide, and/or a nucleic acid that encodes it, is administered. In some embodiments, a viral particle containing an immune-inactivating signaling polypeptide and/or a nucleic acid that encodes and/or expresses it is administered.

Thus some embodiments, a trigger-responsive immune-inactivating signaling polypeptide described herein can be directly administered. As such, in some embodiments, a composition that delivers a trigger-responsive immune-inactivating signaling polypeptide described herein includes a trigger-responsive immune-inactivating signaling polypeptide described herein.

In some embodiments, a trigger-responsive immune-inactivating signaling polypeptide described herein can be delivered by delivering a nucleic acid that encodes a trigger-responsive immune-inactivating signaling polypeptide described herein, a vector that includes such a nucleic acid, a cell that includes a nucleic acid that encodes a trigger-responsive immune-inactivating signaling polypeptide described herein, a cell that includes a vector comprising a nucleic acid that encodes a trigger-responsive immune-inactivating signaling polypeptide described herein, and/or a cell that includes a trigger-responsive immune-inactivating signaling polypeptide described herein. As such, in some embodiments, a composition that delivers a trigger-responsive immune-inactivating signaling polypeptide described herein includes a nucleic acid that encodes a trigger-responsive immune-inactivating signaling polypeptide described herein, a vector that includes such a nucleic acid, a cell that includes a nucleic acid that encodes a trigger-responsive immune-inactivating signaling polypeptide described herein, a cell that includes a vector comprising a nucleic acid that encodes a trigger-responsive immune-inactivating signaling polypeptide described herein, and/or a cell that includes a trigger-responsive immune-inactivating signaling polypeptide described herein.

In some embodiments, a trigger-responsive immune-inactivating signaling polypeptide described herein can be delivered by delivering a viral particle that comprises a nucleic acid that encodes a trigger-responsive immune-inactivating signaling polypeptide described herein, a vector that includes such a nucleic acid, and/or a trigger-responsive immune-inactivating signaling polypeptide described herein. As such, in some embodiments, a composition that delivers a trigger-responsive immune-inactivating signaling polypeptide described herein includes a viral particle that comprises a nucleic acid that encodes a trigger-responsive immune-inactivating signaling polypeptide described herein, a vector that includes such a nucleic acid, and/or a trigger-responsive immune-inactivating signaling polypeptide described herein. Exemplary nucleic acids, vectors, cells and viral particles are described herein.

### Pharmaceutical Compositions

In some embodiments, a composition that delivers a trigger-responsive immune-inactivating signaling polypeptide can be a pharmaceutical composition. In some embodiments, a pharmaceutical composition can include physiologically acceptable carrier or excipient. Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, sugars such as mannitol, sucrose, or others, dextrose, magnesium stearate, talc, silicic acid, viscous paraffin, perfume oil, fatty acid esters, hydroxymethylcellulose, polyvinyl pyrrolidone, etc., as well as combinations thereof. A pharmaceutical composition can, if desired, be mixed with auxiliary agents (e.g., lubricants, preservatives, stabilizers, wetting agents, emulsifiers, salts for influencing osmotic pressure, buffers, coloring, flavoring and/or aromatic substances and the like), which do not deleteriously react with the active compounds or interfere with their activity. In certain embodiments, a water-soluble carrier suitable for intravenous administration is used. In some embodiments, a pharmaceutical composition can be sterile.

A suitable pharmaceutical composition, if desired, can also contain minor amounts of wetting or emulsifying agents, or pH buffering agents. A pharmaceutical composition can be a liquid solution, suspension, emulsion, tablet, pill, capsule, sustained release formulation, or powder. A pharmaceutical composition can also be formulated as a suppository, with traditional binders and carriers such as triglycerides. Oral pharmaceutical compositions can include standard carriers, such as pharmaceutical grades of mannitol, lactose, starch, magnesium stearate, polyvinyl pyrrolidone, sodium saccharine, cellulose, magnesium carbonate, etc.

A pharmaceutical composition can be formulated in accordance with the routine procedures as a pharmaceutical composition adapted for administration to human beings. The formulation of a pharmaceutical composition should suit the mode of administration. For example, in some embodiments, a composition for intravenous administration typically is a solution in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where a pharmaceutical composition is to be administered by infusion, it can be dispensed with an infusion bottle containing sterile pharmaceutical grade water, saline or dextrose/water. Where a pharmaceutical composition is administered by injection, an ampule of sterile water for injection or saline can be provided so that the ingredients may be mixed prior to administration.

A trigger-responsive immune-inactivating signaling polypeptide described herein can be formulated as neutral or salt forms in a pharmaceutical composition. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

### Uses

### Methods of Regulating the Activity of T Cells

The present disclosure recognizes that therapies involving the activation of T cells (e.g., ATCT) show promise in treating various conditions and/or diseases (e.g., cancer). However, the present disclosure also recognizes that, while activated T cells can be a powerful tool in treating various conditions and/or diseases, controlling T cell activation presents a significant challenge and a risk to patient health. For example, uncontrolled T cell activation can result in a “cytokine storm,” a potential lethal outcome. Therefore, there remains a need in the field for methods of treating subjects (e.g., human patients) that utilizes activated T cells, but also able to “dial back” T cell activity, if and when desired. The present disclosure addresses this need and provides methods by which activity of a T cell population (which may be a maintained T cell population) can be reversibly decreased and increased through application and removal of a trigger. Combining trigger-responsiveness with maintenance of T cell levels (and, in at least some embodiments, reversibility and/or tunability through adjustment of trigger “intensity”—e.g., concentration, level and/or frequency of application, etc) provides a remarkably sophisticated and effective system that, moreover, is applicable to any of a variety of T cell populations including, for example, existing ATCT (e.g., CAR-T and/or TCR) T cell populations. In many embodiments, provided methods allow for reversible inhibition of T cell activity.

In addition, the present disclosure provides a variety of other advantages relative to available method for regulating T cell activity including, for example, that methods utilizing a trigger-responsive immune-inactivating signaling peptide described herein can inhibit T cell activity without destroying T cells. This advantage allows for a substantial improvement in patient care. As discussed above, adoptive T Cell Therapy (ATCT) is one current approach that shows promise in treating various conditions and/or diseases (e.g., cancer). ATCT entails collection and isolation of T cells from a subject (e.g., a patient). Isolated T cells are then clonally enriched, modified, and/or engineered to achieve a T cell population having desired properties and/or characteristics. The T cell population can then be expanded through ex-vivo growth and reintroduced into the subject to allow the enriched, modified, and/or engineered T cells to specifically attack cells of interest. Using current methodologies, the reintroduced T cells (e.g., genetically modified T cells, e.g., CAR-T cells) are destroyed if a decrease in T cell activity is necessitated. Consequently, in order for a patient to continue with T cell therapy or undergo a subsequent round of T cell therapy, the patient may need to go through painful, expensive and/or time intensive procedures, to allow for the isolation of T cells, enrichment of T cells, modification and/or engineering of a T cell population, and reintroduction of T cells into the patient. The methods provided herein allow for control of T cell activity without destroying T cells. The provided methods represent a significant improvement in patient care because they reduce the risk of an adverse event involving increased and undesired T cell activity (e.g., a cytokine storm). Regardless, if such an adverse event were to occur, the provided methods eliminate or reduce the need for subsequent procedures needed to continue T cell therapy, which can significantly improve the patient experience and/or patient accessibility to T cell therapies.

In some embodiments, a method of regulating activity of T cells includes introducing a composition that delivers a trigger-responsive immune-inactivating signaling polypeptide as described herein. In some embodiments, introducing a composition that delivers a trigger-responsive immune-inactivating signaling polypeptide as described herein can include introducing the composition that delivers the trigger-responsive immune-inactivating signaling polypeptide to a cell, which can be performed, e.g., in vitro or ex vivo. In some embodiments, such a cell can be a primary T cell, a modified and/or engineered T cell (e.g., a CAR-T cell), or a T cell line (e.g., a Jurkat cell line).

In some embodiments, a primary T cell is obtained from a subject (e.g., a patient, e.g., a human patient). In some embodiments, a primary T cell is modified and/or engineered, e.g., to express a chimeric antigen receptor (CAR).

In some embodiments, a provided method can include introducing the composition that delivers the trigger-responsive immune-inactivating signaling polypeptide to a primary T cell or modified and/or engineered T cell. In some embodiments, following the introduction of the composition that delivers the trigger-responsive immune-inactivating signaling polypeptide into the primary T cell or modified and/or engineered T cell, the resulting T cell is introduced into a subject. In some embodiments, the subject into which the resulting T cell is introduced is the same subject the primary T cell is obtained from. In some embodiments, the subject into which the resulting T cell is introduced is a different subject than the primary T cell is obtained from.

In some embodiments, introducing a composition that delivers a trigger-responsive immune-inactivating signaling polypeptide as described herein can include administering the composition that delivers the trigger-responsive immune-inactivating signaling polypeptide to a subject (e.g., a patient, e.g., a human patient). A composition that delivers a trigger-responsive immune-inactivating signaling polypeptide described herein can be administered by any appropriate route. In some embodiments, a composition that delivers a trigger-responsive immune-inactivating signaling polypeptide described herein is administered systemically. Systemic administration may be intravenous, intradermal, intracranial, intrathecal, inhalation, transdermal (topical), intraocular, intramuscular, subcutaneous, intramuscular, oral, and/or transmucosal administration. In some embodiments, a composition that delivers a trigger-responsive immune-inactivating signaling polypeptide described herein is administered subcutaneously. As used herein, the term “subcutaneous tissue,” is defined as a layer of loose, irregular connective tissue immediately beneath the skin. For example, the subcutaneous administration may be performed by injecting a composition into areas including, but not limited to, the thigh region, abdominal region, gluteal region, or scapular region. In some embodiments, a composition that delivers a trigger-responsive immune-inactivating signaling polypeptide described herein is administered intravenously. In some embodiments, a composition that delivers a trigger-responsive immune-inactivating signaling polypeptide described herein is administered orally. In some embodiments, a composition that delivers a trigger-responsive immune-inactivating signaling polypeptide described herein is administered intracranially. In some embodiments, a composition that delivers a trigger-responsive immune-inactivating signaling polypeptide described herein is administered intrathecally. As used herein, the term “intrathecal administration” or “intrathecal injection” refers to an injection into the spinal canal (intrathecal space surrounding the spinal cord). Various techniques may be used including, without limitation, lateral cerebroventricular injection through a burrhole or cisternal or lumbar puncture or the like. More than one route can be used concurrently, if desired.

The present disclosure contemplates single, as well as, multiple administrations of a therapeutically effective amount of a composition that delivers a trigger-responsive immune-inactivating signaling polypeptide described herein. In some embodiments, a composition that delivers a trigger-responsive immune-inactivating signaling polypeptide described herein can be administered at regular intervals, depending on the nature, severity and extent of the subject's condition.

In many embodiments, a method of regulating activity of T cells includes introducing a trigger described herein. In some embodiments, introducing a trigger as described herein can include introducing the trigger to a cell, which can be performed, e.g., in vitro or ex vivo. In some embodiments, such a cell can be a primary T cell, a modified and/or engineered T cell (e.g., a CAR-T cell), or a T cell line (e.g., a Jurkat cell line).

In some embodiments, introducing a trigger as described herein can include administering the trigger to a subject (e.g., a patient, e.g., a human patient). A trigger described herein can be administered by any appropriate route. In some embodiments, a trigger described herein is administered systemically. Systemic administration may be intravenous, intradermal, intracranial, intrathecal, inhalation, transdermal (topical), intraocular, intramuscular, subcutaneous, intramuscular, oral, and/or transmucosal administration. In some embodiments, a trigger described herein is administered subcutaneously. As used herein, the term “subcutaneous tissue,” is defined as a layer of loose, irregular connective tissue immediately beneath the skin. For example, the subcutaneous administration may be performed by injecting a composition into areas including, but not limited to, the thigh region, abdominal region, gluteal region, or scapular region. In some embodiments, a trigger described herein is administered intravenously. In some embodiments, a trigger is administered orally. In some embodiments, a trigger is administered intracranially. In some embodiments, a trigger is administered intrathecally. As used herein, the term “intrathecal administration” or “intrathecal injection” refers to an injection into the spinal canal (intrathecal space surrounding the spinal cord). Various techniques may be used including, without limitation, lateral cerebroventricular injection through a burrhole or cisternal or lumbar puncture or the like. More than one route of administering a trigger can be used concurrently, if desired.

In some embodiments, a trigger is present in the blood of a subject at a free concentration of greater than 1 picomolar, greater than 10 picomolar, greater than 100 picomolar, greater than 1 nanomolar, greater than 10 nanomolar, greater than 100 nanomolar, or greater than 1 micromolar. In some embodiments, a trigger is present in the blood of a subject at a free concentration of less than 1 micromolar, less than 100 nanomolar, less than 10 nanomolar, less than 1 nanomolar, less than 100 picomolar, or less than 10 picomolar. In some embodiments, a trigger is present in the blood of a subject at a free concentration of between 1 picomolar and 1 nanomolar, between 1 picomolar and 100 picomolar, or between 1 picomolar and 10 picomolar. In some embodiments, a trigger is present in the blood of a subject at a total concentration of greater than 1 picomolar, greater than 10 picomolar, greater than 100 picomolar, greater than 1 nanomolar, greater than 10 nanomolar, greater than 100 nanomolar, greater than 1 micromolar, or greater than 10 micromolar. In some embodiments, a trigger is present in the blood of a subject at a total concentration of less than 100 micromolar, less than 10 micromolar, less than 1 micromolar, less than 100 nanomolar, less than 10 nanomolar, less than 1 nanomolar, less than 100 picomolar, or less than 10 picomolar. In some embodiments, a trigger is present in the blood of a subject at a total concentration of between 10 nanomolar and 100 micromolar, between 100 nanomolar and 10 micromolar, or between 1 micromolar and 10 micromolar.

The present disclosure contemplates single, as well as, multiple administrations of a therapeutically effective amount of a composition that delivers a trigger described herein. In some embodiments, a trigger described herein can be administered at regular intervals, depending on the nature, severity and extent of the subject's condition. In some embodiments, a composition that delivers a trigger-responsive immune-inactivating signaling polypeptide described herein can be administered when T cell activity (as determined by a level of, e.g., a cytokine, e.g., IL-2) exceeds a threshold.

The present disclosure encompasses methods of regulating activity of T cells, which can include endogenous T cells, and/or engineered and/or modified T cells (e.g., CAR-T cells). In some embodiments, provided methods of regulating T cells can include regulating the activity of an endogenous TCR (e.g., a wild-type TCR or an endogenous TCR variant) or the activity of an engineered and/or modified TCR or a CAR. In some embodiments, provided methods of regulating T cells can include regulating the activity of CARs that target CD19, CD20, CD22, Igk light chain, CD30, CD138, BCMA, CD33, CD123, NKG2D ligands, ROR1, EGFR, EFGRvIII, GD2, IL13Ra2, HER2, Mesotheli, PSMA, FAP, GPC3, MET, MUC16, CEA, Lewis-Y, or MUC1. In some embodiments, provided methods of regulating T cells can include regulating the activity of CARs that target various neoantigens.

The present disclosure encompasses methods of regulating activity of T cells, which can include endogenous T cells present in a subject, engineered and/or modified T cells present in a subject (e.g., having been previously administered or introduced), or engineered and/or modified T cells being administered to a subject. In some embodiments, a method of regulating activity of T cells includes administering a modified and/or engineered T cell (e.g., a genetically modified T cell, e.g., a CAR-T cell) to the subject.

### Therapeutic Uses

The present disclosure recognizes that methods of regulating activity of T cells described herein can be useful as methods of treating various conditions (e.g., T cell exhaustion or cytokine dysregulation, e.g., hypercytokinemia) and/or diseases (e.g., cancer).

Among other things, the present disclosure provides a method of preventing or treating cytokine dysregulation. In some embodiments, a method of preventing or treating cytokine dysregulation includes administering a composition that delivers the trigger-responsive immune-inactivating signaling polypeptide as described herein to a subject (e.g., a patient, e.g., a human patient). In some embodiments, a method of preventing or treating cytokine dysregulation includes administering a trigger as described herein. In some embodiments, a composition that delivers the trigger-responsive immune-inactivating signaling polypeptide and/or the trigger are included in pharmaceutical compositions.

In some embodiments, cytokine dysregulation can include hypercytokinemia (e.g., a cytokine storm). In some embodiments, hypercytokinemia can associated with graft-versus-host disease.

T cell exhaustion is a state of T cell dysfunction that can, in some instances, result from stimulation. For instance, chronic or persistent exposure to a T cell pathway activating antigen and/or inflammatory signals can exhaust a T cell. T cell exhaustion can be characterized by, in at least some instances, one or more of poor effector function, elevated or sustained expression of inhibitory receptors, and a transcriptional state distinct from equivalent non-exhausted cells. Exhausted T cells are less effective in interacting with targets. T cell exhaustion may entail exhaustion of a subset of T cells present in a subject. T cell exhaustion can entail partial or complete, exhaustion of one or more T cells, such as a subset of T cells present in a subject. For instance, effector function can be progressively repressed during the development of T cell exhaustion, e.g., leading to a heterogeneous population of T cells at various levels of exhaustion. In some instances, partially exhausted T cells can display sustained expression of a minimal number of immune inhibitory receptors (IRs) and differential expression of T-bet and Eomes (T-bet^{high}Eomes^{low} T cells), whereas, e.g., fully exhausted T cells can be marked by coexpression of multiple IRs in some instances. T cell exhaustion has been reviewed, e.g., in Wherry (2015 Nat. Rev. Immunol. 15(8): 486-499), which is incorporated herein by reference. Various means of detecting, identifying, and/or predicting T cell exhaustion are known in the art. Modulation of one or more immune pathways can result in rejuvenation of partially exhausted T cells.

Among other things, the present disclosure provides a method of treating T cell exhaustion. The present disclosure encompasses the insight that a condition of T cell exhaustion in a T cell, or a subject including an exhausted T cell, can be treated by partial or complete inactivation of an immune activity, signal, or pathway, e.g., by physical or other regulatory interaction with the immune activity pathway (e.g., immune inactivation). The present disclosure encompasses the further insight that such partial or complete inactivation of an immune activity, signal, or pathway can be achieved in a T cell that includes, expresses, or encodes a trigger-responsive immune-inactivating signaling polypeptide, e.g., upon exposure to trigger. Accordingly, the present insight includes the application of methods and compositions described herein for the treatment of T cell exhaustion, e.g., by administration of a trigger to a subject including, identified as including, or identified as at risk of including exhausted T cells including, expressing, or encoding a trigger-responsive immune-inactivating signaling polypeptide.

In certain embodiments, a method of treating T cell exhaustion is a method of treating T cell exhaustion in a subject having been administered an engineered and/or modified T cell (e.g. CAR-T cell) including or encoding a trigger-responsive immune-inactivating signaling polypeptide. In certain embodiments, a method of treating T cell exhaustion is a method of treating T cell exhaustion in a subject having been administered an adoptive T cell therapy regimen, which regimen included administration to the subject of an engineered and/or modified T cell (e.g. CAR-T cell) including or encoding a trigger-responsive immune-inactivating signaling polypeptide. In various embodiments, T cell exhaustion or a risk thereof has been identified with respect to T cells (e.g., engineered and/or modified T cells (e.g. CAR-T cells)) administered to, present in, or to be administered to the subject.

In various instances of treating T cell exhaustion, trigger may be administered in any manner or regimen, or for any duration, in accordance with the present disclosure. In various instances, administration of trigger may be limited to a specified period of time or in limited in accordance with the evaluation of a medical practitioner. For instance, a regimen for administration of a trigger in the treatment of T cell exhaustion may include administration of trigger to a subject in one or more same or different doses over a period such as 6 hours, 12 hours, one day, two days, three days, four days, five days, six days, seven days, eight days, nine days, ten days, eleven days, twelve days, thirteen days, two weeks, three weeks, one month, two months, three months, four months, five months, or six months.

In some embodiments, a method of treating T cell exhaustion can include administering a composition that delivers the trigger-responsive immune-inactivating signaling polypeptide as described herein to a subject (e.g., a patient, e.g., a human patient). In some embodiments, a method of treating T cell exhaustion includes administering a trigger as described herein. In some embodiments, a composition that delivers the trigger-responsive immune-inactivating signaling polypeptide and/or the trigger are included in pharmaceutical compositions. In some embodiments, a method of treating T cell exhaustion can include administering an engineered and/or modified T cell (e.g. CAR-T cell) to the subject.

Among other things, the present disclosure provides a method of treating cancer. In some embodiments, a method of treating cancer can include administering a composition that delivers the trigger-responsive immune-inactivating signaling polypeptide as described herein to a subject (e.g., a patient, e.g., a human patient). In some embodiments, a method of treating cancer includes administering a trigger as described herein. In some embodiments, a composition that delivers the trigger-responsive immune-inactivating signaling polypeptide and/or the trigger are included in pharmaceutical compositions.

In some embodiments, a method of treating cancer can include administering an engineered and/or modified T cell (e.g. CAR-T cell) to the subject.

In some embodiments, a cancer can be carcinoma, sarcoma, melanoma, lymphoma, leukemia, or blastoma. In some embodiments, a carcinoma can be a basal cell carcinoma, squamous cell carcinoma, renal cell carcinoma, ductal carcinoma in situ, invasive ductal carcinoma, and/or adenocarcinoma. In some embodiments, a carcinoma can be a prostate cancer, an ovarian cancer, a uterine cancer, a cervical cancer, a colorectal cancer, a breast cancer, a bladder cancer, a pancreatic cancer, an esophageal cancer, a gastrointestinal cancer, a hepatocellular cancer, a thyroid cancer, or a lung cancer. In some embodiments, a sarcoma can be a angiosarcoma, a chondrosarcoma, an Ewing's sarcoma, fibrosarcoma, a gastrointestinal stromal cancer, a Leiomyosarcoma, a liposarcoma, an osteosarcoma, a pleomorphic sarcoma, a rhabdomyosarcoma, or a synovial sarcoma. In some embodiments, a melanoma can be a superficial spreading melanoma, a nodular melanoma, a lentigo maligna melanoma, or an acral melanoma. In some embodiments, a lymphoma can be a B-cell lymphoma, a T cell lymphoma, or an NK-cell lymphoma. In some embodiments, a leukemia can be an acute myeloid leukemia, a chronic myeloid leukemia, acute lymphocytic leukemia, or a chronic lymphocytic leukemia. In some embodiments, a blastoma can be a hepatoblastoma, a medulloblastoma, a nephroblastoma, a neuroblastoma, a pancreatoblastoma, a pleuropulmonary blastoma, retinoblastoma, or a glioblastoma. In some embodiments, a cancer can be a Stage I, Stage II, Stage III, or Stage IV cancer. In some embodiments, a cancer can be metastatic.

In some embodiments, a trigger-responsive immune-inactivating signaling polypeptide is administered in combination with one or more therapeutic agents (e.g., an anti-cancer agent). In some embodiments, the one or more therapeutic agents have received regulatory approval and are currently used for treatment of a condition and/or disease. In some embodiments, therapeutic agent(s) is/are administered according to its standard or approved dosing regimen and/or schedule. In some embodiments, therapeutic agent(s) is/are administered according to a regimen that is altered as compared with its standard or approved dosing regimen and/or schedule. In some embodiments, such an altered regimen differs from the standard or approved dosing regimen in that one or more unit doses is altered (e.g., reduced or increased) in amount, and/or in that dosing is altered in frequency (e.g., in that one or more intervals between unit doses is expanded, resulting in lower frequency, or is reduced, resulting in higher frequency). In some embodiments, a trigger-responsive immune-inactivating signaling polypeptide is administered at the same time as with one or more therapeutic agents (e.g., an anti-cancer agent). In some embodiments, a trigger-responsive immune-inactivating signaling polypeptide and one or more therapeutic agents (e.g., an anti-cancer agent) are administered as part of the same course of treatment but are not administered together.

The disclosure is further illustrated by the following examples. The examples are provided for illustrative purposes only. They are not to be construed as limiting the scope or content of the disclosure in any way.

### EXAMPLES

Other features, objects, and advantages of the present invention are apparent in the examples that follow. It should be understood, however, that the examples, while indicating embodiments of the present invention, are given by way of illustration only, not limitation. Various changes and modifications within the scope of the invention will become apparent to those skilled in the art from the examples.

### Example 1: Construction of a Vector Encoding a Trigger-Responsive Dominant Negative Signaling Polypeptide

This example illustrates an exemplary DNA construct engineered for delivery and expression of a trigger-responsive dominant negative signaling polypeptide. It will be clear to one skilled in the art that a number of alternative approaches, expression vectors and cloning techniques are available.

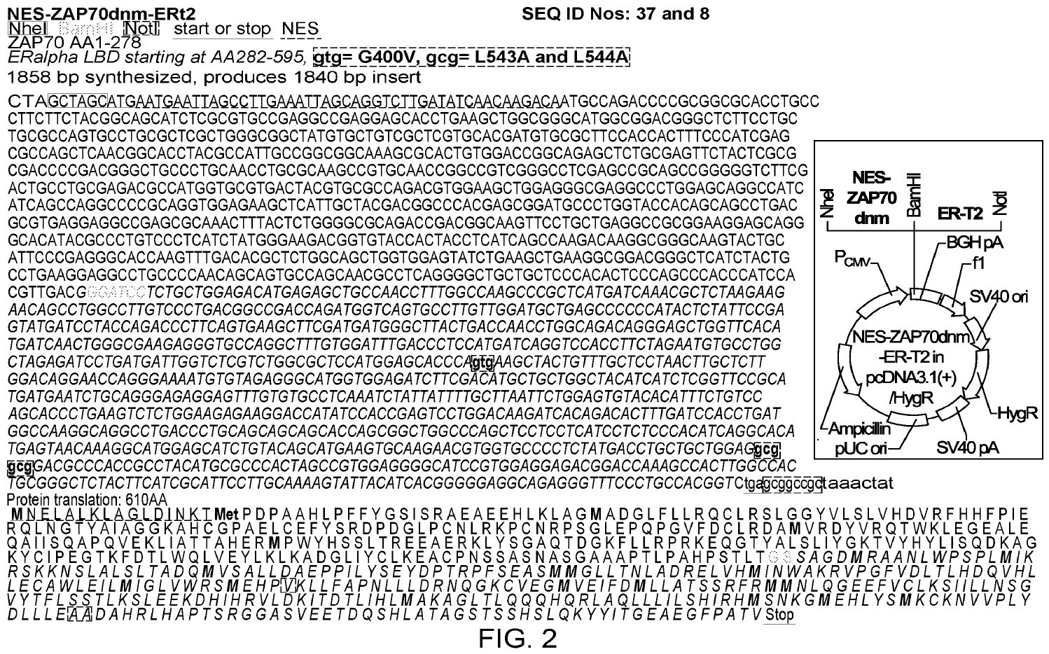

As shown in FIG. 2, a pcDNA3.1 (+) vector (Thermo Fisher Scientific) was engineered to include a DNA sequence encoding a trigger-responsive dominant negative signaling polypeptide, in this case, an endoxifen-responsive dominant negative Zap-70 polypeptide. The endoxifen-responsive dominant negative Zap-70 polypeptide included from 5′ to 3′: a nuclear export signal (NES), a dominant negative Zap-70 moiety, and ER(T2). The DNA sequence encoding the endoxifen-responsive dominant negative Zap-70 polypeptide was under the control of a CMV promoter. Additional control elements helpful for obtaining efficient replication and expression (e.g., control elements for vector replication, transcription, translation, etc.) were also present in the vector.

The DNA encoding the endoxifen-responsive dominant negative Zap-70 polypeptide had a nucleotide sequence according to SEQ ID NO: 7, and the endoxifen-responsive dominant negative Zap-70 polypeptide had an amino acid sequence according to SEQ ID NO: 8.

### Example 2: Construction of a Vector Encoding a Dominant Negative Signaling Moiety

This example illustrates an exemplary DNA construct engineered for delivery and expression of a dominant negative signaling moiety, which can be useful as, among other things, as a control. It will be clear to one skilled in the art that a number of alternative approaches, expression vectors and cloning techniques are available.

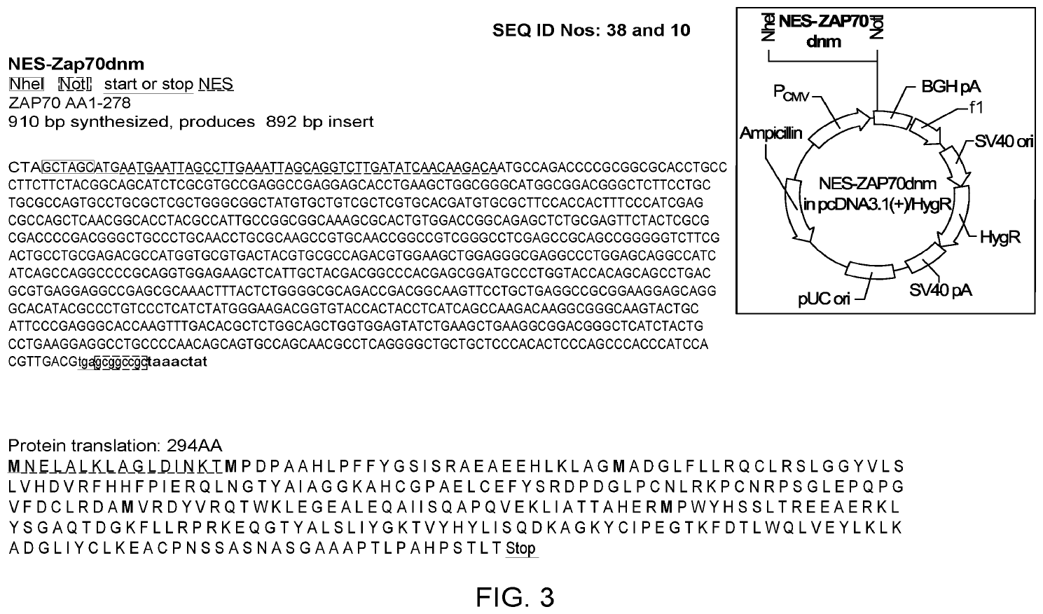

As shown in FIG. 3, a pcDNA3.1 (+) vector (Thermo Fisher Scientific) was engineered to include a DNA sequence encoding polypeptide including a nuclear export signal (NES) and a dominant negative Zap-70 moiety. The DNA sequence encoding a dominant negative signaling moiety was under the control of a CMV promoter. Additional control elements helpful for obtaining efficient replication and expression (e.g., control elements for vector replication, transcription, translation, etc.) were also present in the vector.

The DNA encoding the polypeptide had a nucleotide sequence according to SEQ ID NO: 9, and the polypeptide had an amino acid sequence according to SEQ ID NO: 10.

SEQ ID NO: 9
ATGAATGAATTAGCCTTGAAATTAGCAGGTCTTGA
TATCAACAAGACAATGCCAGACCCCGCGGCGCACC
TGCCCTTCTTCTACGGCAGCATCTCGCGTGCCGAG
GCCGAGGAGCACCTGAAGCTGGCGGGCATGGCGGA
CGGGCTCTTCCTGCTGCGCCAGTGCCTGCGCTCGC
TGGGCGGCTATGTGCTGTCGCTCGTGCACGATGTG
CGCTTCCACCACTTTCCCATCGAGCGCCAGCTCAA
CGGCACCTACGCCATTGCCGGCGGCAAAGCGCACT
GTGGACCGGCAGAGCTCTGCGAGTTCTACTCGCGC
GACCCCGACGGGCTGCCCTGCAACCTGCGCAAGCC
GTGCAACCGGCCGTCGGGCCTCGAGCCGCAGCCGG
GGGTCTTCGACTGCCTGCGAGACGCCATGGTGCGT
GACTACGTGCGCCAGACGTGGAAGCTGGAGGGCGA
GGCCCTGGAGCAGGCCATCATCAGCCAGGCCCCGC
AGGTGGAGAAGCTCATTGCTACGACGGCCCACGAG
CGGATGCCCTGGTACCACAGCAGCCTGACGCGTGA
GGAGGCCGAGCGCAAACTTTACTCTGGGGCGCAGA
CCGACGGCAAGTTCCTGCTGAGGCCGCGGAAGGAG
CAGGGCACATACGCCCTGTCCCTCATCTATGGGAA
GACGGTGTACCACTACCTCATCAGCCAAGACAAGG
CGGGCAAGTACTGCATTCCCGAGGGCACCAAGTTT
GACACGCTCTGGCAGCTGGTGGAGTATCTGAAGCT
GAAGGCGGACGGGCTCATCTACTGCCTGAAGGAGG
CCTGCCCCAACAGCAGTGCCAGCAACGCCTCAGGG
GCTGCTGCTCCCACACTCCCAGCCCACCCATCCAC
GTTGACG
SEQ ID NO: 10
M N E L A L K L A G L D I N K T M
P D P A A H L P F F Y G S I S R A
E A E E H L K L A G M A D G L F L
L R Q C L R S L G G Y V L S L V H
D V R F H H F P I E R Q L N G T Y
A I A G G K A H C G P A E L C E F
Y S R D P D G L P C N L R K P C N
R P S G L E P Q P G V F D C L R D
A M V R D Y V R Q T W K L E G E A
L E Q A I I S Q A P Q V E K L I A
T T A H E R M P W Y H S S L T R E
E A E R K L Y S G A Q T D G K F L
L R P R K E Q G T Y A L S L I Y G
K T V Y H Y L I S Q D K A G K Y C
I P E G T K F D T L W Q L V E Y L
K L K A D G L I Y C L K E A C P N
S S A S N A S G A A A P T L P A H
P S T L T

### Example 3: Inhibition of NFAT-Luciferase Expression by Endoxifen-Responsive Dominant Negative Zap-70 Polypeptide in Jurkat E6.1 Cells

This example demonstrates that a modulating domain of a trigger-responsive dominant can inhibit the activity of an operatively linked dominant negative moiety, and that the inhibition of the dominant negative signaling moiety by the modulating domain can be relieved in the presence of the trigger. This example further shows that, when the inhibition of the dominant negative signaling moiety is relieved by the interaction of the trigger with the modulating domain, the dominant negative signaling moiety can inhibit a T cell activation cascade.

One outcome of T cell activation is the expression of Nuclear Factor of Activated T cells (NFAT) transcription factors. Accordingly, a construct containing a reporter gene, such as firefly luciferase (Luc), under the control of an NFAT response element can be used to assess T cell activity. Such a construct can either be transiently transfected into cells (e.g., Jurkat cells, which are an immortalized line of human T cells) or stably integrated into a cell line (e.g., a derivative Jurkat cell line). These reporter genes can be used to measure activation, or inhibition, of T cell activation pathways.

To examine whether an endoxifen-responsive dominant negative Zap-70 polypeptide could inhibit T cell activity resulting in NFAT-luciferase expression, Jurkat E6.1 cells (purchased from ATCC (Manassas, Va.)) were transiently transfected with an NFAT-Luciferase reporter construct and a test construct of (1) a control vector (“vector”), (2) a vector encoding a dominant negative Zap70 moiety, as described in Example 2 (“Zap70dnm”), or (3) a vector encoding an endoxifen-responsive dominant negative Zap70 polypeptide, including an ER(T2) domain operatively linked to a dominant negative Zap70 moiety, as described in Example 1 (“Zap70dnm-ER(T2)”). The transient transfections were performed using LTX and PLUS transfection reagent (ThermoFisher Scientific) according to manufacturer's protocols.

Transfected cells were treated in the absence or presence of 1:30 Immunocult™ CD3/CD28/CD2 tetrameric antibody mixture (StemCell Technologies, Canada) (“α-TCR”), which stimulates T cell activity. Stimulated cells were then further treated with either RPMI 1640 media plus 4.4% FBS stripped with charcoal dextran (ThermoFisher Scientific) or 100 nM endoxifen in RPMI 1640 media plus 4.4% FBS stripped with charcoal dextran (ThermoFisher Scientific).

A summary of the Jurkat cells samples tested is included in Table 6 below.

TABLE 6

| 1 | 2 | 3 | 4 | 5 | 6 | 7 | 8 | 9 |
|---|---|---|---|---|---|---|---|---|
| NFAT- | NFAT- | NFAT- | NFAT- | NFAT- | NFAT- | NFAT- | NFAT- | NFAT- |
| luciferase | luciferase | luciferase | luciferase | luciferase | luciferase | luciferase | luciferase | luciferase |
| Vector | Vector | Vector | Zap70dnm | Zap70dnm | Zap70dnm | Zap70dnm- | Zap70dnm- | Zap70dnm- |
| | | | | | | ER(T2) | ER(T2) | ER(T2) |
| Vehicle | α-TCR | α-TCR | Vehicle | α-TCR | α-TCR | Vehicle | α-TCR | α-TCR |
| Vehicle | Vehicle | Endoxifen | Vehicle | Vehicle | Endoxifen | Vehicle | Vehicle | Endoxifen |

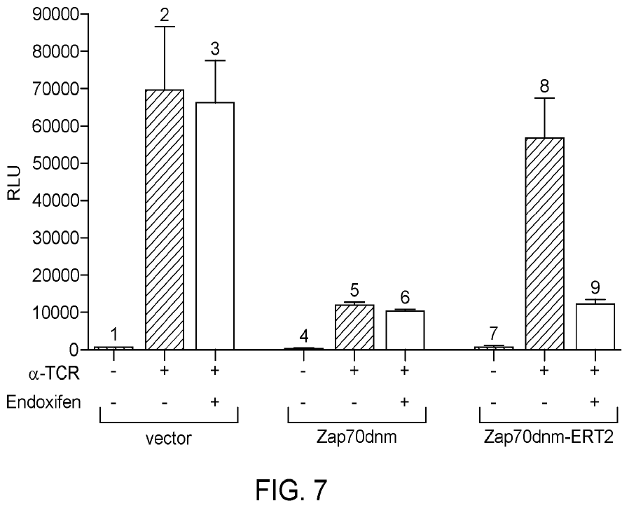

After 19 hours, luciferase activity was assayed with Bright-Glo substrate according to manufacturer's protocols (Promega). Luminescence was detected on the Varioskan Lux (ThermoFisher Scientific) plate reader at a 1 second interval. The luminescence was measured and reported in relative light units (RLU). The results are graphically represented in FIG. 7. Bars represent mean activity from quadruplicate wells with error bars representing SEM.

The control vector was co-transfected into Jurkat cells as a negative control and demonstrated that any effects observed from the co-transfection of the Zap70dnm and Zap70dnm-ER(T2) constructs were result of the encoded dominant negative Zap70 moiety or the encoded endoxifen-responsive dominant negative Zap70 polypeptide, respectively. Accordingly, samples 1-3, which were co-transfected with the control vector, helped to establish that the system was working as expected. For example, as expression of a gene under the control of an NFAT response element should be driven by T cell activation and the subsequent activation of NFAT transcription factors, it was expected that no luciferase expression from the NFAT-luciferase construct would be detected in Jurkat cells that were co-transfected with a control vector and treated with vehicle (i.e., not with α-TCR). Indeed, that is what was observed for sample 1. As shown, no significant luciferase activity was observed when the T cell pathway was not activated by α-TCR.

On the flip side, it was expected that significant luciferase expression would be observed from Jurkat cells that were co-transfected with the control vector and treated with vehicle. Again, the control data matched what was expected. As shown for sample 2, nearly 70,000 RLUs were observed in Jurkat cells co-transfected with a control vector and treated with α-TCR.

It was hypothesized that endoxifen treatment, in the absence of the endoxifen-responsive dominant negative Zap70 polypeptide, would not impact the level of luciferase expression because it should neither promote or inhibit luciferase expression from the NFAT-luciferase construct on its own. As shown, the addition of endoxifen to Jurkat cells co-transfected with the control vector and treated with α-TCR (sample 3) showed similar levels luciferase activity to Jurkat cells co-transfected with the control vector and treated with α-TCR (sample 2). These control data demonstrate that endoxifen on its own was not affecting luciferase expression.

Samples 4-6 include Jurkat cells co-transfected with the NFAT-luciferase construct and Zap70dnm that encodes a dominant negative Zap70 moiety. As shown in the data for sample 4, no luciferase expression was detected when the cells were treated with vehicle (i.e., not treated with α-TCR or endoxifen). In sample 5, the cells were treated with α-TCR, which activated the T cells activation cascade (see, e.g., sample 2). However, the luciferase expression detected was low, which suggested that the dominant negative Zap70 moiety was expressed from the Zap70dnm construct and inhibited the T cell activation cascade. In sample 6, the Jurkat cells were treated with α-TCR, which activated the T cells activation cascade, and treated with endoxifen. As shown in FIG. 7, the luciferase expression detected was low and nearly equivalent to the luciferase expression observed from sample 5. These data demonstrated that the activity of the dominant negative Zap70 moiety expressed from the Zap70dnm construct is not affected by endoxifen on its own.

Samples 7-9 include Jurkat cells co-transfected with the NFAT-luciferase construct and Zap70dnm-ER(T2) that encodes an endoxifen-responsive dominant negative Zap70 polypeptide. As shown in the data for sample 7, no luciferase expression was detected when the cells were treated with vehicle (i.e., not treated with α-TCR or endoxifen). In sample 8, the cells were treated with α-TCR, which activated the T cells activation cascade (see, e.g., sample 2). A significant level of luciferase expression was detected from sample 8, particularly when compared to the luciferase levels observed from sample 5. As discussed above, in sample 5, the dominant negative Zap70 moiety inhibited the T cell activation pathway, and therefore, inhibited expression of luciferase from the NFAT-luciferase construct. In contrast, the data for sample 8 indicated that, in the absence of endoxifen, the dominant negative Zap70 moiety present in the endoxifen-responsive dominant negative Zap70 polypeptide was inhibited or masked by the ER(T2) modulating domain, and therefore, unable to inhibit the T cell activation cascade and resulting NFAT regulated luciferase expression. Finally, in sample 9, the Jurkat cells were treated with α-TCR, which activated the T cells activation cascade, and treated with endoxifen. As shown in FIG. 7, the luciferase expression detected was low and nearly equivalent to the luciferase expression levels observed from samples 5 and 6. The low level of luciferase expression obtained for sample 9 indicates that, in the presence of endoxifen, the dominant negative Zap70 moiety present in the endoxifen-responsive dominant negative Zap70 polypeptide was able to inhibit the T cell activation cascade and resulting NFAT regulated luciferase expression, as it was relieved of the ER(T2) mediated inhibition.

In sum, the data shown in FIG. 7 demonstrated that an ER(T2) domain of an endoxifen-responsive dominant negative Zap70 polypeptide inhibited the activity of the operatively linked dominant negative Zap70 moiety in the absence of endoxifen, and that the inhibition of the dominant negative Zap70 moiety by the ER(T2) domain was relieved in the presence of endoxifen. Further, it was demonstrated that, when the inhibition of the dominant negative Zap70 moiety was relieved by the interaction of endoxifen with the ER(T2) domain, the dominant negative Zap70 moiety inhibited a T cell activation cascade.

### Example 4: Inhibition of NFAT-Luciferase by Endoxifen-Responsive Dominant Negative Zap-70 Polypeptide in Jurkat E6.1 Cells was Controlled by Endoxifen in a Dose Dependent Manner

This example demonstrates that the inhibition or masking of a dominant negative signaling moiety of a trigger-responsive dominant negative signaling polypeptide by a modulating domain can be relieved in a dose-dependent manner by a trigger. This example further shows that, when the inhibition of the dominant negative signaling moiety is relieved by the interaction of the trigger with the modulating domain, the dominant negative signaling moiety can inhibit a T cell activation cascade in a dose dependent manner.

To examine whether the concentration of endoxifen affected the level of inhibition of T cell activity by an endoxifen-responsive dominant negative Zap-70 polypeptide, Jurkat E6.1 cells (purchased from ATCC (Manassas, Va.)) were transiently transfected with an NFAT-Luciferase reporter construct and a test construct of (1) a vector encoding a dominant negative Zap70 moiety, as described in Example 2 (“Zap70dnm”), or (2) a vector encoding an endoxifen-responsive dominant negative Zap70 polypeptide, including an ER(T2) domain operatively linked to a dominant negative Zap70 moiety, as described in Example 1 (“Zap70dnm-ER(T2)”). The transient transfections were performed using LTX and PLUS transfection reagent (ThermoFisher Scientific) according to manufacturer's protocols.

Transfected cells were treated with an anti-TCR antibody, clone 305, which stimulated T cell activity. Stimulated cells were then further treated with 0 nM, 0.05 nM, 0.1 nM, 0.5 nM, 1 nM, 5 nM, 10 nM, 50 nM, 100 nM, 500 nM, or 1000 nM of endoxifen in RPMI 1640 media plus 4.4% FBS stripped with charcoal dextran (ThermoFisher Scientific).

After 6 hours, luciferase activity was assayed with Bright-Glo substrate according to manufacturer's protocols (Promega). Luminescence was detected on the Varioskan Lux (ThermoFisher Scientific) plate reader at a 1 second interval. The luminescence was measured and reported in relative light units (RLU). Points represent mean activity from quadruplicate wells with error bars representing SEM.

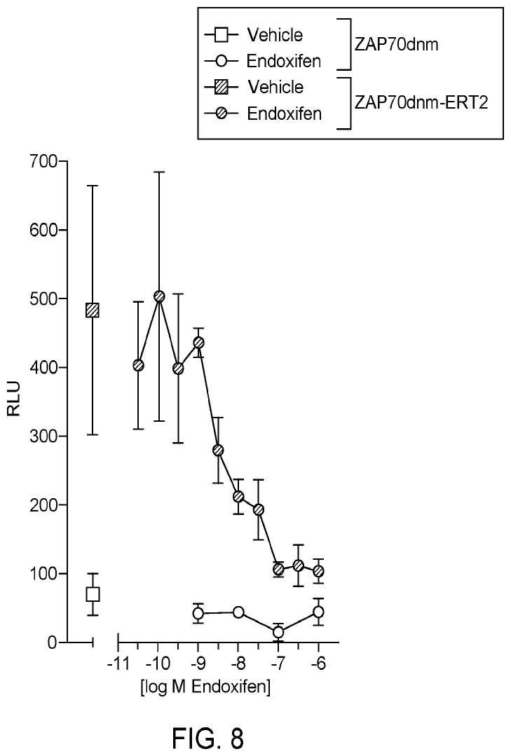

As shown in FIG. 8, the luciferase expression detected was low in cells co-transfected with the Zap70dnm construct, regardless of the endoxifen concentration, which suggested that the dominant negative Zap70 moiety was expressed from the Zap70dnm construct and inhibited the T cell activation cascade. These data also indicated that expression of the dominant negative Zap70 moiety and its inhibition of the T cell activation cascade were not affected by endoxifen concentration. In contrast, the luciferase expression detected was high in cells co-transfected with the Zap70dnm-ER(T2) construct and treated with vehicle. This data indicated that, in the absence of endoxifen, the dominant negative Zap70 moiety present in the endoxifen-responsive dominant negative Zap70 polypeptide was inhibited or masked by the ER(T2) modulating domain, and therefore, unable to inhibit the T cell activation cascade and resulting NFAT regulated luciferase expression. As the concentration of endoxifen was increased, however, the level of luciferase expression detected decreased in a dose dependent manner. These data indicated that the activity of the dominant negative Zap70 moiety included in a trigger-responsive dominant negative Zap70 polypeptide can be regulated by endoxifen in a dose dependent manner, and in turn, that inhibition of the T cell activation cascade by the endoxifen-responsive dominant negative Zap-70 polypeptide was controlled by endoxifen in a dose dependent manner. The ability to control the activity of the dominant negative Zap70 moiety included in a trigger-responsive dominant negative Zap70 polypeptide by regulating the concentration of endoxifen provides a mechanism for tightly regulating T cell activity.

### Example 5: Inhibition of NFAT-Luciferase Expression Increased with the Amount of Endoxifen-Responsive Dominant Negative Zap-70 Polypeptide in Jurkat E6.1 Cells

This example demonstrates that the activity of a dominant negative signaling moiety included in a trigger-responsive dominant negative signaling polypeptide, and in turn, the level of T cell activation cascade inhibition, can be regulated by the amount of a trigger-responsive dominant negative signaling polypeptide.

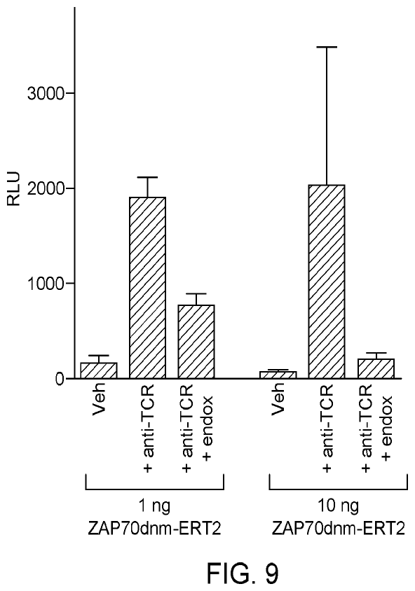

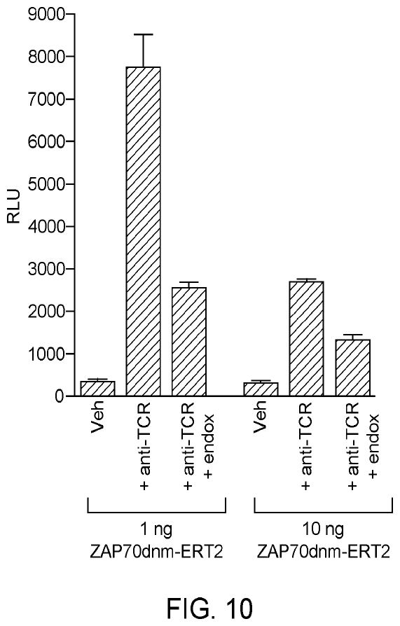

To examine whether the amount of Zap70dnm-ER(T2) construct affected the level of inhibition of T cell activity by an encoded endoxifen-responsive dominant negative Zap-70 polypeptide, Jurkat E6.1 cells (purchased from ATCC (Manassas, Va.)) that were either transiently transfected with an NFAT-Luciferase reporter construct (FIG. 9) or stably transfected with an NFAT-Luciferase reporter construct (FIG. 10) were also transiently transfected with either 1 ng or 10 ng of a vector encoding an endoxifen-responsive dominant negative Zap70 polypeptide, including an ER(T2) domain operatively linked to a dominant negative Zap70 moiety, as described in Example 1 (“Zap70dnm-ER(T2)”). The transient transfections were performed using LTX and PLUS transfection reagent (ThermoFisher Scientific) according to manufacturer's protocols. Jurkat cells stably transfected with NFAT-Luciferase were purchased from Signosis, Inc. (Santa Clara, Calif.). Cells were maintained in RPMI 1640 media (ThermoFisher Scientific) plus 10% FBS (GE Healthcare, Pittsburgh, Pa.) at a density between 1×10⁵ to 3×10⁶ cells/ml.

Transfected cells were then treated with an anti-TCR antibody, clone 305, which stimulated T cell activity. Stimulated cells were then further treated with 100 nM endoxifen in RPMI 1640 media plus 4.4% FBS stripped with charcoal dextran (ThermoFisher Scientific). After incubation for 6 hours, luciferase activity was assayed with Bright-Glo substrate according to manufacturer's protocols (Promega). Luminescence was detected on the Varioskan Lux (ThermoFisher Scientific) plate reader at a 1 second interval. The luminescence was measured and reported in relative light units (RLU). The results are graphically represented in FIGS. 9 and 10. Bars represent mean activity from triplicate wells with error bars representing SEM.

For both the transient and stable transfections, the luciferase expression in Jurkat cells that were transiently transfected with 10 ng of Zap70dnm-ER(T2) construct was lower than the luciferase expression observed from Jurkat cells transiently transfected with 1 ng of Zap70dnm-ER(T2) construct. As a higher amount of Zap70dnm-ER(T2) construct is likely resulted in a higher amount of endoxifen-responsive dominant negative Zap-70 polypeptide, these data suggest that a higher amount of endoxifen-responsive dominant negative Zap-70 polypeptide was able to inhibit the T cell activation cascade and expression of luciferase from the NFAT-luciferase construct.

### Example 6: Inhibition of NFAT-Luciferase Expression was Regulated by the Amount of Endoxifen-Responsive Dominant Negative Zap-70 Polypeptide in a Dose Dependent Manner

This example demonstrates that the activity of a dominant negative signaling moiety included in a trigger-responsive dominant negative signaling polypeptide, and in turn, the level of T cell activation cascade inhibition, can be regulated by the amount of a trigger-responsive dominant negative signaling polypeptide in a dose dependent manner.

To examine whether the amount of Zap70dnm-ER(T2) construct affected the level of inhibition of T cell activity by an encoded endoxifen-responsive dominant negative Zap-70 polypeptide in a dose dependent manner, Jurkat E6.1 cells (purchased from ATCC (Manassas, Va.)) were transiently transfected with an NFAT-Luciferase reporter construct and 0.05 ng, 0.1 ng, 0.5 ng, 1 ng, 5 ng, 10 ng, or 50 ng of a vector encoding an endoxifen-responsive dominant negative Zap70 polypeptide, including an ER(T2) domain operatively linked to a dominant negative Zap70 moiety, as described in Example 1 (“Zap70dnm-ER(T2)”). The transient transfections were performed using LTX and PLUS transfection reagent (ThermoFisher Scientific) according to manufacturer's protocols.

Transfected cells were then treated with an anti-TCR antibody, clone 305, which stimulated T cell activity. Stimulated cells were then further treated with 100 nM endoxifen in RPMI 1640 media plus 4.4% FBS stripped with charcoal dextran (ThermoFisher Scientific). After incubation for 6 hours, luciferase activity was assayed with Bright-Glo substrate according to manufacturer's protocols (Promega). Luminescence was detected on the Varioskan Lux (ThermoFisher Scientific) plate reader at a 1 second interval. The luminescence was measured and reported in relative light units (RLU). Points represent mean activity from quadruplicate wells with error bars representing SEM.

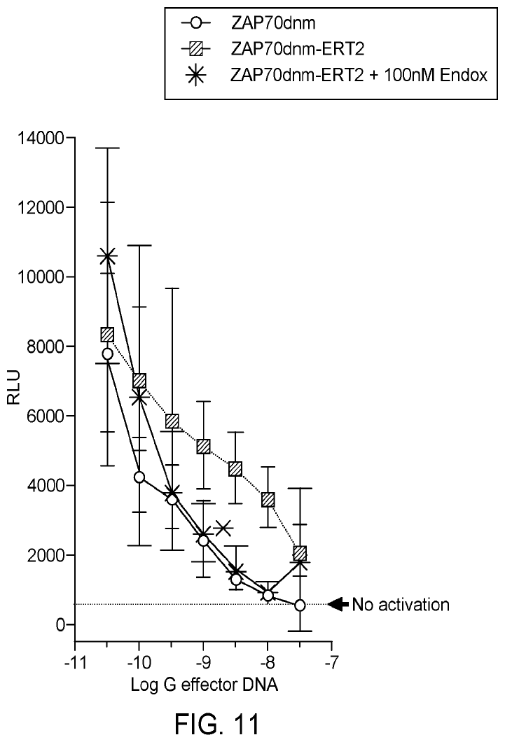

As shown in FIG. 11, for the samples treated with endoxifen, as the amount of Zapdnm-ER(T2) construct was increased, the amount of luciferase detected decreased in a dose dependent manner. In fact, the dose curve for the cells transfected with Zapdnm-ER(T2) construct and treated with endoxifen was highly similar to the dose curve for the cells transfected with the Zap70dnm construct.

### Example 7: Inhibition of NFAT-Luciferase by Endoxifen-Responsive Dominant Negative Zap-70 Polypeptides Including a G400V or G400L Mutation were Controlled by Endoxifen in a Dose Dependent Manner

This example demonstrates that a modulating domain that includes either a G400V or a G400L mutation can inhibit or mask a dominant negative signaling moiety of a trigger-responsive dominant negative signaling polypeptide. This example further demonstrates that the inhibition or masking of a dominant negative signaling moiety of a trigger-responsive dominant negative signaling polypeptide by a modulating domain that includes either a G400V or a G400L mutation can be relieved in a dose-dependent manner by a trigger. Additionally, this example shows that, when the inhibition of the dominant negative signaling moiety is relieved by the interaction of the trigger with such a modulating domain, the dominant negative signaling moiety can inhibit a T cell activation cascade in a dose dependent manner.

To examine whether a modulating domain that includes either a G400V or a G400L mutation can inhibit or mask a dominant negative Zap-70 polypeptide of an endoxifen-responsive dominant negative Zap-70 polypeptide, Jurkat E6.1 cells (purchased from ATCC (Manassas, Va.)) were transiently transfected with an NFAT-Luciferase reporter construct and a test construct of (1) a vector encoding an endoxifen-responsive dominant negative Zap70 polypeptide, comprising an ER(T2) domain, which included a G400V mutation, operatively linked to a dominant negative Zap70 moiety (“G400V”) or (2) a vector encoding an endoxifen-responsive dominant negative Zap70 polypeptide, comprising an ER(T2) domain, which included a G400L mutation, operatively linked to a dominant negative Zap70 moiety (“G400L”). The transient transfections were performed using LTX and PLUS transfection reagent (ThermoFisher Scientific) according to manufacturer's protocols.

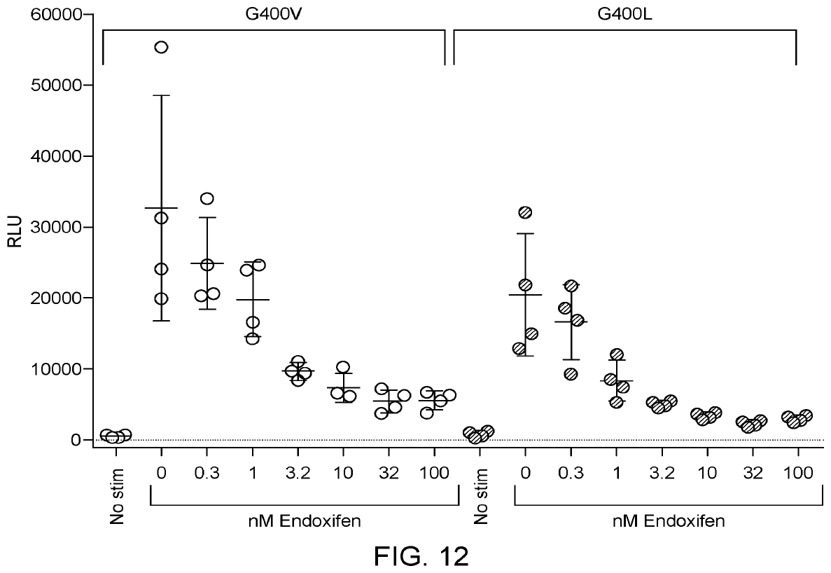

Transfected cells were treated in the absence or presence of 1:30 Immunocult™ CD3/CD28/CD2 tetrameric antibody mixture (StemCell Technologies, Canada) (“α-TCR”), which stimulates T cell activity. Stimulated cells were then further treated with either RPMI 1640 media plus 4.4% FBS stripped with charcoal dextran (ThermoFisher Scientific) or one of 0, 0.3 nM, 1 nM, 3.2 nM, 10 nM, 32 nM and 100 nM endoxifen in RPMI 1640 media plus 4.4% FBS stripped with charcoal dextran (ThermoFisher Scientific). After 19 hours, luciferase activity was assayed with Bright-Glo substrate according to manufacturer's protocols (Promega). Luminescence was detected on the Varioskan Lux (ThermoFisher Scientific) plate reader at a 1 second interval. The luminescence was measured and reported in relative light units (RLU). The results are graphically represented in FIG. 12. Points represent the activity of individual replicates with line denoting the mean from quadruplicate wells and error bars representing SEM.

As shown in FIG. 12, the luciferase expression detected was high in cells co-transfected with either the G400V or G400L construct and treated with vehicle. These data indicated that, in the absence of endoxifen, the dominant negative Zap70 moiety present in the endoxifen-responsive dominant negative Zap70 polypeptides was inhibited or masked by the ER(T2) domain having either a G400V or G400L mutation, and therefore, the dominant negative Zap70 moiety was unable to inhibit the T cell activation cascade and resulting NFAT regulated luciferase expression. As the concentration of endoxifen was increased, however, the level of luciferase expression detected decreased in a dose dependent manner for cells co-transfected with either a G400V or a G400L construct. These data indicated that the activity of the dominant negative Zap70 moiety included in a trigger-responsive dominant negative Zap70 polypeptide having an ER(T2) domain with either a G400V or G400L mutation can be regulated by endoxifen in a dose dependent manner, and in turn, that inhibition of the T cell activation cascade by those endoxifen-responsive dominant negative Zap-70 polypeptides was controlled by endoxifen in a dose dependent manner.

### Example 8: Inhibition of the Activity of Dominant Negative Zap70 in an Endoxifen-Responsive Dominant Negative Zap70 Polypeptide is Effectively Relieved by the Interaction of Endoxifen with an ER(T2) Domain Having Either a G400V or a G400L Mutation

This example demonstrates that the inhibition or masking of a dominant negative signaling moiety of a trigger-responsive dominant negative signaling polypeptide by an ER(T2) domain including either a G400V or a G400L mutation can be relieved by endoxifen at biologically relevant concentrations. This example further demonstrates that, while the inhibition or masking of a dominant negative signaling moiety of a trigger-responsive dominant negative signaling polypeptide by an ER(T2) domain including either a G400V or a G400L mutation can be relieved in a dose-dependent manner by a trigger, the dose curves obtained for trigger-responsive dominant negative signaling polypeptide that include an ER(T2) domain having a G400V or a G400L mutation are different from one another. Additionally, this example demonstrates that the IC50 or pIC50 of a trigger can vary based on a modulating domain (e.g., ER(T2) domain) present in a trigger-responsive dominant negative signaling polypeptide.

To examine how effective endoxifen is at relieving the inhibition or masking of a dominant negative signaling moiety of a trigger-responsive dominant negative signaling polypeptide by an ER(T2) domain including either a G400V or a G400L mutation, Jurkat E6.1 cells were transiently transfected with 90 ng NFAT-Luciferase reporter construct and 10 ng of a G400V or G400L construct. The transfected cells were stimulated the addition of 1:30 Immunocult CD3/CD28/CD2 tetrameric antibody mixture, and treated with or without endoxifen for 19 hours before assaying for luciferase activity. Results were normalized to maximum TCR stimulation with Immunocult alone, with 0% representing the activity of unstimulated transfected cells. Points represent mean activity from sextuplicate wells with error bars representing SEM. pIC50's were calculated using a variable slope sigmoidal dose-response model using GraphPad Prism.

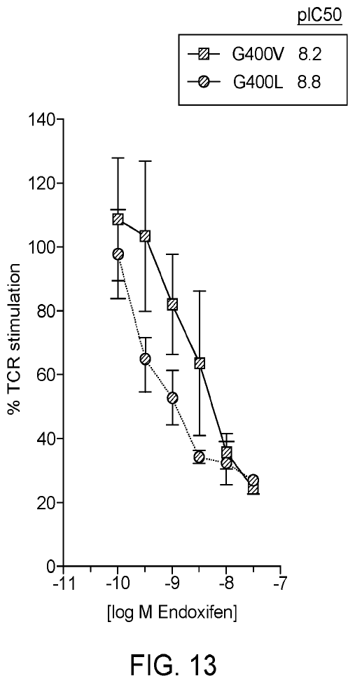

As shown in FIG. 13, the pIC50 of endoxifen, when determined in combination with an endoxifen-responsive dominant negative Zap70 polypeptide including an ER(T2) domain having a G400V mutation, was 8.2. The pIC50 of endoxifen, when determined in combination with an endoxifen-responsive dominant negative Zap70 polypeptide including an ER(T2) domain having a G400L mutation, was 8.8. Both pIC50 values demonstrate that endoxifen was effective at relieving the inhibition or masking of a dominant negative signaling moiety of a trigger-responsive dominant negative signaling polypeptide by an ER(T2) domain including either a G400V or a G400L mutation at concentrations that can be achieved physiologically in a subject who has received endoxifen (e.g., by daily dosing of endoxifen). Moreover, these values show that the endoxifen-responsive dominant negative Zap70 polypeptide including an ER(T2) domain having a G400L mutation was more easily activated (i.e., the inhibition or masking of the dominant negative Zap70 moiety was relieved at a lower concentration) by endoxifen than an endoxifen-responsive dominant negative Zap70 polypeptide including either (i) an ER(T2) domain having a G400V mutation or (ii) a wild-type estrogen receptor fragment. Being able to relieve the inhibition or masking of the dominant negative Zap70 moiety was relieved at a lower concentration of a trigger (e.g., endoxifen) can be advantageous, for example, because a lower dose of a trigger can be administered to a subject, which in turn may result in fewer side effects and less frequent administrations (that can be, e.g., inconvenient, painful and/or costly).

### Example 9: Interleukin 2 Levels can be Measured to Determine T Cell Activation and the Activity of an Endoxifen-Responsive Dominant Negative Zap-70 Polypeptide

This example demonstrates that the level of T cell activity and the activity of a dominant negative signaling moiety included in a trigger-responsive dominant negative signaling polypeptide can be determined by examining the level of interleukin 2 (“IL-2”).

One hundred thousand Jurkat E6.1 cells (ATCC, Manassas, Va.) are plated into individual wells of a 96-well plate with RPMI medium plus 5% FBS stripped with charcoal dextran (ThermoFisher Scientific). Cells are pre-treated with 1:30 ImmunoCult CD3/CD28/CD2 tetrameric antibody mixture (STEMCELL Technologies, Vancouver, BC) for 2 days to pre-stimulate T cell activation. Cells are washed and rested for 2 days before the cells are transduced with a vector containing trigger-responsive dominant negative signaling polypeptide. Efficient stable transduction of this vector is achieved utilizing a lentiviral vector, or alternatively, by liposome-mediated transfection with co-expression of a selective marker containing a gene resistant to ouabain or antibiotic. At least 4 hours later cells are re-exposed to 1:30 Immunocult plus an appropriate ligand trigger in RPMI medium plus 5% FBS stripped with charcoal dextran. A 1×cocktail of phorbol myristate acetate and ionomycin are used as a positive control for IL-2 induction (BioLegend, San Diego, Calif.). At least 16 hours later, supernatants are collected for immediate use, or are stored at −80° C. Supernatants are diluted 1:10 and used in an ELISA for IL-2 using the manufacturer's recommended protocol (BD Bioscience, San Jose, Calif.). Absorbance is read using the Varioskan LUX plate reader (ThermoFisher Scientific).

### Example 10: Modulation of Dominant-Negative Signaling Moiety Activity by an ER(T12) Modulating Domain

This example demonstrates that modulating domain ER(T12) can inhibit activity of a dominant-negative signaling moiety to which it is operatively linked, and that the inhibition of the dominant negative signaling moiety by the ER(T12) modulating domain can be relieved in the presence of a trigger. This example further shows that, when inhibition of the dominant negative signaling moiety by the ER(T12) modulating domain is relieved by the interaction of trigger with modulating domain, the dominant negative signaling moiety can inhibit a T cell activation cascade.

ER(T12) is an endoxifen-responsive modulating domain that is a variant of a ligand binding domain (LBD) fragment (amino acids 282-595) of human estrogen receptor-α having an amino acid sequence as set forth in SEQ ID NO: 13, including G400L (ctg), M543A (gcg), and L544A (gcg). This modulating domain can be encoded by the nucleic acid sequence set forth in SEQ ID NO: 14.

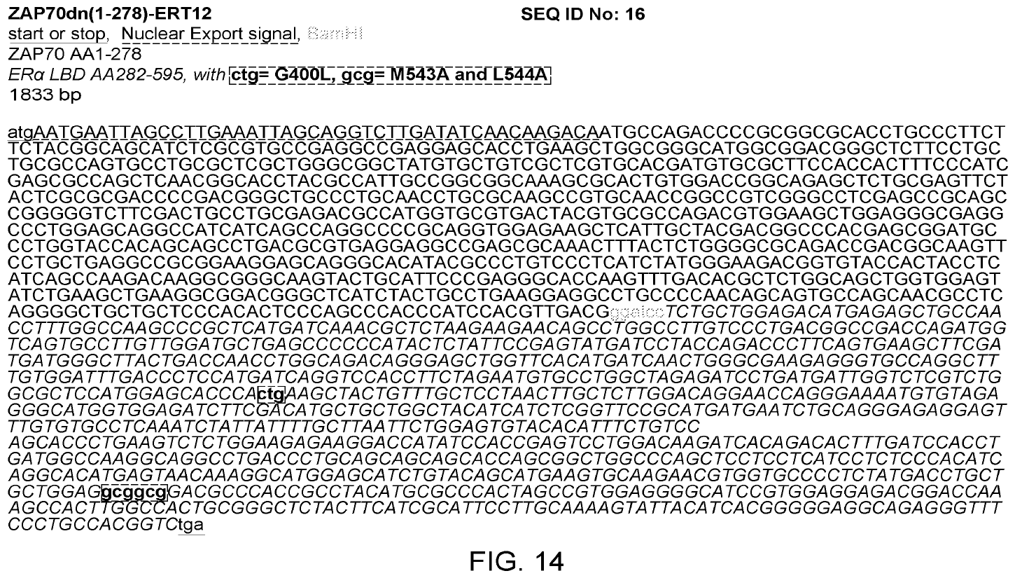

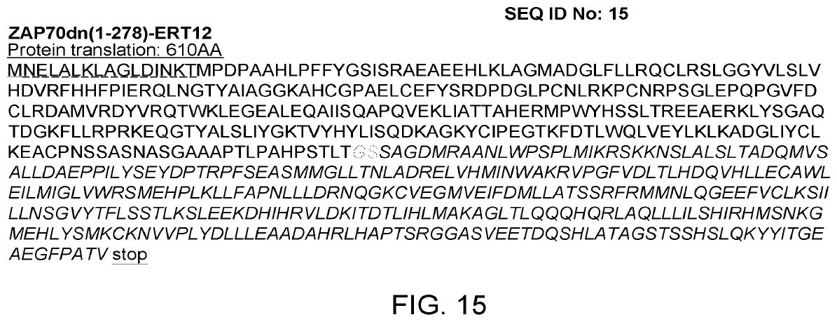

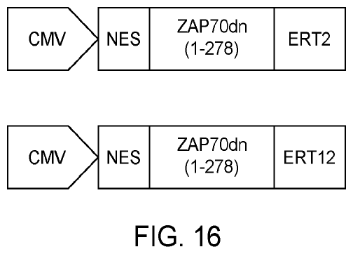

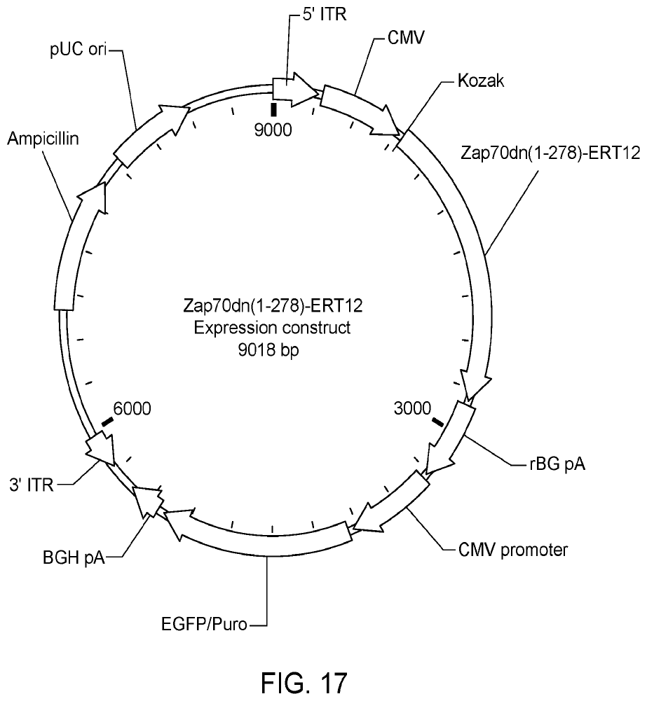

To examine the ability of ER(T12) to modulate activity of a dominant-negative signaling moiety to which it is operatively linked, a nucleic acid that can express a polypeptide including a LAC70dn domain operatively linked to ER(T12) (“ZAP70dn(1-278)-ER(T12)”) was generated (FIGS. 14-16). ZAP70dn(1-278) lacks a functional kinase domain. The ZAP70dn(1-278)-ER(T12) polypeptide includes a nuclear export signal (SEQ ID NO: 6; encoded by SEQ ID NO: 5), a ZAP70dn domain (amino acids 1-278; SEQ ID NO: 2; encoded by SEQ ID NO: 1), and ER(T12) (SEQ ID NO: 13; encoded by SEQ ID NO: 14). The complete amino acid sequence of a trigger-responsive dominant negative signaling polypeptide ZAP70dn(1-278)-ER(T12) is set forth in SEQ ID NO: 15, and is encoded by SEQ ID NO: 16. The nucleic acid of SEQ ID NO: 15 can be operatively linked to a CMV promoter for expression (FIG. 16). An expression construct for expression of ZAP70dn(1-278)-ER(T12) is shown in FIG. 17. Similar expression constructs were prepared for ZAP70dn(1-278) and ZAP70dn(1-278)-ER(T2).

To provide a means for assessing immune pathway activity, a construct containing a reporter gene, such as firefly luciferase (Luc), under the control of an NFAT response element can be used, as described, e.g., in Example 3. In the present Example, Jurkat E6.1 cells were transiently transfected with an NFAT-Luciferase reporter construct as well as one of (a) empty vector; (b) an expression construct encoding ZAP70dn(1-278); (c) an expression construct encoding ZAP70dn(1-278)-ER(T2); and (d) an expression construct encoding ZAP70dn(1-278)-ER(T12). Transient transfections were performed using LTX and PLUS transfection reagent (ThermoFisher Scientific) according to manufacturer's protocols.

A first set of transfected cells was treated in the absence or presence of 1:30 dilution of Immunocult CD3/CD28/CD2 tetrameric antibody mixture (StemCell Technologies, Canada), which stimulated T-cell activity. Stimulated cells were then further treated with RPMI 1640 media plus 4.8% FBS stripped with charcoal dextran (ThermoFisher Scientific), either without Z-endoxifen or with a designated amount of Z-endoxifen (Axon-Medchem). Luciferase activity (luminescence) was measured and reported as a percentage induction of NFAT-Luciferase.

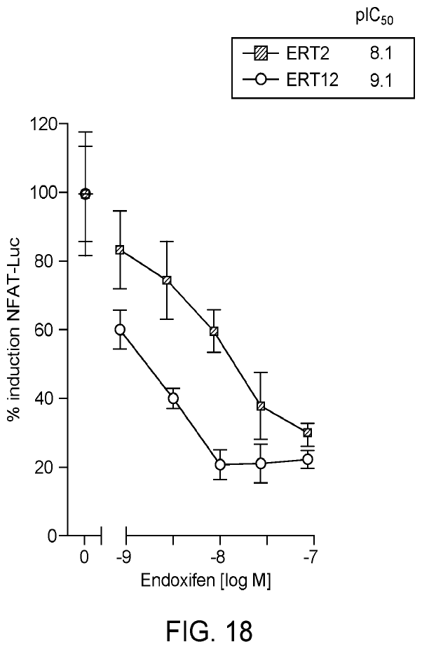

In Jurkat E6.1 cells transiently transfected with NFAT-Luciferase reporter construct as well as either an expression construct encoding ZAP70dn(1-278)-ER(T2) or an expression construct encoding ZAP70dn(1-278)-ER(T12), luciferase induction was generally inverse to the exposure of cells to endoxifen trigger, at least in that both ZAP70dn(1-278)-ER(T2) and ZAP70dn(1-278)-ER(T12) demonstrated greater inhibition of luciferase induction at certain relatively higher concentrations of endoxifen than at certain relatively lower concentrations of endoxifen (FIG. 18). These data confirm that both ER(T2) and ER(T12) are effective modulatory domains for trigger-responsive modulation of the activity of a dominant negative signaling moiety. While the effect of endoxifen was potent with respect to both ZAP70dn(1-278)-ER(T2) activity and ZAP70dn(1-278)-ER(T12) activity, the modulatory effect of Endoxifen was especially potent with respect to ZAP70dn(1-278)-ER(T12).

A further set of transfected cells was treated in the absence or presence of 0.25 μg/ml C305 (αTCR, Sigma-Aldrich) plus 2 μg/ml αCD28.2 (Biolegend), which stimulated T-cell activity. Stimulated cells were then further treated with RPMI 1640 media plus 4.8% FBS stripped with charcoal dextran (ThermoFisher Scientific), either without Z-endoxifen or with a designated amount of Z-endoxifen (Axon-Medchem; 0.05 to 50 nM). Luciferase activity (luminescence) was measured and reported in relative light units (RLU).

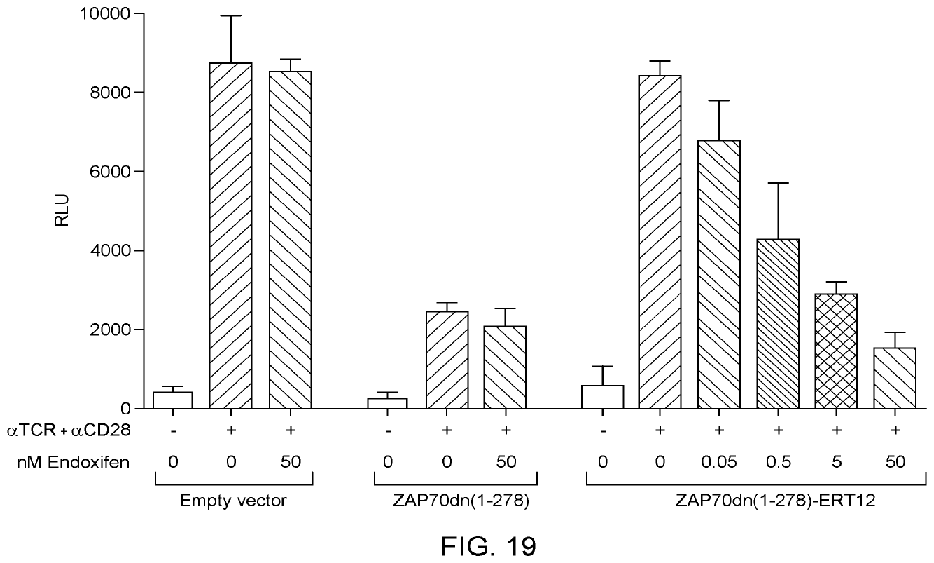

Results shown in FIG. 19 further confirm that ER(T12) is an effective modulatory domain for modulating activity of a dominant negative signaling moiety. In studied cells, αTCR+αCD28 induced expression of the luciferase reporter, as measured in RLU. As expected, endoxifen alone (50 nM) did not significantly effect luciferase expression in cells treated with αTCR+αCD28 to induce luciferase expression (absent a trigger-responsive dominant negative signaling polypeptide). Further, expression of the dominant negative signaling moiety ZAP70dn(1-278), absent a modulating domain, substantially reduced expression of luciferase reporter, with or without exposure to endoxifen as compared to empty vector controls. Finally, in cells expressing trigger-responsive dominant negative signalling polypeptide ZAP70dn(1-278)-ER(T12), the inhibitory effect of ZAP70dn(1-278) on luciferase reporter expression was dose-dependent on treatment with endoxifen. Absent endoxifen, the ZAP70dn(1-278) inhibitory effect was substantially blocked by the ER(T12) modulatory domain, which blockage was substantially relieved in the presence of 50 nM endoxifen.

### Example 11: Inhibition of Expression by Trigger-Responsive Dominant Negative LCK Polypeptides

This example demonstrates that modulating domain ER(T12) can inhibit activity of a dominant-negative signaling moiety to which it is operatively linked, and that the inhibition of the dominant negative signaling moiety by the ER(T12) modulating domain can be relieved in the presence of a trigger. This example further shows that, when inhibition of the dominant negative signaling moiety by the ER(T12) modulating domain is relieved by the interaction of trigger with modulating domain, the dominant negative signaling moiety can inhibit a T cell activation cascade. Moreover, this example shows that LCK(1-266) is a dominant negative signaling moiety that can inhibit a T cell activation cascade. Further, this example shows that LCK(1-266) is a dominant negative signaling moiety that can be trigger-responsive when operatively linked to a modulating domain in a trigger-responsive dominant negative signaling polypeptide.

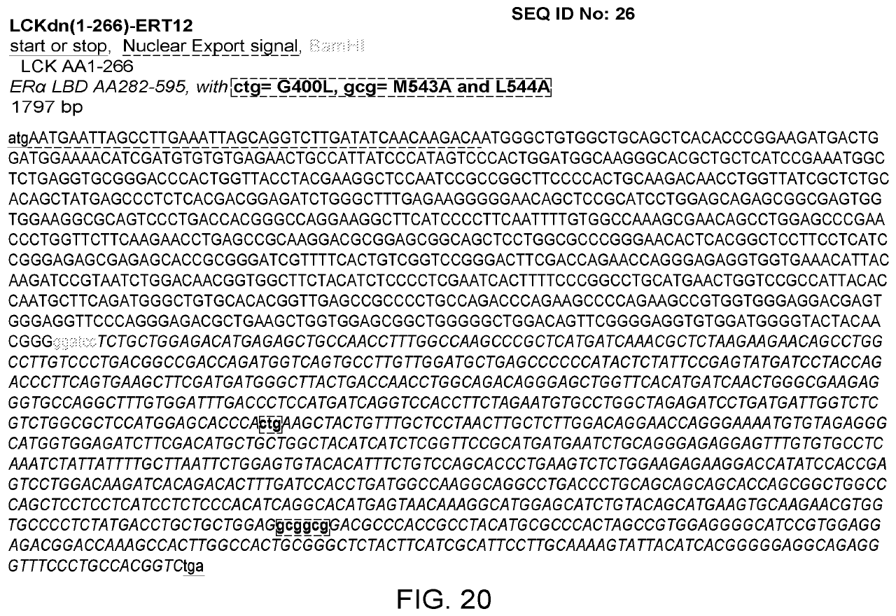

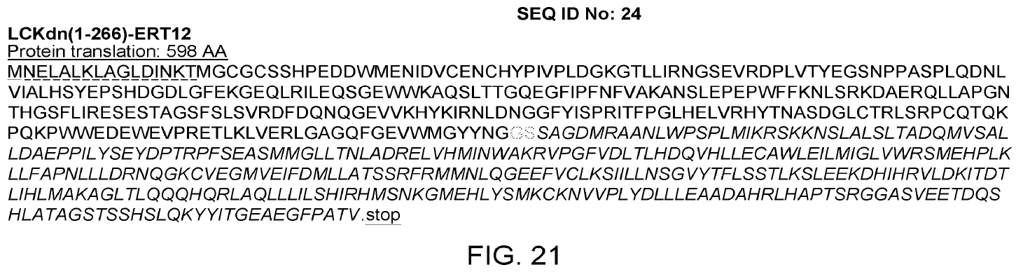

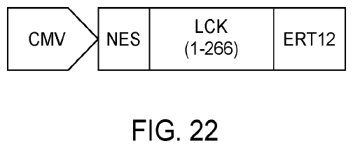

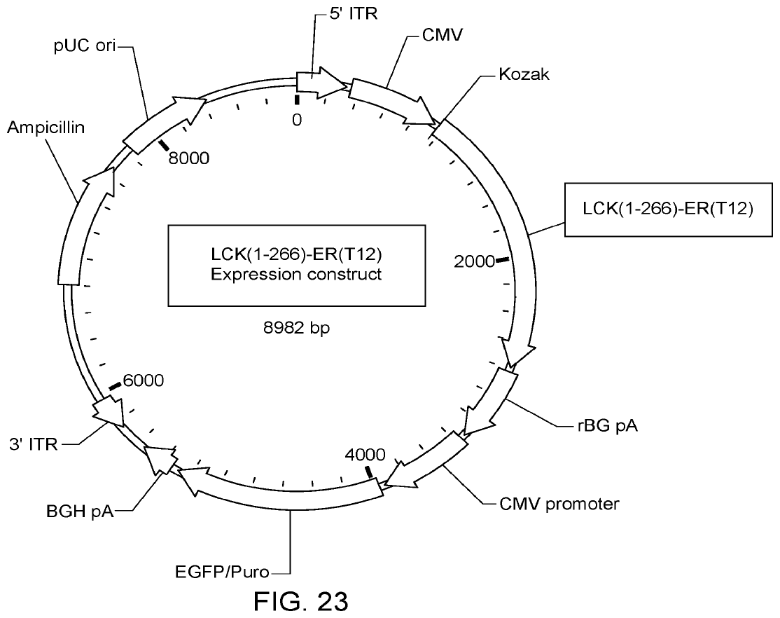

To examine the ability of LCK(1-266) to modulate activity of an immune pathway in a dominant-negative manner, and further to show that LCK(1-266) is a dominant negative signaling moiety that can be trigger-responsive when operatively linked to a modulating domain, various constructs were prepared. These constructs included, among other constructs and components, a construct capable of expressing a LCK(1-266)-ER(T12) trigger-responsive dominant negative signaling polypeptide (FIGS. 20-22). LCK(1-266)-ER(T12) polypeptide includes a nuclear export signal (SEQ ID NO: 5; encoded by SEQ ID NO: 6), a LCK(1-266) domain (SEQ ID NO: 17; encoded by SEQ ID NO: 19), and ER(T12) (SEQ ID NO: 13; encoded by SEQ ID NO: 14). The complete amino acid sequence of a trigger-responsive dominant negative signaling polypeptide LCK(1-266)-ER(T12) is set forth in SEQ ID NO: 18, and is encoded by SEQ ID NO: 22. The nucleic acid of SEQ ID NO: 18 can be operatively linked to a CMV promoter for expression (FIG. 22). An expression construct for expression of LCK(1-266)-ER(T12) is shown in FIG. 23. A similar expression construct was prepared for LCK(1-266).

To provide a means for assessing T cell activity, a construct containing a reporter gene, such as firefly luciferase (Luc), under the control of an NFAT response element can be used, as described, e.g., in Example 3. In the present Example, Jurkat E6.1 cells were transiently transfected with an NFAT-Luciferase reporter construct, as well as one of (a) empty vector control; (b) an expression construct encoding LCK(1-266); and (c) an expression construct encoding LCK(1-266)-ER(T12). Transient transfections were performed using LTX and PLUS transfection reagent (ThermoFisher Scientific) according to manufacturer's protocols.

Transfected cells were treated in the absence or presence of 1:80 dilution of Immunocult CD3/CD28 tetrameric antibody mixture (StemCell Technologies, Canada), which stimulated T-cell activity. Stimulated cells were then further treated with RPMI 1640 media plus 4.8% FBS stripped with charcoal dextran (ThermoFisher Scientific), either without Z-endoxifen or with 100 nM Z-endoxifen (Axon-Medchem). Luciferase activity (luminescence) was measured and reported as percent induction of NFAT-Luciferase.

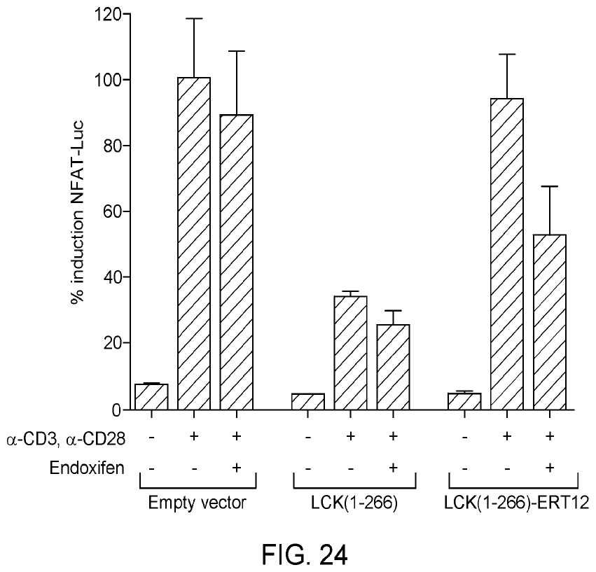

FIG. 24 confirms that LCK(1-266) is an effective dominant negative signaling polypeptide for modulating activity of an immune pathway. In studied cells, α-CD3 and α-CD28 induced expression of the luciferase reporter. As expected, endoxifen alone did not significantly effect luciferase expression in cells treated with α-CD3 and α-CD28 (absent a trigger-responsive dominant negative signaling polypeptide). Further, expression of the dominant negative signaling moiety LCK(1-266), absent a modulating domain, substantially reduced expression of luciferase reporter, with or without exposure to endoxifen, as compared to empty vector controls. Finally, in cells expressing trigger-responsive dominant negative signaling polypeptide LCK(1-266)-ER(T12), the inhibitory effect of LCK(1-266) on luciferase reporter expression was dependent on treatment with endoxifen. Absent endoxifen, the LCK(1-266) inhibitory effect was substantially blocked by the ER(T12) modulatory domain, which blockage was substantially relieved in the presence of endoxifen.

### Example 12: Inhibition of Expression by Trigger-Responsive Constitutively Active SHP1 Polypeptides

This example demonstrates that modulating domain ER(T12) can inhibit activity of a constitutively active signaling moiety to which it is operatively linked, and that the inhibition of the constitutively active signaling moiety by the ER(T12) modulating domain can be relieved in the presence of a trigger. This example further shows that, when inhibition of the constitutively active signaling moiety by the ER(T12) modulating domain is relieved by the interaction of trigger with modulating domain, the constitutively active signaling moiety can inhibit a T cell activation cascade. Moreover, this example shows that SHP1(210-595) is a constitutively active signaling moiety that can inhibit a T cell activation pathway. It is hypothesized, without wishing to be bound by any particular scientific theory, that deletion of SH2 domains renders SHP-1 constitutively active, in which state SHP1 inhibits T-cell activation, e.g., by removing phosphates from LCK, from Zeta chains, and from endogenous ZAP70. Further, this example shows that SHP1(210-595) is a constitutively active signaling moiety that can be trigger-responsive when operatively linked to a modulating domain in a trigger-responsive dominant negative signaling polypeptide.

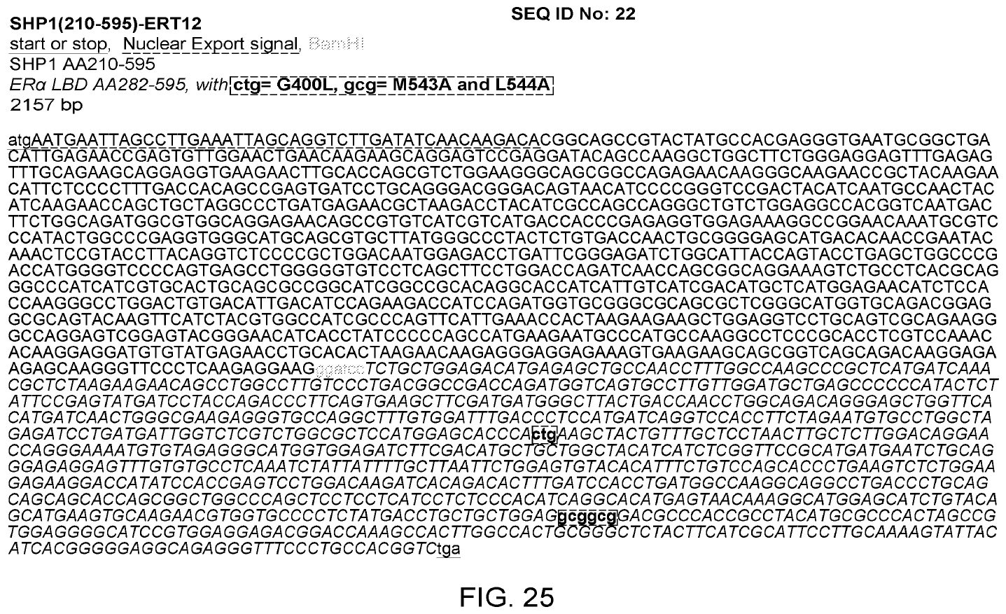

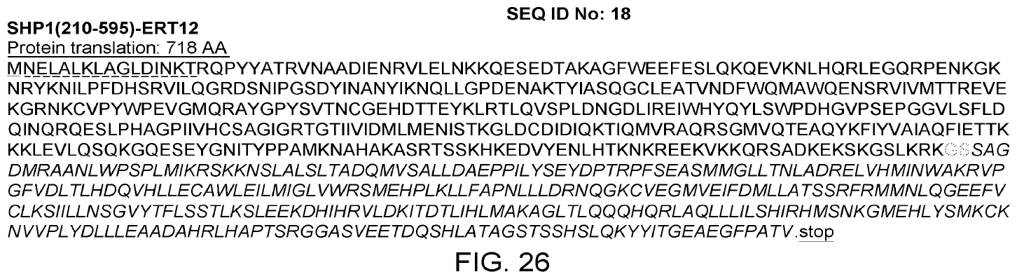

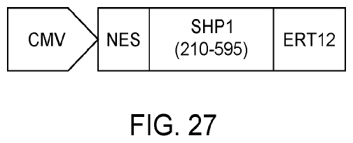

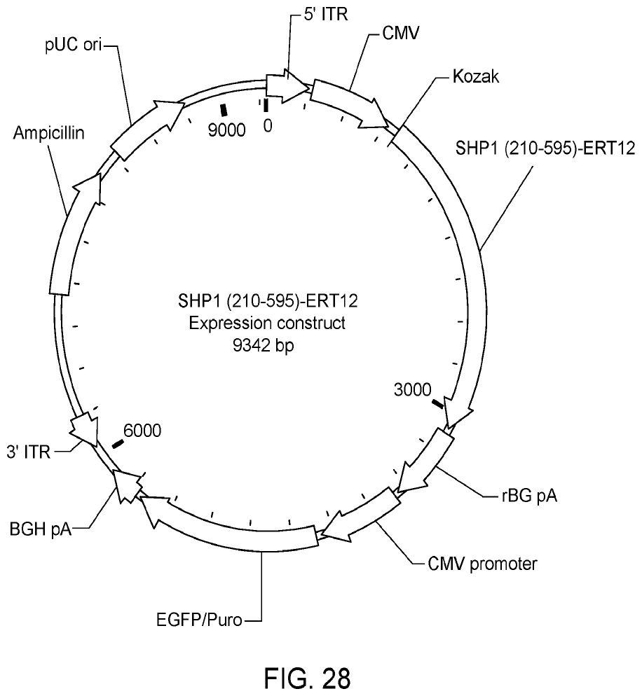

To examine the ability of SHP1(210-595) to modulate activity of an immune pathway in a constitutive manner, and further to show that SHP1(210-595) is a constitutively active signaling moiety that can be trigger-responsive when operatively linked to a modulating domain, various constructs were prepared. These constructs included, among other constructs and components, a construct capable of expressing a SHP1(210-595)-ER(T12) trigger-responsive constitutively active signaling polypeptide (FIGS. 25-27). SHP1(210-595)-ER(T12) polypeptide included a nuclear export signal (SEQ ID NO: 6; encoded by SEQ ID NO: 5), a SHP1(210-595) domain (SEQ ID NO: 23; encoded by SEQ ID NO: 25), and ER(T12) (SEQ ID NO: 13; encoded by SEQ ID NO: 14). The complete amino acid sequence of a trigger-responsive constitutively active signaling polypeptide SHP1(210-595)-ER(T12) is set forth in SEQ ID NO: 24, and is encoded by SEQ ID NO: 26. The nucleic acid of SEQ ID NO: 24 can be operatively linked to a CMV promoter for expression (FIG. 27). An expression construct for expression of SHP1(210-595)-ER(T12) is shown in FIG. 28. A similar expression construct was prepared for SHP1(210-595).

To provide a means for assessing T cell activity, a construct containing a reporter gene, such as firefly luciferase (Luc), under the control of an IL2 response element can be used. In the present Example, Jurkat E6.1 cells cells were transiently transfected with an IL2-Luciferase reporter construct, as well as one of (a) empty vector; (b) an expression construct encoding SHP1(210-595); and (c) an expression construct encoding SHP1(210-595)-ER(T12). Transient transfections were performed using LTX and PLUS transfection reagent (ThermoFisher Scientific) according to manufacturer's protocols.

Transfected cells were treated in the absence or presence of 1:30 dilution of Immunocult CD3/CD28 tetrameric antibody mixture (StemCell Technologies, Canada), which stimulated T-cell activity. Stimulated cells were then further treated with RPMI 1640 media plus 4.8% FBS stripped with charcoal dextran (ThermoFisher Scientific), without Z-endoxifen or with 50 nM Z-endoxifen (Axon-Medchem). Luciferase activity (luminescence) was measured and reported as percent induction of IL2-Luciferase.

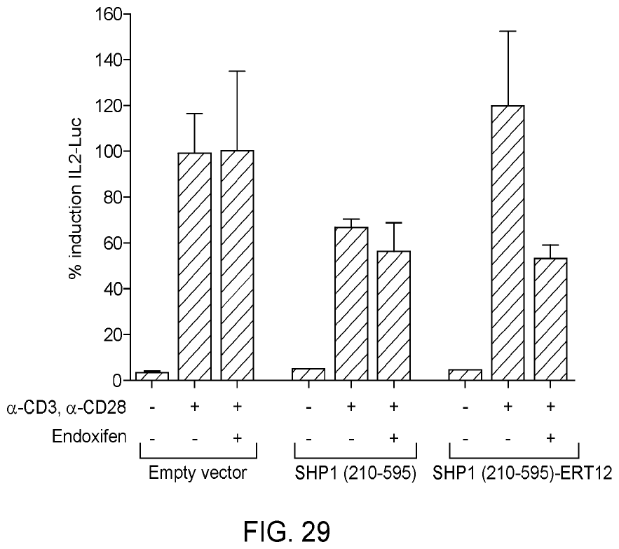

FIG. 29 confirms that SHP1(210-595) is an effective constitutively active signaling polypeptide for modulating activity of an immune pathway. In studied cells, α-CD3 and α-CD28 induced expression of the luciferase reporter. As expected, endoxifen alone did not significantly effect luciferase expression in cells treated with α-CD3 and α-CD28 (absent a trigger-responsive dominant negative signaling polypeptide). Further, expression of the constitutively active signaling moiety SHP1(210-595), absent a modulating domain, substantially reduced expression of luciferase reporter, with or without exposure to endoxifen, as compared to empty vector controls. Finally, in cells expressing trigger-responsive constitutively active signaling polypeptide SHP1(210-595)-ER(T12), the inhibitory effect of SHP1(210-595) on luciferase reporter expression was dependent on treatment with endoxifen. Absent endoxifen, the SHP1(210-595) inhibitory effect was substantially blocked by the ER(T12) modulatory domain, which blockage was substantially relieved in the presence of endoxifen.

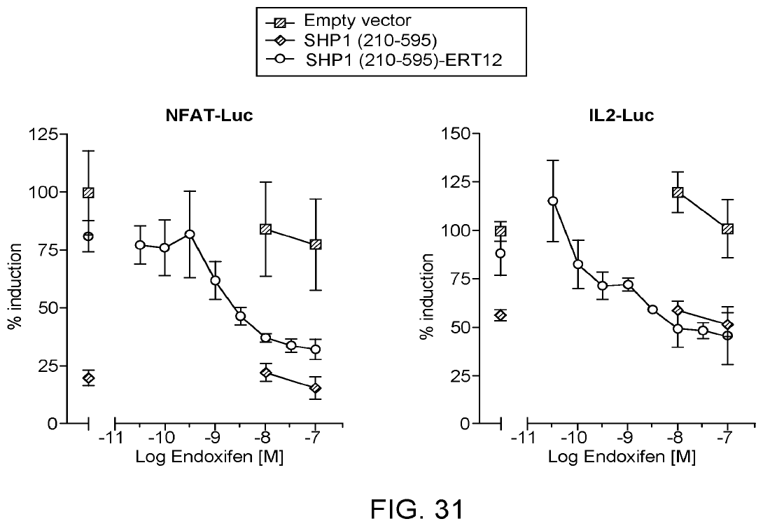

FIG. 31 confirmed that activity of SHP1(210-595) inhibited expression of reporter constructs for assessing T cell activity in an endoxifen-independent manner, while SHP1(210-595)-ER(T12) inhibited expression of reporter constructs for assessing T cell activity in a manner responsive to concentration of endoxifen. Reporter constructs included firefly luciferase (Luc) under the control of an NFAT response element and firefly luciferase (Luc) under the control of an IL2 response element. Data presented in the first panel is from Jurkat E6.1 cells transiently transfected with an NFAT-Luciferase reporter construct, as well as one of (a) empty vector; (b) an expression construct encoding SHP1(210-595); and (c) an expression construct encoding SHP1(210-595)-ER(T12). Data presented in the second panel is from Jurkat E6.1 cells transiently transfected with an IL2-Luciferase reporter construct, as well as one of (a) empty vector; (b) an expression construct encoding SHP1(210-595); and (c) an expression construct encoding SHP1(210-595)-ER(T12). Transient transfections were performed using LTX and PLUS transfection reagent (ThermoFisher Scientific) according to manufacturer's protocols. Transfected cells were treated in the presence of 1:40 dilution of Immunocult CD3/CD28 tetrameric antibody mixture (StemCell Technologies, Canada), which stimulated T-cell activity. Stimulated cells were then further treated with RPMI 1640 media plus 4.8% FBS stripped with charcoal dextran (ThermoFisher Scientific), with varying amounts of Z-endoxifen (Axon-Medchem) as indicated in FIG. 31. Luciferase activity (luminescence) was measured and reported as percent induction of IL2-Luciferase. SHP1(210-595)-ER(T12) was dose-responsive to concentration of endoxifen as measured by percentage indication of either NFAT-Luciferase reporter or IL2-Luciferase reporter.

SEQUENCES
Exemplary nucleotide sequence encoding
a dominant negative Zap70
SEQ ID NO: 1
ATGCCAGACCCCGCGGCGCACCTGCCCTTCTTCTA
CGGCAGCATCTCGCGTGCCGAGGCCGAGGAGCACC
TGAAGCTGGCGGGCATGGCGGACGGGCTCTTCCTG
CTGCGCCAGTGCCTGCGCTCGCTGGGCGGCTATGT
GCTGTCGCTCGTGCACGATGTGCGCTTCCACCACT
TTCCCATCGAGCGCCAGCTCAACGGCACCTACGCC
ATTGCCGGCGGCAAAGCGCACTGTGGACCGGCAGA
GCTCTGCGAGTTCTACTCGCGCGACCCCGACGGGC
TGCCCTGCAACCTGCGCAAGCCGTGCAACCGGCCG
TCGGGCCTCGAGCCGCAGCCGGGGGTCTTCGACTG
CCTGCGAGACGCCATGGTGCGTGACTACGTGCGCC
AGACGTGGAAGCTGGAGGGCGAGGCCCTGGAGCAG
GCCATCATCAGCCAGGCCCCGCAGGTGGAGAAGCT
CATTGCTACGACGGCCCACGAGCGGATGCCCTGGT
ACCACAGCAGCCTGACGCGTGAGGAGGCCGAGCGC
AAACTTTACTCTGGGGCGCAGACCGACGGCAAGTT
CCTGCTGAGGCCGCGGAAGGAGCAGGGCACATACG
CCCTGTCCCTCATCTATGGGAAGACGGTGTACCAC
TACCTCATCAGCCAAGACAAGGCGGGCAAGTACTG
CATTCCCGAGGGCACCAAGTTTGACACGCTCTGGC
AGCTGGTGGAGTATCTGAAGCTGAAGGCGGACGGG
CTCATCTACTGCCTGAAGGAGGCCTGCCCCAACAG
CAGTGCCAGCAACGCCTCAGGGGCTGCTGCTCCCA
CACTCCCAGCCCACCCATCCACGTTGACG
Exemplary amino acid sequence of a
dominant negative ZAP70
SEQ ID NO: 2
MPDPAAHLPFFYGSISRAEAEEHLKLAGMADGLFL
LRQCLRSLGGYVLSLVHDVRFHHFPIERQLNGTYA
IAGGKAHCGPAELCEFYSRDPDGLPCNLRKPCNRP
SGLEPQPGVFDCLRDAMVRDYVRQTWKLEGEALEQ
AIISQAPQVEKLIATTAHERMPWYHSSLTREEAER
KLYSGAQTDGKFLLRPRKEQGTYALSLIYGKTVYH
YLISQDKAGKYCIPEGTKFDTLWQLVEYLKLKADG
LIYCLKEACPNSSASNASGAAAPTLPAHPSTLT
Exemplary nucleotide sequence
encoding ER(T2) polypeptide
SEQ ID NO: 3
TCTGCTGGAGACATGAGAGCTGCCAACCTTTGGCC
AAGCCCGCTCATGATCAAACGCTCTAAGAAGAACA
GCCTGGCCTTGTCCCTGACGGCCGACCAGATGGTC
AGTGCCTTGTTGGATGCTGAGCCCCCCATACTCTA
TTCCGAGTATGATCCTACCAGACCCTTCAGTGAAG
CTTCGATGATGGGCTTACTGACCAACCTGGCAGAC
AGGGAGCTGGTTCACATGATCAACTGGGCGAAGAG
GGTGCCAGGCTTTGTGGATTTGACCCTCCATGATC
AGGTCCACCTTCTAGAATGTGCCTGGCTAGAGATC
CTGATGATTGGTCTCGTCTGGCGCTCCATGGAGCA
CCCAGTGAAGCTACTGTTTGCTCCTAACTTGCTCT
TGGACAGGAACCAGGGAAAATGTGTAGAGGGCATG
GTGGAGATCTTCGACATGCTGCTGGCTACATCATC
TCGGTTCCGCATGATGAATCTGCAGGGAGAGGAGT
TTGTGTGCCTCAAATCTATTATTTTGCTTAATTCT
GGAGTGTACACATTTCTGTCCAGCACCCTGAAGTC
TCTGGAAGAGAAGGACCATATCCACCGAGTCCTGG
ACAAGATCACAGACACTTTGATCCACCTGATGGCC
AAGGCAGGCCTGACCCTGCAGCAGCAGCACCAGCG
GCTGGCCCAGCTCCTCCTCATCCTCTCCCACATCA
GGCACATGAGTAACAAAGGCATGGAGCATCTGTAC
AGCATGAAGTGCAAGAACGTGGTGCCCCTCTATGA
CCTGCTGCTGGAGGCGGCGGACGCCCACCGCCTAC
ATGCGCCCACTAGCCGTGGAGGGGCATCCGTGGAG
GAGACGGACCAAAGCCACTTGGCCACTGCGGGCTC
TACTTCATCGCATTCCTTGCAAAAGTATTACATCA
CGGGGGAGGCAGAGGGTTTCCCTGCCACGGTC
Exemplary amino acid sequence of
ER(T2) polypeptide
SEQ ID NO: 4
SAGDMRAANLWPSPLMIKRSKKNSLALSLTADQMV
SALLDAEPPILYSEYDPTRPFSEASMMGLLTNLAD
RELVHMINWAKRVPGFVDLTLHDQVHLLECAWLEI
LMIGLVWRSMEHPVKLLFAPNLLLDRNQGKCVEGM
VEIFDMLLATSSRFRMMNLQGEEFVCLKSIILLNS
GVYTFLSSTLKSLEEKDHIHRVLDKITDTLIHLMA
KAGLTLQQQHQRLAQLLLILSHIRHMSNKGMEHLY
SMKCKNVVPLYDLLLEAADAHRLHAPTSRGGASVE
ETDQSHLATAGSTSSHSLQKYYITGEAEGFPATV
Exemplary nucleotide sequence of a
Nuclear Export Signal
SEQ ID NO: 5
AATGAATTAGCCTTGAAATTAGCAGGTCTTGAT
ATCAACAAGACA
Exemplary amino acid sequence of a
Nuclear Export Signal
SEQ ID NO: 6
NELALKLAGLDINKT
Exemplary nucleotide sequence encoding
an endoxifen-responsive dominant
negative Zap-70 polypeptide
(NES-ZAP70dn-ER(T2)
SEQ ID NO: 7
ATGAATGAATTAGCCTTGAAATTAGCAGGTCTTGA
TATCAACAAGACAATGCCAGACCCCGCGGCGCACC
TGCCCTTCTTCTACGGCAGCATCTCGCGTGCCGAG
GCCGAGGAGCACCTGAAGCTGGCGGGCATGGCGGA
CGGGCTCTTCCTGCTGCGCCAGTGCCTGCGCTCGC
TGGGCGGCTATGTGCTGTCGCTCGTGCACGATGTG
CGCTTCCACCACTTTCCCATCGAGCGCCAGCTCAA
CGGCACCTACGCCATTGCCGGCGGCAAAGCGCACT
GTGGACCGGCAGAGCTCTGCGAGTTCTACTCGCGC
GACCCCGACGGGCTGCCCTGCAACCTGCGCAAGCC
GTGCAACCGGCCGTCGGGCCTCGAGCCGCAGCCGG
GGGTCTTCGACTGCCTGCGAGACGCCATGGTGCGT
GACTACGTGCGCCAGACGTGGAAGCTGGAGGGCGA
GGCCCTGGAGCAGGCCATCATCAGCCAGGCCCCGC
AGGTGGAGAAGCTCATTGCTACGACGGCCCACGAG
CGGATGCCCTGGTACCACAGCAGCCTGACGCGTGA
GGAGGCCGAGCGCAAACTTTACTCTGGGGCGCAGA
CCGACGGCAAGTTCCTGCTGAGGCCGCGGAAGGAG
CAGGGCACATACGCCCTGTCCCTCATCTATGGGAA
GACGGTGTACCACTACCTCATCAGCCAAGACAAGG
CGGGCAAGTACTGCATTCCCGAGGGCACCAAGTTT
GACACGCTCTGGCAGCTGGTGGAGTATCTGAAGCT
GAAGGCGGACGGGCTCATCTACTGCCTGAAGGAGG
CCTGCCCCAACAGCAGTGCCAGCAACGCCTCAGGG
GCTGCTGCTCCCACACTCCCAGCCCACCCATCCAC
GTTGACGGGATCCTCTGCTGGAGACATGAGAGCTG
CCAACCTTTGGCCAAGCCCGCTCATGATCAAACGC
TCTAAGAAGAACAGCCTGGCCTTGTCCCTGACGGC
CGACCAGATGGTCAGTGCCTTGTTGGATGCTGAGC
CCCCCATACTCTATTCCGAGTATGATCCTACCAGA
CCCTTCAGTGAAGCTTCGATGATGGGCTTACTGAC
CAACCTGGCAGACAGGGAGCTGGTTCACATGATCA
ACTGGGCGAAGAGGGTGCCAGGCTTTGTGGATTTG
ACCCTCCATGATCAGGTCCACCTTCTAGAATGTGC
CTGGCTAGAGATCCTGATGATTGGTCTCGTCTGGC
GCTCCATGGAGCACCCAGTGAAGCTACTGTTTGCT
CCTAACTTGCTCTTGGACAGGAACCAGGGAAAATG
TGTAGAGGGCATGGTGGAGATCTTCGACATGCTGC
TGGCTACATCATCTCGGTTCCGCATGATGAATCTG
CAGGGAGAGGAGTTTGTGTGCCTCAAATCTATTAT
TTTGCTTAATTCTGGAGTGTACACATTTCTGTCCA
GCACCCTGAAGTCTCTGGAAGAGAAGGACCATATC
CACCGAGTCCTGGACAAGATCACAGACACTTTGAT
CCACCTGATGGCCAAGGCAGGCCTGACCCTGCAGC
AGCAGCACCAGCGGCTGGCCCAGCTCCTCCTCATC
CTCTCCCACATCAGGCACATGAGTAACAAAGGCAT
GGAGCATCTGTACAGCATGAAGTGCAAGAACGTGG
TGCCCCTCTATGACCTGCTGCTGGAGGCGGCGGAC
GCCCACCGCCTACATGCGCCCACTAGCCGTGGAGG
GGCATCCGTGGAGGAGACGGACCAAAGCCACTTGG
CCACTGCGGGCTCTACTTCATCGCATTCCTTGCAA
AAGTATTACATCACGGGGGAGGCAGAGGGTTTCCC
TGCCACGGTC
Exemplary amino acid sequence of an
endoxifen-responsive dominant negative
Zap-70 polypeptide (NES-ZAP70dn-ER(T2)
SEQ ID NO: 8
MNELALKLAGLDINKTMPDPAAHLPFFYGSISRAE
AEEHLKLAGMADGLFLLRQCLRSLGGYVLSLVHDV
RFHHFPIERQLNGTYAIAGGKAHCGPAELCEFYSR
DPDGLPCNLRKPCNRPSGLEPQPGVFDCLRDAMVR
DYVRQTWKLEGEALEQAIISQAPQVEKLIATTAHE
RMPWYHSSLTREEAERKLYSGAQTDGKFLLRPRKE
QGTYALSLIYGKTVYHYLISQDKAGKYCIPEGTKF
DTLWQLVEYLKLKADGLIYCLKEACPNSSASNASG
AAAPTLPAHPSTLTGSSAGDMRAANLWPSPLMIKR
SKKNSLALSLTADQMVSALLDAEPPILYSEYDPTR
PFSEASMMGLLTNLADRELVHMINWAKRVPGFVDL
TLHDQVHLLECAWLEILMIGLVWRSMEHPVKLLFA
PNLLLDRNQGKCVEGMVEIFDMLLATSSRFRMMNL
QGEEFVCLKSIILLNSGVYTFLSSTLKSLEEKDHI
HRVLDKITDTLIHLMAKAGLTLQQQHQRLAQLLLI
LSHIRHMSNKGMEHLYSMKCKNVVPLYDLLLEAAD
AHRLHAPTSRGGASVEETDQSHLATAGSTSSHSLQ
KYYITGEAEGFPATV
Exemplary nucleotide sequence encoding
polypeptide including a nuclear export
signal (NES) and a dominant negative
Zap-70 moiety
SEQ ID NO: 9
ATGAATGAATTAGCCTTGAAATTAGCAGGTCTTGA
TATCAACAAGACAATGCCAGACCCCGCGGCGCACC
TGCCCTTCTTCTACGGCAGCATCTCGCGTGCCGAG
GCCGAGGAGCACCTGAAGCTGGCGGGCATGGCGGA
CGGGCTCTTCCTGCTGCGCCAGTGCCTGCGCTCGC
TGGGCGGCTATGTGCTGTCGCTCGTGCACGATGTG
CGCTTCCACCACTTTCCCATCGAGCGCCAGCTCAA
CGGCACCTACGCCATTGCCGGCGGCAAAGCGCACT
GTGGACCGGCAGAGCTCTGCGAGTTCTACTCGCGC
GACCCCGACGGGCTGCCCTGCAACCTGCGCAAGCC
GTGCAACCGGCCGTCGGGCCTCGAGCCGCAGCCGG
GGGTCTTCGACTGCCTGCGAGACGCCATGGTGCGT
GACTACGTGCGCCAGACGTGGAAGCTGGAGGGCGA
GGCCCTGGAGCAGGCCATCATCAGCCAGGCCCCGC
AGGTGGAGAAGCTCATTGCTACGACGGCCCACGAG
CGGATGCCCTGGTACCACAGCAGCCTGACGCGTGA
GGAGGCCGAGCGCAAACTTTACTCTGGGGCGCAGA
CCGACGGCAAGTTCCTGCTGAGGCCGCGGAAGGAG
CAGGGCACATACGCCCTGTCCCTCATCTATGGGAA
GACGGTGTACCACTACCTCATCAGCCAAGACAAGG
CGGGCAAGTACTGCATTCCCGAGGGCACCAAGTTT
GACACGCTCTGGCAGCTGGTGGAGTATCTGAAGCT
GAAGGCGGACGGGCTCATCTACTGCCTGAAGGAGG
CCTGCCCCAACAGCAGTGCCAGCAACGCCTCAGGG
GCTGCTGCTCCCACACTCCCAGCCCACCCATCCAC
GTTGACG
Exemplary peptide sequence of a polypeptide
including a nuclear export signal (NES) and
a dominant negative Zap-70 moiety
SEQ ID NO: 10
MNELALKLAGLDINKTMPDPAAHLPFFYGSISRAE
AEEHLKLAGMADGLFLLRQCLRSLGGYVLSLVHDV
RFHHFPIERQLNGTYAIAGGKAHCGPAELCEFYSR
DPDGLPCNLRKPCNRPSGLEPQPGVFDCLRDAMVR
DYVRQTWKLEGEALEQAIISQAPQVEKLIATTAHE
RMPWYHSSLTREEAERKLYSGAQTDGKFLLRPRKE
QGTYALSLIYGKTVYHYLISQDKAGKYCIPEGTKF
DTLWQLVEYLKLKADGLIYCLKEACPNSSASNASG
AAAPTLPAHPSTLT
Nucleotide sequence of the wild-type
human Estrogen Receptor-alpha (ESRI) cDNA
SEQ ID NO: 11
ATGACCATGACCCTCCACACCAAAGCATCTGGGAT
GGCCCTACTGCATCAGATCCAAGGGAACGAGCTGG
AGCCCCTGAACCGTCCGCAGCTCAAGATCCCCCTG
GAGCGGCCCCTGGGCGAGGTGTACCTGGACAGCAG
CAAGCCCGCCGTGTACAACTACCCCGAGGGCGCCG
CCTACGAGTTCAACGCCGCGGCCGCCGCCAACGCG
CAGGTCTACGGTCAGACCGGCCTCCCCTACGGCCC
CGGGTCTGAGGCTGCGGCGTTCGGCTCCAACGGCC
TGGGGGGTTTCCCCCCACTCAACAGCGTGTCTCCG
AGCCCGCTGATGCTACTGCACCCGCCGCCGCAGCT
GTCGCCTTTCCTGCAGCCCCACGGCCAGCAGGTGC
CCTACTACCTGGAGAACGAGCCCAGCGGCTACACG
GTGCGCGAGGCCGGCCCGCCGGCATTCTACAGGCC
AAATTCAGATAATCGACGCCAGGGTGGCAGAGAAA
GATTGGCCAGTACCAATGACAAGGGAAGTATGGCT
ATGGAATCTGCCAAGGAGACTCGCTACTGTGCAGT
GTGCAATGACTATGCTTCAGGCTACCATTATGGAG
TCTGGTCCTGTGAGGGCTGCAAGGCCTTCTTCAAG
AGAAGTATTCAAGGACATAACGACTATATGTGTCC
AGCCACCAACCAGTGCACCATTGATAAAAACAGGA
GGAAGAGCTGCCAGGCCTGCCGGCTCCGCAAATGC
TACGAAGTGGGAATGATGAAAGGTGGGATACGAAA
AGACCGAAGAGGAGGGAGAATGTTGAAACACAAGC
GCCAGAGAGATGATGGGGAGGGCAGGGGTGAAGTG
GGGTCTGCTGGAGACATGAGAGCTGCCAACCTTTG
GCCAAGCCCGCTCATGATCAAACGCTCTAAGAAGA
ACAGCCTGGCCTTGTCCCTGACGGCCGACCAGATG
GTCAGTGCCTTGTTGGATGCTGAGCCCCCCATACT
CTATTCCGAGTATGATCCTACCAGACCCTTCAGTG
AAGCTTCGATGATGGGCTTACTGACCAACCTGGCA
GACAGGGAGCTGGTTCACATGATCAACTGGGCGAA
GAGGGTGCCAGGCTTTGTGGATTTGACCCTCCATG
ATCAGGTCCACCTTCTAGAATGTGCCTGGCTAGAG
ATCCTGATGATTGGTCTCGTCTGGCGCTCCATGGA
GCACCCAGGGAAGCTACTGTTTGCTCCTAACTTGC
TCTTGGACAGGAACCAGGGAAAATGTGTAGAGGGC
ATGGTGGAGATCTTCGACATGCTGCTGGCTACATC
ATCTCGGTTCCGCATGATGAATCTGCAGGGAGAGG
AGTTTGTGTGCCTCAAATCTATTATTTTGCTTAAT
TCTGGAGTGTACACATTTCTGTCCAGCACCCTGAA
GTCTCTGGAAGAGAAGGACCATATCCACCGAGTCC
TGGACAAGATCACAGACACTTTGATCCACCTGATG
GCCAAGGCAGGCCTGACCCTGCAGCAGCAGCACCA
GCGGCTGGCCCAGCTCCTCCTCATCCTCTCCCACA
TCAGGCACATGAGTAACAAAGGCATGGAGCATCTG
TACAGCATGAAGTGCAAGAACGTGGTGCCCCTCTA
TGACCTGCTGCTGGAGATGCTGGACGCCCACCGCC
TACATGCGCCCACTAGCCGTGGAGGGGCATCCGTG
GAGGAGACGGACCAAAGCCACTTGGCCACTGCGGG
CTCTACTTCATCGCATTCCTTGCAAAAGTATTACA
TCACGGGGGAGGCAGAGGGTTTCCCTGCCACGGTC
TGA
Amino acid sequence of the wild-type
human Estrogen Receptor-alpha (ESR1)
SEQ ID NO: 12
MTMTLHTKASGMALLHQIQGNELEPLNRPQLKIPL
ERPLGEVYLDSSKPAVYNYPEGAAYEFNAAAAANA
QVYGQTGLPYGPGSEAAAFGSNGLGGFPPLNSVSP
SPLMLLHPPPQLSPFLQPHGQQVPYYLENEPSGYT
VREAGPPAFYRPNSDNRRQGGRERLASTNDKGSMA
MESAKETRYCAVCNDYASGYHYGVWSCEGCKAFFK
RSIQGHNDYMCPATNQCTIDKNRRKSCQACRLRKC
YEVGMMKGGIRKDRRGGRMLKHKRQRDDGEGRGEV
GSAGDMRAANLWPSPLMIKRSKKNSLALSLTADQM
VSALLDAEPPILYSEYDPTRPFSEASMMGLLTNLA
DRELVHMINWAKRVPGFVDLTLHDQVHLLECAWLE
ILMIGLVWRSMEHPGKLLFAPNLLLDRNQGKCVEG
MVEIFDMLLATSSRFRMMNLQGEEFVCLKSIILLN
SGVYTFLSSTLKSLEEKDHIHRVLDKITDTLIHLM
AKAGLTLQQQHQRLAQLLLILSHIRHMSNKGMEHL
YSMKCKNVVPLYDLLLEMLDAHRLHAPTSRGGASV
EETDQSHLATAGSTSSHSLQKYYITGEAEGFPATV
Exemplary amino acid sequence of
ER(T12) modulating domain
SEQ ID NO: 13
SAGDMRAANLWPSPLMIKRSKKNSLALSLTADQMV
SALLDAEPPILYSEYDPTRPFSEASMMGLLTNLAD
RELVHMINWAKRVPGFVDLTLHDQVHLLECAWLEI
LMIGLVWRSMEHPLKLLFAPNLLLDRNQGKCVEGM
VEIFDMLLATSSRFRMMNLQGEEFVCLKSIILLNS
GVYTFLSSTLKSLEEKDHIHRVLDKITDTLIHLMA
KAGLTLQQQHQRLAQLLLILSHIRHMSNKGMEHLY
SMKCKNVVPLYDLLLEAADAHRLHAPTSRGGASVE
ETDQSHLATAGSTSSHSLQKYYITGEAEGFPATV
Exemplary nucleic acid sequence encoding
ER(T12) modulating domain
SEQ ID NO: 14
TCTGCTGGAGACATGAGAGCTGCCAACCTTTGGCC
AAGCCCGCTCATGATCAAACGCTCTAAGAAGAACA
GCCTGGCCTTGTCCCTGACGGCCGACCAGATGGTC
AGTGCCTTGTTGGATGCTGAGCCCCCCATACTCTA
TTCCGAGTATGATCCTACCAGACCCTTCAGTGAAG
CTTCGATGATGGGCTTACTGACCAACCTGGCAGAC
AGGGAGCTGGTTCACATGATCAACTGGGCGAAGAG
GGTGCCAGGCTTTGTGGATTTGACCCTCCATGATC
AGGTCCACCTTCTAGAATGTGCCTGGCTAGAGATC
CTGATGATTGGTCTCGTCTGGCGCTCCATGGAGCA
CCCActgAAGCTACTGTTTGCTCCTAACTTGCTCT
TGGACAGGAACCAGGGAAAATGTGTAGAGGGCATG
GTGGAGATCTTCGACATGCTGCTGGCTACATCATC
TCGGTTCCGCATGATGAATCTGCAGGGAGAGGAGT
TTGTGTGCCTCAAATCTATTATTTTGCTTAATTCT
GGAGTGTACACATTTCTGTCCAGCACCCTGAAGTC
TCTGGAAGAGAAGGACCATATCCACCGAGTCCTGG
ACAAGATCACAGACACTTTGATCCACCTGATGGCC
AAGGCAGGCCTGACCCTGCAGCAGCAGCACCAGCG
GCTGGCCCAGCTCCTCCTCATCCTCTCCCACATCA
GGCACATGAGTAACAAAGGCATGGAGCATCTGTAC
AGCATGAAGTGCAAGAACGTGGTGCCCCTCTATGA
CCTGCTGCTGGAGgcggcgGACGCCCACCGCCTAC
ATGCGCCCACTAGCCGTGGAGGGGCATCCGTGGAG
GAGACGGACCAAAGCCACTTGGCCACTGCGGGCTC
TACTTCATCGCATTCCTTGCAAAAGTATTACATCA
CGGGGGAGGCAGAGGGTTTCCCTGCCACGGTC
Exemplary amino acid sequence of
NES-ZAP70dn(1-278)-ER(T12)
SEQ ID NO: 15
MNELALKLAGLDINKTMPDPAAHLPFFYGSISRAE
AEEHLKLAGMADGLFLLRQCLRSLGGYVLSLVHDV
RFHHFPIERQLNGTYAIAGGKAHCGPAELCEFYSR
DPDGLPCNLRKPCNRPSGLEPQPGVFDCLRDAMVR
DYVRQTWKLEGEALEQAIISQAPQVEKLIATTAHE
RMPWYHSSLTREEAERKLYSGAQTDGKFLLRPRKE
QGTYALSLIYGKTVYHYLISQDKAGKYCIPEGTKF
DTLWQLVEYLKLKADGLIYCLKEACPNSSASNASG
AAAPTLPAHPSTLTGSSAGDMRAANLWPSPLMIKR
SKKNSLALSLTADQMVSALLDAEPPILYSEYDPTR
PFSEASMMGLLTNLADRELVHMINWAKRVPGFVDL
TLHDQVHLLECAWLEILMIGLVWRSMEHPLKLLFA
PNLLLDRNQGKCVEGMVEIFDMLLATSSRFRMMNL
QGEEFVCLKSIILLNSGVYTFLSSTLKSLEEKDHI
HRVLDKITDTLIHLMAKAGLTLQQQHQRLAQLLLI
LSHIRHMSNKGMEHLYSMKCKNVVPLYDLLLEAAD
AHRLHAPTSRGGASVEETDQSHLATAGSTSSHSLQ
KYYITGEAEGFPATV
Exemplary nucleic acid sequence
encoding NES-ZAP70dn(1-278)-ER(T12)
SEQ ID NO: 16
ATGAATGAATTAGCCTTGAAATTAGCAGGTCTTGA
TATCAACAAGACAATGCCAGACCCCGCGGCGCACC
TGCCCTTCTTCTACGGCAGCATCTCGCGTGCCGAG
GCCGAGGAGCACCTGAAGCTGGCGGGCATGGCGGA
CGGGCTCTTCCTGCTGCGCCAGTGCCTGCGCTCGC
TGGGCGGCTATGTGCTGTCGCTCGTGCACGATGTG
CGCTTCCACCACTTTCCCATCGAGCGCCAGCTCAA
CGGCACCTACGCCATTGCCGGCGGCAAAGCGCACT
GTGGACCGGCAGAGCTCTGCGAGTTCTACTCGCGC
GACCCCGACGGGCTGCCCTGCAACCTGCGCAAGCC
GTGCAACCGGCCGTCGGGCCTCGAGCCGCAGCCGG
GGGTCTTCGACTGCCTGCGAGACGCCATGGTGCGT
GACTACGTGCGCCAGACGTGGAAGCTGGAGGGCGA
GGCCCTGGAGCAGGCCATCATCAGCCAGGCCCCGC
AGGTGGAGAAGCTCATTGCTACGACGGCCCACGAG
CGGATGCCCTGGTACCACAGCAGCCTGACGCGTGA
GGAGGCCGAGCGCAAACTTTACTCTGGGGCGCAGA
CCGACGGCAAGTTCCTGCTGAGGCCGCGGAAGGAG
CAGGGCACATACGCCCTGTCCCTCATCTATGGGAA
GACGGTGTACCACTACCTCATCAGCCAAGACAAGG
CGGGCAAGTACTGCATTCCCGAGGGCACCAAGTTT
GACACGCTCTGGCAGCTGGTGGAGTATCTGAAGCT
GAAGGCGGACGGGCTCATCTACTGCCTGAAGGAGG
CCTGCCCCAACAGCAGTGCCAGCAACGCCTCAGGG
GCTGCTGCTCCCACACTCCCAGCCCACCCATCCAC
GTTGACGGGATCCTCTGCTGGAGACATGAGAGCTG
CCAACCTTTGGCCAAGCCCGCTCATGATCAAACGC
TCTAAGAAGAACAGCCTGGCCTTGTCCCTGACGGC
CGACCAGATGGTCAGTGCCTTGTTGGATGCTGAGC
CCCCCATACTCTATTCCGAGTATGATCCTACCAGA
CCCTTCAGTGAAGCTTCGATGATGGGCTTACTGAC
CAACCTGGCAGACAGGGAGCTGGTTCACATGATCA
ACTGGGCGAAGAGGGTGCCAGGCTTTGTGGATTTG
ACCCTCCATGATCAGGTCCACCTTCTAGAATGTGC
CTGGCTAGAGATCCTGATGATTGGTCTCGTCTGGC
GCTCCATGGAGCACCCACTGAAGCTACTGTTTGCT
CCTAACTTGCTCTTGGACAGGAACCAGGGAAAATG
TGTAGAGGGCATGGTGGAGATCTTCGACATGCTGC
TGGCTACATCATCTCGGTTCCGCATGATGAATCTG
CAGGGAGAGGAGTTTGTGTGCCTCAAATCTATTAT
TTTGCTTAATTCTGGAGTGTACACATTTCTGTCCA
GCACCCTGAAGTCTCTGGAAGAGAAGGACCATATC
CACCGAGTCCTGGACAAGATCACAGACACTTTGAT
CCACCTGATGGCCAAGGCAGGCCTGACCCTGCAGC
AGCAGCACCAGCGGCTGGCCCAGCTCCTCCTCATC
CTCTCCCACATCAGGCACATGAGTAACAAAGGCAT
GGAGCATCTGTACAGCATGAAGTGCAAGAACGTGG
TGCCCCTCTATGACCTGCTGCTGGAGGCGGCGGAC
GCCCACCGCCTACATGCGCCCACTAGCCGTGGAGG
GGCATCCGTGGAGGAGACGGACCAAAGCCACTTGG
CCACTGCGGGCTCTACTTCATCGCATTCCTTGCAA
AAGTATTACATCACGGGGGAGGCAGAGGGTTTCCC
TGCCACGGTCTGA
Exemplary amino acid sequence of
LCK(1-266)
SEQ ID NO: 17
MGCGCSSHPEDDWMENIDVCENCHYPIVPLDGKGT
LLIRNGSEVRDPLVTYEGSNPPASPLQDNLVIALH
SYEPSHDGDLGFEKGEQLRILEQSGEWWKAQSLTT
GQEGFIPFNFVAKANSLEPEPWFFKNLSRKDAERQ
LLAPGNTHGSFLIRESESTAGSFSLSVRDFDQNQG
EVVKHYKIRNLDNGGFYISPRITFPGLHELVRHYT
NASDGLCTRLSRPCQTQKPQKPWWEDEWEVPRETL
KLVERLGAGQFGEVWMGYYNG
Exemplary amino acid sequence of
NES-LCK(1-266)-ER(T12)
SEQ ID NO: 18
MNELALKLAGLDINKTMGCGCSSHPEDDWMENIDV
CENCHYPIVPLDGKGTLLIRNGSEVRDPLVTYEGS
NPPASPLQDNLVIALHSYEPSHDGDLGFEKGEQLR
ILEQSGEWWKAQSLTTGQEGFIPFNFVAKANSLEP
EPWFFKNLSRKDAERQLLAPGNTHGSFLIRESEST
AGSFSLSVRDFDQNQGEVVKHYKIRNLDNGGFYIS
PRITFPGLHELVRHYTNASDGLCTRLSRPCQTQKP
QKPWWEDEWEVPRETLKLVERLGAGQFGEVWMGYY
NGGSSAGDMRAANLWPSPLMIKRSKKNSLALSLTA
DQMVSALLDAEPPILYSEYDPTRPFSEASMMGLLT
NLADRELVHMINWAKRVPGFVDLTLHDQVHLLECA
WLEILMIGLVWRSMEHPLKLLFAPNLLLDRNQGKC
VEGMVEIFDMLLATSSRFRMMNLQGEEFVCLKSII
LLNSGVYTFLSSTLKSLEEKDHIHRVLDKITDTLI
HLMAKAGLTLQQQHQRLAQLLLILSHIRHMSNKGM
EHLYSMKCKNVVPLYDLLLEAADAHRLHAPTSRGG
ASVEETDQSHLATAGSTSSHSLQKYYITGEAEGFP
ATV
Exemplary nucleic acid sequence
encoding LCK(1-266)
SEQ ID NO: 19
ATGGGCTGTGGCTGCAGCTCACACCCGGAAGATGA
CTGGATGGAAAACATCGATGTGTGTGAGAACTGCC
ATTATCCCATAGTCCCACTGGATGGCAAGGGCACG
CTGCTCATCCGAAATGGCTCTGAGGTGCGGGACCC
ACTGGTTACCTACGAAGGCTCCAATCCGCCGGCTT
CCCCACTGCAAGACAACCTGGTTATCGCTCTGCAC
AGCTATGAGCCCTCTCACGACGGAGATCTGGGCTT
TGAGAAGGGGGAACAGCTCCGCATCCTGGAGCAGA
GCGGCGAGTGGTGGAAGGCGCAGTCCCTGACCACG
GGCCAGGAAGGCTTCATCCCCTTCAATTTTGTGGC
CAAAGCGAACAGCCTGGAGCCCGAACCCTGGTTCT
TCAAGAACCTGAGCCGCAAGGACGCGGAGCGGCAG
CTCCTGGCGCCCGGGAACACTCACGGCTCCTTCCT
CATCCGGGAGAGCGAGAGCACCGCGGGATCGTTTT
CACTGTCGGTCCGGGACTTCGACCAGAACCAGGGA
GAGGTGGTGAAACATTACAAGATCCGTAATCTGGA
CAACGGTGGCTTCTACATCTCCCCTCGAATCACTT
TTCCCGGCCTGCATGAACTGGTCCGCCATTACACC
AATGCTTCAGATGGGCTGTGCACACGGTTGAGCCG
CCCCTGCCAGACCCAGAAGCCCCAGAAGCCGTGGT
GGGAGGACGAGTGGGAGGTTCCCAGGGAGACGCTG
AAGCTGGTGGAGCGGCTGGGGGCTGGACAGTTCGG
GGAGGTGTGGATGGGGTACTACAACGGG
Exemplary amino acid sequence encoding
polypeptide including a nuclear export
signal (NES) and a dominant negative
LCK1(1-266) moiety
SEQ ID NO: 20
MNELALKLAGLDINKTMGCGCSSHPEDDWMENIDV
CENCHYPIVPLDGKGTLLIRNGSEVRDPLVTYEGS
NPPASPLQDNLVIALHSYEPSHDGDLGFEKGEQLR
ILEQSGEWWKAQSLTTGQEGFIPFNFVAKANSLEP
EPWFFKNLSRKDAERQLLAPGNTHGSFLIRESEST
AGSFSLSVRDFDQNQGEVVKHYKIRNLDNGGFYIS
PRITFPGLHELVRHYTNASDGLCTRLSRPCQTQKP
QKPWWEDEWEVPRETLKLVERLGAGQFGEVWMGYY
NG
Exemplary nucleotide sequence encoding
polypeptide including a nuclear export
signal (NES) and a dominant negative
LCK1(1-266) moiety
SEQ ID NO: 21
ATGAATGAATTAGCCTTGAAATTAGCAGGTCTTGA
TATCAACAAGACAATGGGCTGTGGCTGCAGCTCAC
ACCCGGAAGATGACTGGATGGAAAACATCGATGTG
TGTGAGAACTGCCATTATCCCATAGTCCCACTGGA
TGGCAAGGGCACGCTGCTCATCCGAAATGGCTCTG
AGGTGCGGGACCCACTGGTTACCTACGAAGGCTCC
AATCCGCCGGCTTCCCCACTGCAAGACAACCTGGT
TATCGCTCTGCACAGCTATGAGCCCTCTCACGACG
GAGATCTGGGCTTTGAGAAGGGGGAACAGCTCCGC
ATCCTGGAGCAGAGCGGCGAGTGGTGGAAGGCGCA
GTCCCTGACCACGGGCCAGGAAGGCTTCATCCCCT
TCAATTTTGTGGCCAAAGCGAACAGCCTGGAGCCC
GAACCCTGGTTCTTCAAGAACCTGAGCCGCAAGGA
CGCGGAGCGGCAGCTCCTGGCGCCCGGGAACACTC
ACGGCTCCTTCCTCATCCGGGAGAGCGAGAGCACC
GCGGGATCGTTTTCACTGTCGGTCCGGGACTTCGA
CCAGAACCAGGGAGAGGTGGTGAAACATTACAAGA
TCCGTAATCTGGACAACGGTGGCTTCTACATCTCC
CCTCGAATCACTTTTCCCGGCCTGCATGAACTGGT
CCGCCATTACACCAATGCTTCAGATGGGCTGTGCA
CACGGTTGAGCCGCCCCTGCCAGACCCAGAAGCCC
CAGAAGCCGTGGTGGGAGGACGAGTGGGAGGTTCC
CAGGGAGACGCTGAAGCTGGTGGAGCGGCTGGGGG
CTGGACAGTTCGGGGAGGTGTGGATGGGGTACTAC
AACGGG
Exemplary nucleic acid sequence
encoding NES-LCK(1-266)-ER(T12)
SEQ ID NO: 22
ATGAATGAATTAGCCTTGAAATTAGCAGGTCTTGA
TATCAACAAGACAATGGGCTGTGGCTGCAGCTCAC
ACCCGGAAGATGACTGGATGGAAAACATCGATGTG
TGTGAGAACTGCCATTATCCCATAGTCCCACTGGA
TGGCAAGGGCACGCTGCTCATCCGAAATGGCTCTG
AGGTGCGGGACCCACTGGTTACCTACGAAGGCTCC
AATCCGCCGGCTTCCCCACTGCAAGACAACCTGGT
TATCGCTCTGCACAGCTATGAGCCCTCTCACGACG
GAGATCTGGGCTTTGAGAAGGGGGAACAGCTCCGC
ATCCTGGAGCAGAGCGGCGAGTGGTGGAAGGCGCA
GTCCCTGACCACGGGCCAGGAAGGCTTCATCCCCT
TCAATTTTGTGGCCAAAGCGAACAGCCTGGAGCCC
GAACCCTGGTTCTTCAAGAACCTGAGCCGCAAGGA
CGCGGAGCGGCAGCTCCTGGCGCCCGGGAACACTC
ACGGCTCCTTCCTCATCCGGGAGAGCGAGAGCACC
GCGGGATCGTTTTCACTGTCGGTCCGGGACTTCGA
CCAGAACCAGGGAGAGGTGGTGAAACATTACAAGA
TCCGTAATCTGGACAACGGTGGCTTCTACATCTCC
CCTCGAATCACTTTTCCCGGCCTGCATGAACTGGT
CCGCCATTACACCAATGCTTCAGATGGGCTGTGCA
CACGGTTGAGCCGCCCCTGCCAGACCCAGAAGCCC
CAGAAGCCGTGGTGGGAGGACGAGTGGGAGGTTCC
CAGGGAGACGCTGAAGCTGGTGGAGCGGCTGGGGG
CTGGACAGTTCGGGGAGGTGTGGATGGGGTACTAC
AACGGGGGATCCTCTGCTGGAGACATGAGAGCTGC
CAACCTTTGGCCAAGCCCGCTCATGATCAAACGCT
CTAAGAAGAACAGCCTGGCCTTGTCCCTGACGGCC
GACCAGATGGTCAGTGCCTTGTTGGATGCTGAGCC
CCCCATACTCTATTCCGAGTATGATCCTACCAGAC
CCTTCAGTGAAGCTTCGATGATGGGCTTACTGACC
AACCTGGCAGACAGGGAGCTGGTTCACATGATCAA
CTGGGCGAAGAGGGTGCCAGGCTTTGTGGATTTGA
CCCTCCATGATCAGGTCCACCTTCTAGAATGTGCC
TGGCTAGAGATCCTGATGATTGGTCTCGTCTGGCG
CTCCATGGAGCACCCACTGAAGCTACTGTTTGCTC
CTAACTTGCTCTTGGACAGGAACCAGGGAAAATGT
GTAGAGGGCATGGTGGAGATCTTCGACATGCTGCT
GGCTACATCATCTCGGTTCCGCATGATGAATCTGC
AGGGAGAGGAGTTTGTGTGCCTCAAATCTATTATT
TTGCTTAATTCTGGAGTGTACACATTTCTGTCCAG
CACCCTGAAGTCTCTGGAAGAGAAGGACCATATCC
ACCGAGTCCTGGACAAGATCACAGACACTTTGATC
CACCTGATGGCCAAGGCAGGCCTGACCCTGCAGCA
GCAGCACCAGCGGCTGGCCCAGCTCCTCCTCATCC
TCTCCCACATCAGGCACATGAGTAACAAAGGCATG
GAGCATCTGTACAGCATGAAGTGCAAGAACGTGGT
GCCCCTCTATGACCTGCTGCTGGAGGCGGCGGACG
CCCACCGCCTACATGCGCCCACTAGCCGTGGAGGG
GCATCCGTGGAGGAGACGGACCAAAGCCACTTGGC
CACTGCGGGCTCTACTTCATCGCATTCCTTGCAAA
AGTATTACATCACGGGGGAGGCAGAGGGTTTCCCT
GCCACGGTCTGA
Exemplary amino acid sequence of
SHP1(210-595)
SEQ ID NO: 23
RQPYYATRVNAADIENRVLELNKKQESEDTAKAGF
WEEFESLQKQEVKNLHQRLEGQRPENKGKNRYKNI
LPFDHSRVILQGRDSNIPGSDYINANYIKNQLLGP
DENAKTYIASQGCLEATVNDFWQMAWQENSRVIVM
TTREVEKGRNKCVPYWPEVGMQRAYGPYSVTNCGE
HDTTEYKLRTLQVSPLDNGDLIREIWHYQYLSWPD
HGVPSEPGGVLSFLDQINQRQESLPHAGPIIVHCS
AGIGRTGTIIVIDMLMENISTKGLDCDIDIQKTIQ
MVRAQRSGMVQTEAQYKFIYVAIAQFIETTKKKLE
VLQSQKGQESEYGNITYPPAMKNAHAKASRTSSKH
KEDVYENLHTKNKREEKVKKQRSADKEKSKGSLKR
K
Exemplary amino acid sequence of
NES-SHP1(210-595)-ER(T12)
SEQ ID NO: 24
MNELALKLAGLDINKTRQPYYATRVNAADIENRVL
ELNKKQESEDTAKAGFWEEFESLQKQEVKNLHQRL
EGQRPENKGKNRYKNILPFDHSRVILQGRDSNIPG
SDYINANYIKNQLLGPDENAKTYIASQGCLEATVN
DFWQMAWQENSRVIVMTTREVEKGRNKCVPYWPEV
GMQRAYGPYSVTNCGEHDTTEYKLRTLQVSPLDNG
DLIREIWHYQYLSWPDHGVPSEPGGVLSFLDQINQ
RQESLPHAGPIIVHCSAGIGRTGTIIVIDMLMENI
STKGLDCDIDIQKTIQMVRAQRSGMVQTEAQYKFI
YVAIAQFIETTKKKLEVLQSQKGQESEYGNITYPP
AMKNAHAKASRTSSKHKEDVYENLHTKNKREEKVK
KQRSADKEKSKGSLKRKGSSAGDMRAANLWPSPLM
IKRSKKNSLALSLTADQMVSALLDAEPPILYSEYD
PTRPFSEASMMGLLTNLADRELVHMINWAKRVPGF
VDLTLHDQVHLLECAWLEILMIGLVWRSMEHPLKL
LFAPNLLLDRNQGKCVEGMVEIFDMLLATSSRFRM
MNLQGEEFVCLKSIILLNSGVYTFLSSTLKSLEEK
DHIHRVLDKITDTLIHLMAKAGLTLQQQHQRLAQL
LLILSHIRHMSNKGMEHLYSMKCKNVVPLYDLLLE
AADAHRLHAPTSRGGASVEETDQSHLATAGSTSSH
SLQKYYITGEAEGFPATV
Exemplary nucleic acid sequence
encoding SHP1(210-595)
SEQ ID NO: 25
CGGCAGCCGTACTATGCCACGAGGGTGAATGCGGC
TGACATTGAGAACCGAGTGTTGGAACTGAACAAGA
AGCAGGAGTCCGAGGATACAGCCAAGGCTGGCTTC
TGGGAGGAGTTTGAGAGTTTGCAGAAGCAGGAGGT
GAAGAACTTGCACCAGCGTCTGGAAGGGCAGCGGC
CAGAGAACAAGGGCAAGAACCGCTACAAGAACATT
CTCCCCTTTGACCACAGCCGAGTGATCCTGCAGGG
ACGGGACAGTAACATCCCCGGGTCCGACTACATCA
ATGCCAACTACATCAAGAACCAGCTGCTAGGCCCT
GATGAGAACGCTAAGACCTACATCGCCAGCCAGGG
CTGTCTGGAGGCCACGGTCAATGACTTCTGGCAGA
TGGCGTGGCAGGAGAACAGCCGTGTCATCGTCATG
ACCACCCGAGAGGTGGAGAAAGGCCGGAACAAATG
CGTCCCATACTGGCCCGAGGTGGGCATGCAGCGTG
CTTATGGGCCCTACTCTGTGACCAACTGCGGGGAG
CATGACACAACCGAATACAAACTCCGTACCTTACA
GGTCTCCCCGCTGGACAATGGAGACCTGATTCGGG
AGATCTGGCATTACCAGTACCTGAGCTGGCCCGAC
CATGGGGTCCCCAGTGAGCCTGGGGGTGTCCTCAG
CTTCCTGGACCAGATCAACCAGCGGCAGGAAAGTC
TGCCTCACGCAGGGCCCATCATCGTGCACTGCAGC
GCCGGCATCGGCCGCACAGGCACCATCATTGTCAT
CGACATGCTCATGGAGAACATCTCCACCAAGGGCC
TGGACTGTGACATTGACATCCAGAAGACCATCCAG
ATGGTGCGGGCGCAGCGCTCGGGCATGGTGCAGAC
GGAGGCGCAGTACAAGTTCATCTACGTGGCCATCG
CCCAGTTCATTGAAACCACTAAGAAGAAGCTGGAG
GTCCTGCAGTCGCAGAAGGGCCAGGAGTCGGAGTA
CGGGAACATCACCTATCCCCCAGCCATGAAGAATG
CCCATGCCAAGGCCTCCCGCACCTCGTCCAAACAC
AAGGAGGATGTGTATGAGAACCTGCACACTAAGAA
CAAGAGGGAGGAGAAAGTGAAGAAGCAGCGGTCAG
CAGACAAGGAGAAGAGCAAGGGTTCCCTCAAGAGG
AAG
Exemplary nucleic acid sequence encoding
NES-SHP1(210-595)-ER(T12)
SEQ ID NO: 26
ATGAATGAATTAGCCTTGAAATTAGCAGGTCTTGA
TATCAACAAGACACGGCAGCCGTACTATGCCACGA
GGGTGAATGCGGCTGACATTGAGAACCGAGTGTTG
GAACTGAACAAGAAGCAGGAGTCCGAGGATACAGC
CAAGGCTGGCTTCTGGGAGGAGTTTGAGAGTTTGC
AGAAGCAGGAGGTGAAGAACTTGCACCAGCGTCTG
GAAGGGCAGCGGCCAGAGAACAAGGGCAAGAACCG
CTACAAGAACATTCTCCCCTTTGACCACAGCCGAG
TGATCCTGCAGGGACGGGACAGTAACATCCCCGGG
TCCGACTACATCAATGCCAACTACATCAAGAACCA
GCTGCTAGGCCCTGATGAGAACGCTAAGACCTACA
TCGCCAGCCAGGGCTGTCTGGAGGCCACGGTCAAT
GACTTCTGGCAGATGGCGTGGCAGGAGAACAGCCG
TGTCATCGTCATGACCACCCGAGAGGTGGAGAAAG
GCCGGAACAAATGCGTCCCATACTGGCCCGAGGTG
GGCATGCAGCGTGCTTATGGGCCCTACTCTGTGAC
CAACTGCGGGGAGCATGACACAACCGAATACAAAC
TCCGTACCTTACAGGTCTCCCCGCTGGACAATGGA
GACCTGATTCGGGAGATCTGGCATTACCAGTACCT
GAGCTGGCCCGACCATGGGGTCCCCAGTGAGCCTG
GGGGTGTCCTCAGCTTCCTGGACCAGATCAACCAG
CGGCAGGAAAGTCTGCCTCACGCAGGGCCCATCAT
CGTGCACTGCAGCGCCGGCATCGGCCGCACAGGCA
CCATCATTGTCATCGACATGCTCATGGAGAACATC
TCCACCAAGGGCCTGGACTGTGACATTGACATCCA
GAAGACCATCCAGATGGTGCGGGCGCAGCGCTCGG
GCATGGTGCAGACGGAGGCGCAGTACAAGTTCATC
TACGTGGCCATCGCCCAGTTCATTGAAACCACTAA
GAAGAAGCTGGAGGTCCTGCAGTCGCAGAAGGGCC
AGGAGTCGGAGTACGGGAACATCACCTATCCCCCA
GCCATGAAGAATGCCCATGCCAAGGCCTCCCGCAC
CTCGTCCAAACACAAGGAGGATGTGTATGAGAACC
TGCACACTAAGAACAAGAGGGAGGAGAAAGTGAAG
AAGCAGCGGTCAGCAGACAAGGAGAAGAGCAAGGG
TTCCCTCAAGAGGAAGGGATCCTCTGCTGGAGACA
TGAGAGCTGCCAACCTTTGGCCAAGCCCGCTCATG
ATCAAACGCTCTAAGAAGAACAGCCTGGCCTTGTC
CCTGACGGCCGACCAGATGGTCAGTGCCTTGTTGG
ATGCTGAGCCCCCCATACTCTATTCCGAGTATGAT
CCTACCAGACCCTTCAGTGAAGCTTCGATGATGGG
CTTACTGACCAACCTGGCAGACAGGGAGCTGGTTC
ACATGATCAACTGGGCGAAGAGGGTGCCAGGCTTT
GTGGATTTGACCCTCCATGATCAGGTCCACCTTCT
AGAATGTGCCTGGCTAGAGATCCTGATGATTGGTC
TCGTCTGGCGCTCCATGGAGCACCCACTGAAGCTA
CTGTTTGCTCCTAACTTGCTCTTGGACAGGAACCA
GGGAAAATGTGTAGAGGGCATGGTGGAGATCTTCG
ACATGCTGCTGGCTACATCATCTCGGTTCCGCATG
ATGAATCTGCAGGGAGAGGAGTTTGTGTGCCTCAA
ATCTATTATTTTGCTTAATTCTGGAGTGTACACAT
TTCTGTCCAGCACCCTGAAGTCTCTGGAAGAGAAG
GACCATATCCACCGAGTCCTGGACAAGATCACAGA
CACTTTGATCCACCTGATGGCCAAGGCAGGCCTGA
CCCTGCAGCAGCAGCACCAGCGGCTGGCCCAGCTC
CTCCTCATCCTCTCCCACATCAGGCACATGAGTAA
CAAAGGCATGGAGCATCTGTACAGCATGAAGTGCA
AGAACGTGGTGCCCCTCTATGACCTGCTGCTGGAG
GCGGCGGACGCCCACCGCCTACATGCGCCCACTAG
CCGTGGAGGGGCATCCGTGGAGGAGACGGACCAAA
GCCACTTGGCCACTGCGGGCTCTACTTCATCGCAT
TCCTTGCAAAAGTATTACATCACGGGGGAGGCAGA
GGGTTTCCCTGCCACGGTCTGA
Exemplary amino acid sequence encoding
polypeptide including a nuclear export
signal (NES) and a dominant negative
SHP1(210-595) moiety
SEQ ID NO: 27
MNELALKLAGLDINKTRQPYYATRVNAADIENRVL
ELNKKQESEDTAKAGFWEEFESLQKQEVKNLHQRL
EGQRPENKGKNRYKNILPFDHSRVILQGRDSNIPG
SDYINANYIKNQLLGPDENAKTYIASQGCLEATVN
DFWQMAWQENSRVIVMTTREVEKGRNKCVPYWPEV
GMQRAYGPYSVTNCGEHDTTEYKLRTLQVSPLDNG
DLIREIWHYQYLSWPDHGVPSEPGGVLSFLDQINQ
RQESLPHAGPIIVHCSAGIGRTGTIIVIDMLMENI
STKGLDCDIDIQKTIQMVRAQRSGMVQTEAQYKFI
YVAIAQFIETTKKKLEVLQSQKGQESEYGNITYPP
AMKNAHAKASRTSSKHKEDVYENLHTKNKREEKVK
KQRSADKEKSKGSLKRK
Exemplary nucleotide sequence encoding
polypeptide including a nuclear export
signal (NES) and a dominant negative
LCK1(1-266) moiety
SEQ ID NO: 28
ATGAATGAATTAGCCTTGAAATTAGCAGGTCTTGA
TATCAACAAGACACGGCAGCCGTACTATGCCACGA
GGGTGAATGCGGCTGACATTGAGAACCGAGTGTTG
GAACTGAACAAGAAGCAGGAGTCCGAGGATACAGC
CAAGGCTGGCTTCTGGGAGGAGTTTGAGAGTTTGC
AGAAGCAGGAGGTGAAGAACTTGCACCAGCGTCTG
GAAGGGCAGCGGCCAGAGAACAAGGGCAAGAACCG
CTACAAGAACATTCTCCCCTTTGACCACAGCCGAG
TGATCCTGCAGGGACGGGACAGTAACATCCCCGGG
TCCGACTACATCAATGCCAACTACATCAAGAACCA
GCTGCTAGGCCCTGATGAGAACGCTAAGACCTACA
TCGCCAGCCAGGGCTGTCTGGAGGCCACGGTCAAT
GACTTCTGGCAGATGGCGTGGCAGGAGAACAGCCG
TGTCATCGTCATGACCACCCGAGAGGTGGAGAAAG
GCCGGAACAAATGCGTCCCATACTGGCCCGAGGTG
GGCATGCAGCGTGCTTATGGGCCCTACTCTGTGAC
CAACTGCGGGGAGCATGACACAACCGAATACAAAC
TCCGTACCTTACAGGTCTCCCCGCTGGACAATGGA
GACCTGATTCGGGAGATCTGGCATTACCAGTACCT
GAGCTGGCCCGACCATGGGGTCCCCAGTGAGCCTG
GGGGTGTCCTCAGCTTCCTGGACCAGATCAACCAG
CGGCAGGAAAGTCTGCCTCACGCAGGGCCCATCAT
CGTGCACTGCAGCGCCGGCATCGGCCGCACAGGCA
CCATCATTGTCATCGACATGCTCATGGAGAACATC
TCCACCAAGGGCCTGGACTGTGACATTGACATCCA
GAAGACCATCCAGATGGTGCGGGCGCAGCGCTCGG
GCATGGTGCAGACGGAGGCGCAGTACAAGTTCATC
TACGTGGCCATCGCCCAGTTCATTGAAACCACTAA
GAAGAAGCTGGAGGTCCTGCAGTCGCAGAAGGGCC
AGGAGTCGGAGTACGGGAACATCACCTATCCCCCA
GCCATGAAGAATGCCCATGCCAAGGCCTCCCGCAC
CTCGTCCAAACACAAGGAGGATGTGTATGAGAACC
TGCACACTAAGAACAAGAGGGAGGAGAAAGTGAAG
AAGCAGCGGTCAGCAGACAAGGAGAAGAGCAAGGG
TTCCCTCAAGAGGAAG
Exemplary nucleotide sequence encoding
an endoxifen-responsive dominant negative
Zap-70 polypeptide (ZAP70dn-ER(T2)
SEQ ID NO: 29
ATGCCAGACCCCGCGGCGCACCTGCCCTTCTTCTA
CGGCAGCATCTCGCGTGCCGAGGCCGAGGAGCACC
TGAAGCTGGCGGGCATGGCGGACGGGCTCTTCCTG
CTGCGCCAGTGCCTGCGCTCGCTGGGCGGCTATGT
GCTGTCGCTCGTGCACGATGTGCGCTTCCACCACT
TTCCCATCGAGCGCCAGCTCAACGGCACCTACGCC
ATTGCCGGCGGCAAAGCGCACTGTGGACCGGCAGA
GCTCTGCGAGTTCTACTCGCGCGACCCCGACGGGC
TGCCCTGCAACCTGCGCAAGCCGTGCAACCGGCCG
TCGGGCCTCGAGCCGCAGCCGGGGGTCTTCGACTG
CCTGCGAGACGCCATGGTGCGTGACTACGTGCGCC
AGACGTGGAAGCTGGAGGGCGAGGCCCTGGAGCAG
GCCATCATCAGCCAGGCCCCGCAGGTGGAGAAGCT
CATTGCTACGACGGCCCACGAGCGGATGCCCTGGT
ACCACAGCAGCCTGACGCGTGAGGAGGCCGAGCGC
AAACTTTACTCTGGGGCGCAGACCGACGGCAAGTT
CCTGCTGAGGCCGCGGAAGGAGCAGGGCACATACG
CCCTGTCCCTCATCTATGGGAAGACGGTGTACCAC
TACCTCATCAGCCAAGACAAGGCGGGCAAGTACTG
CATTCCCGAGGGCACCAAGTTTGACACGCTCTGGC
AGCTGGTGGAGTATCTGAAGCTGAAGGCGGACGGG
CTCATCTACTGCCTGAAGGAGGCCTGCCCCAACAG
CAGTGCCAGCAACGCCTCAGGGGCTGCTGCTCCCA
CACTCCCAGCCCACCCATCCACGTTGACGGGATCC
TCTGCTGGAGACATGAGAGCTGCCAACCTTTGGCC
AAGCCCGCTCATGATCAAACGCTCTAAGAAGAACA
GCCTGGCCTTGTCCCTGACGGCCGACCAGATGGTC
AGTGCCTTGTTGGATGCTGAGCCCCCCATACTCTA
TTCCGAGTATGATCCTACCAGACCCTTCAGTGAAG
CTTCGATGATGGGCTTACTGACCAACCTGGCAGAC
AGGGAGCTGGTTCACATGATCAACTGGGCGAAGAG
GGTGCCAGGCTTTGTGGATTTGACCCTCCATGATC
AGGTCCACCTTCTAGAATGTGCCTGGCTAGAGATC
CTGATGATTGGTCTCGTCTGGCGCTCCATGGAGCA
CCCAGTGAAGCTACTGTTTGCTCCTAACTTGCTCT
TGGACAGGAACCAGGGAAAATGTGTAGAGGGCATG
GTGGAGATCTTCGACATGCTGCTGGCTACATCATC
TCGGTTCCGCATGATGAATCTGCAGGGAGAGGAGT
TTGTGTGCCTCAAATCTATTATTTTGCTTAATTCT
GGAGTGTACACATTTCTGTCCAGCACCCTGAAGTC
TCTGGAAGAGAAGGACCATATCCACCGAGTCCTGG
ACAAGATCACAGACACTTTGATCCACCTGATGGCC
AAGGCAGGCCTGACCCTGCAGCAGCAGCACCAGCG
GCTGGCCCAGCTCCTCCTCATCCTCTCCCACATCA
GGCACATGAGTAACAAAGGCATGGAGCATCTGTAC
AGCATGAAGTGCAAGAACGTGGTGCCCCTCTATGA
CCTGCTGCTGGAGGCGGCGGACGCCCACCGCCTAC
ATGCGCCCACTAGCCGTGGAGGGGCATCCGTGGAG
GAGACGGACCAAAGCCACTTGGCCACTGCGGGCTC
TACTTCATCGCATTCCTTGCAAAAGTATTACATCA
CGGGGGAGGCAGAGGGTTTCCCTGCCACGGTC
Exemplary amino acid sequence of an
endoxifen-responsive dominant negative
Zap-70 polypeptide (ZAP70dn-ER(T2)
SEQ ID NO: 30
MPDPAAHLPFFYGSISRAEAEEHLKLAGMADGLFL
LRQCLRSLGGYVLSLVHDVRFHHFPIERQLNGTYA
IAGGKAHCGPAELCEFYSRDPDGLPCNLRKPCNRP
SGLEPQPGVFDCLRDAMVRDYVRQTWKLEGEALEQ
AIISQAPQVEKLIATTAHERMPWYHSSLTREEAER
KLYSGAQTDGKFLLRPRKEQGTYALSLIYGKTVYH
YLISQDKAGKYCIPEGTKFDTLWQLVEYLKLKADG
LIYCLKEACPNSSASNASGAAAPTLPAHPSTLTGS
SAGDMRAANLWPSPLMIKRSKKNSLALSLTADQMV
SALLDAEPPILYSEYDPTRPFSEASMMGLLTNLAD
RELVHMINWAKRVPGFVDLTLHDQVHLLECAWLEI
LMIGLVWRSMEHPVKLLFAPNLLLDRNQGKCVEGM
VEIFDMLLATSSRFRMMNLQGEEFVCLKSIILLNS
GVYTFLSSTLKSLEEKDHIHRVLDKITDTLIHLMA
KAGLTLQQQHQRLAQLLLILSHIRHMSNKGMEHLY
SMKCKNVVPLYDLLLEAADAHRLHAPTSRGGASVE
ETDQSHLATAGSTSSHSLQKYYITGEAEGFPATV
Exemplary amino acid sequence
of ZAP70dn(1-278)-ER(T12)
SEQ ID NO: 31
MPDPAAHLPFFYGSISRAEAEEHLKLAGMADGLFL
LRQCLRSLGGYVLSLVHDVRFHHFPIERQLNGTYA
IAGGKAHCGPAELCEFYSRDPDGLPCNLRKPCNRP
SGLEPQPGVFDCLRDAMVRDYVRQTWKLEGEALEQ
AIISQAPQVEKLIATTAHERMPWYHSSLTREEAER
KLYSGAQTDGKFLLRPRKEQGTYALSLIYGKTVYH
YLISQDKAGKYCIPEGTKFDTLWQLVEYLKLKADG
LIYCLKEACPNSSASNASGAAAPTLPAHPSTLTGS
SAGDMRAANLWPSPLMIKRSKKNSLALSLTADQMV
SALLDAEPPILYSEYDPTRPFSEASMMGLLTNLAD
RELVHMINWAKRVPGFVDLTLHDQVHLLECAWLEI
LMIGLVWRSMEHPLKLLFAPNLLLDRNQGKCVEGM
VEIFDMLLATSSRFRMMNLQGEEFVCLKSIILLNS
GVYTFLSSTLKSLEEKDHIHRVLDKITDTLIHLMA
KAGLTLQQQHQRLAQLLLILSHIRHMSNKGMEHLY
SMKCKNVVPLYDLLLEAADAHRLHAPTSRGGASVE
ETDQSHLATAGSTSSHSLQKYYITGEAEGFPATV
Exemplary nucleic acid sequence
encoding ZAP70dn(1-278)-ER(T12)
SEQ ID NO: 32
ATGCCAGACCCCGCGGCGCACCTGCCCTTCTTCTA
CGGCAGCATCTCGCGTGCCGAGGCCGAGGAGCACC
TGAAGCTGGCGGGCATGGCGGACGGGCTCTTCCTG
CTGCGCCAGTGCCTGCGCTCGCTGGGCGGCTATGT
GCTGTCGCTCGTGCACGATGTGCGCTTCCACCACT
TTCCCATCGAGCGCCAGCTCAACGGCACCTACGCC
ATTGCCGGCGGCAAAGCGCACTGTGGACCGGCAGA
GCTCTGCGAGTTCTACTCGCGCGACCCCGACGGGC
TGCCCTGCAACCTGCGCAAGCCGTGCAACCGGCCG
TCGGGCCTCGAGCCGCAGCCGGGGGTCTTCGACTG
CCTGCGAGACGCCATGGTGCGTGACTACGTGCGCC
AGACGTGGAAGCTGGAGGGCGAGGCCCTGGAGCAG
GCCATCATCAGCCAGGCCCCGCAGGTGGAGAAGCT
CATTGCTACGACGGCCCACGAGCGGATGCCCTGGT
ACCACAGCAGCCTGACGCGTGAGGAGGCCGAGCGC
AAACTTTACTCTGGGGCGCAGACCGACGGCAAGTT
CCTGCTGAGGCCGCGGAAGGAGCAGGGCACATACG
CCCTGTCCCTCATCTATGGGAAGACGGTGTACCAC
TACCTCATCAGCCAAGACAAGGCGGGCAAGTACTG
CATTCCCGAGGGCACCAAGTTTGACACGCTCTGGC
AGCTGGTGGAGTATCTGAAGCTGAAGGCGGACGGG
CTCATCTACTGCCTGAAGGAGGCCTGCCCCAACAG
CAGTGCCAGCAACGCCTCAGGGGCTGCTGCTCCCA
CACTCCCAGCCCACCCATCCACGTTGACGGGATCC
TCTGCTGGAGACATGAGAGCTGCCAACCTTTGGCC
AAGCCCGCTCATGATCAAACGCTCTAAGAAGAACA
GCCTGGCCTTGTCCCTGACGGCCGACCAGATGGTC
AGTGCCTTGTTGGATGCTGAGCCCCCCATACTCTA
TTCCGAGTATGATCCTACCAGACCCTTCAGTGAAG
CTTCGATGATGGGCTTACTGACCAACCTGGCAGAC
AGGGAGCTGGTTCACATGATCAACTGGGCGAAGAG
GGTGCCAGGCTTTGTGGATTTGACCCTCCATGATC
AGGTCCACCTTCTAGAATGTGCCTGGCTAGAGATC
CTGATGATTGGTCTCGTCTGGCGCTCCATGGAGCA
CCCACTGAAGCTACTGTTTGCTCCTAACTTGCTCT
TGGACAGGAACCAGGGAAAATGTGTAGAGGGCATG
GTGGAGATCTTCGACATGCTGCTGGCTACATCATC
TCGGTTCCGCATGATGAATCTGCAGGGAGAGGAGT
TTGTGTGCCTCAAATCTATTATTTTGCTTAATTCT
GGAGTGTACACATTTCTGTCCAGCACCCTGAAGTC
TCTGGAAGAGAAGGACCATATCCACCGAGTCCTGG
ACAAGATCACAGACACTTTGATCCACCTGATGGCC
AAGGCAGGCCTGACCCTGCAGCAGCAGCACCAGCG
GCTGGCCCAGCTCCTCCTCATCCTCTCCCACATCA
GGCACATGAGTAACAAAGGCATGGAGCATCTGTAC
AGCATGAAGTGCAAGAACGTGGTGCCCCTCTATGA
CCTGCTGCTGGAGGCGGCGGACGCCCACCGCCTAC
ATGCGCCCACTAGCCGTGGAGGGGCATCCGTGGAG
GAGACGGACCAAAGCCACTTGGCCACTGCGGGCTC
TACTTCATCGCATTCCTTGCAAAAGTATTACATCA
CGGGGGAGGCAGAGGGTTTCCCTGCCACGGTCTGA
Exemplary amino acid sequence of
LCK(1-266)-ER(T12)
SEQ ID NO: 33
MGCGCSSHPEDDWMENIDVCENCHYPIVPLDGKGT
LLIRNGSEVRDPLVTYEGSNPPASPLQDNLVIALH
SYEPSHDGDLGFEKGEQLRILEQSGEWWKAQSLTT
GQEGFIPFNFVAKANSLEPEPWFFKNLSRKDAERQ
LLAPGNTHGSFLIRESESTAGSFSLSVRDFDQNQG
EVVKHYKIRNLDNGGFYISPRITFPGLHELVRHYT
NASDGLCTRLSRPCQTQKPQKPWWEDEWEVPRETL
KLVERLGAGQFGEVWMGYYNGGSSAGDMRAANLWP
SPLMIKRSKKNSLALSLTADQMVSALLDAEPPILY
SEYDPTRPFSEASMMGLLTNLADRELVHMINWAKR
VPGFVDLTLHDQVHLLECAWLEILMIGLVWRSMEH
PLKLLFAPNLLLDRNQGKCVEGMVEIFDMLLATSS
RFRMMNLQGEEFVCLKSIILLNSGVYTFLSSTLKS
LEEKDHIHRVLDKITDTLIHLMAKAGLTLQQQHQR
LAQLLLILSHIRHMSNKGMEHLYSMKCKNVVPLYD
LLLEAADAHRLHAPTSRGGASVEETDQSHLATAGS
TSSHSLQKYYITGEAEGFPATV
Exemplary nucleic acid sequence
encoding LCK(1-266)-ER(T12)
SEQ ID NO: 34
ATGGGCTGTGGCTGCAGCTCACACCCGGAAGATGA
CTGGATGGAAAACATCGATGTGTGTGAGAACTGCC
ATTATCCCATAGTCCCACTGGATGGCAAGGGCACG
CTGCTCATCCGAAATGGCTCTGAGGTGCGGGACCC
ACTGGTTACCTACGAAGGCTCCAATCCGCCGGCTT
CCCCACTGCAAGACAACCTGGTTATCGCTCTGCAC
AGCTATGAGCCCTCTCACGACGGAGATCTGGGCTT
TGAGAAGGGGGAACAGCTCCGCATCCTGGAGCAGA
GCGGCGAGTGGTGGAAGGCGCAGTCCCTGACCACG
GGCCAGGAAGGCTTCATCCCCTTCAATTTTGTGGC
CAAAGCGAACAGCCTGGAGCCCGAACCCTGGTTCT
TCAAGAACCTGAGCCGCAAGGACGCGGAGCGGCAG
CTCCTGGCGCCCGGGAACACTCACGGCTCCTTCCT
CATCCGGGAGAGCGAGAGCACCGCGGGATCGTTTT
CACTGTCGGTCCGGGACTTCGACCAGAACCAGGGA
GAGGTGGTGAAACATTACAAGATCCGTAATCTGGA
CAACGGTGGCTTCTACATCTCCCCTCGAATCACTT
TTCCCGGCCTGCATGAACTGGTCCGCCATTACACC
AATGCTTCAGATGGGCTGTGCACACGGTTGAGCCG
CCCCTGCCAGACCCAGAAGCCCCAGAAGCCGTGGT
GGGAGGACGAGTGGGAGGTTCCCAGGGAGACGCTG
AAGCTGGTGGAGCGGCTGGGGGCTGGACAGTTCGG
GGAGGTGTGGATGGGGTACTACAACGGGGGATCCT
CTGCTGGAGACATGAGAGCTGCCAACCTTTGGCCA
AGCCCGCTCATGATCAAACGCTCTAAGAAGAACAG
CCTGGCCTTGTCCCTGACGGCCGACCAGATGGTCA
GTGCCTTGTTGGATGCTGAGCCCCCCATACTCTAT
TCCGAGTATGATCCTACCAGACCCTTCAGTGAAGC
TTCGATGATGGGCTTACTGACCAACCTGGCAGACA
GGGAGCTGGTTCACATGATCAACTGGGCGAAGAGG
GTGCCAGGCTTTGTGGATTTGACCCTCCATGATCA
GGTCCACCTTCTAGAATGTGCCTGGCTAGAGATCC
TGATGATTGGTCTCGTCTGGCGCTCCATGGAGCAC
CCACTGAAGCTACTGTTTGCTCCTAACTTGCTCTT
GGACAGGAACCAGGGAAAATGTGTAGAGGGCATGG
TGGAGATCTTCGACATGCTGCTGGCTACATCATCT
CGGTTCCGCATGATGAATCTGCAGGGAGAGGAGTT
TGTGTGCCTCAAATCTATTATTTTGCTTAATTCTG
GAGTGTACACATTTCTGTCCAGCACCCTGAAGTCT
CTGGAAGAGAAGGACCATATCCACCGAGTCCTGGA
CAAGATCACAGACACTTTGATCCACCTGATGGCCA
AGGCAGGCCTGACCCTGCAGCAGCAGCACCAGCGG
CTGGCCCAGCTCCTCCTCATCCTCTCCCACATCAG
GCACATGAGTAACAAAGGCATGGAGCATCTGTACA
GCATGAAGTGCAAGAACGTGGTGCCCCTCTATGAC
CTGCTGCTGGAGGCGGCGGACGCCCACCGCCTACA
TGCGCCCACTAGCCGTGGAGGGGCATCCGTGGAGG
AGACGGACCAAAGCCACTTGGCCACTGCGGGCTCT
ACTTCATCGCATTCCTTGCAAAAGTATTACATCAC
GGGGGAGGCAGAGGGTTTCCCTGCCACGGTCTGA
Exemplary amino acid sequence of
SHP1(210-595)-ER(T12)
SEQ ID NO: 35
RQPYYATRVNAADIENRVLELNKKQESEDTAKAGF
WEEFESLQKQEVKNLHQRLEGQRPENKGKNRYKNI
LPFDHSRVILQGRDSNIPGSDYINANYIKNQLLGP
DENAKTYIASQGCLEATVNDFWQMAWQENSRVIVM
TTREVEKGRNKCVPYWPEVGMQRAYGPYSVTNCGE
HDTTEYKLRTLQVSPLDNGDLIREIWHYQYLSWPD
HGVPSEPGGVLSFLDQINQRQESLPHAGPIIVHCS
AGIGRTGTIIVIDMLMENISTKGLDCDIDIQKTIQ
MVRAQRSGMVQTEAQYKFIYVAIAQFIETTKKKLE
VLQSQKGQESEYGNITYPPAMKNAHAKASRTSSKH
KEDVYENLHTKNKREEKVKKQRSADKEKSKGSLKR
KGSSAGDMRAANLWPSPLMIKRSKKNSLALSLTAD
QMVSALLDAEPPILYSEYDPTRPFSEASMMGLLTN
LADRELVHMINWAKRVPGFVDLTLHDQVHLLECAW
LEILMIGLVWRSMEHPLKLLFAPNLLLDRNQGKCV
EGMVEIFDMLLATSSRFRMMNLQGEEFVCLKSIIL
LNSGVYTFLSSTLKSLEEKDHIHRVLDKITDTLIH
LMAKAGLTLQQQHQRLAQLLLILSHIRHMSNKGME
HLYSMKCKNVVPLYDLLLEAADAHRLHAPTSRGGA
SVEETDQSHLATAGSTSSHSLQKYYITGEAEGFPA
TV
Exemplary nucleic acid sequence encoding
SHP1(210-595)-ER(T12)
SEQ ID NO: 36
CGGCAGCCGTACTATGCCACGAGGGTGAATGCGGC
TGACATTGAGAACCGAGTGTTGGAACTGAACAAGA
AGCAGGAGTCCGAGGATACAGCCAAGGCTGGCTTC
TGGGAGGAGTTTGAGAGTTTGCAGAAGCAGGAGGT
GAAGAACTTGCACCAGCGTCTGGAAGGGCAGCGGC
CAGAGAACAAGGGCAAGAACCGCTACAAGAACATT
CTCCCCTTTGACCACAGCCGAGTGATCCTGCAGGG
ACGGGACAGTAACATCCCCGGGTCCGACTACATCA
ATGCCAACTACATCAAGAACCAGCTGCTAGGCCCT
GATGAGAACGCTAAGACCTACATCGCCAGCCAGGG
CTGTCTGGAGGCCACGGTCAATGACTTCTGGCAGA
TGGCGTGGCAGGAGAACAGCCGTGTCATCGTCATG
ACCACCCGAGAGGTGGAGAAAGGCCGGAACAAATG
CGTCCCATACTGGCCCGAGGTGGGCATGCAGCGTG
CTTATGGGCCCTACTCTGTGACCAACTGCGGGGAG
CATGACACAACCGAATACAAACTCCGTACCTTACA
GGTCTCCCCGCTGGACAATGGAGACCTGATTCGGG
AGATCTGGCATTACCAGTACCTGAGCTGGCCCGAC
CATGGGGTCCCCAGTGAGCCTGGGGGTGTCCTCAG
CTTCCTGGACCAGATCAACCAGCGGCAGGAAAGTC
TGCCTCACGCAGGGCCCATCATCGTGCACTGCAGC
GCCGGCATCGGCCGCACAGGCACCATCATTGTCAT
CGACATGCTCATGGAGAACATCTCCACCAAGGGCC
TGGACTGTGACATTGACATCCAGAAGACCATCCAG
ATGGTGCGGGCGCAGCGCTCGGGCATGGTGCAGAC
GGAGGCGCAGTACAAGTTCATCTACGTGGCCATCG
CCCAGTTCATTGAAACCACTAAGAAGAAGCTGGAG
GTCCTGCAGTCGCAGAAGGGCCAGGAGTCGGAGTA
CGGGAACATCACCTATCCCCCAGCCATGAAGAATG
CCCATGCCAAGGCCTCCCGCACCTCGTCCAAACAC
AAGGAGGATGTGTATGAGAACCTGCACACTAAGAA
CAAGAGGGAGGAGAAAGTGAAGAAGCAGCGGTCAG
CAGACAAGGAGAAGAGCAAGGGTTCCCTCAAGAGG
AAGGGATCCTCTGCTGGAGACATGAGAGCTGCCAA
CCTTTGGCCAAGCCCGCTCATGATCAAACGCTCTA
AGAAGAACAGCCTGGCCTTGTCCCTGACGGCCGAC
CAGATGGTCAGTGCCTTGTTGGATGCTGAGCCCCC
CATACTCTATTCCGAGTATGATCCTACCAGACCCT
TCAGTGAAGCTTCGATGATGGGCTTACTGACCAAC
CTGGCAGACAGGGAGCTGGTTCACATGATCAACTG
GGCGAAGAGGGTGCCAGGCTTTGTGGATTTGACCC
TCCATGATCAGGTCCACCTTCTAGAATGTGCCTGG
CTAGAGATCCTGATGATTGGTCTCGTCTGGCGCTC
CATGGAGCACCCACTGAAGCTACTGTTTGCTCCTA
ACTTGCTCTTGGACAGGAACCAGGGAAAATGTGTA
GAGGGCATGGTGGAGATCTTCGACATGCTGCTGGC
TACATCATCTCGGTTCCGCATGATGAATCTGCAGG
GAGAGGAGTTTGTGTGCCTCAAATCTATTATTTTG
CTTAATTCTGGAGTGTACACATTTCTGTCCAGCAC
CCTGAAGTCTCTGGAAGAGAAGGACCATATCCACC
GAGTCCTGGACAAGATCACAGACACTTTGATCCAC
CTGATGGCCAAGGCAGGCCTGACCCTGCAGCAGCA
GCACCAGCGGCTGGCCCAGCTCCTCCTCATCCTCT
CCCACATCAGGCACATGAGTAACAAAGGCATGGAG
CATCTGTACAGCATGAAGTGCAAGAACGTGGTGCC
CCTCTATGACCTGCTGCTGGAGGCGGCGGACGCCC
ACCGCCTACATGCGCCCACTAGCCGTGGAGGGGCA
TCCGTGGAGGAGACGGACCAAAGCCACTTGGCCAC
TGCGGGCTCTACTTCATCGCATTCCTTGCAAAAGT
ATTACATCACGGGGGAGGCAGAGGGTTTCCCTGCC
ACGGTCTGA

### EQUIVALENTS

It is to be understood that while the disclosure has been described in conjunction with the detailed description thereof, the foregoing description is intended to illustrate and not limit the scope of the invention, which is defined by the scope of the appended claims. Other aspects, advantages, and modifications are within the scope of the following claims.

1. A trigger-responsive immune-inactivating signaling polypeptide comprising:
a modulating domain characterized by an ability to adopt a first state and a second state, and to transition between the first state and the second state when exposed to a trigger; and
an immune-inactivating moiety;
wherein, when the modulating domain is in its first state, the immune-inactivating moiety is inhibited, and when the modulating domain is in its second state, the inhibition is relieved.

2. The trigger-responsive immune-inactivating signaling polypeptide of claim 1, wherein the modulating domain comprises a nuclear receptor or a fragment thereof.

3. (canceled)

4. The trigger-responsive immune-inactivating signaling polypeptide of claim 1, wherein the modulating domain comprises a hormone receptor, retinoic acid receptor, vitamin D receptor, peroxisome proliferator-activated receptor, farnesoid X receptor, or liver X receptor.

5.-7. (canceled)

8. The trigger-responsive immune-inactivating signaling polypeptide of claim 4, wherein the hormone receptor is an estrogen receptor.

9. The trigger-responsive immune-inactivating signaling polypeptide of claim 8, wherein the estrogen receptor is estrogen receptor-α.

10. The trigger-responsive immune-inactivating signaling polypeptide of claim 8, wherein the modulating domain includes an amino acid sequence that has at least 90% sequence identity with an amino acid sequence that starts at residue 251, 282, or 305 of SEQ ID NO: 12 and ends at residue 545 or 595 of SEQ ID NO: 12.

11. The trigger-responsive immune-inactivating signaling polypeptide of claim 8, wherein the modulating domain includes an amino acid sequence that has 90% sequence identity with SEQ ID NO: 4 or wherein the modulating domain includes an amino acid sequence that has 90% sequence identity with SEQ ID NO: 13.

12.-13. (canceled)

14. The trigger-responsive immune-inactivating signaling polypeptide of claim 8, wherein the modulating domain includes one or more mutations that (i) confer on the modulating domain a reduced affinity to at least one naturally occurring estrogen; (ii) confer on the modulating domain a preferential binding to at least one synthetic estrogen receptor ligand; or (iii) confer increased affinity for at least one chaperone protein.

15. The trigger-responsive immune-inactivating signaling polypeptide of claim 14, wherein the at least one naturally occurring estrogen includes an estradiol.

16.-17. (canceled)

18. The trigger-responsive immune-inactivating signaling polypeptide of claim 14, wherein the at least one synthetic estrogen receptor ligand includes tamoxifen, endoxifen, 4-hydroxytamoxifen, fulvestrant, OP-1250, OP-1074, or OP-1124.

19. (canceled)

20. The trigger-responsive immune-inactivating signaling polypeptide of claim 14, wherein the at least one chaperone protein includes HSP90.

21. The trigger-responsive immune-inactivating signaling polypeptide of claim 14, wherein the modulating domain includes at least one mutation selected from the group consisting of G400V, G400M, G400A, G400L, G521R, G521T, L539A, L540A, M543A and L544A, wherein the residue numbering is based on SEQ ID NO: 12.

22. The trigger-responsive immune-inactivating signaling polypeptide of claim 14, wherein the modulating domain includes at least one mutation selected from the group consisting of G400V, G400M, G400A, G400L, G521R, and G521T, and at least one mutation selected from the group consisting of L539A, L540A, M543A, and L544A, wherein the residue numbering is based on SEQ ID NO: 12.

23. The trigger-responsive immune-inactivating signaling polypeptide of claim 14, wherein the modulating domain includes at least one mutation selected from the group consisting of G400V, G400M, G400A, G400L, G521R, and G521T, and at least one mutation selected from (i) the group consisting of L539A and L540A, or (ii) the group consisting of M543A and L544A, wherein the residue numbering is based on SEQ ID NO: 12.

24. The trigger-responsive immune-inactivating signaling polypeptide of claim 1, wherein the trigger-responsive immune-inactivating signaling polypeptide is a trigger-responsive dominant negative signaling polypeptide and the immune-inactivating moiety is a dominant negative signaling moiety.

25.-33. (canceled)

34. The trigger-responsive immune-inactivating signaling polypeptide of claim 24, wherein the dominant negative signaling moiety includes a dominant negative kinase moiety that is a dominant negative variant of a Zap70 kinase.

35. The trigger-responsive immune-inactivating signaling polypeptide of claim 34, wherein the dominant negative Zap70 kinase moiety has a sequence that has at least 90% sequence identity to SEQ ID NO: 2.

36. (canceled)

37. The trigger-responsive immune-inactivating signaling polypeptide of claim 24, wherein the dominant negative signaling moiety includes a dominant negative kinase moiety that is a dominant negative variant of a LCK kinase.

38. The trigger-responsive immune-inactivating signaling polypeptide of claim 37, wherein the dominant negative LCK kinase moiety has a sequence that has at least 90% sequence identity to SEQ ID NO: 17.

39. The trigger-responsive immune-inactivating signaling polypeptide of claim 1, wherein the trigger-responsive immune-inactivating signaling polypeptide is a trigger-responsive constitutively active signaling polypeptide and the immune-inactivating moiety is a constitutively active signaling moiety.

40.-48. (canceled)

49. The trigger-responsive immune-inactivating signaling polypeptide of claim 39, wherein the constitutively active signaling moiety includes a constitutively active phosphatase moiety that is a constitutively active variant of a SHP1 phosphatase.

50. The trigger-responsive immune-inactivating signaling polypeptide of claim 49, wherein the constitutively active SHP1 phosphatase moiety has a sequence that has at least 90% sequence identity to SEQ ID NO: 23.

51.-53. (canceled)

54. A nucleic acid encoding the trigger-responsive immune-inactivating signaling polypeptide of claim 1.

55. (canceled)

56. A cell including the nucleic acid of claim 54.

57.-65. (canceled)

66. A pharmaceutical composition that delivers the trigger-responsive dominant negative signaling polypeptide of claim 1.

67. A method of regulating activity of T cells in vivo comprising the step of administering a composition that delivers the trigger-responsive dominant negative signaling polypeptide of claim 1 to a subject.

68.-70. (canceled)

71. A method of treating cancer, the method comprising the step of administering a composition that delivers the trigger-responsive dominant negative signaling polypeptide of claim 1 to a subject.

72.-76. (canceled)

77. A method of manufacturing a trigger-responsive dominant negative signaling polypeptide of claim 1, comprising expressing the trigger-responsive dominant negative signaling polypeptide in a host cell.

78. (canceled)

79. A method of manufacturing a genetically modified T cell, comprising introducing the nucleic acid of claim 54 into a T cell.

80. (canceled)
